(19) United States
(12) Patent Application Publication
(10) Pub. No.: US 2012/0270875 A1
(43) Pub. Date: Oct. 25, 2012

(54) PYRIMIDINE AMIDE COMPOUNDS

(76) Inventors: Paul Gillespie, Westfield, NJ (US); Christophe Michoud, New York, NY (US); Kenneth Carey Rupert, Bedminster, NJ (US); Kshitij Chhabilbhai Thakkar, Clifton, NJ (US); Lin Yi, Basking Ridge, NJ (US)

(21) Appl. No.: 13/399,117

(22) Filed: Feb. 17, 2012

Related U.S. Application Data
(60) Provisional application No. 61/453,229, filed on Mar. 16, 2011.

(51) Int. Cl. A61K 31/5377; A61K 31/506; C07D 413/14; C07D 409/14; C07D 239/42; C07D 409/12; C07D 401/12; C07D 417/14; A61P 11/06; A61P 11/00; A61K 31/505; A61K 31/497; C07D 403/12

(52) U.S. Cl. 514/235.8; 514/255.05; 514/275; 544/122; 544/331; 544/332

(57) ABSTRACT

Provided herein are compounds of the formula (I):

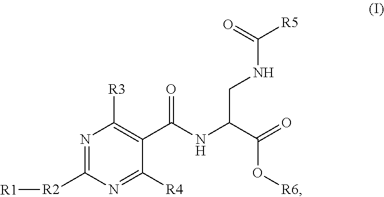

as well as pharmaceutically acceptable salts thereof, wherein the substituents are as those disclosed in the specification. These compounds, and the pharmaceutical compositions containing them, are useful for the treatment of inflammatory diseases and disorders such as, for example, asthma and COPD.

### PRIORITY TO RELATED APPLICATION(S)

This application claims the benefit of U.S. Provisional Application No. 61/453,229, filed Mar. 16, 2011, which is hereby incorporated by reference in its entirety.

### FIELD OF THE INVENTION

The present invention relates to organic compounds useful for therapy and/or prophylaxis in a mammal, and in particular to LFA-1 antagonists and dual LFA-1/MAC-1 antagonists useful for treating inflammatory diseases and disorders.

### SUMMARY OF THE INVENTION

In an embodiment of the present invention, provided are compounds of general formula (I):

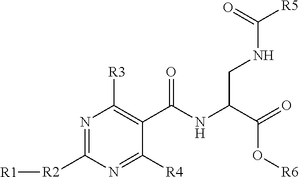

wherein:

### R1 is

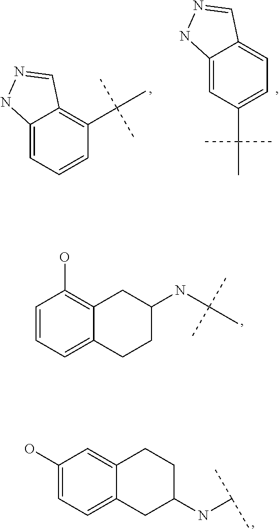 [0005] phenyl, mono- or bi-substituted independently with hydroxy, halogen, lower alkyl, alkoxy —OC(O)CH₃ or —OC(O)CH(CH₃)₂, or
heteroaryl, mono- or bi-substituted with hydroxy;

### R2 is:

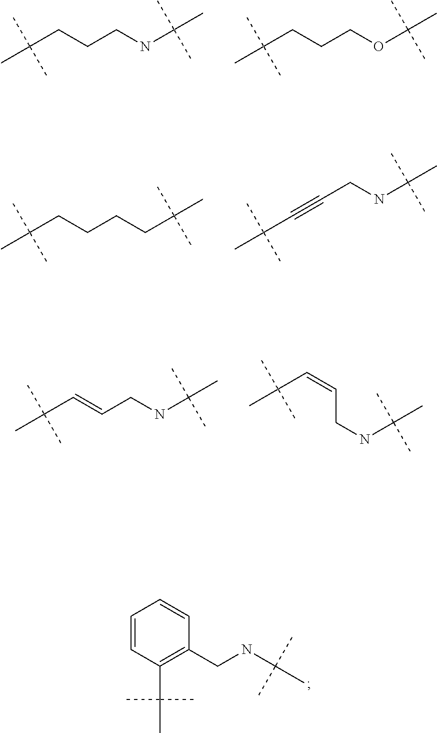

or absent if R1 is

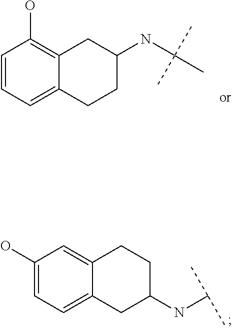

R3 and R4, independently or each other, are H, methyl, trifluoromethyl or ethyl;
R5 is lower alkyl, cycloalkyl, lower alkyl-cycloalkyl, heterocycloalkyl, isoquinoline, quinoline, adamantane, NR7R8, OR9, unsubstituted heteroaryl, heteroaryl substituted with phenyl, unsubstituted phenyl or phenyl substituted with hydroxy or methyl;
R6 is hydrogen, lower alkyl, alkoxy, cycloalkyl, aryl, heteroaryl,

### —(CH₂)₂—O—(CH₂)₂OCH₂CH₃—,

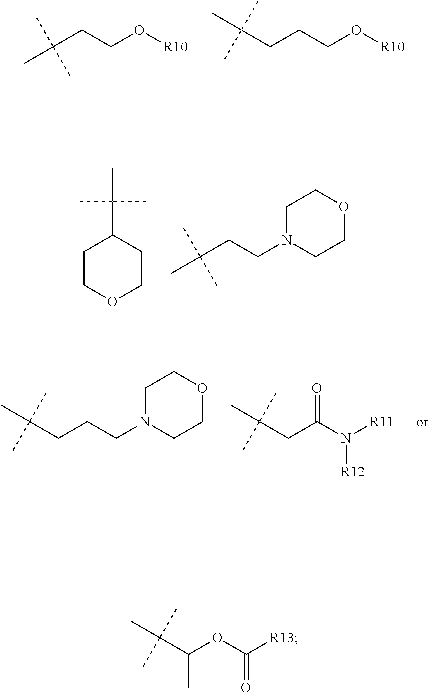

R7 and R8, independently of each other, are hydrogen, lower alkyl, cycloalkyl, aryl or heteroaryl;
R9 is lower alkyl, cycloalkyl, phenyl or heteroaryl; and
R10, R11, R12 and R13, independently of each other, are hydrogen or lower alkyl,
or a pharmaceutically acceptable salt thereof.

In a further embodiment of the invention, provided is a pharmaceutical composition comprising a therapeutically effective amount of a compound according to formula (I) and a therapeutically inert carrier.

In a still further embodiment of the invention, provided is a method for the treatment or prophylaxis of asthma or COPD, which method comprises the step of administering an effective amount of a compound according to formula (I) to a patient in need thereof.

All documents cited to or relied upon below are expressly incorporated herein by reference.

### DETAILED DESCRIPTION OF THE INVENTION

Asthmatic and COPD symptoms such as restricted breathing and airway inflammation can be linked to the migration and activation of leukocytes into the lung. LFA-1 on neutrophils and MAC-1 on macrophages are major receptors that upon activation promote leukocyte infiltration and activation into the lung. Therefore, LFA-1 antagonists and dual LFA-1/MAC-1 antagonists are desirable therapeutics for the treatment of inflammatory diseases and disorders.

Provided herein are LFA-1 antagonist and dual LFA-1/MAC-1 antagonist compounds. The compounds of the invention are useful for the treatment of inflammatory diseases and disorders such as, for example, asthma and COPD.

It is to be understood that the terminology employed herein is for the purpose of describing particular embodiments, and is not intended to be limiting. Further, although any methods, devices and materials similar or equivalent to those described herein can be used in the practice or testing of the invention, the preferred methods, devices and materials are now described.

As used herein, the term “alkyl”, alone or in combination with other groups, refers to a branched or straight-chain monovalent saturated aliphatic hydrocarbon radical of one to twenty carbon atoms, preferably one to sixteen carbon atoms, more preferably one to ten carbon atoms.

The term “cycloalkyl” refers to a monovalent mono- or polycarbocyclic radical of three to ten, preferably three to six carbon atoms. This term is further exemplified by radicals such as cyclopropyl, cyclobutyl, cyclopentyl, cyclohexyl, cycloheptyl, norbornyl, adamantyl, indanyl and the like. In a preferred embodiment, the “cycloalkyl” moieties can optionally be substituted with one, two, three or four substituents, with the understanding that said substituents are not, in turn, substituted further. Each substituent can independently be, alkyl, alkoxy, halogen, amino, hydroxyl or oxygen (O═) unless otherwise specifically indicated. Examples of cycloalkyl moieties include, but are not limited to, optionally substituted cyclopropyl, optionally substituted cyclobutyl, optionally substituted cyclopentyl, optionally substituted cyclopentenyl, optionally substituted cyclohexyl, optionally substituted cyclohexylene, optionally substituted cycloheptyl, and the like or those which are specifically exemplified herein.

The term “heterocycloalkyl” denotes a mono- or polycyclic alkyl ring, wherein one, two or three of the carbon ring atoms is replaced by a heteroatom such as N, O or S. Examples of heterocycloalkyl groups include, but are not limited to, morpholinyl, thiomorpholinyl, piperazinyl, piperidinyl, pyrrolidinyl, tetrahydropyranyl, tetrahydrofuranyl, 1,3-dioxanyl and the like. The heterocycloalkyl groups may be unsubstituted or substituted and attachment may be through their carbon frame or through their heteroatom(s) where appropriate, with the understanding that said substituents are not, in turn, substituted further.

The term “lower alkyl”, alone or in combination with other groups, refers to a branched or straight-chain alkyl radical of one to nine carbon atoms, preferably one to six carbon atoms, more preferably one to four carbon atoms. This term is further exemplified by radicals such as methyl, ethyl, n-propyl, isopropyl, n-butyl, s-butyl, isobutyl, t-butyl, n-pentyl, 3-methylbutyl, n-hexyl, 2-ethylbutyl and the like.

The term “aryl” refers to an aromatic mono- or polycarbocyclic radical of 6 to 12 carbon atoms having at least one aromatic ring. Examples of such groups include, but are not limited to, phenyl, naphthyl, 1,2,3,4-tetrahydronaphthalene, 1,2-dihydronaphthalene, indanyl, 1H-indenyl and the like.

The term “heteroaryl,” refers to an aromatic mono- or polycyclic radical of 5 to 12 atoms having at least one aromatic ring containing one, two, or three ring heteroatoms selected from N, O, and S, with the remaining ring atoms being C. One or two ring carbon atoms of the heteroaryl group may be replaced with a carbonyl group.

The alkyl, lower alkyl, aryl and heteroaryl groups described above may be substituted independently with one, two, or three substituents, with the understanding that said substituents are not, in turn, substituted further. These substituents may optionally form a ring with the heteroaryl group to which they are connected. Substituents may include, for example: carbon-containing groups such as alkyl, aryl, arylalkyl (e.g. substituted and unsubstituted phenyl, substituted and unsubstituted benzyl); halogen atoms and halogen-containing groups such as haloalkyl (e.g. trifluoromethyl); oxygen-containing groups such as alcohols (e.g. hydroxyl, hydroxyalkyl, aryl(hydroxyl)alkyl), ethers (e.g. alkoxy, aryloxy, alkoxyalkyl, aryloxyalkyl), aldehydes (e.g. carboxaldehyde), ketones (e.g. alkylcarbonyl, alkylcarbonylalkyl, arylcarbonyl, arylalkylcarbonyl, arycarbonylalkyl), acids (e.g. carboxy, carboxyalkyl), acid derivatives such as esters (e.g. alkoxycarbonyl, alkoxycarbonylalkyl, alkylcarbonyloxy, alkylcarbonyloxyalkyl), amides (e.g. aminocarbonyl, mono- or di-alkylaminocarbonyl, aminocarbonylalkyl, mono- or di-alkylaminocarbonylalkyl, arylaminocarbonyl), carbamates (e.g. alkoxycarbonylamino, aryloxycarbonylamino, aminocarbonyloxy, mono- or di-alkylaminocarbonyloxy, arylminocarbonloxy) and ureas (e.g. mono- or di-alkylaminocarbonylamino or arylaminocarbonylamino); nitrogen-containing groups such as amines (e.g. amino, mono- or di-alkylamino, aminoalkyl, mono- or di-alkylaminoalkyl), azides, nitriles (e.g. cyano, cyanoalkyl), nitro; sulfur-containing groups such as thiols, thioethers, sulfoxides and sulfones (e.g. alkylthio, alkylsulfinyl, alkylsulfonyl, alkylthioalkyl, alkylsulfinylalkyl, alkylsulfonylalkyl, arylthio, arysulfinyl, arysulfonyl, arythioalkyl, arylsulfinylalkyl, arylsulfonylalkyl); and heterocyclic groups containing one or more heteroatoms, (e.g. thienyl, furanyl, pyrrolyl, imidazolyl, pyrazolyl, thiazolyl, isothiazolyl, oxazolyl, oxadiazolyl, thiadiazolyl, aziridinyl, azetidinyl, pyrrolidinyl, pyrrolinyl, imidazolidinyl, imidazolinyl, pyrazolidinyl, tetrahydrofuranyl, pyranyl, pyronyl, pyridyl, pyrazinyl, pyridazinyl, piperidyl, hexahydroazepinyl, piperazinyl, morpholinyl, thianaphthyl, benzofuranyl, isobenzofuranyl, indolyl, oxyindolyl, isoindolyl, indazolyl, indolinyl, 7-azaindolyl, benzopyranyl, coumarinyl, isocoumarinyl, quinolinyl, isoquinolinyl, naphthridinyl, cinnolinyl, quinazolinyl, pyridopyridyl, benzoxazinyl, quinoxalinyl, chromenyl, chromanyl, isochromanyl, phthalazinyl, benzothiazoyl and carbolinyl).

As used herein, the term “alkoxy” means alkyl-O—; and “alkoyl” means alkyl-CO—. Alkoxy substituent groups or alkoxy-containing substituent groups may be substituted by, for example, one or more alkyl groups, with the understanding that said substituents are not, in turn, substituted further.

As used herein, the term “halogen” means a fluorine, chlorine, bromine or iodine radical, preferably a fluorine, chlorine or bromine radical, and more preferably a fluorine or chlorine radical.

Compounds of formula I can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates or mixtures of diastereoisomeric racemates. The optically active forms can be obtained for example by resolution of the racemates, by asymmetric synthesis or asymmetric chromatography (chromatography with a chiral adsorbents or eluant). The invention embraces all of these forms.

As used herein, the term “pharmaceutically acceptable salt” means any pharmaceutically acceptable salt of the compound of formula (I). Salts may be prepared from pharmaceutically acceptable non-toxic acids and bases including inorganic and organic acids and bases. Such acids include, for example, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfuric, tartaric, oxalic, p-toluenesulfonic and the like. Particularly preferred are fumaric, hydrochloric, hydrobromic, phosphoric, succinic, sulfuric and methanesulfonic acids. Acceptable base salts include alkali metal (e.g. sodium, potassium), alkaline earth metal (e.g. calcium, magnesium) and aluminum salts.

In the practice of the method of the present invention, an effective amount of any one of the compounds of this invention or a combination of any of the compounds of this invention or a pharmaceutically acceptable salt thereof, is administered via any of the usual and acceptable methods known in the art, either singly or in combination. The compounds or compositions can thus be administered orally (e.g., buccal cavity), sublingually, parenterally (e.g., intramuscularly, intravenously, or subcutaneously), rectally (e.g., by suppositories or washings), transdermally (e.g., skin electroporation) or by inhalation (e.g., by aerosol), and in the form or solid, liquid or gaseous dosages, including tablets and suspensions. The administration can be conducted in a single unit dosage form with continuous therapy or in a single dose therapy ad libitum. The therapeutic composition can also be in the form of an oil emulsion or dispersion in conjunction with a lipophilic salt such as pamoic acid, or in the form of a biodegradable sustained-release composition for subcutaneous or intramuscular administration.

Useful pharmaceutical carriers for the preparation of the compositions hereof, can be solids, liquids or gases. Thus, the compositions can take the form of tablets, pills, capsules, suppositories, powders, enterically coated or other protected formulations (e.g. binding on ion-exchange resins or packaging in lipid-protein vesicles), sustained release formulations, solutions, suspensions, elixirs, aerosols, and the like. The carrier can be selected from the various oils including those of petroleum, animal, vegetable or synthetic origin, e.g., peanut oil, soybean oil, mineral oil, sesame oil, and the like. Water, saline, aqueous dextrose, and glycols are preferred liquid carriers, particularly (when isotonic with the blood) for injectable solutions. For example, formulations for intravenous administration comprise sterile aqueous solutions of the active ingredient(s) which are prepared by dissolving solid active ingredient(s) in water to produce an aqueous solution, and rendering the solution sterile. Suitable pharmaceutical excipients include starch, cellulose, talc, glucose, lactose, talc, gelatin, malt, rice, flour, chalk, silica, magnesium stearate, sodium stearate, glycerol monostearate, sodium chloride, dried skim milk, glycerol, propylene glycol, water, ethanol, and the like. The compositions may be subjected to conventional pharmaceutical additives such as preservatives, stabilizing agents, wetting or emulsifying agents, salts for adjusting osmotic pressure, buffers and the like. Suitable pharmaceutical carriers and their formulation are described in Remington's Pharmaceutical Sciences by E. W. Martin. Such compositions will, in any event, contain an effective amount of the active compound together with a suitable carrier so as to prepare the proper dosage form for proper administration to the recipient.

The dose of a compound of the present invention depends on a number of factors, such as, for example, the manner of administration, the age and the body weight of the subject, and the condition of the subject to be treated, and ultimately will be decided by the attending physician or veterinarian. Such an amount of the active compound as determined by the attending physician or veterinarian is referred to herein, and in the claims, as a “therapeutically effective amount”. For example, the dose of a compound of the present invention is typically in the range of about 1 to about 1000 mg per day. Preferably, the therapeutically effective amount is in an amount of from about 1 mg to about 500 mg per day.

It will be appreciated, that the compounds of general formula I in this invention may be derivatized at functional groups to provide derivatives which are capable of conversion back to the parent compound in vivo. Physiologically acceptable and metabolically labile derivatives, which are capable of producing the parent compounds of general formula I in vivo are also within the scope of this invention.

The present invention provides novel compounds of general formula (I):

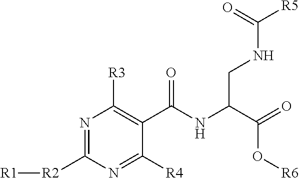

wherein:

### R1 is

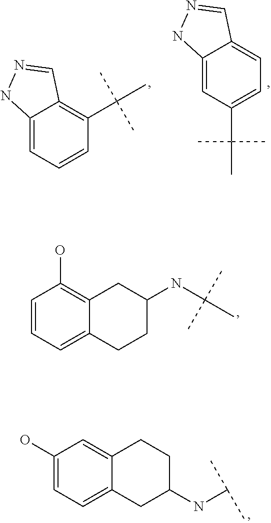 [0032] phenyl, mono- or bi-substituted independently with hydroxy, halogen, alkyl, alkoxy —OC(O)CH₃ or —OC(O)CH(CH₃)₂, or
heteroaryl, mono- or bi-substituted with hydroxy;

### R2 is:

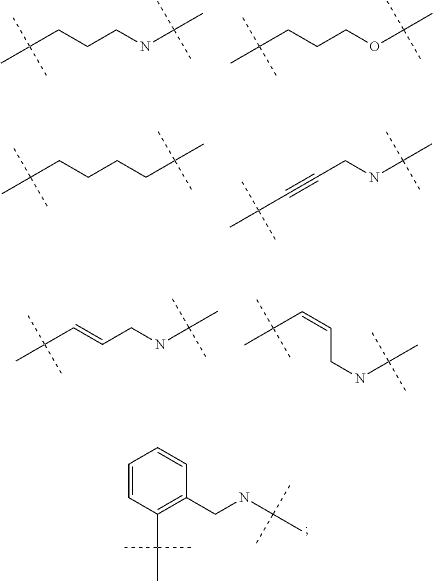

or absent if R1 is

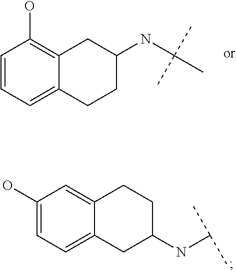

R3 and R4, independently or each other, are H, methyl, trifluoromethyl or ethyl;
R5 is lower alkyl, cycloalkyl, lower alkyl-cycloalkyl, heterocycloalkyl, isoquinoline, quinoline, adamantane, NR7R8, OR9, unsubstituted heteroaryl, heteroaryl substituted with phenyl, unsubstituted phenyl or phenyl substituted with hydroxy or methyl;
R6 is hydrogen, lower alkyl, alkoxy, cycloalkyl, aryl, heteroaryl,

### —(CH₂)₂—O—(CH₂)₂OCH₂CH₃—,

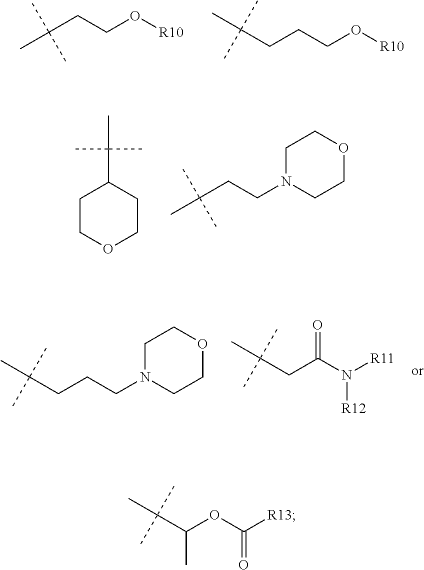

R7 and R8, independently of each other, are hydrogen, lower alkyl, cycloalkyl, aryl or heteroaryl;
R9 is lower alkyl, cycloalkyl, phenyl or heteroaryl; and
R10, R11, R12 and R13, independently of each other, are hydrogen or lower alkyl,
or a pharmaceutically acceptable salt thereof.

In another embodiment of the invention, provided is a compound according to formula (I), wherein R1 is

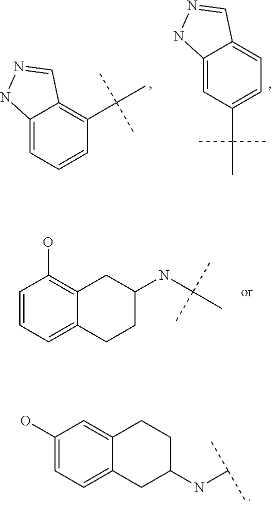

In another embodiment of the invention, provided is a compound according to formula (I), wherein R1 is phenyl, mono- or bi-substituted independently with hydroxy, halogen, lower alkyl, alkoxy, —OC(O)CH₃ or —OC(O)CH(CH₃)₂.

In another embodiment of the invention, provided is a compound according to formula (I), wherein R1 is heteroaryl, mono- or bi-substituted with hydroxy.

In another embodiment of the invention, provided is a compound according to formula (I), wherein R2 is absent.

In another embodiment of the invention, provided is a compound according to formula (I), wherein R3 is methyl.

In another embodiment of the invention, provided is a compound according to formula (I), wherein R4 is methyl.

In another embodiment of the invention, provided is a compound according to formula (I), wherein R5 is lower alkyl, —C(O)CH₃, cycloalkyl, lower alkyl-cycloalkyl, heterocycloalkyl, isoquinoline, quinoline, adamantane or NR7R8, OR9.

In another embodiment of the invention, provided is a compound according to formula (I), wherein R5 is unsubstituted heteroaryl, heteroaryl substituted with phenyl, unsubstituted phenyl or phenyl substituted with hydroxy or methyl.

In another embodiment of the invention, provided is a compound according to formula (I), wherein R5 is adamantane, chlorothiophene, pyrazine, thiazole, thiophene, —C(O)CH3, thiazole-phenyl, pyridine, dimethyl-thiazole, isoquinoline, pyridine-thiazole, methyl-thiophene, methyl-pyrazine or ethyl-thiophene.

In another embodiment of the invention, provided is a compound according to formula (I), wherein R6 is hydrogen or lower alkyl.

In another embodiment of the invention, provided is a compound according to formula (I), wherein R6 is methyl.

In another embodiment of the invention, provided is a compound according to formula (I), wherein R6 is:

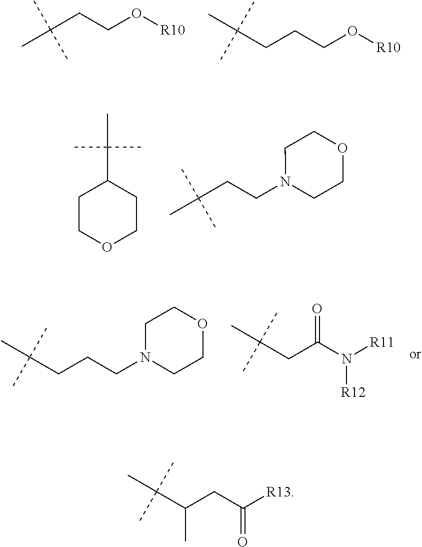

In another embodiment of the invention, provided is a compound according to formula (I), wherein R7 is hydrogen or lower alkyl.

In another embodiment of the invention, provided is a compound according to formula (I), wherein R8 is hydrogen or lower alkyl.

In another embodiment of the invention, provided is a compound according to formula (I), wherein R9 is lower alkyl or phenyl.

In another embodiment of the invention, provided is a compound according to formula (I), wherein R10 is hydrogen or methyl.

In another embodiment of the invention, provided is a compound according to formula (I), wherein R11 is hydrogen or methyl.

In another embodiment of the invention, provided is a compound according to formula (I), wherein R12 is hydrogen or methyl.

In another embodiment of the invention, provided is a compound according to formula (I), wherein R13 is hydrogen or methyl.
294) Particular compounds of formula I include the following:
(S)-2-({2-[4-(3-hydroxy-phenyl)-butyl]-4,6-dimethyl-pyrimidine-5-carbonyl}-amino)-3-[thiophene-2-carbonyl)-amino]-propionic acid;
(S)-2-({2-[3-(3-hydroxy-phenyl)-propoxy]-4,6-dimethyl-pyrimidine-5-carbonyl}-amino)-3-[(thiophene-2-carbonyl)-amino]-propionic acid methyl ester;
(S)-2-({2-[3-(3-hydroxy-phenyl)-propoxy]-4,6-dimethyl-pyrimidine-5-carbonyl}-amino)-3-[(thiophene-2-carbonyl)-amino]-propionic acid;
(S)-3-acetylamino-2-({2-[3-(1H-indazol-4-yl)-propylamino]-4,6-dimethyl-pyrimidine-5-carbonyl}-amino)-propionic acid;
(S)-2-({2-[3-(1H-indazol-4-yl)-propylamino]-4,6-dimethyl-pyrimidine-5-carbonyl}-amino)₃-[(thiophene-2-carbonyl)-amino]-propionic acid methyl ester;
(S)-2-({2-[3-(1H-indazol-4-yl)-propylamino]-4,6-dimethyl-pyrimidine-5-carbonyl}-amino)₃-[(thiophene-2-carbonyl)-amino]-propionic acid;
(S)-2-({2-[3-(3-hydroxy-phenyl)-propylamino]-4,6-dimethyl-pyrimidine-5-carbonyl}-amino)-3-[(thiophene-2-carbonyl)-amino]-propionic acid methyl ester;
(S)-2-({2-[3-(3-hydroxy-phenyl)-propylamino]-4,6-dimethyl-pyrimidine-5-carbonyl}-amino)-3-[(thiophene-2-carbonyl)-amino]-propionic acid;
(S)-2-({2-[(3′-hydroxy-biphenyl-2-ylmethyl)-amino]-4,6-dimethyl-pyrimidine-5-carbonyl}-amino)-3-[(thiophene-2-carbonyl)-amino]-propionic acid methyl ester;
(S)-2-({2-[(4′-hydroxy-biphenyl-2-ylmethyl)-amino]-4,6-dimethyl-pyrimidine-5-carbonyl}-amino)-3-[(thiophene-2-carbonyl)-amino]-propionic acid methyl ester;
(S)-2-({2-[(2′-hydroxy-biphenyl-2-ylmethyl)-amino]-4,6-dimethyl-pyrimidine-5-carbonyl}-amino)-3-[(thiophene-2-carbonyl)-amino]-propionic acid methyl ester;
(S)-2-({2-[(3′-hydroxy-biphenyl-2-ylmethyl)-amino]-4,6-dimethyl-pyrimidine-5-carbonyl}-amino)-3-[(thiophene-2-carbonyl)-amino]-propionic acid;
(S)-2-({2-[(3′-hydroxy-biphenyl-2-ylmethyl)-amino]-4,6-dimethyl-pyrimidine-5-carbonyl}-amino)-3-[(thiophene-2-carbonyl)-amino]-propionic acid;
(S)-2-({2-[(2′-hydroxy-biphenyl-2-ylmethyl)-amino]-4,6-dimethyl-pyrimidine-5-carbonyl}-amino)-3-[(thiophene-2-carbonyl)-amino]-propionic acid;
(S) 2-(2-[6-hydroxy-1,2,3,4-tetrahydro-naphthalen-2-ylamino]-4,6-dimethyl-pyrimidine-5-carbonyl-amino)-3-[(thiophene-2-carbonyl)-amino]-propionic acid methyl ester;
(S) 2-(2-[6-hydroxy-1,2,3,4-tetrahydro-naphthalen-2-ylamino]-4,6-dimethyl-pyrimidine-5-carbonyl-amino)-3-[(thiophene-2-carbonyl)-amino]-propionic acid;
(S) 2-(2-[8-hydroxy-1,2,3,4-tetrahydro-naphthalen-2-ylamino]-4,6-dimethyl-pyrimidine-5-carbonyl-amino)-3-[(thiophene-2-carbonyl)-amino]-propionic acid;
(S)-2-({2-[3-(3-Hydroxy-phenyl)-prop-2-ynylamino]-4,6-dimethyl-pyrimidine-5-carbonyl}-amino)-3-[(thiophene-2-carbonyl)-amino]-propionic acid methyl ester;
(S)-2-({2-[(Z)-3-(hydroxy-phenyl)-allylamino]-4,6-dimethyl-pyrimidine-5-carbonyl}-amino)-3-[(thiophene-2-carbonyl)-amino]-propionic acid methyl ester;
(S)-2-({2-[3-(1H-indazol-4-yl)-prop-2-ynylamino]-4,6-dimethyl-pyrimidine-5-carbonyl}-amino)-3-[(thiophene-2-carbonyl)-amino]-propionic acid methyl ester;
(S)-2-({2-[3-(1H-indazol-6-yl)-prop-2-ynylamino]-4,6-dimethyl-pyrimidine-5-carbonyl}-amino)-3-[(thiophene-2-carbonyl)-amino]-propionic acid methyl ester;
(S)-2-({2-[3-(1H-indazol-6-yl)-propylamino]-4,6-dimethyl-pyrimidine-5-carbonyl}-amino)-3-[(thiophene-2-carbonyl)-amino]-propionic acid methyl ester;
(S)-2-({2-[3-(3-Fluoro-5-hydroxy-phenyl)-prop-2-ynylamino]-4,6-dimethyl-pyrimidine-5-carbonyl}-amino)-3-[(thiophene-2-carbonyl)-amino]propionic acid methyl ester;
(S)-2-({2-[3-(3-fluoro-5-hydroxy-phenyl)-propylamino]-4,6-dimethyl-pyrimidine-5-carbonyl}-amino)-3-[(thiophene-2-carbonyl)-amino]propionic acid methyl ester;
(S)-2-({2-[3-(4-Fluoro-3-hydroxy-phenyl)-prop-2-ynylamino]-4,6-dimethyl-pyrimidine-5-carbonyl}-amino)-3-[(thiophene-2-carbonyl)-amino]propionic acid methyl ester;
(S)-2-({2-[3-(4-fluoro-3-hydroxy-phenyl)-propylamino]-4,6-dimethyl-pyrimidine-5-carbonyl}-amino)-3-[(thiophene-2-carbonyl)-amino]propionic acid methyl ester;
(S)-2-({2-[3-(3-Hydroxy-2-methyl-phenyl)-prop-2-ynylamino]-4,6-dimethyl-pyrimidine-5-carbonyl}-amino)-3-[(thiophene-2-carbonyl)-amino]propionic acid methyl ester;
(S)-2-({2-[3-(3-Hydroxy-2-methyl-phenyl)-propylamino]-4,6-dimethyl-pyrimidine-5-carbonyl}-amino)-3-[(thiophene-2-carbonyl)-amino]propionic acid methyl ester;
(S)-2-({2-[3-(2-chloro-5-methoxy-phenyl)-prop-2-ynylamino]-4,6-dimethyl-pyrimidine-5-carbonyl}-amino)-3-[(thiophene-2-carbonyl)-amino]propionic acid methyl ester;
(S)-2-({2-[3-(2-chloro-5-methoxy-phenyl)-propylamino]-4,6-dimethyl-pyrimidine-5-carbonyl}-amino)-3-[(thiophene-2-carbonyl)-amino]propionic acid methyl ester;
(S)-2-({2-[3-(4-Hydroxy-phenyl)-prop-2-ynylamino]-4,6-dimethyl-pyrimidine-5-carbonyl}-amino)-3-[(thiophene-2-carbonyl)-amino]propionic acid methyl ester;
(S)-2-({2-[3-(4-hydroxy-phenyl)-propylamino]-4,6-dimethyl-pyrimidine-5-carbonyl}-amino)-3-[(thiophene-2-carbonyl)-amino]propionic acid methyl ester;
(S)-2-({2-[3-(3-hydroxy-phenyl)-propylamino]-4,6-dimethyl-pyrimidine-5-carbonyl}-amino)-3-[(2-phenyl-thiazole-4-carbonyl)-amino]-propionic acid methyl ester;
(S)-2-({2-[3-(3-hydroxy-phenyl)-propylamino]-4,6-dimethyl-pyrimidine-5-carbonyl}-amino)-3-[(2,4-dimethyl-thiazole-5-carbonyl)-amino]-propionic acid methyl Ester;
(S)-2-({2-[3-(3-hydroxy-phenyl)-propylamino]-4,6-dimethyl-pyrimidine-5-carbonyl}-amino)-3-[2-cyclohexyl-acetylamino]-propionic acid methyl Ester;
(S)-2-({2-[3-(3-hydroxy-phenyl)-propylamino]-4,6-dimethyl-pyrimidine-5-carbonyl}-amino)-3-[(isoquinoline-1-carbonyl)-amino]-propionic acid methyl Ester;
(S)-2-({2-[3-(3-hydroxy-phenyl)-propylamino]-4,6-dimethyl-pyrimidine-5-carbonyl}-amino)-3-[(thiazole-4-carbonyl)-amino]-propionic acid methyl Ester;
(S)-2-({2-[3-(3-hydroxy-phenyl)-propylamino]-4,6-dimethyl-pyrimidine-5-carbonyl}-amino)-3-[(2-pyridin-4-yl-thiazole-4-carbonyl)-amino]-propionic acid methyl Ester;
(S)-2-({2-[3-(3-hydroxy-phenyl)-propylamino]-4,6-dimethyl-pyrimidine-5-carbonyl}-amino)-3-[(2-pyridin-4-yl-thiazole-4-carbonyl)-amino]-propionic acid;
(S)-2-({2-[3-(3-hydroxy-phenyl)-propylamino]-4,6-dimethyl-pyrimidine-5-carbonyl}-amino)-3-[(pyridine-2-carbonyl)-amino]-propionic acid;
(S)-2-({2-[3-(3-hydroxy-phenyl)-propylamino]-4,6-dimethyl-pyrimidine-5-carbonyl}-amino)-3-[(2,4-dimethyl-thiazole-5-carbonyl)-amino]-propionic acid;
(S)-2-({2-[3-(3-hydroxy-phenyl)-propylamino]-4,6-dimethyl-pyrimidine-5-carbonyl}-amino)-3-[2-cyclohexyl-acetylamino]-propionic acid;
(S)-2-({2-[3-(3-hydroxy-phenyl)-propylamino]-4,6-dimethyl-pyrimidine-5-carbonyl}-amino)-3-[(isoquinoline-1-carbonyl)-amino]-propionic acid;
(S)-2-({2-[3-(3-hydroxy-phenyl)-propylamino]-4,6-dimethyl-pyrimidine-5-carbonyl}-amino)-3-[(thiazole-4-carbonyl)-amino]-propionic acid;
(S)-2-({2-[3-(3-hydroxy-phenyl)-propylamino]-4,6-dimethyl-pyrimidine-5-carbonyl}-amino)-3-[(2-pyridin-4-yl-thiazole-4-carbonyl)-amino]-propionic acid;
(S)-2-({2-[3-(3-hydroxy-4-methyl-phenyl)-propylamino]-4,6-dimethyl-pyrimidine-5-carbonyl}-amino)-3-[(thiophene-2-carbonyl)-amino]-propionic acid methyl ester;
(S)-2-({2-[(E)-3-(3-hydroxy-phenyl)-allylamino]-4,6-dimethyl-pyrimidine-5-carbonyl}-amino)-3-[(thiophene-2-carbonyl)-amino]-propionic acid methyl ester;
(S)-2-({2-[(E)-3-(6-benzyloxy-pyridin-2-yl)-allylamino]-4,6-dimethyl-pyrimidine-5-carbonyl}-amino)-propionic acid methyl ester;
(S)-2-({2-[(E)-3-(6-Benzyloxy-pyridin-2-yl)-allyamino]-4,6-dimethyl-pyrimidine-5-carbonyl}-amino)-3-[(thiophene-2-carbonyl)-amino]-propionic acid;
(S)-2-({2-[3-(6-Hydroxy-pyridin-2-yl)-propylamino]-4,6-dimethyl-pyrimidine-5-carbonyl}-amino)-3-[(thiophene-2-carbonyl)-amino]-propionic acid;
(S)-2-({2-[3-(2-Fluoro-3-hydroxy-phenyl)-propylamino]-4,6-dimethyl-pyrimidine-5-carbonyl}-amino)-3-[(thiophene-2-carbonyl)-amino]-propionic acid methyl ester;
(S)-2-({2-[3-(2-Fluoro-5-hydroxy-phenyl)-propylamino]-4,6-dimethyl-pyrimidine-5-carbonyl}-amino)-3-[(thiophene-2-carbonyl)-amino]-propionic acid methyl ester;
(S)-2-({2-[3-(2-Methyl-5-hydroxy-phenyl)-propylamino]-4,6-dimethyl-pyrimidine-5-carbonyl}-amino)-3-[(thiophene-2-carbonyl)-amino]-propionic acid methyl ester;
(S)-2-({2-[3-(3-hydroxy-phenyl)-propylamino]-pyrimidine-5-carbonyl}-amino)-3-[(thiophene-2-carbonyl)-amino]-propionic acid methyl ester;
(S)-2-({2-[3-(3-hydroxy-phenyl)-propylamino]-4-methyl-pyrimidine-5-carbonyl}-amino)-3-[(thiophene-2-carbonyl)-amino]-propionic acid methyl ester;
(S)-2-({2-[3-(3-hydroxy-phenyl)-propylamino]-4-trifluoromethyl-pyrimidine-5-carbonyl}-amino)-3-[(thiophene-2-carbonyl)-amino]-propionic acid methyl ester;
(S)-2-({4-Ethyl-2-[3-(3-hydroxy-phenyl)-propylamino]-6-methyl-pyrimidine-5-carbonyl}-amino)-3-[(thiophene-2-carbonyl)-amino]-propionic acid methyl ester;
(S)-2-({4,6-diethyl-2-[3-(3-hydroxy-phenyl)-propylamino]-pyrimidine-5-carbonyl}-amino)-3-[(thiophene-2-carbonyl)-amino]-propionic acid methyl ester;
(S)-2-({2-[3-(3-Hydroxy-phenyl)-prop-2-ynylamino]-4,6-dimethyl-pyrimidine-5-carbonyl}-amino)-3-[(thiophene-2-carbonyl)-amino]-propionic acid;
(S)-2-({2-[3-(1H-Indazol-4-yl)-prop-2-ynylamino]-4,6-dimethyl-pyrimidine-5-carbonyl}-amino)-3-[(thiophene-2-carbonyl)-amino]-propionic acid;
(S)-2-({2-[3-(1H-Indazol-6-yl)-propylamino]-4,6-dimethyl-pyrimidine-5-carbonyl}-amino)-3-[(thiophene-2-carbonyl)-amino]-propionic acid;
(S)-2-({2-[3-(3-Fluoro-5-hydroxy-phenyl)-propylamino]-4,6-dimethyl-pyrimidine-5-carbonyl}-amino)-3-[(thiophene-2-carbonyl)-amino]-propionic acid;
(S)-2-({2-[3-(4-Fluoro-3-hydroxy-phenyl)-propylamino]-4,6-dimethyl-pyrimidine-5-carbonyl}-amino)-3-[(thiophene-2-carbonyl)-amino]-propionic acid;
(S)-2-({2-[3-(3-Hydroxy-2-methyl-phenyl)-propylamino]-4,6-dimethyl-pyrimidine-5-carbonyl}-amino)-3-[(thiophene-2-carbonyl)-amino]-propionic acid;
(S)-2-({2-[3-(3-Hydroxy-4-methyl-phenyl)-propylamino]-4,6-dimethyl-pyrimidine-5-carbonyl}-amino)-3-[(thiophene-2-carbonyl)-amino]-propionic acid;
(S)-2-({2-[3-(2-Chloro-5-hydroxy-phenyl)-propylamino]-4,6-dimethyl-pyrimidine-5-carbonyl}-amino)-3-[(thiophene-2-carbonyl)-amino]-propionic acid;
(S)-2-({2-[3-(4-Hydroxy-phenyl)-propylamino]-4,6-dimethyl-pyrimidine-5-carbonyl}-amino)-3-[(thiophene-2-carbonyl)-amino]-propionic acid;
(S)-2-({2-[3-(2-Fluoro-3-hydroxy-phenyl)-propylamino]-4,6-dimethyl-pyrimidine-5-carbonyl}-amino)-3-[(thiophene-2-carbonyl)-amino]-propionic acid;
(S)-2-({2-[3-(2-Fluoro-5-hydroxy-phenyl)-propylamino]-4,6-dimethyl-pyrimidine-5-carbonyl}-amino)-3-[(thiophene-2-carbonyl)-amino]-propionic acid;
(S)-2-({2-[3-(2-Methyl-5-hydroxy-phenyl)-propylamino]-4,6-dimethyl-pyrimidine-5-carbonyl}-amino)-3-[(thiophene-2-carbonyl)-amino]-propionic acid;
(S)-2-({2-[(Z)-3-(3-Hydroxy-phenyl)-allylamino]-4,6-dimethyl-pyrimidine-5-carbonyl}-amino)-3-[(thiophene-2-carbonyl)-amino]-propionic acid;
(S)-2-({2-[(E)-3-(3-Hydroxy-phenyl)-allylamino]-4,6-dimethyl-pyrimidine-5-carbonyl}-amino)-3-[(thiophene-2-carbonyl)-amino]-propionic acid;
(S)-2-({2-[3-(3-Hydroxy-phenyl)-propylamino]-pyrimidine-5-carbonyl}-amino)-3-[(thiophene-2-carbonyl)-amino]-propionic acid;
(S)-2-({2-[3-(3-Hydroxy-phenyl)-propylamino]-4-methyl-pyrimidine-5-carbonyl}-amino)-3-[(thiophene-2-carbonyl)-amino]-propionic acid;
(S)-2-({2-[3-(3-Hydroxy-phenyl)-propylamino]-4-trifluoromethyl-pyrimidine-5-carbonyl}-amino)-3-[(thiophene-2-carbonyl)-amino]-propionic acid;
(S)-2-({4-Ethyl-2-[3-(3-hydroxy-phenyl)-propylamino]-6-methyl-pyrimidine-5-carbonyl}-amino)-3-[(thiophene-2-carbonyl)-amino]-propionic acid;
(S)-2-({4,6-Diethyl-2-[3-(3-hydroxy-phenyl)-propylamino]-pyrimidine-5-carbonyl}-amino)-3-[(thiophene-2-carbonyl)-amino]-propionic acid;
(S)-3-(3,5-Dihydroxy-benzoylamino-2-({2-[3-(3-hydroxy-phenyl)-propylamino]-4,6-dimethyl-pyrimidine-5-carbonyl}-amino)-propionic acid methyl ester;
(S)-3-(3,5-Dihydroxy-benzoylamino-2-({2-[3-(3-hydroxy-phenyl)-propylamino]-4,6-dimethyl-pyrimidine-5-carbonyl}-amino)-propionic acid;
(S)-3-(3-Hydroxy-benzoylamino-2-({2-[3-(3-hydroxy-phenyl)-propylamino]-4,6-dimethyl-pyrimidine-5-carbonyl}-amino)-propionic acid methyl ester;
(S)-3-(3-Hydroxy-benzoylamino-2-({2-[3-(3-hydroxy-phenyl)-propylamino]-4,6-dimethyl-pyrimidine-5-carbonyl}-amino)-propionic acid;
(S)-2-({2-[3-(3-hydroxy-phenyl)-propylamino]-4,6-dimethyl-pyrimidine-5-carbonyl}-amino)-3-[(thiophene-3-carbonyl)-amino]-propionic acid methyl ester;
(S)-2-({2-[3-(3-hydroxy-phenyl)-propylamino]-4,6-dimethyl-pyrimidine-5-carbonyl}-amino)-3-[(thiophene-3-carbonyl)-amino]-propionic acid;
(S)-2-({2-[3-(3-hydroxy-phenyl)-propylamino]-4,6-dimethyl-pyrimidine-5-carbonyl}-amino)-3-[(5-methyl-thiophene-2-carbonyl)-amino]-propionic acid methyl ester;
(S)-2-({2-[3-(3-hydroxy-phenyl)-propylamino]-4,6-dimethyl-pyrimidine-5-carbonyl}-amino)-3-[(5-methyl-thiophene-2-carbonyl)-amino]-propionic acid;
(S)-2-({2-[3-(3-hydroxy-phenyl)-propylamino]-4,6-dimethyl-pyrimidine-5-carbonyl}-amino)-3-[(4-methyl-thiophene-2-carbonyl)-amino]-propionic acid methyl ester;
(S)-2-({2-[3-(3-hydroxy-phenyl)-propylamino]-4,6-dimethyl-pyrimidine-5-carbonyl}-amino)-3-[(4-methyl-thiophene-2-carbonyl)-amino]-propionic acid;
(S)-2-({2-[3-(3-hydroxy-phenyl)-propylamino]-4,6-dimethyl-pyrimidine-5-carbonyl}-amino)-3-[(3-methyl-thiophene-2-carbonyl)-amino]-propionic acid methyl ester;
(S)-2-({2-[3-(3-hydroxy-phenyl)-propylamino]-4,6-dimethyl-pyrimidine-5-carbonyl}-amino)-3-[(3-methyl-thiophene-2-carbonyl)-amino]-propionic acid;
(S)-2-({2-[3-(3-hydroxy-phenyl)-propylamino]-4,6-dimethyl-pyrimidine-5-carbonyl}-amino)-3-[(pyrazine-2-carbonyl)-amino]-propionic acid methyl ester;
(S)-2-({2-[3-(3-hydroxy-phenyl)-propylamino]-4,6-dimethyl-pyrimidine-5-carbonyl}-amino)-3-[(pyrazine-2-carbonyl)-amino]-propionic acid;
(S)-3-[(5-Chloro-thiophene-2-carbonyl)-amino]-2({2-[3-(3-hydroxy-phenyl)-propylamino]-4,6-dimethyl-pyrimidine-5-carbonyl}-amino)-propionic acid methyl ester;
(S)-3-[(5-Chloro-thiophene-2-carbonyl)-amino]-2-({2-[3-(3-hydroxy-phenyl)-propylamino]-4,6-dimethyl-pyrimidine-5-carbonyl}-amino)-propionic acid;
(S)-2-({2-[3-(3-hydroxy-phenyl)-propylamino]-4,6-dimethyl-pyrimidine-5-carbonyl}-amino)-3-(3-methyl-benzoylamino)-propionic acid methyl ester;
(S)-2-({2-[3-(3-hydroxy-phenyl)-propylamino]-4,6-dimethyl-pyrimidine-5-carbonyl}-amino)-3-(3-methyl-benzoylamino)-propionic acid;
(S)-3-[(Adamantane-1-carbonyl)-amino]-2-({2-[3-(3-hydroxy-phenyl)-propylamino]-4,6-dimethyl-pyrimidine-5-carbonyl}-amino)-propionic acid methyl ester;
(S)-3-[(Adamantane-1-carbonyl)-amino]-2-({2-[3-(3-hydroxy-phenyl)-propylamino]-4,6-dimethyl-pyrimidine-5-carbonyl}-amino)-propionic acid;
(S)-2-({2-[3-(3-Hydroxy-phenyl)-propylamino]-4,6-dimethyl-pyrimidine-5-carbonyl}-amino)-3-[(5-methyl-pyrazine-2-carbonyl)-amino]-propionic acid methyl ester;
(S)-2-({2-[3-(3-Hydroxy-phenyl)-propylamino]-4,6-dimethyl-pyrimidine-5-carbonyl}-amino)-3-[(5-methyl-pyrazine-2-carbonyl)-amino]-propionic acid;
(S)-3-[(3-Chloro-thiophene-2-carbonyl)-amino]-2-({2-[3-(3-hydroxy-phenyl)-propylamino]-4,6-dimethyl-pyrimidine-5-carbonyl}-amino)-propionic acid methyl ester;
(S)-3-[(3-Chloro-thiophene-2-carbonyl)-amino]-2-({2-[3-(3-hydroxy-phenyl)-propylamino]-4,6-dimethyl-pyrimidine-5-carbonyl}-amino)-propionic acid;
(S)-[(5-Ethyl-thiophene-2-carbonyl)-amino]-2-({2-[3-(3-hydroxy-phenyl)-propylamino]-4,6-dimethyl-pyrimidine-5-carbonyl}-amino)-3-propionic acid methyl ester;
(S)-[(5-Ethyl-thiophene-2-carbonyl)-amino]-2-({2-[3-(3-hydroxy-phenyl)-propylamino]-4,6-dimethyl-pyrimidine-5-carbonyl}-amino)-3-propionic acid;
(S)-2-({4,6-Dimethyl-2-[3-(3-phenyl carb amoyloxy-phenyl)-propylamino]-pyrimidine-5-carbonyl}-amino)-3-(3-phenyl-ureido)-propionic acid methyl ester;
(S)-2-({2-[3-(3-hydroxy-phenyl)-propylamino]-4,6-dimethyl-pyrimidine-5-carbonyl}-amino)-3-(3-phenyl-ureido)-propionic acid;
(S)-2-({2-[3-(3-Hydroxy-phenyl)-propylamino]-4,6-dimethyl-pyrimidine-5-carbonyl}-amino)-3-methoxycarbonylamino-propionic acid methyl ester;
(S)-2-({2-[3-(3-Hydroxy-phenyl)-propylamino]-4,6-dimethyl-pyrimidine-5-carbonyl}-amino)-3-methoxycarbonylamino-propionic acid;
(S)-3-Benzyloxycarbonylamino-2-({2-[3-(3-Hydroxy-phenyl)-propylamino]-4,6-dimethyl-pyrimidine-5-carbonyl}-amino)-propionic acid methyl ester;
(S)-3-Benzyloxycarbonylamino-2-({2-[3-(3-Hydroxy-phenyl)-propylamino]-4,6-dimethyl-pyrimidine-5-carbonyl}-amino)-propionic acid;
(S)-3-(3,3-Dimethyl-ureido)-2-({2-[3-(3-Hydroxy-phenyl)-propylamino]-4,6-dimethyl-pyrimidine-5-carbonyl}-amino)-propionic acid methyl ester;
(S)-3-(3,3-Dimethyl-ureido)-2-({2-[3-(3-Hydroxy-phenyl)-propylamino]-4,6-dimethyl-pyrimidine-5-carbonyl}-amino)-propionic acid;
(S)-3-[(3,4-Dihydro-2H-quino line-1-carbonyl)-amino]-2-({2-[3-(3-hydroxy-phenyl)-propylamino]-4,6-dimethyl-pyrimidine-5-carbonyl}-amino)-propionic acid methyl ester;
(S)-3-[(3,4-Dihydro-2H-quino line-1-carbonyl)-amino]-2-({2-[3-(3-hydroxy-phenyl)-propylamino]-4,6-dimethyl-pyrimidine-5-carbonyl}-amino)-prop ionic acid;
(S)-2-{[2-(8-Hydroxy-1,2,3,4-tetrahydro-naphthalen-2-ylamino)-4,6-dimethyl-pyrimidine-5-carbonyl]-amino}-3-[(pyrazine-2-carbonyl)-amino]-propionic acid methyl ester;
(S)-2-{[2-(8-Hydroxy-1,2,3,4-tetrahydro-naphthalen-2-ylamino)-4,6-dimethyl-pyrimidine-5-carbonyl]-amino}-3-[(pyrazine-2-carbonyl)-amino]-propionic acid;
(S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid 2,2-dimethyl-propyl ester;
(S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid butyl ester;
(S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid 3-morpholin-4-yl-propyl ester;
(S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid 2-morpholin-4-yl-ethyl ester;
(S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid propyl ester;
(S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid isobutyl ester;
(S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid isopropyl ester;
(S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid 1-ethyl-propyl ester;
(S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid cyclopentyl ester;
(S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid dimethylcarbamoylmethyl ester;
(S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid 3-ethoxy-propyl ester;
(S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid tetrahydro-pyran-4-yl ester;
(S)-2-({2-[3-(3-Hydroxy-phenyl)-propylamino]-4,6-dimethyl-pyrimidine-5-carbonyl}-amino)-3-[(thiophen-2-carbonyl)-amino]-propionic acid 2,2-dimethyl-propionyloxymethyl ester;
(S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid 1-(2,2-dimethyl-propionyloxy)-ethyl ester;
(S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid 1-isobutyryloxy-ethyl ester;
(S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid 2-(2-ethoxy-ethoxy)-ethyl ester;
(S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid 2-ethoxy-ethyl ester;
(S)-2-({2-[3-(3-Acetoxy-phenyl)-propylamino]-4,6-dimethyl-pyrimidine-5-carbonyl}-amino)-3-[(thiophene-2-carbonyl)-amino]-propionic acid 3-ethoxy-propyl ester;
(S)-2-({2-[3-(3-Isobutyryloxy-phenyl)-propylamino]-4,6-dimethyl-pyrimidine-5-carbonyl}-amino)-3-[(thiophene-2-carbonyl)-amino]-propionic acid 3-ethoxy-propyl ester;
(S)-2-({2-[3-(1H-Indazol-4-yl)-propylamino]-4,6-dimethyl-pyrimidine-5-carbonyl}-amino)-3-[(thiophene-2-carbonyl)-amino]-propionic acid butyl ester;
(S)-2-({2-[3-(1H-Indazol-4-yl)-propylamino]-4,6-dimethyl-pyrimidine-5-carbonyl}-amino)-3-[(thiophene-2-carbonyl)-amino]-propionic acid ethyl ester;
(S)-2-({2-[3-(1H-Indazol-4-yl)-propylamino]-4,6-dimethyl-pyrimidine-5-carbonyl}-amino)-3-[(thiophene-2-carbonyl)-amino]-propionic acid cyclopentyl ester;
(S)-2-({2-[3-(1H-Indazol-4-yl)-propylamino]-4,6-dimethyl-pyrimidine-5-carbonyl}-amino)-3-[(thiophene-2-carbonyl)-amino]-propionic acid isobutyl ester;
(S)-2-({2-[3-(1H-Indazol-4-yl)-propylamino]-4,6-dimethyl-pyrimidine-5-carbonyl}-amino)-3-[(thiophene-2-carbonyl)-amino]-propionic acid 1-ethyl-propyl ester;
(S)-2-({2-[3-(1H-Indazol-4-yl)-propylamino]-4,6-dimethyl-pyrimidine-5-carbonyl}-amino)-3-[(thiophene-2-carbonyl)-amino]-propionic acid; dimethylcarbamoylmethyl ester;
(S)-2-({2-[3-(1H-Indazol-4-yl)-propylamino]-4,6-dimethyl-pyrimidine-5-carbonyl}-amino)-3-[(thiophene-2-carbonyl)-amino]-propionic acid isopropyl ester;
(S)-2-({2-[3-(1H-Indazol-4-yl)-propylamino]-4,6-dimethyl-pyrimidine-5-carbonyl}-amino)-3-[(thiophene-2-carbonyl)-amino]-propionic acid propyl ester;
(S)-2-({2-[3-(1H-Indazol-4-yl)-propylamino]-4,6-dimethyl-pyrimidine-5-carbonyl}-amino)-3-[(thiophene-2-carbonyl)-amino]-propionic acid pentyl ester;
(S)-2-({2-[3-(1H-Indazol-4-yl)-propylamino]-4,6-dimethyl-pyrimidine-5-carbonyl}-amino)-3-[(thiophene-2-carbonyl)-amino]-propionic acid 3-morpholin-4-yl-propyl ester; and
(S)-2-({2-[3-(1H-Indazol-4-yl)-propylamino]-4,6-dimethyl-pyrimidine-5-carbonyl}-amino)-3-[(thiophene-2-carbonyl)-amino]-propionic acid 3-ethoxy-propyl ester.
295) In another embodiment of the invention, provided is a process for the preparation of a compound according formula, comprising the steps of:
   reacting compounds of formula B1 and A1 with compounds B2 and A2 to produce a compound of formula AB:

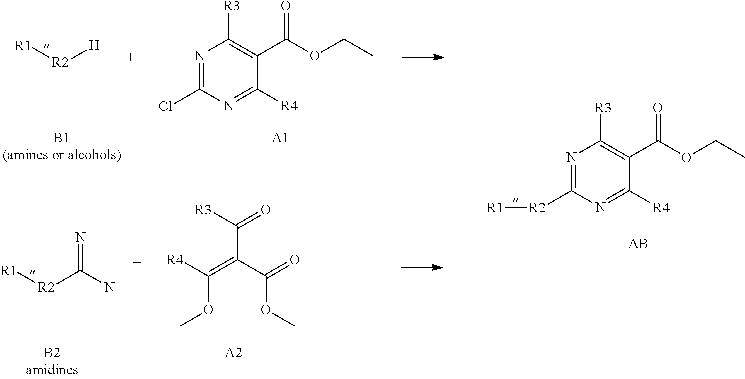

and forming a compound of formula (I):

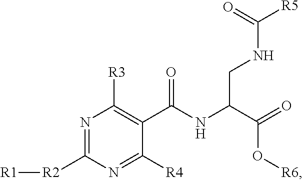

or a pharmaceutically acceptable salt thereof.
296) In another embodiment of the invention, provided is a compound of formula (I) for use as a therapeutically active substance.
297) In another embodiment of the invention, provided is pharmaceutical composition comprising a therapeutically effective amount of a compound of formula (I) and a therapeutically inert carrier.
298) In another embodiment of the invention, provided is a use of a compound according to formula (I) for the treatment or prophylaxis of asthma or COPD.
299) In another embodiment of the invention, provided is a use of a compound according to formula (I) for the preparation of a medicament for the treatment or prophylaxis of asthma or COPD.
300) In another embodiment of the invention, provided is a compound according to formula (I) for the treatment or prophylaxis of asthma or COPD.
301) In another embodiment of the invention, provided is compound according formula (I), when manufactured according to the process above.
302) In another embodiment of the invention, provided is a method for the treatment or prophylaxis of asthma or COPD, which method comprises the step of administering a therapeutically effective amount of a compound of formula (I) to a patient in need thereof.
303) In another embodiment of the invention, provided is an invention as hereinbefore described.
304) Compounds of the present invention can be prepared beginning with commercially available starting materials, or utilizing general synthetic techniques and procedures known to those skilled in the art. Chemicals may be purchased from companies such as for example Aldrich, Argonaut Technologies, VWR, Lancaster, Princeton, Alfa, Oakwood, TCI, Fluorochem, Apollo, Matrix, Maybridge or Meinoah. Chromatography supplies and equipment may be purchased from such companies as for example Anal.ogix, Inc, Burlington, Wis.; Biotage AB, Charlottesville, Va.; Analytical Sales and Services, Inc., Pompton Plains, N.J.; Teledyne Isco, Lincoln, Nebr.; VWR International, Bridgeport, N.J.; Varian Inc., Palo Alto, Calif., and Multigram II Mettler Toledo Instrument Newark, Del. Biotage, ISCO and Analogix columns are pre-packed silica gel columns used in standard chromatography. Final compounds and intermediates were named using the AutoNom2000 feature in the MDL ISIS Draw application.
305) Preferably, the compounds of formula I, can be prepared by the following general reaction scheme.

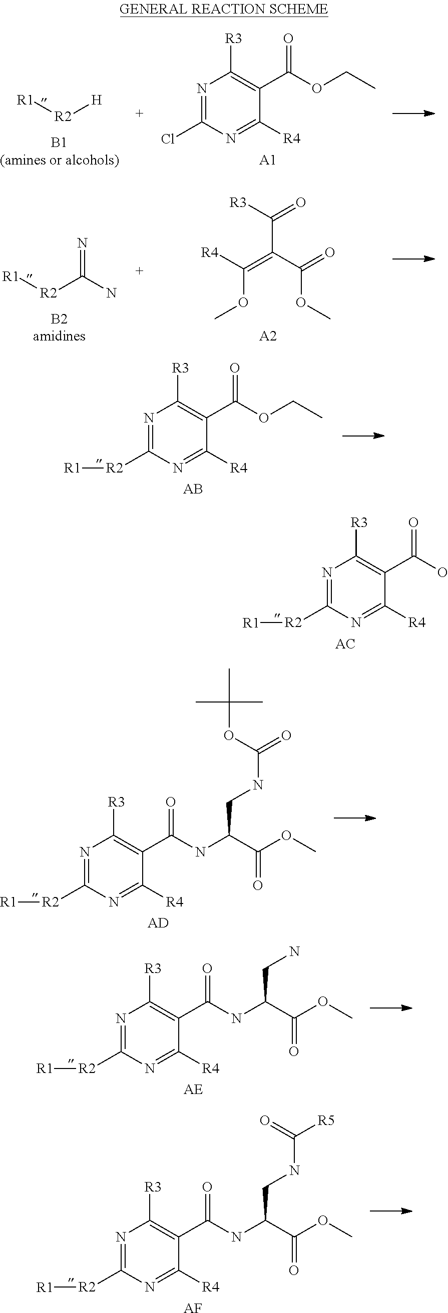

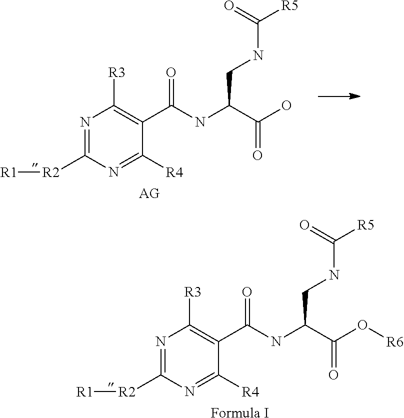

306) Compounds of this invention (formula I) can be synthesized according to general schemes above. Starting materials are available from commercial sources or their preparation is described herein.
307) As seen in the scheme, R1 can be:

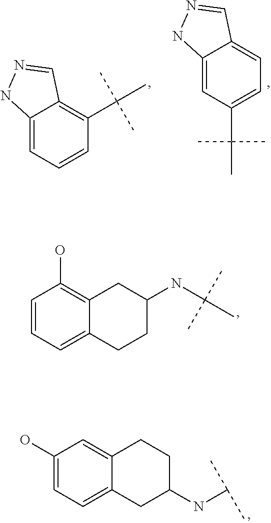

308) phenyl, mono- or bi-substituted independently with hydroxy, halogen, lower alkyl, alkoxy, —OC(O)CH₃ or —OC(O)CH(CH₃)₂, or heteroaryl, mono- or bi-substituted with hydroxy;

### R2 can be:

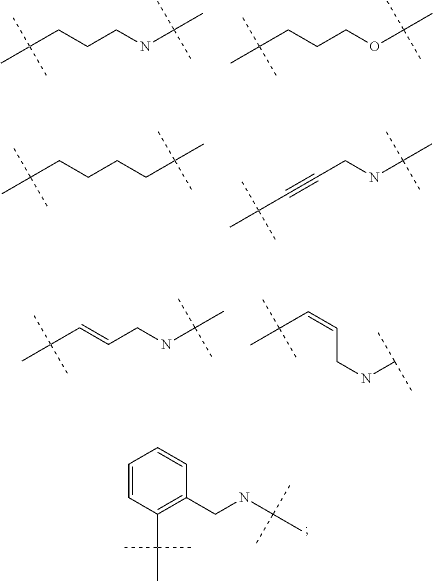

or absent if R1 is

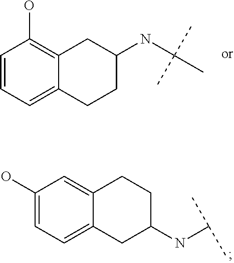

R3 and R4, independently or each other, can be H or lower alkyl;
R5 can be lower alkyl, —C(O)CH₃, cycloalkyl, lower alkyl-cycloalkyl, heterocycloalkyl, isoquinoline, quinoline, adamantane, NR7R8, OR9, substituted or unsubstituted heteroaryl or substituted or unsubstituted phenyl;
R6 can be hydrogen, lower alkyl, alkoxy, cycloalkyl, aryl, heteroaryl, —(CH₂)₂O(CH₂)₂OCH₂CH₃—,

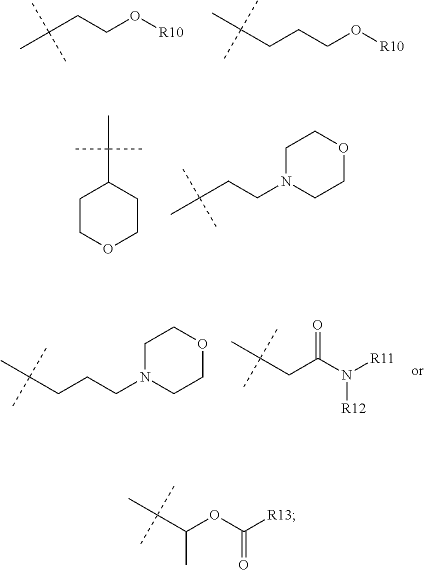

R7 and R8, independently of each other, can be hydrogen, lower alkyl, cycloalkyl, aryl or heteroaryl;
R9 can be lower alkyl, cycloalkyl, phenyl or heteroaryl; and
R10, R11, R12 and R13, independently of each other, can be hydrogen, lower alkyl or alkoxy.
309) Compounds of formula AB can be prepared starting from compound A1 by nucleophilic substitution of the chloro atom of the appropriately substituted pyrimidine with an appropriate nucleophile B1, an amine or an alcohol, in the presence of a base such as potassium acetate and solvent such as ethanol at elevated temperatures such as 120-160° C., the rate of reaction often being enhanced by the use of a microwave reactor to produce intermediates of formula AB. With C-linked pyrimidines, compound AB can be prepared by condensation of an amidine with an unsaturated β-keto ester such as A2. This reaction can be carried out at reflux in methanol, for example.
310) Saponification of compounds of the formula AB to compounds of the formula AC can be carried out with a suitable base such as a metal hydroxide, preferably lithium hydroxide in an appropriate solvent such as THF and mixtures thereof with water preferably at room temperature or higher as needed.
311) Compounds of the general formula AD can be prepared by coupling of compounds of the general formula AC with H-DAP-BOC-OMe using methods for the formation of peptide bonds such as activation of the carboxylic acid with HBTU/HOBt and coupling with the amine in an inert solvent such as DMF in the presence of a suitable base such as Triethylamine.
312) Compounds of the general formula AE can be prepared by deprotection of the amine of compounds of general formula AD by treatment with a mineral acid, preferably HCl in an inert solvent such as Dioxane or mixtures thereof with methanol.
313) Compounds of the general formula AF can be prepared by coupling of compounds of the general formula AE with a desired carboxylic acid using methods for the formation of peptide bonds such as activation of the carboxylic acid with HBTU/HOBt and coupling with the amine in an inert solvent such as DMF in the presence of a suitable base such as Triethylamine.
314) Saponification of compounds of the formula AF to compounds of the formula AG can be carried out with a suitable base such as a metal hydroxide, preferably lithium hydroxide in an appropriate solvent such as THF and mixtures thereof with water preferably at room temperature.
315) Compounds of Formula I can be prepared by alkylation of the carboxylic acid of compounds of the general formula AG with an alkyl halide, preferably an alkyl bromide or iodide in an inert solvent such as DMF, in the presence of a base such as potassium carbonate at room temperature or by reaction in a microwave reactor at temperatures between 100-160° C. Alternatively carboxylic acids of general formula AG can be treated with a chlorinating agent, such as thionyl chloride and then reacting with an appropriately chosen alcohol in the presence of an inert solvent such as dioxane.
316) The invention will now be further described in the Examples below, which are intended as an illustration only and do not limit the scope of the invention.

### EXAMPLES

### Part 1: Preferred Intermediates

### Preparation of 3-(3-methoxy-phenyl)-propionamide

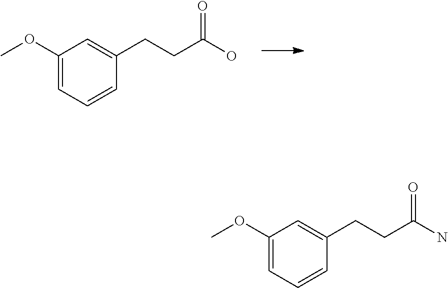 [0225] 317) A solution of 3-(3-methoxy-phenyl)-propionic acid (15 g, 83.2 mmol) and 4-methyl-morpholine (10.1 ml, 91.56 mmol) in THF (150 ml) was cooled to 0° C. (ice-water bath), and iso-propyl chloroformate (1M in toluene, 91.6 ml, 91.56 mmol) was added over 20 minutes. The mixture was stirred for another 30 minute at 0° C., followed by dropwise addition of 7N NH₃/MeOH (24 ml, 168 mmol). The mixture was allowed to warm up to room temperature and stirred for 2 h. It was quenched with 10% aqK₂CO₃ and extracted with EtOAc. The organic extracts were combined, washed with water and brine, dried over sodium sulfate, filtered and evaporated to give the desired amide (11.15 g, 75% yield). MS m/e 179.9 (M+H⁺).

### Preparation of 3-(3-methoxy-phenyl)-propylamine

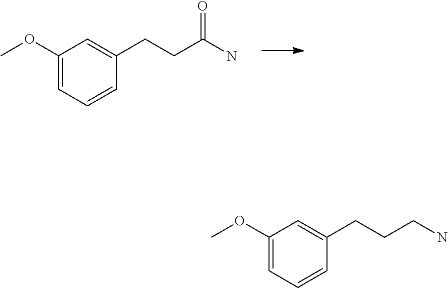 [0227] 318) BH₃ in THF (2.2 g, 188 mmol) was added at room temperature to a solution of 3-(3-methoxy-phenyl)-propionamide (11.15 g, 62.26 mmol) in THF (100 ml). The solution was heated to reflux for 4 h, cooled to room temperature and quenched with MeOH (50 ml). The solution was heated to reflux for 30 min, concentrated, treated with water, and extracted with EtOAc. The extract was washed with 10% aqK₂CO₃, water and brine, dried over Na₂SO₄, filtered and evaporated to give title compound (9.26 g, 90% yield). MS m/e 165.9 (M+H⁺).

### Preparation of 1-acetyl-1H-indazol-4-yl-methyl acetate

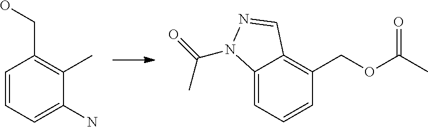 [0229] 319) A solution of 3-amino-2-methyl benzyl alcohol (15.0 g, 109.3 mmol), Ac₂O (33.49 g, 328.04 mmol), and KOAc (21.46 g, 218.68 mmol) in chloroform (200 ml) was stirred at room temperature for 2 h, refluxed for 3 h and stirred at room temperature for 2 days. N-amylnitrite (28.82 g, 246.02 mmol) and 18-crown-6 (1.45 g, 5.47 mmol) were added and the mixture was refluxed overnight. After cooling to room temperature, Ac₂O (30 ml) was added and the mixture was stirred at room temperature overnight. The reaction mixture was diluted with water (200 ml). After one hour the layers was separated, and the organic layer was successively washed with water (200 ml), saturated aqNaHCO₃ (200 ml), water (200 mL), and brine (200 mL). The organic layer was dried over sodium sulfate, filtered, and concentrated. The residue was purified by flash chromatography with a 0-20% EtOAc in hexane gradient to afford the desired compound (16.88 g, 66% yield).

### Preparation of 4-bromomethyl-1H-indazole hydrobromide

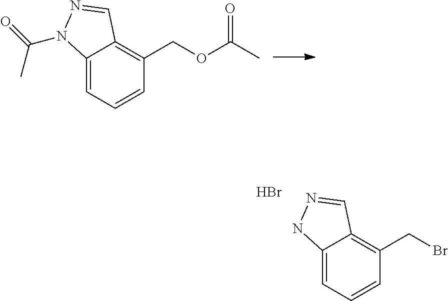 [0231] 320) A mixture of 1-acetyl-1H-indazol-4-yl-methyl acetate (12.76 g, 54.94 mmol) and 48% hydrobromic acid (100 ml) was heated to 70° C. for 18 h, then cooled to room temperature. The resulting precipitate was collected by filtration, then washed with water and ether to afford the title compound (10.4 g, 65% yield).

### Preparation of 2-cyano-3-(1H-indazol-4-yl)-propionic acid tert-butyl ester

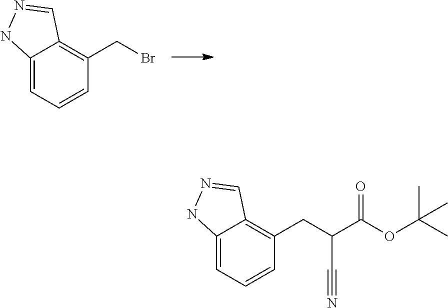 [0233] 321) A solution of 4-bromomethyl-1H-indazole hydrobromide (2.48 g, 8.49 mmol) in DMF (10 ml) was added dropwise to a mixture of tert-butylcyanoacetate (12.01 g, 84.94 mmol) and K₂CO₃ (11.74 g, 84.94 mmol) in DMF (90 mL). The mixture was stirred for 3.5 h, then diluted with EtOAc (500 mL) and washed with brine (4×300 ml). The organic layer was dried over sodium sulfate, filtered, and concentrated. The residue was purified by flash chromatography with a 0-60% EtOAc in hexane gradient to afford the desired compound (2.05 g, 89% yield).

### Preparation of 2-[3-(1H-indazol-4-yl)]-propionitrile

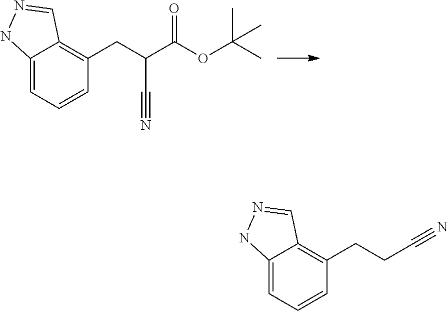 [0235] 322) A solution of 2-cyano-3-(1H-indazol-4-yl)-propionic acid tert-butyl ester (9.22 g, 34 mmol), NaCl (5.96 g, 102 mmol), and water (2 ml) in DMSO (80 ml) was heated to 150° C. for 4.5 h, then stirred at room temperature overnight. The mixture was diluted with brine (300 ml) and extracted with EtOAc (3×300 ml). The combined organic layers were washed with brine (3×250 mL), dried over sodium sulfate, filtered and concentrated The residue was purified by flash chromatography with a 10-60% EtOAc in hexane gradient to afford the desired product (4.27 g, 73% yield).

### Preparation of 2-[3-(1H-indazol-4-yl)]-propylamine dihydrochloride

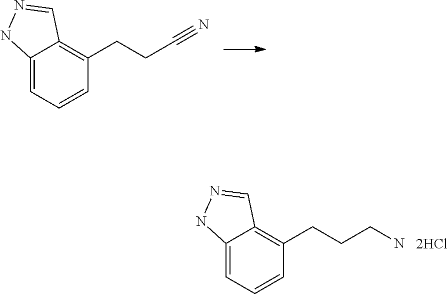 [0237] 323) A mixture of 3-(1H-indazol-4-yl)-propionitrile (2.0 g, 11.6 mmol), platinum (IV) oxide (0.45 g), and concentrated HCl (6 ml) in EtOH (100 ml) was hydrogenated at room temperature under 50 psi for 18 h. The mixture was filtered through celite, then concentrated and azeotroped twice with MeOH. The residue was triturated with ether and collected by filtration to afford the title compound (2.72 g, 94% yield).

### Preparation of 3-(3-methoxy-phenyl)-propan-1-ol

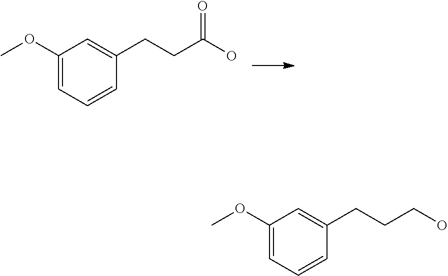 [0239] 324) A solution of 3-(3-methoxy-phenyl)-propionic acid (6.0 g, 33.3 mmol) in THF (160 ml) was cooled to 0° C. (ice water bath). BH3/THF (1M in THF, 100 ml, 100 mmol) was added dropwise at 0° C. over 35 min. The solution was allowed to warm up to room temperature before heating under reflux for 1 h. The mixture was cooled to 0° C. and then quenched with methanol (30 ml). The mixture was concentrated under reduced pressure and the crude residue was partitioned between EtOAc and 1NHCl. The organic phase was isolated, washed with water, 0.5NNaOH and brine, then dried over MgSO₄, filtered and concentrated to afford the desired alcohol as a colorless oil (5.7 g, 100% yield).

### Preparation of Methanesulfonic acid 3-(3-methoxy-phenyl)-propyl ester

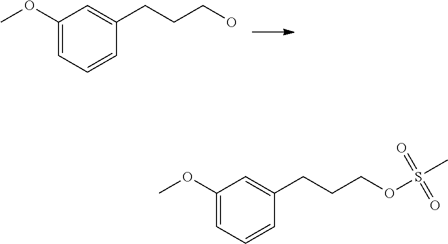 [0241] 325) A solution of 3-(3-methoxy-phenyl)-propan-1-ol (5.7 g, 34.4 mmol) in DCM (160 ml) was treated with triethylamine (4.78 ml, 34.4 mmol) and methanesulfonyl chloride (2.66 ml, 34.4 mmol) at room temperature for 2 h. The mixture was poured into water and the organic phase was washed with saturated aqNaHCO₃, water, 1NHCl and brine. The organic phase was separated, dried over MgSO₄, filtered and concentrated under reduced pressure to afford the desired methane sulfonate as a colorless oil (8.3 g, 98% yield).

### Preparation of 3-iodo-2-methyl-phenol

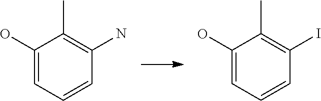 [0243] 326) A 250 ml round-bottom flask containing BF₃.Et₂O (2 ml, 16.24 mmol) was cooled to 0° C. and 3-amino-2-methyl-phenol (1 g, 8.12 mmol) in THF (30 ml) was added slowly. The solution was cooled in salt-ice-water bath and isoamyl nitrite (1.4 ml, 10.55 mmol) in THF (5 ml) was added dropwise. Stirring was continued at −10° C. for 30 min., followed by addition of cold diethyl ether. The red precipitate was collected by filtration. The solid was then added portion-wise at room temperature to a solution of sodium iodide (1.6 g, 10.55 mmol) in acetone (40 ml). Stirring was continued for 12 h at room temperature. Acetone was removed by evaporation and the residue was purified by flash chromatography with 30% EtOAc in hexane to afford the desired product (0.98 g, 52% yield). MS m/e 233.1 (M+H⁺).

### Preparation of 1-chloro-2-iodo-4-methoxy-benzene

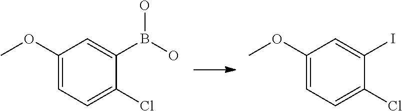 [0245] 327) To a 250 ml round-bottom flask wrapped with aluminum foil was added a solution of 2-chloro-5-methoxy-phenyl boronic acid (5 g, 26.82 mmol) in 50% MeOH/H₂O (100 ml). A solution of NaI (5.57 g, 33.5 mmol) in water (34 ml) and a solution of Chloramine-T (18.9 g, 67.1 mmol) in 50% MeOH/H₂O (34 ml) were added to the mixture while stirring. Stirring was continued for 15 min at room temperature, followed by treatment with water and extraction with diethyl ether. The organic extraction was washed with water and brine, dried over sodium sulfate, filtered and evaporated. The residue was purified by flash chromatography with 10% EtOAc in hexane to afford the desired product contaminated with EtOAc (8.6 g).

### Preparation of 1-Fluoro-2-bromo-4-methoxy-benzene

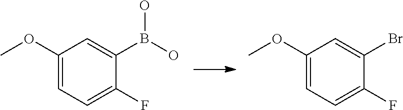 [0247] 328) To a solution of 2-fluoro-5-methoxy-phenyl boronic acid (5 g, 29.42 mmol) in MeOH (200 ml) was added copper (II) bromide (19.7 g, 88.26 mmol) and water (200 ml). The mixture was stirred at reflux overnight, then cooled to room temperature, and extracted with diethyl ether. The organic extract was washed with water and brine, dried over sodium sulfate, filtered, and concentrated. The residue was purified by flash chromatography with 20% EtOAc in hexane to give the desired product (4.6 g, 76% yield).

### 2-benzylamino-6-methoxy-1,2,3,4-tetrahydro-naphthane hydrochloride

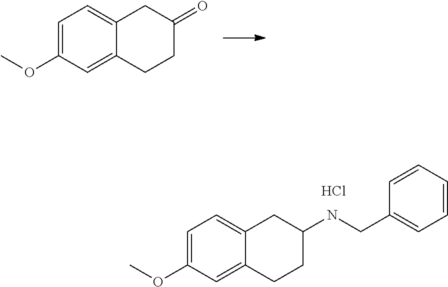 [0249] 329) To a solution of 6-methoxy-2-tetralone (2.15 g, 12.04 mmol) and benzylamine (1.33 ml, 12.04 mmol) in DCM (40 ml) was successively added at room temperature NaBH₃(OAc)₃ (3.63 g, 16.86 mmol) and AcOH (700 ul, 12.04 mmol). The mixture was stirred at room temperature for 2.5 h, quenched with 1NNaOH and extracted with EtOAc. The organic layer was washed with brine, dried over Na₂SO₄, filtered and concentrated. The residue was dissolved in 4NHO/dioxane and concentrated under reduced pressure to afford the HCl salt of the amine as a dark brown solid (3.4 g, 94% yield).

### 2-amino-6-methoxy-1,2,3,4-tetrahydro-naphthalene hydrochloride

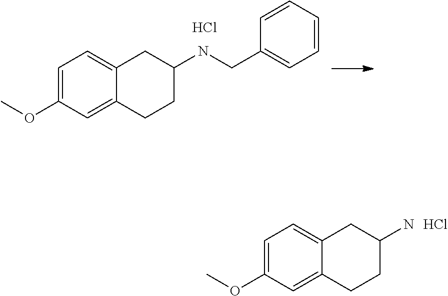 [0251] 330) The starting benzylamine (200 mg, 0.66 mmol) in THF/EtOH (10 ml/10 ml) was hydrogenated at room temperature over 10% Pd/C (150 mg) at 60 PSI for 24 h (Parr apparatus). The mixture was filtered through celite and concentrated under reduced pressure to afford the desired amine HCl salt as a light grey solid (90 mg, 70% yield).

### Preparation of 5-(3-Allyloxy-phenyl)-pentanoic acid

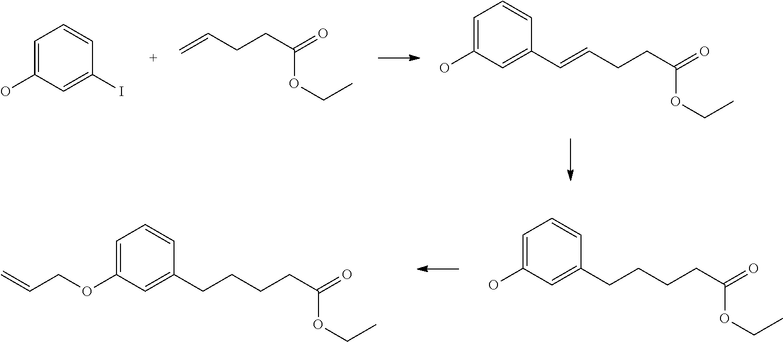 [0253] 331) To a solution of 3-iodo-phenol (1.1 g, 5 mmol) and 4-pentenoic acid ethyl ester (770 mg, 6 mmol) in DMF (20 ml) was successively added at room temperature K₂CO₃ (3.45 g, 5 mmol), nBu₄NCl (1.4 g, 5 mmol) and Pd(OAc)₂ (115 mg, 0.5 mmol). The mixture was heated to 70° C. and stirred for 1 h. The mixture was cooled to room temperature, quenched with 1N HCl and extracted with EtOAc. The combined organic layers were washed with brine, dried over Na₂SO₄, filtered and concentrated. The crude residue was purified by flash chromatography with a 10-50% ethyl acetate in hexane gradient to afford 5-(3-hydroxy-phenol)-4-pentenoic acid ethyl ester (794 mg, 72% yield). This ester (420 mg, 1.9 mmol) was dissolved in methanol (20 ml) and hydrogenated over 10% Pd/C (150 mg) at 50 PSI in a Parr apparatus to provide 5-(3-hydroxy-phenol)-4-petanoic acid ethyl ester after filtration and evaporation of the solvent (364 mg, 86% yield). To a solution of 5-(3-hydroxy-phenol)-4-petanoic acid ethyl ester (362 mg 1.63 mmol) in DMF was successively added at room temperature K₂CO₃ (1.12 g, 8.15 mmol) and allyl bromide (710 ul, 8.15 mmol). The mixture was stirred at 60° C. for 12 h then filtered and quenched with 1N HCl. The mixture was extracted with EtOAc. The layers were separated and the organic layer was washed with brine, dried over Na₂SO₄, filtered and concentrated under reduced pressure to afford 5-(3-allyloxy-phenyl)-petanoic acid ethyl ester (356 mg, 83% yield).

### 2-(3′-methoxy-phenyl)-benzylamine hydrochloride

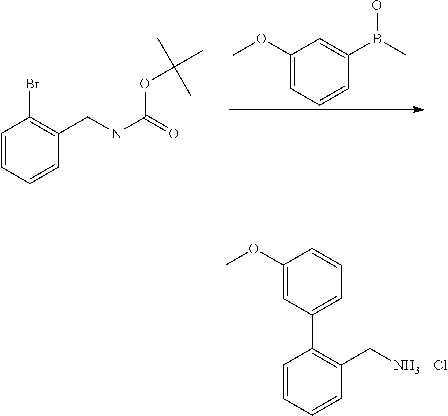 [0255] 332) A mixture of BOC-2-bromo-benzylamine (207 mg, 0.72 mmol), 3-methoxy-phenylboronic acid (165 mg, 1.08 mmol) and Pd[PPh₃]₄ (167 mg, 0.144 mmol) in DME/2MNa₂CO₃ (6 ml/1.26 ml) was microwaved at 150° C. for 10 min. The mixture was cooled to room temperature, quenched with 1NHCl and extracted with EtOAc. The organic layers were combined, successively washed with 1NNaOH and brine, then dried over Na₂SO₄, filtered and concentrated. The crude residue was purified by flash chromatography with a 5-30% EtOAc in hexane gradient to afford the desired product as a colorless oil. This material was treated with 4N HCl in dioxane (10 ml) at room temperature for 1 hr and concentrated to dryness to afford the desired amine HCl salt as a beige solid (73 mg, 40% overall yield).
333) A similar procedure was used to prepare the following analog:

### 2-(4′-methoxy-phenyl)-benzylamine hydrochloride

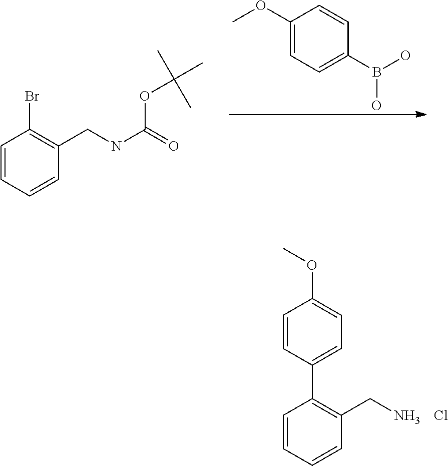

### Preparation of 2,2-dimethyl-propionic acid 1-chloro-ethyl ester

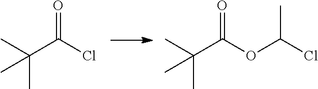 [0259] 334) A mixture containing anhydrous ZnCl₂ (1 g, 7.34 mmol) and trimethylacetyl chloride (37 ml, 300 mmol) was stirred at room temperature for 15 min, then cooled in a salt-ice-water bath. Acetaldehyde (26 ml, 450 mmol) was slowly added so that the reaction temperature did not exceed 0° C. The mixture was warmed up to room temperature, and quenched with ice water, The pH was adjusted to ˜7 with 10% NaHCO₃, and the mixture was extracted with EtOAc. The organic extracts were combined, washed with water and brine, dried over MgSO₄, filtered, and concentrated. The crude residue was distilled under vacuum to afford the desired chloro-ethyl ester as a colorless liquid (21 g, 43% yield).

### Preparation of 4,6-Dimethyl-2-hydroxy-1,6-dihydro-pyrimidine-5-carboxylic acid ethyl ester

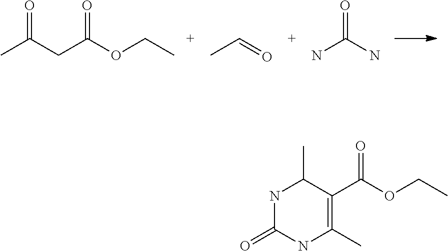 [0261] 335) A mixture of 3-oxo-butanoic acid ethyl ester (16.27 g, 125 mmol), acetaldehyde (5.51 g, 125 mmol), urea (7.51 g, 125 mmol), and glacial acetic acid (20 drops) in ethanol (35 ml) was heated to 90° C. overnight in a 350 ml pressure flask. The mixture was diluted with water. The precipitate was collected by filtration, washed with water and air-dried to afford the desired product (17.68 g, 71% yield). MS m/e 198.8 (M+H⁺).

### Preparation of 4,6-dimethyl-2-hydroxy-pyrimidine-5-carboxylic acid ethyl ester

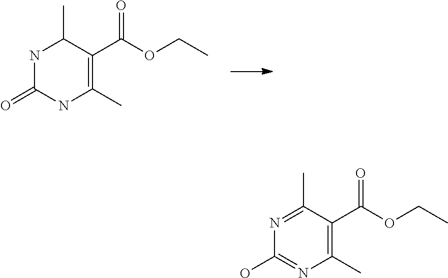 [0263] 336) 4,6-Dimethyl-2-hydroxy-1,6-dihydro-pyrimidine-5-carboxylic acid ethyl ester (34.63 g, 174.7 mmol) was added in portions to an ice-cooled solution of 50% nitric acid (120 ml) over minutes. The solution was stirred at 0° C. for 10 minutes, poured into ice water (500 ml), neutralized with solid K₂CO₃ and extracted with chloroform. The combined organic layers were washed with water and brine, dried over Na₂SO₄, filtered, and concentrated to afford title compound (21.9 g, 71% yield). MS m/e 197.1 (M+H⁺).

### Preparation of 2-Chloro-4,6-dimethyl-pyrimidine-5-carboxylic acid ethyl ester

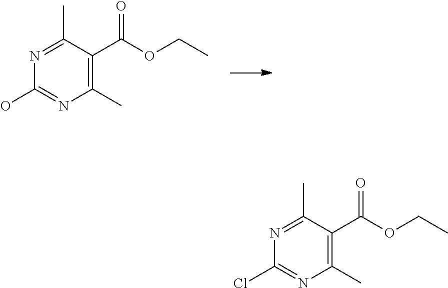 [0265] 337) To a solution of POCl₃ (106 ml) and DIEA (65 ml) was added 4,6-dimethyl-2-hydroxy-pyrimidine-5-carboxylic acid ethyl ester (21.9 mg, 111.6 mmol). The mixture was heated to 110° C. for 2 h. Excess POCl₃ and DIEA were removed by evaporation under reduced pressure. The residue was dissolved in EtOAc (1.21) and treated with decolorizing carbon. After filtration, the solution was washed with 1N NaOH, water and brine. The organic layer was dried over Na₂SO₄. filtered and concentrated. The crude residue was purified by flash chromatography with a 0-30% EtOAc in hexane gradient to afford the desired product (9.33 g, 39% yield).

### Preparation of 4-ethyl-2-hydroxy-6-methyl-1,6-dihydro-pyrimidine-5-carboxylic acid ethyl ester

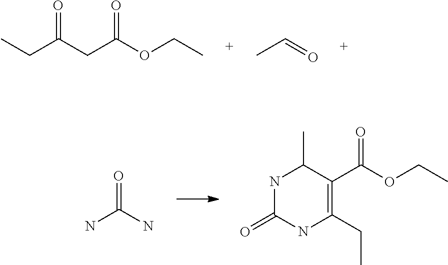 [0267] 338) A mixture of 3-oxo-pentanoic acid ethyl ester (5 g, 34.7 mmol), acetaldehyde (1.3 ml, 23.1 mmol), urea (1.4 g, 23.1 mmol), and concentrated HCl (10 drops) in ethanol (10 ml) was heated to 80° C. in a sealed tube for 24 h. The solution was loaded directly onto a column and purified by flash chromatography with a 20-80% EtOAC in hexane gradient to give the desired heterocycle (1.83 g, 25% yield). MS m/e 212.9 (M+H⁺).

### Preparation of 4-ethyl-2-hydroxy-6-methyl-pyrimidine-5-carboxylic acid ethyl ester

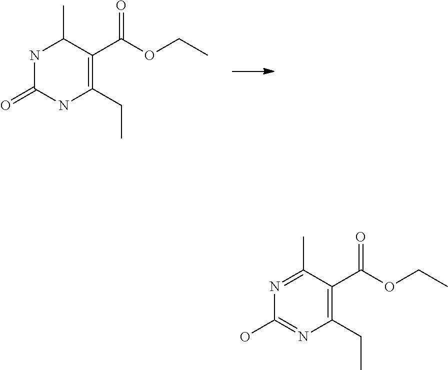 [0269] 339) To a solution of 50% nitric acid (5.5 ml, 86 mmol) cooled in ice-water bath was added in portions 4-ethyl-2-hydroxy-6-methyl-1,6-dihydro-pyrimidine-5-carboxylic acid ethyl ester (1.83 g, 8.6 mmol). The solution was stirred at 0° C. for 5 min, followed by treatment with K₂CO₃ at 0° C. to pH 5-6. The mixture was extracted with EtOAc. The organic extracts were combined, washed with water and brine, dried over Na₂SO₄, filtered and concentrated to give the product (579.5 mg, 32% yield). MS m/e 210.8 (M+H⁺).

### Preparation of 2-Chloro-4-ethyl-6-methyl-pyrimidine-5-carboxylic acid ethyl ester

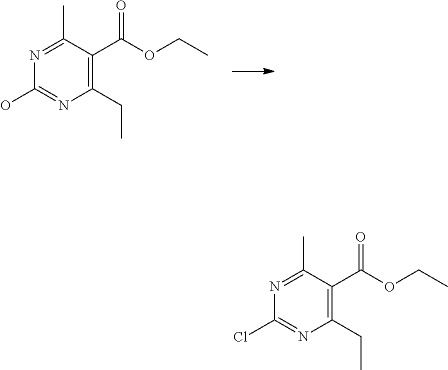 [0271] 340) To a reaction vessel containing POCl₃ (2.6 ml, 27.6 mmol) were successively added at room temperature diethyl-isopropyl-amine (1.6 ml, 9.2 mmol), and 4-ethyl-2-hydroxy-6-methyl-pyrimidine-5-carboxylic acid ethyl ester (579.5 mg, 2.76 mmol). The mixture was heated to 110° C. for 2 h. Excess POCl₃ and DIEA were removed by evaporation under reduced pressure. The residue was treated with EtOAc (200 ml) and basified with 10% NaOH at 0° C. The organic layer was separated, washed with brine, dried over sodium sulfate, filtered and evaporated to afford the desired product (289 mg, 46% yield).

### Preparation of 4,6-diethyl-2-hydroxy-1,6-dihydro-pyrimidine-5-carboxylic acid ethyl ester

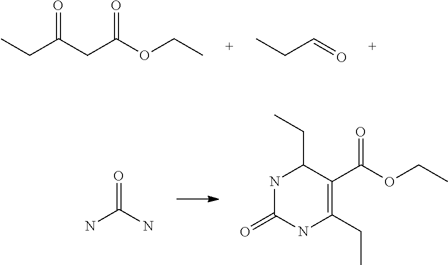 [0273] 341) A mixture of 3-oxo-pentanoic acid ethyl ester (5.3 g, 36.8 mmol), propionaldehyde (2.8 g, 55.14 mmol), urea (3.1 g, 55.14 mmol), and concentrated HCl (20 drops) in ethanol (10 ml) was heated to 160° C. for 1 h in a microwave oven. The solution was diluted with diethyl ether and the precipitate was removed by filtration. The filtrate was evaporated and the residue was purified by flash chromatography with a 50-100% EtOAC in hexane gradient to give the desired product (1.16 g, 14% yield). MS m/e 226.8 (M+H⁺).

### Preparation of 4,6-diethyl-2-hydroxy-pyrimidine-5-carboxylic acid ethyl ester

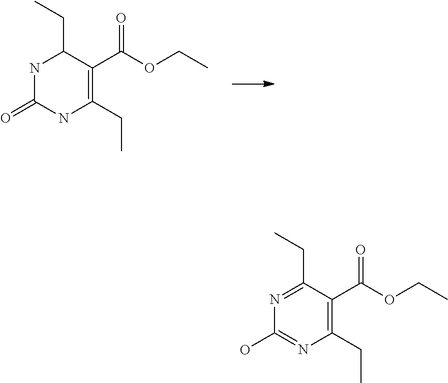 [0275] 342) A solution of 4,6-diethyl-2-hydroxy-1,6-dihydro-pyrimidine-5-carboxylic acid ethyl ester (359 mg, 1.59 mmol) in acetic acid (5 ml) was cooled in an ice-water bath and 50% nitric acid (8.6 ml) was added dropwise. The solution was stirred at 0° C. for 2 h. No desired product was detected by LC-MS. The solution was then treated with sodium nitrate (20 mg) at 0° C. for 30 min, followed by treatment with K₂CO₃ at 0° C. to raise the pH to ˜5-6. The mixture was extracted with EtOAc. The organic extracts were combined, washed with water and brine, dried over sodium sulfate, filtered and evaporated to give the desired product (195 mg, 55% yield). MS m/e 224.8 (M+H⁺).

### Preparation of 2-chloro-4,6-diethyl-pyrimidine-5-carboxylic acid ethyl ester

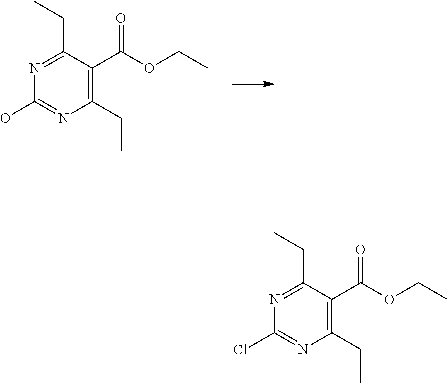 [0277] 343) A mixture of POCl₃ (2 ml) and 4,6-diethyl-2-hydroxy-pyrimidine-5-carboxylic acid ethyl ester (195 mg, 0.87 mmol) was heated to 120° C. for 6 h. The mixture was then cooled to room temperature, poured into ice, treated with aqNa₂CO₃, and extracted with EtOAc. The organic extract was washed with water and brine, dried over Na₂SO₄, filtered and concentrated to give the desired product (108 mg, 51% yield). MS m/e 242.8 (M+H⁺).

### Preparation of 2-Chloro-4-methyl-pyrimidine-5-carboxylic acid ethyl ester

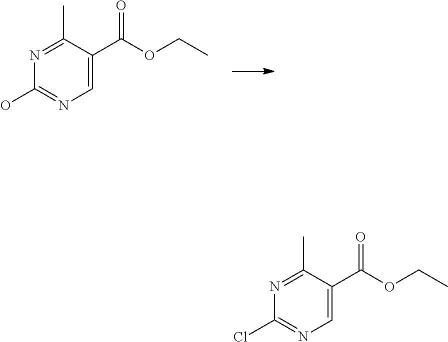 [0279] 344) A mixture of POCl₃ (10 ml) and 2-hydroxy-4-methyl-pyrimidine-5-carboxylic acid ethyl ester (Frontier, 1 g, 5.49 mmol) was heated to reflux for 2 h. Excess POCl₃ and diethyl iso-propylamine were removed by evaporation under vacuum. The residue was partitioned between 10% NaOH and EtOAc. The organic extract was washed with water and brine, dried over Na₂SO₄, filtered and concentrated to give the desired product (498 mg, 45% yield).

### Preparation of 2-[3-(3-Methoxy-phenyl)-propylamino]-pyrimidine-5-carboxylic acid methyl ester

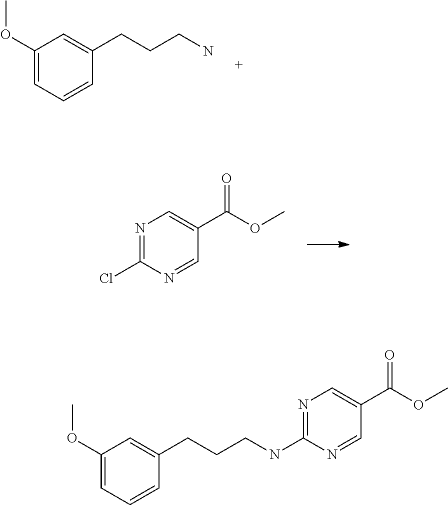 [0281] 345) A microwave reaction vessel was charged with 3-(methoxy-phenyl)-propylamine (1.44 g, 8.7 mmol), 2-chloro-pyrimidine-5-carboxylic acid methyl ester (Maybridge, 1 g, 5.8 mmol), potassium acetate (1.62 g, 16.5 mmol) and methanol (10 ml). The mixture was heated to 150° C. for 1 h in a microwave oven, then cooled to room temperature and quenched with cold water (4 ml). The precipitate was collected by filtration and washed with water and cold methanol to afford the desired product (1.73 g, 99% yield).

### Preparation of 2-[3-(3-Hydroxy-phenyl)-propylamino)-pyrimidine-5-carboxylic acid methyl ester

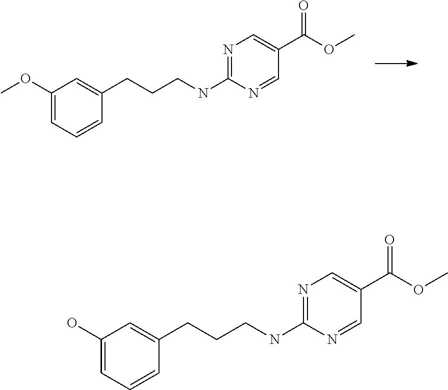 [0283] 346) A solution of 2-[3-(3-methoxy-phenyl)-propylamino]-pyrimidine-5-carboxylic acid ethyl ester (1.16 g, 3.84 mmol) in anhydrous DCM (10 ml) was cooled in an ice-water bath and BBr3/DCM (1M, 7.7 ml, 7. mmol) was added dropwise. The resulting solution was allowed to warm up to room temperature and stirred for 2 h, then quenched with ice water. The organic layer was separated and the aqueous layer was extracted by DCM. The organic extracts were combined, washed with water and brine, dried over magnesium sulfate, filtered, and evaporated to afford the desired product (1 g, 91% yield). MS m/e 287.9 (M+H⁺).

### Preparation of 2-[3-(3-hydroxy-phenyl)-propylamino]-pyrimidine-5-carboxylic acid

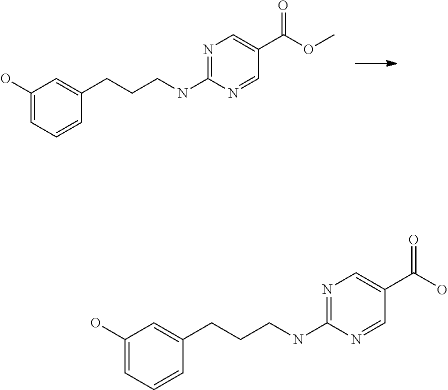 [0285] 347) A mixture of 2-[3-(3-hydroxy-phenyl)-propylamino]-pyrimidine-5-carboxylic acid methyl ester (1 g, 3.4 mmol) and lithium hydroxide monohydrate (2.9 g, 69.7 mmol), in dioxane/water (50 ml/50 ml) was stirred at reflux overnight, then cooled to room temperature and quenched with aqueous potassium hydrogen sulfate to adjust the pH to ˜2-4. The resulting solution was extracted with EtOAc. The organic extracts were combined, washed with brine, dried over sodium sulfate, filtered and evaporated to give the title compound (900 mg, 95% yield). MS m/e 273.9 (M+H⁺).

### Preparation of 2-[3-(3-methoxy-phenyl)-propylamino]-4,6-dimethyl-pyrimidine-5-carboxylic acid ethyl ester

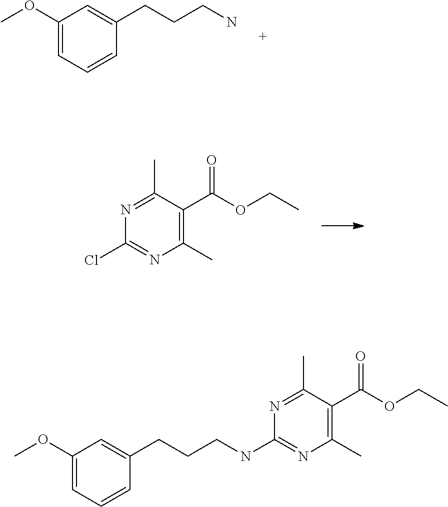 [0287] 348) A mixture of 3-(3-methoxy-phenyl)-propylamine (2.31 g, 13.98 mmol), 2-chloro-4,6-dimethyl-pyrimidine-5-carboxylic acid ethyl ester (2 g, 9.32 mmol) in EtOH (12 ml) was microwaved at 160° C. for 1.5 h. The reaction mixture was cooled to room temperature, quenched with 10% K₂CO₃ and extracted with EtOAc. The organic layer was washed with brine, dried over Na₂SO₄, filtered and concentrated. The residue was purified by flash chromatography with 30% EtOAC in hexane to afford the desired product (2.42 g, 76% yield). MS m/e 344.1 (M+H⁺).

### Preparation of 2-[3-(3-hydroxy-phenyl)-propylamino]-4,6-dimethyl-pyrimidine-5-carboxylic acid ethyl ester

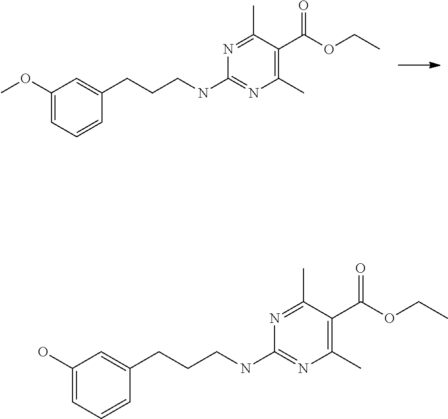

A solution of 2-[3-(3-methoxy-phenyl)-propylamino]-4,6-dimethyl-pyrimidine-5-carboxylic acid ethyl ester (2.42 g, 7.05 mmol) DCM (50 ml) was cooled in an ice-water bath and BBr3/DCM (1M, 14.1 ml, 14.1 mmol) was added dropwise. The resulting solution was allowed to warm up to room temperature and stirred at room temperature for 2 h. The solution was quenched with ice water and extracted with DCM. The organic layers were combined, washed with water and brine, dried over MgSP₄, filtered, and concentrated to afford the desired product (2 g, 86% yield). MS m/e 330.1 (M+H⁺).

### Preparation of 4-Ethyl-2-[3-(3-methoxy-phenyl)-propylamino]-6-methyl-pyrimidine-5-carboxylic acid ethyl ester

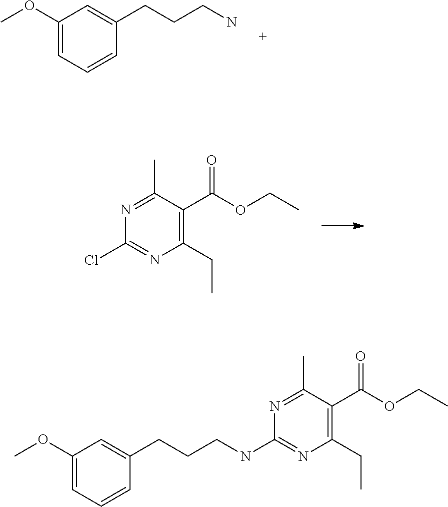

A mixture of 3-(methoxy-phenyl)-propylamine hydrochloride (254 mg, 1.26 mmol), 2-chloro-4,6-dimethyl-pyrimidine-5-carboxylic acid ethyl ester (288 g, 1.26 mmol) and KOAc (495 mg, 5.04 mmol) in ethanol (10 ml) was microwaved at 120° C. for 1 h. The reaction mixture was cooled to room temperature, quenched with 20% NaHCO₃ and extracted with EtOAc. The organic layer was washed with brine, dried over Na₂SO₄, filtered and concentrated. The residue was purified flash chromatography with 30% EtOAC in hexane to afford the desired product (303.3 mg, 67% yield). MS m/e 358.0 (M+H⁺).

### Preparation of 4-ethyl-2-[3-(3-hydroxy-phenyl)-propylamino]-6-methyl-pyrimidine-5-carboxylic acid ethyl ester

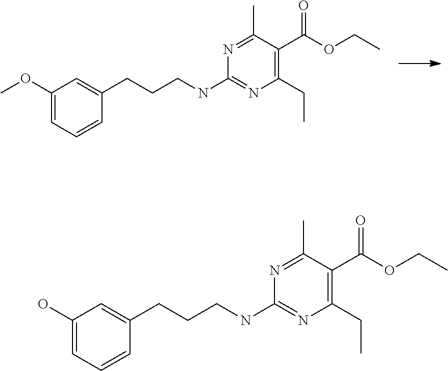

A solution of 4-ethyl-2-[3-(3-methoxy-phenyl)-propylamino]-6-methyl-pyrimidine-5-carboxylic acid ethyl ester (303.3 mg, 0.85 mmol) in DCM (5 ml) was cooled in an ice-water bath and BBr3/DCM (1M, 1.7 ml, 1.7 mmol) was added dropwise. The resulting solution was allowed to warm up to room temperature and stirred for a further 2 h. The solution was quenched with ice water and extracted with DCM. The organic layers were combined, washed with water and brine, dried over MgSO₄, filtered, and concentrated to afford the desired product (243.8 mg, (83% yield). MS m/e 344.0 (M+H⁺).

### Preparation of 4-ethyl-2-[3-(3-hydroxy-phenyl)-propylamino]-6-methyl-pyrimidine-5-carboxylic acid

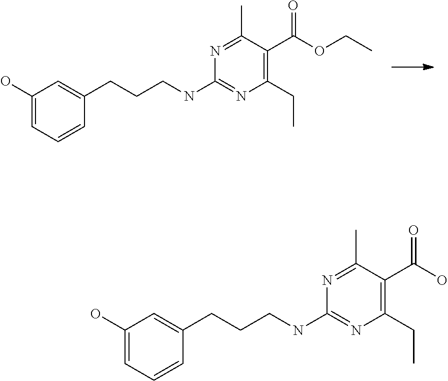

A mixture of 4-ethyl-2-[3-(3-hydroxy-phenyl)-propylamino]-6-methyl-pyrimidine-5-carboxylic acid ethyl ester (244 mg, 0.71 mmol) and LiOH.H₂O (745 mg, 17.8 mmol) in dioxane/water (10 ml/10 ml) was heated to 90° C. for 6 h. The solution was cooled to room temperature, treated with aqKHSO₃ to bring the pH to 2-4 and extracted with EtOAc. The organic extracts were combined, washed with brine, dried over sodium sulfate, filtered and evaporated to give the title compound (219 mg, 98% yield). MS m/e 315.9 (M+H⁺).

### Preparation of 4,6-diethyl-2-[3-(3-methoxy-phenyl)-propylamino]-pyrimidine-5-carboxylic acid ethyl ester

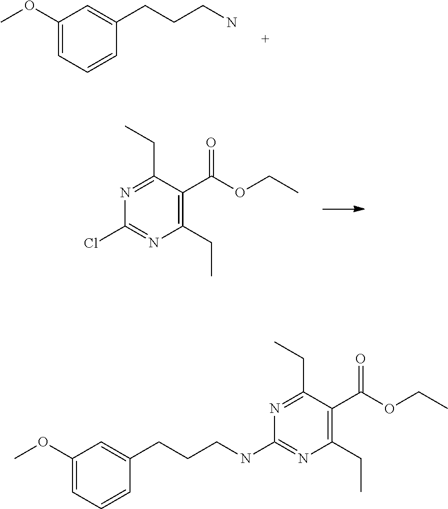

In a microwave reaction vessel, was charged with 3-(methoxy-phenyl)-propylamine hydrochloride (135 mg, 0.67 mmol), 2-chloro-4,6-diethyl-pyrimidine-5-carboxylic acid ethyl ester (108 mg, 0.45 mmol), potassium acetate (177 mg, 1.8 mmol) and ethanol (4 ml). The mixture was heated to 150° C. for 1 h in a microwave oven. The mixture was diluted with EtOAc (150 ml), washed with 20% aqNaHCO₃, water and brine, dried over sodium sulfate, filtered, and concentrated. The residue was purified by flash chromatography with 30% EtOAc in hexane to afford the desired product (45 mg, 27% yield). MS m/e 372.0 (M+H⁺).

### Preparation of 4,6-Diethyl-2-[3-(3-hydroxy-phenyl)-propylamino]-pyrimidine-5-carboxylic acid ethyl ester

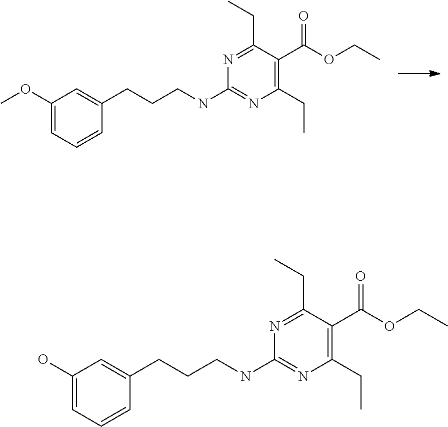

A solution of 4,6-diethyl-2-[3-(3-methoxy-phenyl)-propylamino]-pyrimidine-5-carboxylic acid ethyl ester (45 mg, 0.121 mmol) in DCM (1 ml) was cooled in an ice-water bath and BBr3/DCM (1M, 0.24 ml, 0.24 mmol) was added dropwise. The resulting solution was allowed to warm up to room temperature and stirred for a further 2 h. The solution was quenched with ice water and extracted with DCM. The organic layers were combined, washed with water and brine, dried over MgSO₄, filtered, and concentrated to give the desired product (30 mg, 69% yield). MS m/e 357.9 (M+H⁺).

### Preparation of 4,6-diethyl-2-[3-(3-hydroxy-phenyl)-propylamino]-pyrimidine-5-carboxylic acid

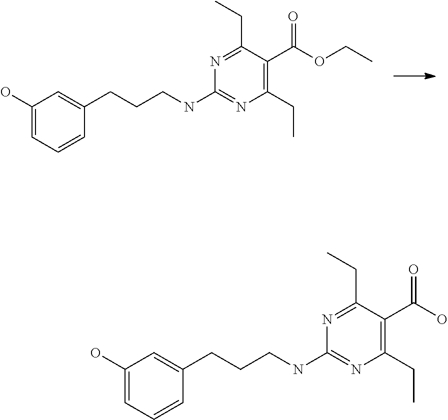

A mixture of 4,6-diethyl-2-[3-(3-hydroxy-phenyl)-propylamino]-pyrimidine-5-carboxylic acid ethyl ester (30 mg, 0.084 mmol), and LiOH.H₂O (88 mg, 2.1 mmol), in dioxane/water (2 ml/2 ml) was heated to 90° C. overnight. The solution was cooled to room temperature, treated with aqKHSO₄ to bring the pH to 2-4 and extracted with EtOAc. The organic extracts were combined, washed with brine, dried over sodium sulfate, filtered and concentrated to give the title compound (26 mg, 94% yield). MS m/e 329.9 (M+H⁺).

### Preparation of 2-[4-(3-Allyloxy-phenyl)-butyl]-4,6-dimethyl-pyrimidine-5-carboxylic acid

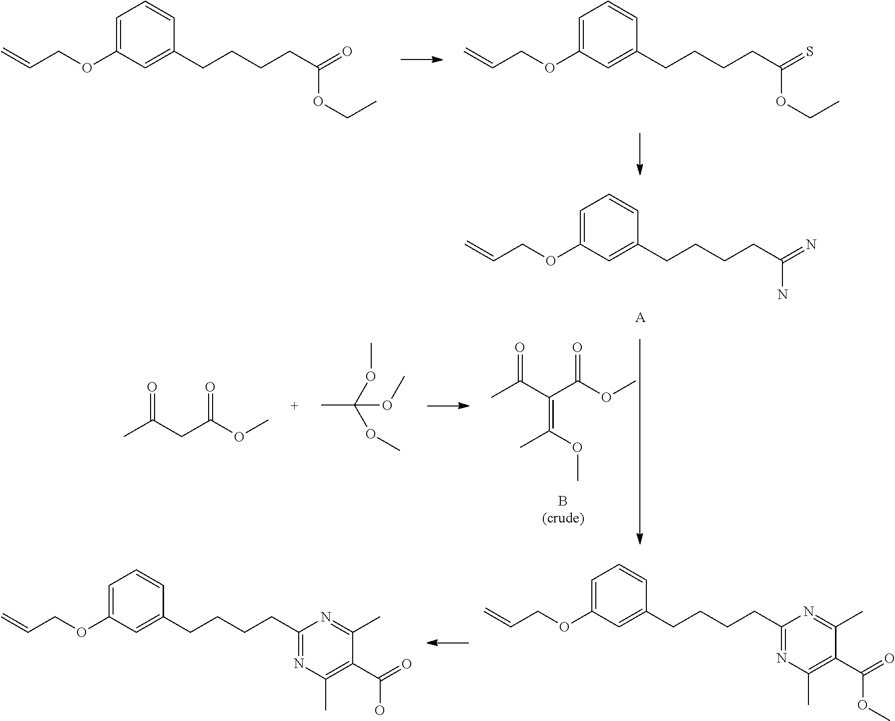

To a solution of 5-(3-allyloxy-phenyl)-pentanoic acid ethyl ester (356 mg, 1.35 mmol) in xylenes (15 ml) was added Lawesson's reagent (655 mg, 1.62 mmol). The mixture was refluxed for 3 h. After cooling to room temperature, a precipitate was removed by filtration and the solution was concentrated to dryness. The crude residue was purified by flash chromatography with a 5-10% EtOAc in hexane gradient to afford the corresponding thioester (92 mg, 24% yield). This thioester was dissolved in MeOH (2 ml) and treated with ammonia (7N in MeOH, 462 ul, 3.23 mmol) and ammonium chloride (173 mg, 3.23 mmol). The mixture was refluxed for 40 min, cooled to room temperature, quenched with 1N NaOH and extracted with EtOAc. The organic layer was concentrated under reduced pressure to afford the desired amidine as a yellow paste (67 mg, 89% yield).

A solution of methyl acetoacetate (10.8 ml, 0.1 mmol), trimethyl orthoformate (15.2 ml, 0.12 mmol), pyridine (0.8 ml, 0.01 mmol) and acetic acid (0.6 ml, 0.01 mmol) in toluene (30 ml) was stirred at 80° C. for 18 h. The mixture was concentrated under reduced pressure to afford crude keto ester B as an orange oil. This crude reagent (400 ul) was dissolved in methanol (1 ml) and the amidine A (0.29 mmol, 67 mg) was added in one portion. The mixture was refluxed for 26 h, cooled to room temperature and concentrated under reduced pressure. The crude residue was purified by flash chromatography with a 10-40% EtOAc in hexane gradient to afford 2-[4-(3-Allyloxy-phenyl)-butyl]-4,6-dimethyl-pyrimidine-5-carboxylic acid ethyl ester. This ester was then hydrolyzed with LiOH.H₂O (0.35 mmol, 15 mg) in dioxane/H₂O (2 ml/0.5 ml) at reflux for 2 h. The mixture was quenched with pH=3 buffer and extracted with EtOAc. The layers were separated; the organic layer was dried over Na₂SO₄, filtered and concentrated to afford the desired acid as a white solid (67 mg, 65% overall yield from amidine A).

### Preparation of 2-[3-(3-methoxy-phenyl)-propoxy]-pyrimidine-5-carboxylic acid ethyl ester

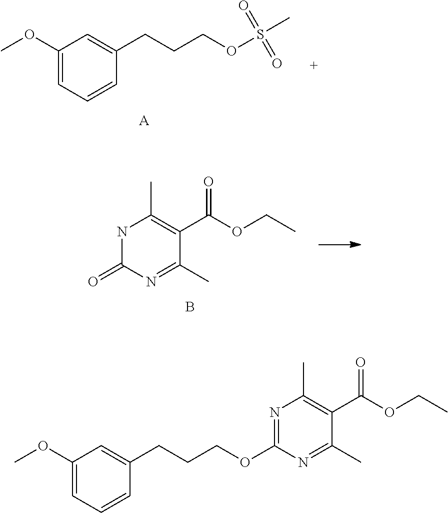

The pyrimidinone B (5.96 g, 30.4 mmol) and the mesylate A (4.95 g, 20.26 mmol) were dissolved in dry DMF (100 ml) containing Cs₂CO₃ (13.2 g, 40 mmol) and heated to 75° C. for 6 h. The mixture was cooled to room temperature, poured into water and extracted with EtOAc. The combined organic layers were successively washed with 1N HCl, water and brine, then dried over MgSO₄, filtered and concentrated under reduced pressure. The crude was purified by flash chromatography (10% EtOAc/Hexanes) to afford the desired product as a yellow oil (5.6 g, 54% yield)

### Preparation of 2-[3-(3-hydroxy-phenyl)-propoxy]-Pyrimidine-5-carboxylic acid

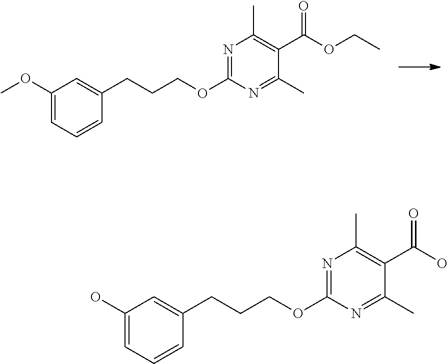

A solution of 2-[3-(3-methoxy-phenyl)-propoxy]-pyrimidine-5-carboxylic acid ethyl ester (2.5 g, 7.3 mmol) in dry DCM (25 ml) was cooled to 0° C. in a ice bath and BBr3/DCM (1M, 14.6 ml, 14.6 ml) was added dropwise at 0° C. After 5 min, the cooling bath was removed and the mixture was allowed to warm up to room temperature and was stirred for 2 h. The mixture was quenched with water and extracted with DCM. The combined organic layers were washed with saturated aqueous NaHCO₃ and brine, then dried over MgSO₄, filtered and concentrated under reduced pressure to afford a yellow oil (2.3 g). This material was dissolved in MeOH (25 ml) and treated with 1N NaOH (15 ml) at 70° C. for 8 h. The mixture was concentrated under reduced pressure and partitioned between an aqueous buffer (pH=5) and EtOAc. The organic phases was washed with water and brine, then dried over MgSO₄, filtered and concentrated under reduced pressure to afford the desired acid as a beige solid (1.25 g, 57% yield).

### 2-[(3% methoxy-biphenyl-2-ylmethyl)-amino)]-4,6-dimethyl-pyrimidine-5-carboxylic acid ethyl ester

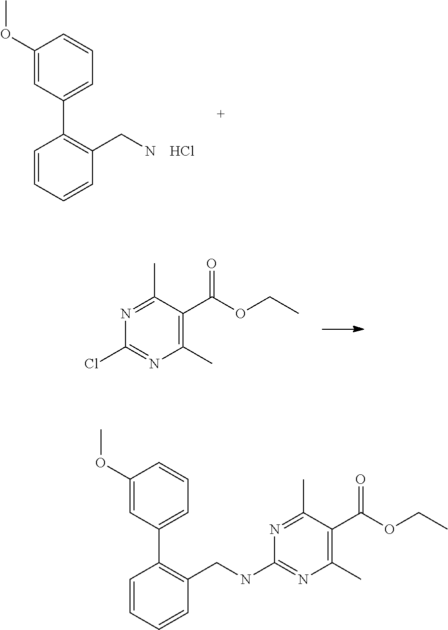

A mixture of 3′-methoxy-biphenyl-2-ylmethyl-amine hydrochloride salt (73 mg, 0.29 mmol), 2-chloro-4,6-dimethyl-pyrimidine-5-carboxylic acid ethyl ester (42 mg, 0.19 mmol) and KOAc (66 mg, 0.66 mmol) in ethanol (3 ml) was microwaved at 120° C. for 1 h. The reaction mixture was cooled to room temperature, quenched with 1NHCl and extracted with EtOAc. The organic layer was washed with brine, dried over Na₂SO₄, filtered and concentrated to afford the desired product (80 mg, 100% yield).

A similar procedure was used to make the following two intermediates:

### 2-[(4% methoxy-biphenyl-2-ylmethyl)-amino)]-4,6-dimethyl-pyrimidine-5-carboxylic acid ethyl ester

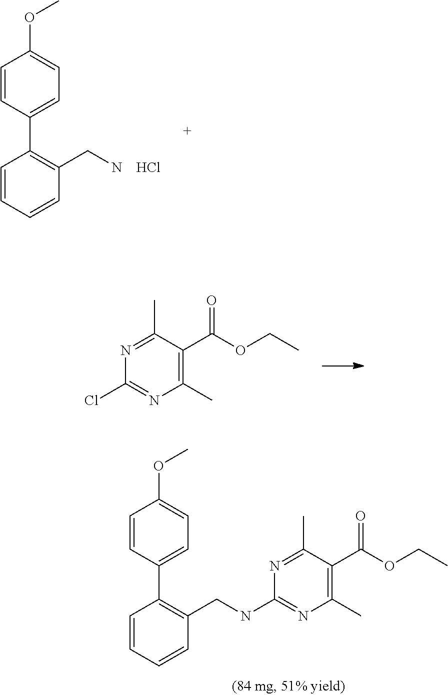

### 2-[(2% methoxy-biphenyl-2-ylmethyl)-amino)]-4,6-dimethyl-pyrimidine-5-carboxylic acid ethyl ester

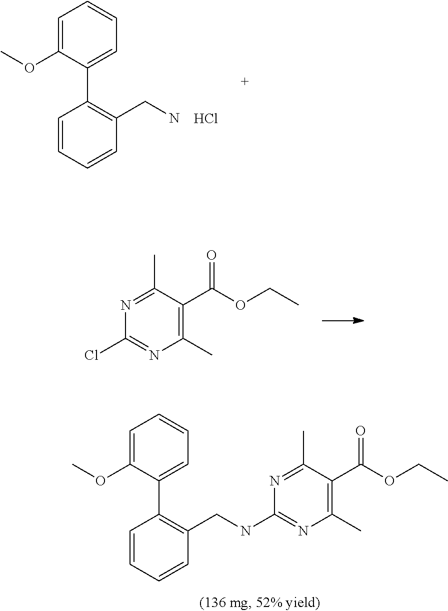

### 2-[(3′-hydroxy-biphenyl-2-ylmethyl)-amino]-4,6-dimethyl-pyrimidine-5-carboxylic acid

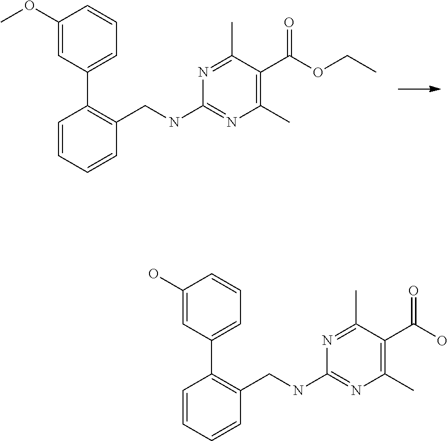

A solution of 2-[(3′-methoxy-biphenyl-2-ylmethyl)-amino]-4,6-dimethyl-pyrimidine-5-carboxylic acid ethyl ester (80 mg, 0.19 mmol) in dry DCM (2 ml) was cooled to 0° C. in a ice bath, and BBr3/DCM (1M, 380 ul, 0.38 mmol) was added dropwise at 0° C. The reaction mixture was stirred at 0° C. for 1 h, then quenched with water and extracted with DCM. The combined organic layers were washed with water and brine, then dried over Na₂SO₄, filtered and concentrated under pressure to afford the de-methylated compound which was not purified. This material was dissolved in H₂O/dioxane (1.5 ml/1.5 ml) and treated with LiOH.H₂O (80 mg, 1.9 mmol)) at 60° C. overnight. The mixture was quenched with concentrated HCl to pH=1, concentrated under reduced pressure to ½ its volume, then partitioned between brine and EtOAc. The combined organic phases were dried over Na₂SO₄, filtered and concentrated under reduced pressure to afford the desired acid (75 mg, 100% overall yield).

A similar procedure was used to make the following two intermediates:

### 2-[(4′-hydroxy-biphenyl-2-ylmethyl)-amino]-4,6-dimethyl-pyrimidine-5-carboxylic acid

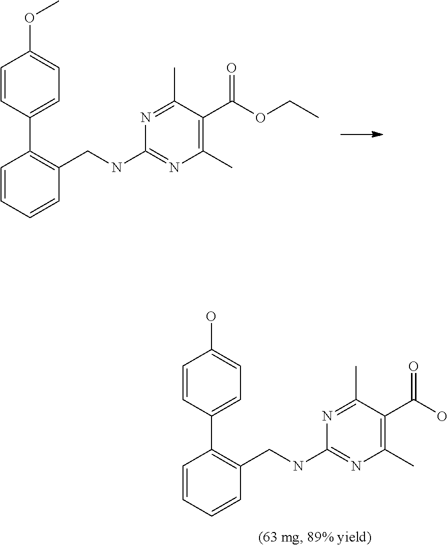

### 2-[(2′-hydroxy-biphenyl-2-ylmethyl)-amino]-4,6-dimethyl-pyrimidine-5-carboxylic acid

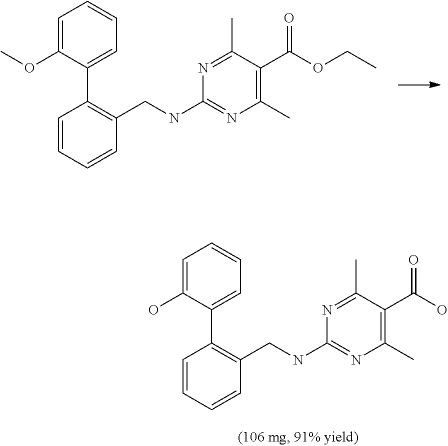

### 2-(6-methoxy-1,2,3,4-tetrahydro-naphthalen-2-ylamino)-4,6-dimethyl-pyrimidine-5-carboxylic acid ethyl ester

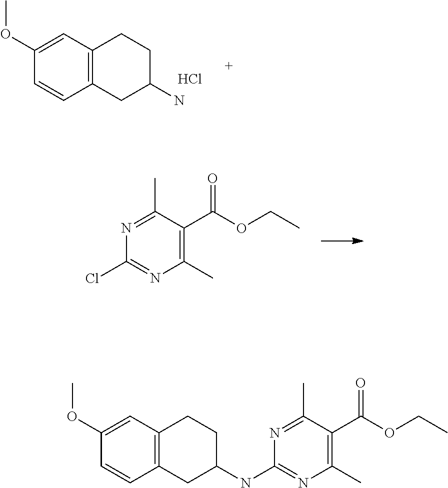

A mixture of 2-amino-6-methoxy-1,2,3,4-tetrahydro-naphthalene hydrochloride (87 mg, 0.43 mmol), 2-chloro-4,6-dimethyl-pyrimidine-5-carboxylic acid ethyl ester (71 mg, 0.33 mmol) and KOAc (150 mg, 1.5 mmol) in ethanol (3 ml) was microwaved at 120° C. for 1 h. The reaction mixture was cooled to room temperature, quenched with 1NHCl and extracted with EtOAc. The organic layer was washed with brine, dried over Na₂SO₄, filtered and concentrated. The crude residue was purified by flash chromatography with a 5-60% EtOAC in hexane gradient to afford the desired product (69 mg, 60% yield).

A similar procedure was used to make the following intermediate:

### 2-(8-methoxy-1,2,3,4-tetrahydro-naphthalen-2-ylamino)-4,6-dimethyl-pyrimidine-5-carboxylic acid ethyl ester

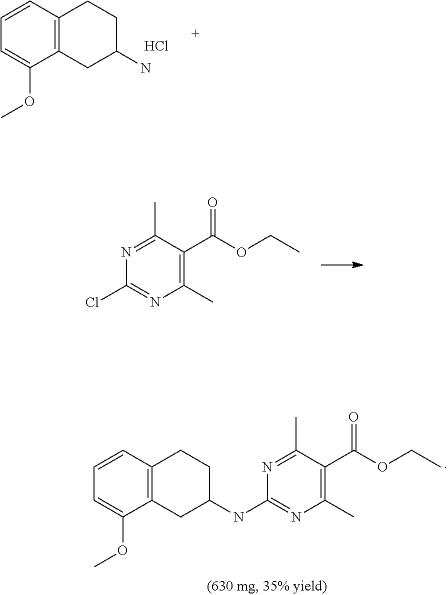

### 2-(6-hydroxy-1,2,3,4-tetrahydro-naphthalen-2-ylamino)-4,6-dimethyl-pyrimidine-5-carboxylic acid

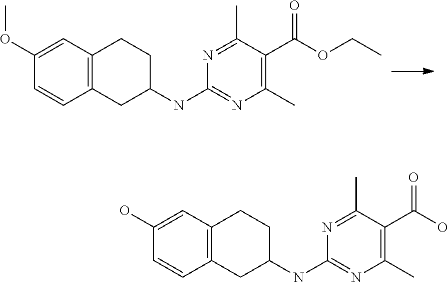

A solution of 2-(6-methoxy-1,2,3,4-tetrahydro-naphthalen-2-ylamino)-4,6-dimethyl-pyrimidine-5-carboxylic acid ethyl ester (36 mg, 0.1 mmol) in dry DCM (2 ml) was cooled to 0° C. in a ice bath, and BBr3/DCM (1M, 200 ul, 0.2 mmol) was added in one portion at 0° C. The reaction mixture was stirred at 0° C. for 1 h, then quenched with ice/water and extracted with DCM. The combined organic layers were dried over Na₂SO₄, filtered and concentrated under pressure to afford the de-methylated compound which was not purified. This material was dissolved in H₂O/dioxane (1.0 ml/1.0 ml) and treated with LiOH.H₂O (42 mg, 1.0 mmol)) at 60° C. overnight. The mixture was quenched with 1NHCl/brine (1:1) and extracted with EtOAc. The combined organic phases were dried over Na₂SO₄, filtered and concentrated under reduced pressure to afford the desired acid (30 mg, 100% overall yield).

### 2-(8-hydroxy-1,2,3,4-tetrahydro-naphthalen-2-ylamino)-4,6-dimethyl-pyrimidine-5-carboxylic acid

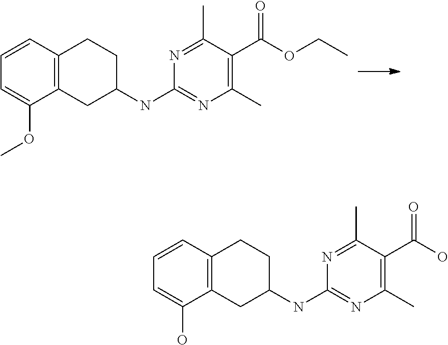

A solution of 2-(8-methoxy-1,2,3,4-tetrahydro-naphthalen-2-ylamino)-4,6-dimethyl-pyrimidine-5-carboxylic acid ethyl ester (261 mg, 0.73 mmol) in dry DCM (7 ml) was cooled to 0° C. in a ice bath, and BBr3/DCM (1M, 1.5 ml, 1.47 mmol) was added in one portion at 0° C. The reaction mixture was stirred at 0° C. for 1 h, then quenched with ice/water and extracted with DCM. The combined organic layers were dried over Na₂SO₄, filtered and concentrated under pressure to afford the de-methylated compound which was not purified. This material was dissolved in H₂O/dioxane (5.0 ml/5.0 ml) and treated with LiOH.H₂O (300 mg, 7.3 mmol)) at 60° C. overnight. The mixture was quenched with 1NHCl/brine (1:1) and extracted with EtOAc. The combined organic phases were dried over Na₂SO₄, filtered and concentrated under reduced pressure to afford the desired acid (166 mg, 72% overall yield).

### Preparation of 2-[2-(4-Hydroxy-phenyl)-ethylamino]-4,6-dimethyl-pyrimidine-5-carboxyic acid ethyl ester

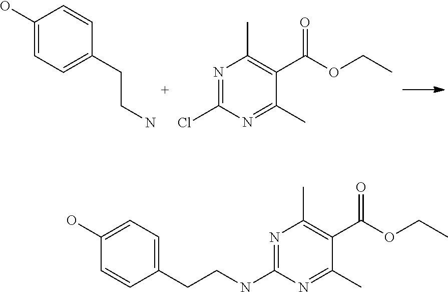

A microwave reaction vessel was charged with 2-chloro-4,6-dimethyl-pyrimidine-5-carboxylic acid ethyl ester (208 mg, 0.857 mmol), 4-(2-amino-ethyl)-phenol hydrochloride (223 mg, 1.286 mmol), potassium acetate (336 mg, 3.43 mmol), and ethanol (4 ml), then heated to 150° C. for 1 h in a microwave oven. The mixture was cooled to room temperature and quenched with cold water. The precipitate was collected by filtration, washed with water and dried over sodium sulfate to afford the desired product (285 mg, 100% yield). MS m/e 315.9 (M+H⁺).

### Preparation of 2-[2-(4-Hydroxy-phenyl)-ethylamino]-4,6-dimethyl-pyrimdine-5-carboxylic acid

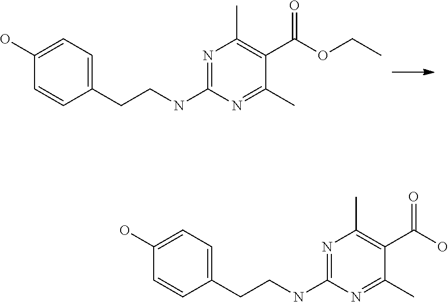

A solution of 2-[2-(4-hydroxy-phenyl)-ethylamino]-4,6-dimethyl-pyrimidine-5-carboxylic acid ethyl ester (285 mg, 0.90 mmol) in dioxane (10 ml) was treated with a solution of lithium hydroxide monohydrate (948 mg, 22.59 mmol) in water (10 ml). The mixture was stirred at 90° C. for 12 h, then cooled to room temperature and quenched with aqueous potassium hydrogen sulfate to adjust the pH to ˜2-4. The resulting solution was extracted with EtOAc. The organic extracts were combined, washed with brine, dried over sodium sulfate, filtered and concentrated to give the title compound (298 mg, 100% yield). MS m/e 287.9 (M+H⁺).

### Preparation of 4,6-dimethyl-2-prop-2-ynylamino-pyrimidine-5-carboxylic acid ethyl ester

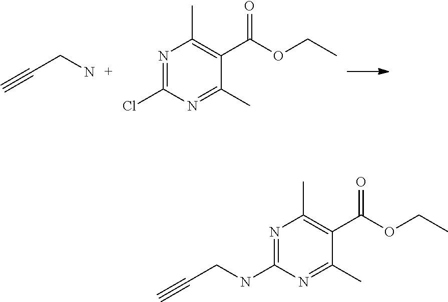

A solution of 2-chloro-4,6-dimethyl-pyrimidine-5-carboxylic acid ethyl ester (1 g, 4.66 mmol) and propargylamine (0.45 ml, 7.0 mmol) in EtOH (10 ml) was microwaved at 160° C. for 1 h. The mixture was cooled to room temperature, quenched with 10% aqK₂CO₃ and extracted with EtOAc. The combined organic extracts were washed water and brine, dried over Na₂SO₄, filtered and concentrated to give the product as a yellow solid which was not further purified (1.05 g, 92% yield). MS m/e 234.0 (M+H⁺).

### Preparation of 4,6-dimethyl-2-prop-2-ynylamino-pyrimidine-5-carboxylic acid

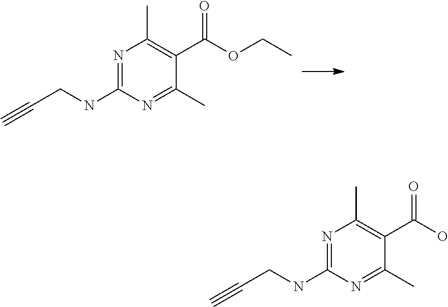

To a solution of 4,6-dimethyl-2-prop-2-ynylamino-pyrimidine-5-carboxylic acid ethyl ester (4.79 g, 20.53 mmol) in dioxane (100 ml) was added a solution of lithium hydroxide monohydrate (21.53 g, 513.25 mmol) in water (100 ml). The mixture was stirred at 90° C. for 6 h, then cooled to room temperature and quenched with a solution of KHSO₄ (80 g, 588 mmol) in water (1000 ml) to bring the pH to 2-4. The mixture was extracted with EtOAc. The organic extracts were combined, washed with brine, dried over sodium sulfate, and concentrated to give a the desired product as a white solid (3 g, 71% yield). MS m/e 205.9 (M+H⁺).

### Preparation of 4,6-dimethyl-2-[(E)-3-(4,4,5,5-tetramethyl-[1,3,2]dioxaborolan-2-yl)-allylamino]-pyrimidine-5-carboxylic acid ethyl ester

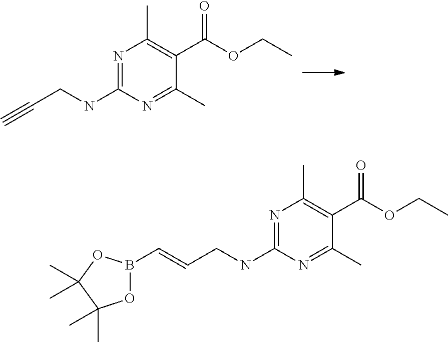

A solution of 4,6-dimethyl-2-prop-2-ynylamino-pyrimidine-5-carboxylic acid (1 g, 4.3 mmol), 4,4,5,5-tetramethyl-[1,3,2]dioxaborolane (0.75 ml, 5.16 mmol), bis(cyclopentadienyl)zirconium(IV) chloride hydride (11 mg, 0.43 mmol), and triethylamine (0.6 ml, 0.43 mmol) in THF (10 ml) was microwaved at 150° C. for 20 min Excess 4,4,5,5-tetramethyl-[1,3,2]dioxaborolane (1.5 ml, 10.32 mmol) was added and the solution was microwaved again at 150° C. for 20 h. The mixture was directly loaded onto a silica gel column and purified by flash chromatography with 40% EtOAc in hexane to give the desired product (1.19 g, 77% yield). MS m/e 362.0 (M+H⁺).

### Preparation of 2-[(E)-3-(3-Hydroxy-4-methyl-phenyl)-allylamino]-4,6-dimethyl-pyrimidine-5-carboxylic acid ethyl ester

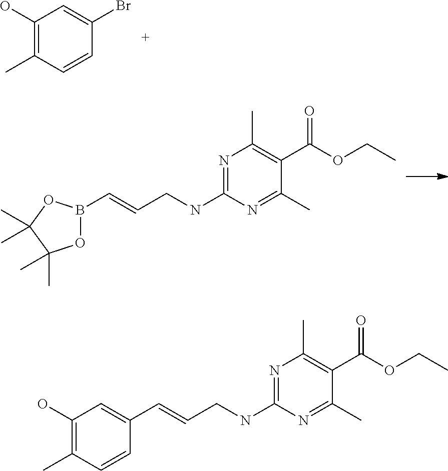

A microwave reaction vessel was charged with 4,6-dimethyl-2-[(E)-3-(4,4,5,5-tetramethyl-[1,3,2]dioxaborolan-2-yl)-allylamino]-pyrimidine-5-carboxylic acid ethyl ester (300 mg, 0.83 mmol), 5-bromo-2-methyl-phenol (155 mg, 0.83 mmol), [1,1′-bis (diphenylphosphinoferrocene)dichloropalladium(II) DCM complex (34 mg, 0.042 mmol), potassium carbonate (229 mg, 1.66 mmol), DMF (6 ml), and water (1 ml). The mixture was degassed and flushed with nitrogen, followed by heating to 135° C. for 15 min in a microwave oven. The mixture was partitioned between EtOAc and water. The organic layer was separated, washed with brine, dried over sodium sulfate, filtered, and concentrated. The residue was purified by flash chromatography with 30% EtOAc in hexane to give the desired product (145 mg, 51% yield). MS m/e 342.0 (M+H⁺).

### Preparation of 2-[3-(3-hydroxy-4-methyl-phenyl)-propylamino]-4,6-dimethyl-pyrimidine-5-carboxylic acid ethyl ester

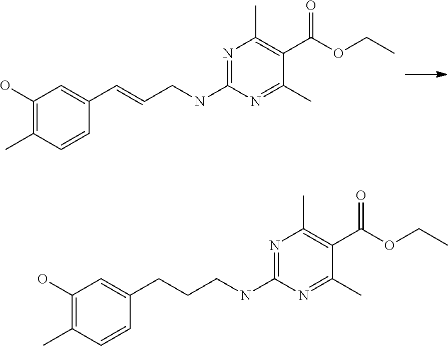

A solution of 2-[(E)-3-(3-hydroxy-4-methyl-phenyl)-allylamino]-4,6-dimethyl-pyrimidine-5-carboxylic acid ethyl ester (145 mg, 0.42 mmol) in EtOH (80 ml) was passed through H-Cube with 10% Pd/C small cartridge at 10 bar, room temperature and 1 ml per min. Solvent was removed by evaporation and the crude product was used directly in the next step. MS m/e 344.0 (M+H⁺).

### Preparation of 2-[3-(3-hydroxy-4-methyl-phenyl)-propylamino]-4,6-dimethyl-pyrimidine-5-carboxylic acid

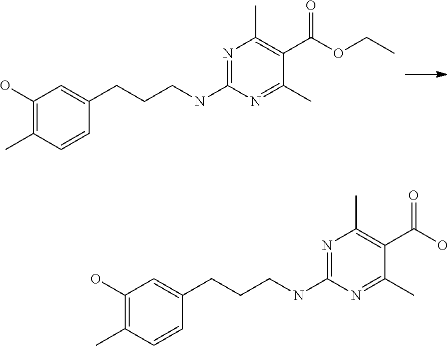

A mixture of 2-[3-(3-hydroxy-4-methyl-phenyl)-propylamino]-4,6-dimethyl-pyrimidine-5-carboxylic acid ethyl ester, lithium hydroxide monohydrate (440 mg, 10.5 mmol), in dioxane/water (25 ml/25 ml) was stirred at 95° C. for 4 h, then cooled to room temperature and quenched with aqueous potassium hydrogen sulfate to pH 2-4. The resulting solution was extracted with EtOAc. The organic extracts were combined, washed with brine, dried over sodium sulfate, filtered and evaporated to give the title compound (110 mg, 83% yield) MS m/e 316.0 (M+H⁺).

### Preparation of 2-[(E)-3-(3-Hydroxy-phenyl)-allylamino]-4,6-dimethyl-pyrimidine-5-carboxylic acid ethyl ester

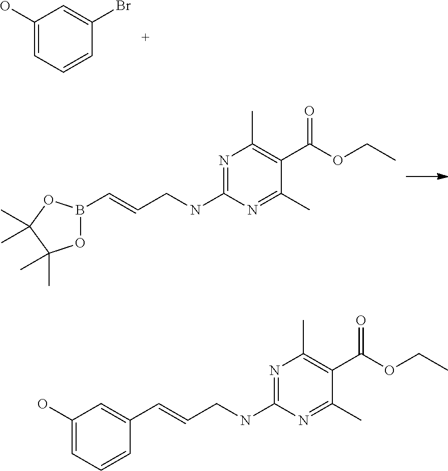

A microwave reaction vessel was charged with 4,6-dimethyl-2-[(E)-3-(4,4,5,5-tetramethyl-[1,3,2]dioxaborolan-2-yl)-allylamino]-pyrimidine-5-carboxylic acid ethyl ester (300 mg, 0.83 mmol), 3-bromo-phenol (172 mg, 1.0 mmol), [1,1′-bis(diphenylphosphinoferrocene)-dichloropalladium(II) DCM complex (68 mg, 0.083 mmol), potassium carbonate (229 mg, 1.6 mmol), DMF (4 ml), and water (0.5 ml). The mixture was degassed and flushed with nitrogen, followed by heating to 140° C. for 10 min in a microwave oven. The mixture was directly loaded onto column. Eluting with a 0-50% EtOAc in hexane gradient provided pure product (136.4 mg, 50% yield). MS m/e 328.0 (M+H⁺).

### Preparation of 2-[(E)-3-(3-hydroxy-phenyl)-allylamino]-4,6-dimethyl-pyrimidine-5-carboxylic acid

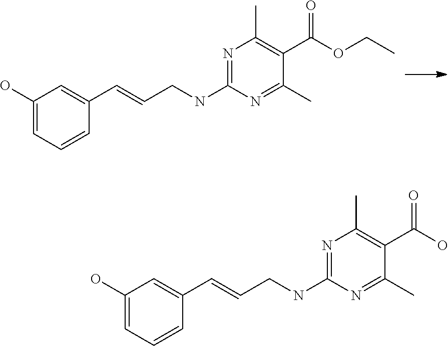

A mixture of 2-[(E)-3-(3-hydroxy-phenyl)-allylamino]-4,6-dimethyl-pyrimidine-5-carboxylic acid ethyl ester (136 mg, 0.41 mmol), lithium hydroxide monohydrate (430 mg, 10.25 mmol), dioxane (10 ml) and water (10 ml) was heated to 90° C. for 4 h, then cooled to room temperature and quenched with aqueous potassium hydrogen sulfate to pH 2-4. The resulting solution was extracted with EtOAc. The organic extracts were combined, washed with brine, dried over sodium sulfate, filtered and evaporated to give the title compound (111 mg, 90% yield). MS m/e 299.9 (M+H⁺).

### Preparation of 2-[(E)-3-(6-Benzyloxy-pyridin-2-yl)-allylamino]-4,6-dimethyl-pyrimidine-5-carboxylic acid ethyl ester

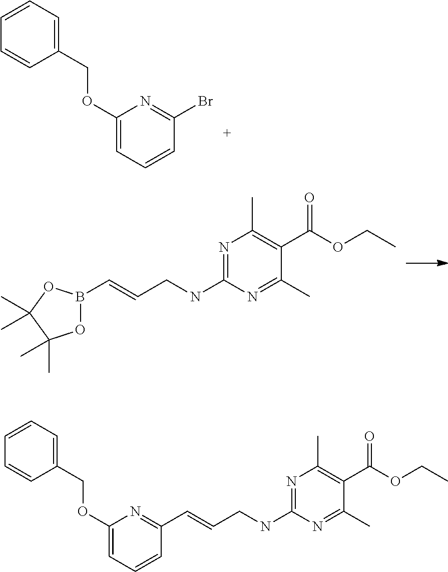

A microwave reaction vessel was charged with 4,6-dimethyl-2-[(E)-3-(4,4,5,5-tetramethyl-[1,3,2]dioxaborolan-2-yl)-allylamino]-pyrimidine-5-carboxylic acid ethyl ester (250 mg, 0.69 mmol), 6-benzyloxy-2-bromopyridine (183 mg, 0.69 mmol), [1,1′-bis(diphenylphosphinoferrocene)-dichloropalladium(II) DCM complex (28.2 mg, 0.035 mmol), potassium carbonate (190 mg, 1.38 mmol), DMF (4 ml), and water (1 ml). The mixture was degassed and flushed with nitrogen, followed by heating to 135° C. for 15 min in a microwave oven. The mixture was directly loaded onto column. Eluting with 30% EtOAc in hexane provided the desired product (140 mg, 48% yield). MS m/e 419.0 (M+H⁺).

### Preparation of 2-[(E)-3-(6-Benzoxy-pyridin-2-yl)-allylamino]-4,6-dimethyl-pyrimidine-5-carboxylic acid

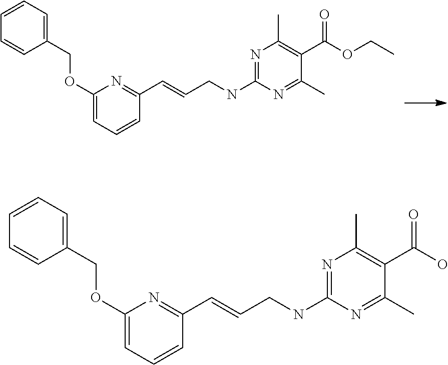

A mixture of 2-[(E)-3-(6-benzyloxy-pyridin-2-yl)-allylamino]-4,6-dimethyl-pyrimidine-5-carboxylic acid ethyl ester (140 mg, 0.33 mmol), and lithium hydroxide monohydrate (351 mg, 8.36 mmol), in dioxane/water (10 ml/10 ml) was stirred at 90° C. for 9 h, then cooled to room temperature and quenched with aqueous potassium hydrogen sulfate to pH 2-4. The resulting solution was extracted with EtOAc. The organic extracts were combined, washed with brine, dried over sodium sulfate, filtered and concentrated to give the title compound (111 mg, 86% yield). MS m/e 391.0 (M+H⁺).

### Preparation of 2-[3-(2-Fluoro-3-methoxy-phenyl)-prop-2-ynylamino]-4,6-dimethyl-pyrimidine-5-carboxylic acid ethyl ester

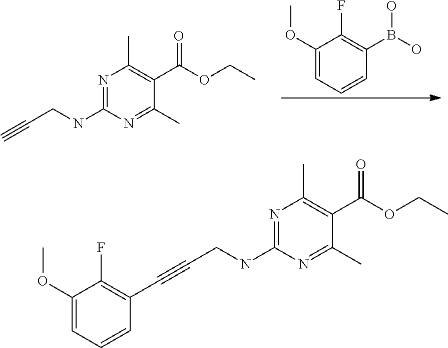

To a solution of 2-fluoro-3-methoxy-phenyl boronic acid (219 mg, 1.29 mmol) and 4,6-dimethyl-2-ynylamino-pyrimidine-5-carboxylic acid ethyl ester (200 mg, 0.87 mmol) in anhydrous DCM (15 ml) was added [1,1′-bis(diphenylphosphinoferrocene)-dichloropalladium(II) DCM complex (71 mg, 0.087 mmol), potassium carbonate (60.1 mg, 4.25 mmol) and silver (II) oxide (504 mg, 2.18 mmol). The mixture was stirred at room temperature for 3 days, then directly loaded onto a silicagel column. Eluting with 30% EtOAc in hexane provided the desired product (22 mg, 7% yield).

### Preparation of 2-[3-(2-fluoro-3-methoxy-phenyl)-propylamino]-4,6-dimethyl-pyrimidine-5-carboxylic acid ethyl ester

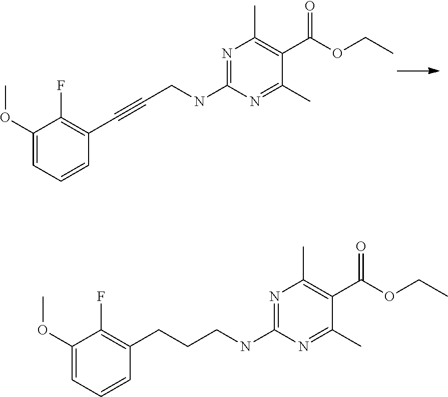

A solution of 2-[3-(2-Fluoro-3-methoxy-phenyl)-prop-2-ynylamino]-4,6-dimethyl-pyrimidine-5-carboxylic acid ethyl ester (95 mg, 0.27 mmol) in EtOH (200 ml) was passed through H-Cube with a 10% Pd/C small cartridge at 40 bar, 40° C. and 1 ml per min. Solvent was then removed by evaporation to give the desired compound (90 mg, 94% yield). MS m/e 362.0 (M+H⁺).

### Preparation of 2-[3-(2-Fluoro-3-hydroxy-phenyl)-propylamino]-4,6-dimethyl-pyrimidine-5-carboxylic acid ethyl ester

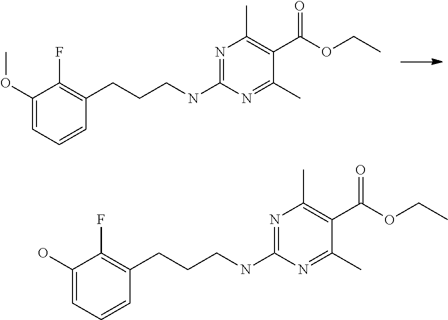

To a solution of 2-[3-(2-fluoro-3-methoxy-phenyl)-propylamino]-4,6-dimethyl-pyrimidine-5-carboxylic acid ethyl ester (90 mg, 0.249 mmol) in anhydrous DCM was added at 0° C. BBr3/DCM (1M, 0.5 ml, 0.5 mmol). The mixture was stirred at room temperature for 2 h, then quenched with ice water. The mixture was diluted with DCM. The organic layer was separated, washed with brine, dried over sodium sulfate, filtered and evaporated to give the desired product (70 mg, 81% yield). MS m/e 347.9 (M+H⁺).

### Preparation of 2-[3-(2-Fluoro-3-hydroxy-phenyl)-propylamino]-4,6-dimethyl-pyrimidine-5-carboxylic acid

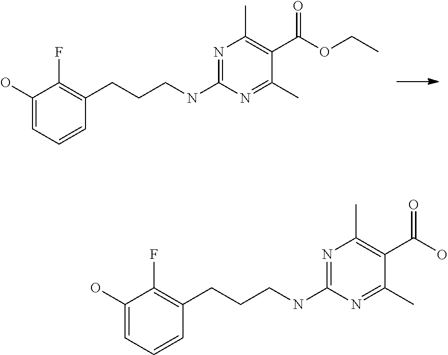

A mixture of 2-[3-(2-fluoro-3-hydroxy-phenyl)-propylamino]-4,6-dimethyl-pyrimidine-5-carboxylic acid ethyl ester (70 mg, 0.202 mmol), and lithium hydroxide monohydrate (21 mg, 5.037 mmol) in dioxane/water (3 ml/3 ml) was stirred at 90° C. for 18 h, cooled to room temperature and quenched with aqueous potassium hydrogen sulfate to adjust the pH to 2-4. The mixture was extracted with EtOAc. The organic extracts were combined, washed with brine, dried over sodium sulfate, filtered and concentrated to give the title compound (60 mg, 93% yield). MS m/e 319.9 (M+H⁺).

### Preparation of 2-[(E)-3-(2-Fluoro-5-methoxy-phenyl)-allylamino]-4,6-dimethyl-pyrimidine-5-carboxylic acid ethyl ester

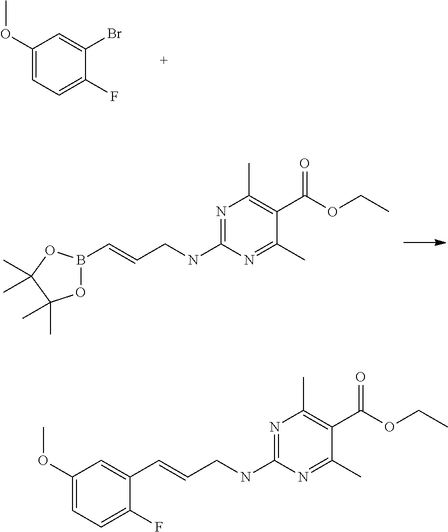

A microwave reaction vessel was charged with 4,6-dimethyl-2-[(E)-3-(4,4,5,5-tetramethyl-[1,3,2]dioxaborolan-2-yl)-allylamino]-pyrimidine-5-carboxylic acid ethyl ester (300 mg, 0.83 mmol), 3-bromo-6-fluoro-phenol (340 mg, 1.66 mmol), dichloro-bis(triphenylphosphine)palladium (II) (38 mg, 0.042 mmol), 1,2,3,4,5-pentaphenyl-1′-(di-tert-butyl-phosphino)-ferrocene (58 mg, 0.083 mmol), cesium fluoride (388 mg, 2.49 mmol), and THF (20 ml). The mixture was degassed, flushed with nitrogen and heated to 120° C. for 20 min in a microwave oven. The mixture was directly loaded onto a silicagel column Eluting with a 0-30% EtOAc in hexane gradient provided pure product (139 mg, 47% yield). MS m/e 359.9 (M+H⁺).

### Preparation of 2-[3-(2-Fluoro-5-methoxy-phenyl)-propylamino]-4,6-dimethyl-pyrimidine-5-carboxylic acid ethyl ester

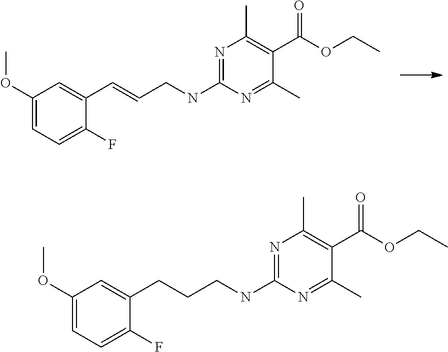

A solution of 2-[(E)-3-(2-fluoro5-methoxy-phenyl)-allylamino]-4,6-dimethyl-pyrimidine-5-carboxylic acid ethyl ester (139 mg, 0.39 mmol) in EtOH (250 ml) was passed through H-Cube with 10% Pd/C small cartridge at 20 bar, room temperature and 1 ml per min. Solvent was removed by evaporation to give the desired product (123 mg, 88% yield). MS m/e 361.9 (M+H⁺).

### Preparation of 2-[3-(2-Fluoro-5-hydroxy-phenyl)-propylamino]-4,6-dimethyl-pyrimidine-5-carboxylic acid ethyl ester

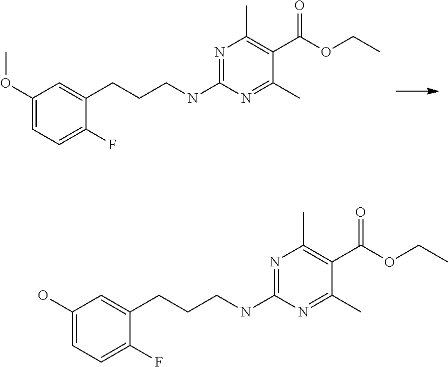

A solution of 2-[3-(2-fluoro-5-methoxy-phenyl)-propylamino]-4,6-dimethyl-pyrimidine-5-carboxylic acid ethyl ester (123 mg, 0.34 mmol) in anhydrous DCM was treated at 0° C. with BBr3/DCM (1M, 0.7 ml, 0.7 mmol). The mixture was allowed to warm up to room temperature and stirred for 2 h, then quenched with ice water. The mixture was extracted with DCM. The organic layer was separated, washed with brine, dried over sodium sulfate, filtered and concentrated to give the desired product (72 mg, 61% yield). MS m/e 347.9 (M+H⁺).

### Preparation of 2-[3-(2-Fluoro-5-hydroxy-phenyl)-propylamino]-4,6-dimethyl-pyrimidine-5-carboxylic acid

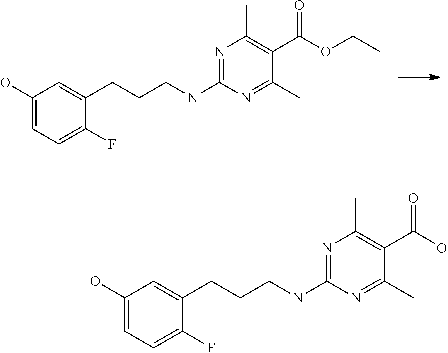

A mixture of 2-[3-(2-fluoro-5-hydroxy-phenyl)-propylamino]-4,6-dimethyl-pyrimidine-5-carboxylic acid ethyl ester (72 mg, 0.21 mmol), and lithium hydroxide monohydrate (217 mg, 5.25 mmol) in dioxane/water (5 ml/5 ml) was stirred at 90° C. overnight, then cooled to room temperature and quenched with aqueous potassium hydrogen sulfate to adjust the pH to 2-4. The mixture was extracted with EtOAc. The organic extracts were combined, washed with brine, dried over sodium sulfate, filtered and concentrated to give the title compound, (62.5 mg, 93% yield). MS m/e 319.9 (M+H⁺).

### Preparation of 2-[(E)-3-(3-bromo-5-hydroxy-2-methyl-phenyl)-allylamino]-4,6-dimethyl-pyrimidine-5-carboxylic acid ethyl ester

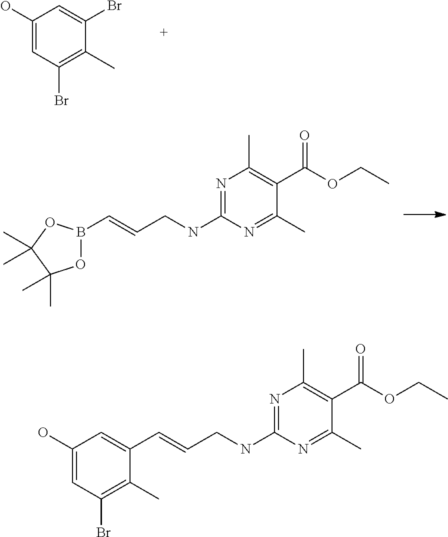

A mixture of 4,6-dimethyl-2-[(E)-3-(4,4,5,5-tetramethyl-[1,3,2]dioxaborolan-2-yl)-allylamino]-pyrimidine-5-carboxylic acid ethyl ester (150 mg, 0.415 mmol), 3,5-dibromo-4-methyl-phenol (221 mg, 0.83 mmol), dichloro-bis(triphenylphosphine)palladium (II) (19 mg, 0.021 mmol), 1,2,3,4,5-pentaphenyl-1′-(di-tert-butyl-phosphino)-ferrocene (29 mg, 0.042 mmol), and cesium fluoride (189 mg, 1.245 mmol) in THF (10 ml) was stirred at room temperature for 24 h. The mixture was directly loaded onto a silicagel column Eluting with a 0-30% EtOAc in hexane gradient afforded the desired product (94 mg, 54% yield). MS m/e 421.7 (M+H⁺).

### Preparation of 2-[3-(2-Methyl-5-methoxy-phenyl)-propylamino]-4,6-dimethyl-pyrimidine-5-carboxylic acid ethyl ester

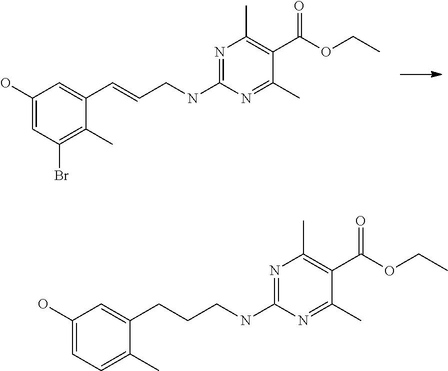

A solution of 2-[(E)-3-(3-bromo-5-hydroxy-2-methyl-phenyl)-allylamino]-4,6-dimethyl-pyrimidine-5-carboxylic acid ethyl ester (94 mg, 0.22 mmol) in EtOH (250 ml) was passed through H-Cube with 10% Pd/C small cartridge at 1 atmosphere, room temperature and 1 ml per min. After concentration, the residue was purified by flash chromatography with a 0-50% EtOAc in hexane gradient to afford the desired product (19 mg, 25% yield). MS m/e 344.0 (M+H⁺).

### Preparation of 2-[3-(2-Methyl-5-hydroxy-phenyl)-propylamino]-4,6-dimethyl-pyrimidine-5-carboxylic acid

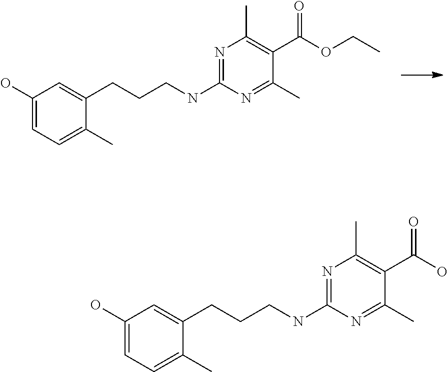

A solution of 2-[3-(2-methyl-5-hydroxy-phenyl)-propylamino]-4,6-dimethyl-pyrimidine-5-carboxylic acid ethyl ester (19 mg, 0.055 mmol) in dioxane (3 ml) was treated with a solution of lithium hydroxide monohydrate (58 mg, 1.38 mmol) in water (3 ml). The mixture was stirred at 90° C. for 4 h, then cooled to room temperature and quenched with aqueous potassium hydrogen sulfate to adjust the pH to ˜2-4. The resulting solution was extracted with EtOAc. The organic extracts were combined, washed with brine, dried over sodium sulfate, filtered and evaporated to give the title compound (15.8 mg, 91% yield). MS m/e 315.9 (M+H⁺).

### Preparation of 2-[3-(3-Methoxy-phenyl)-propylamino]-4-methyl-pyrimidine-5-carboxylic acid ethyl ester

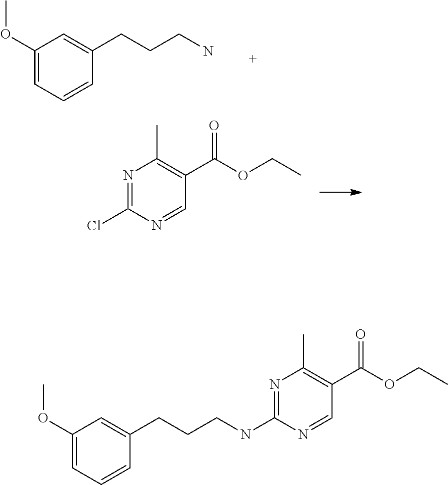

A microwave reaction vessel was charged with 3-(methoxy-phenyl)-propylamine hydrochloride (404 mg, 2.0 mmol), 2-chloro-4-methyl-pyrimidine-5-carboxylic acid ethyl ester (400 mg, 2.0 mmol), potassium acetate (785 mg, 8.0 mmol) and ethanol (10 ml), then heated to 150° C. for 1 h in a microwave oven. The mixture was cooled to room temperature and extracted with EtOAc. The organic phase was washed with 20% sodium bicarbonate, water and brine, then dried over sodium sulfate, filtered, and evaporated. The residue was purified by flash chromatography with 30% EtOAc in hexane to afford the desired compound (568.4 mg, 86% yield). MS m/e 330.0 (M+H⁺).

### Preparation of 2-[3-(3-Hydroxy-phenyl)-propylamino]-4-methyl-pyrimidine-5-carboxylic acid ethyl ester

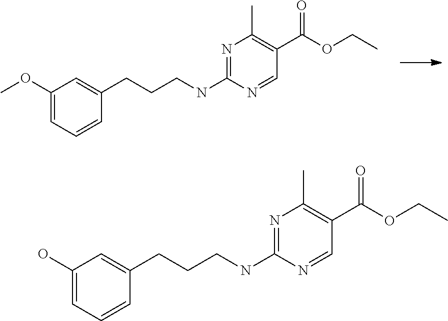

A solution of 2-[3-(3-methoxy-phenyl)-propylamino]-4-methyl-pyrimidine-5-carboxylic acid ethyl ester (568 mg, 1.72 mmol) in anhydrous DCM (10 ml) was treated at 0° C. with BBr3/DCM (1M, 3.4 ml, 3.4 mmol). The mixture was allowed to warm up to room temperature and stirred for 2 h, then quenched with ice water. The organic layer was separated and the aqueous layer was extracted by DCM. The organic layer was separated, washed with brine, dried over sodium sulfate, filtered and concentrated to give the desired product (530.6 mg, 98% yield). MS m/e 315.9 (M+H⁺).

### Preparation of 2-[3-(3-hydroxy-phenyl)-propylamino]-4-methyl-pyrimidine-5-carboxylic acid

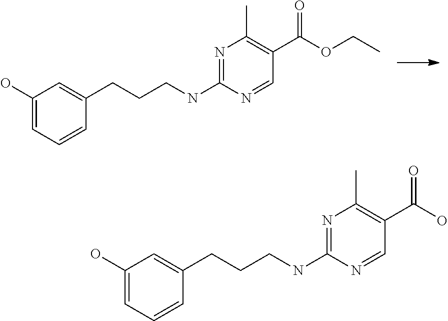

A mixture of 2-[3-(3-hydroxy-phenyl)-propylamino]-4-methyl-pyrimidine-5-carboxylic acid ethyl ester (530 mg, 1.68 mmol), and lithium hydroxide monohydrate (1.76 g, 42 mmol), in dioxane/water (5 ml/5 ml) was stirred at 60° C. overnight, then cooled to room temperature and quenched with aqueous potassium hydrogen sulfate to adjust the pH to ˜2-4. The resulting solution was extracted with EtOAc. The organic extracts were combined, washed with brine, dried over sodium sulfate, filtered and evaporated to give the title compound (415 mg, 86% yield). MS m/e 287.9 (M+H⁺).

### Preparation of 2-[3-(3-Methoxy-phenyl)-propylamino]-4-trifluoromethyl-pyrimidine-5-carboxylic acid methyl ester

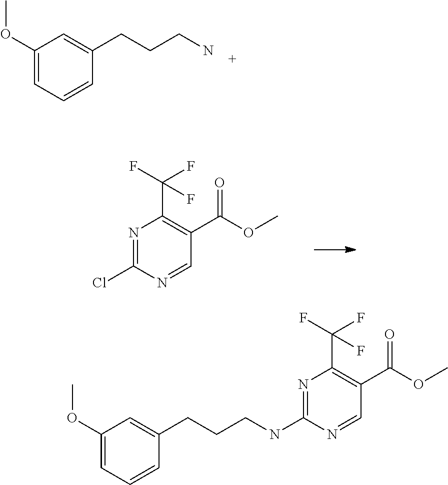

A microwave reaction vessel was charged with 3-(methoxy-phenyl)-propylamine (1.03 g, 6.24 mmol), 2-chloro-4-trifluoromethyl-pyrimidine-5-carboxylic acid methyl ester (1 g, 4.16 mmol), potassium acetate (1.22 g, 12.48 mmol) and methanol (10 ml). The mixture was heated to 150° C. for 1 h in a microwave oven. The mixture was cooled to room temperature and extracted with EtOAc. The organic phase washed with water and brine, dried over sodium sulfate, filtered, and concentrated. The residue was purified by flash chromatography with 25% EtOAc in hexane to afford the desired product (917 mg, 60% yield). MS m/e 369.9 (M+H⁺).

### Preparation of 2-[3-(3-Hydroxy-phenyl)-propylamino]-4-trifluoromethyl-pyrimidine-5-carboxylic acid methyl ester

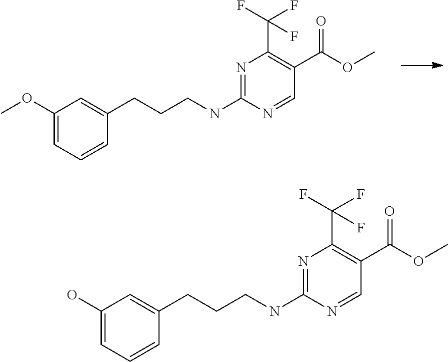

A solution of 2-[3-(3-methoxy-phenyl)-propylamino]-4-trifluoromethyl-pyrimidine-5-carboxylic acid ethyl ester (917 mg, 2.48 mmol) in anhydrous DCM (10 ml) was treated at 0° C. with BBr3/DCM (1M, 5 ml, 5 mmol). The mixture was allowed to warm up to room temperature and stirred for 2 h, then quenched with ice water. The organic layer was separated and the aqueous layer was extracted by DCM. The organic layer was separated, washed with brine, dried over sodium sulfate, filtered and concentrated to give the desired product (0.9 g, 100% yield). MS m/e 355.9 (M+H⁺).

### Preparation of 2-[3-(3-hydroxy-phenyl)-propylamino]-4-trifluoromethyl-pyrimidine-5-carboxylic acid

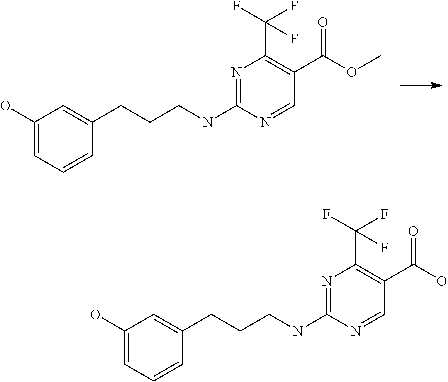

A mixture of 2-[3-(3-hydroxy-phenyl)-propylamino]-4-trifluoromethyl-pyrimidine-5-carboxylic acid methyl ester (0.9 g, 2.53 mmol), and lithium hydroxide monohydrate (2.13 g, 50.66 mmol), in dioxane/water (20 ml/20 ml) was stirred at 0° C. overnight, then cooled to room temperature and quenched with aqueous potassium hydrogen sulfate to adjust the pH to ˜2-4. The resulting solution was extracted with EtOAc. The organic extracts were combined, washed with brine, dried over sodium sulfate, filtered and evaporated to give the title compound (770 mg, 89% yield). MS m/e 341.9 (M+H⁺).

### Preparation of 2-[3-(1H-indazol-4-yl)-propylamino]-4,6-dimethyl-pyrimidine-5-carboxylic acid ethyl ester

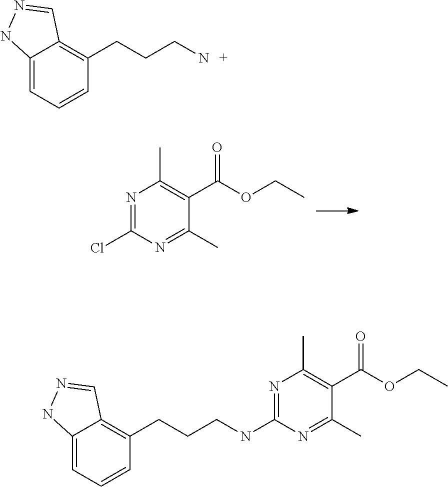

A mixture of 3-(1H-indazol-4-yl)-propylamine dihydrochloride (1.61 g, 7.5 mmol), 2-chloro-4,6-dimethyl-pyrimidine-5-carboxylic acid ethyl ester (2.23 g, 9.0 mmol) and EtOH (30 ml) was heated in a microwave reactor at 160° C. for 1.0 h, diluted with EtOAc (200 ml), washed with brine (200 ml) and re-extracted with EtOAc (200 ml). The combined organic layers were washed with brine, dried over sodium sulfate, filtered, concentrated in the presence of silica gel and chromatographed (10-80% EtOAc in hexane) to give the desired product (2.28 g, 81% yield).

### Preparation of 2-[3-(3-(1H-indazol-4-yl)-propylamino)-4,6-dimethyl-pyrimidine-5-carboxylic acid

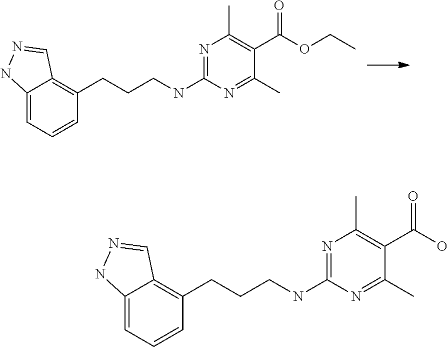

To a solution of 2-[3-(3-(1H-indazol-4-yl)-propylamino)-4,6-dimethyl-pyrimidine-5-carboxylic acid ethyl ester (1.79 g, 5.06 mmol) in dioxane (35 ml) was added a solution of lithium hydroxide (4.24 g, 101.2 mmol) in water (45 ml). The mixture was heated to reflux for 2 hours, cooled to room temperature, acidified with 2N KHSO₄, concentrated, filtered, and the precipitate was washed with water to afford the title compound (1.64 g, 99% yield).

### Preparation of (S)-3-tert-butoxycarbonylamino-2-({2-[3-(3-hydroxy-phenyl)-propylamino]-4,6-dimethyl-pyrimidine-5-carbonyl}-amino)-propionic acid methyl ester

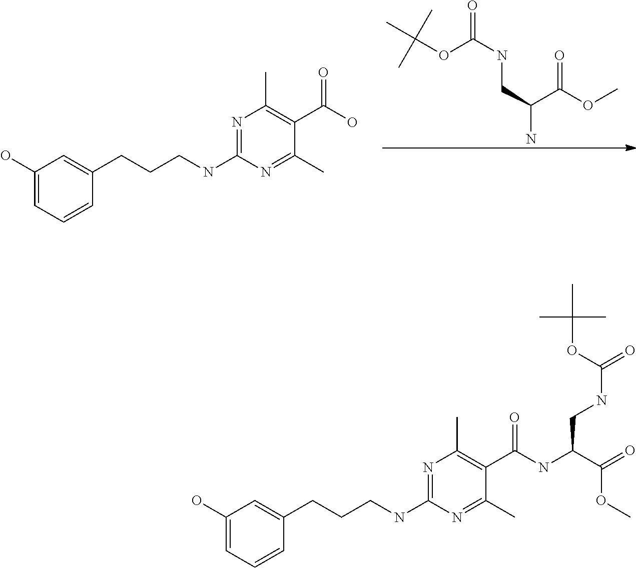

To a solution of 2-[3-(3-hydroxy-phenyl)-propylamino]-4,6-dimethyl-pyrimidine-5-carboxylic acid (1.76 g, 5.84 mmol) in anhydrous DMF (60 ml) was added Et₃N (2.5 ml, 7.0 mmol), HBTU (2.66 g, 7.01 mmol), HOBT (0.95 g, 7.01 mmol), and H-DAP(Boc)OMe hydrochloride (1.79 g, 7.01 mmol). The mixture was stirred at room temperature for 3 h, diluted with brine (200 ml) and extracted with ethyl acetate. The combined organic layers were washed with 1:1 saturated sodium bicarbonate/brine and brine, then dried over Na₂SO₄, filtered, and concentrated under reduced pressure. The crude residue was purified by flash chromatography with a 40-100% EtOAc in hexane gradient to give the title compound (2.66 g, 91% yield). MS m/e 501.9 (M+H⁺).

### Preparation of (S)-3-Amino-2-({2-[3-(3-hydroxy-phenyl)-propylamino]-4,6-dimethyl-pyrimidine-5-carbonyl}-amino)-propionic acid methyl ester hydrochloride

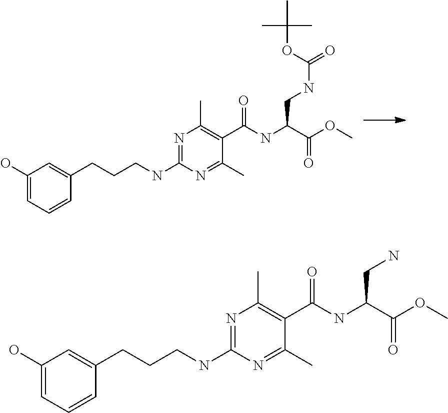

To a solution of (S)-3-tert-Butoxycarbonylamino-2-({2-[3-(3-hydroxy-phenyl)-propylamino]-4,6-dimethyl-pyrimidine-5-carbonyl}-amino)-propionic acid methyl ester (2.66 g, 5.30 mmol) in MeOH (10 ml) was added 4.0 M HCl in dioxane (20 mL). After one hour the mixture was concentrated and azeotroped with MeOH. The product was triturated with ether, filtered, and washed with ether to afford the title compound (2.16 g, 93% yield). MS m/e 401.9 (M+H⁺).

### Preparation of (S)-3-tert-butoxycarbonylamino-2-({2-[3-(3-hydroxy-4-methyl-phenyl)-propylamino]-4,6-dimethyl-pyrimidine-5-carbonyl}-amino)-propionic acid methyl ester

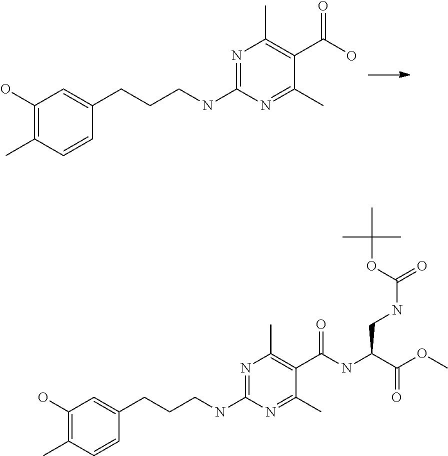

To a solution of 2-[3-(3-hydroxy-4-methyl-phenyl)-propylamino]-4,6-dimethyl-pyrimidine-5-carboxylic acid (110 mg, 0.35 mmol) in anhydrous DMF (5 ml) was added triethylamine (0.15 ml, 1.05 mmol), HBTU (137 mg, 0.36 mmol), HOBT (49 mg, 0.36 mmol), and H-DAP(Boc)OMe hydrochloride (92 mg, 0.36 mmol). The mixture was stirred at room temperature for 2 h, followed by direct loading onto a column. Eluting with a 0-80% EtOAc in hexane gradient afforded the title compound contaminated with DMF (250 mg). MS m/e 516.1 (M+H⁺).

### Preparation of (S)-3-tert-Butoxycarbonylamino-2-({2-[(E)-3-(3-hydroxy-phenyl)-allylamino]-4,6-dimethyl-pyrimidine-5-carbonyl}-amino)-propionic acid methyl ester

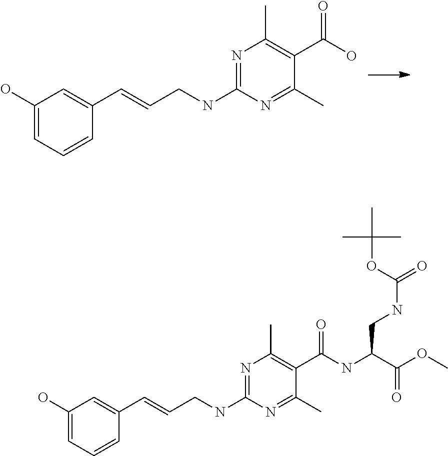

To a solution of 2-[(E)-3-(3-hydroxy-phenyl)-allylamino]-4,6-dimethyl-pyrimidine-5-carboxylic acid (111 mg, 0.37 mmol) in anhydrous DMF (3 ml) was added at room temperature triethylamine (0.15 ml, 1.11 mmol), HBTU (144 mg, 0.38 mmol), HOBT (51 mg, 0.38 mmol), and H-DAP(Boc)OMe hydrochloride (96.3 mg, 0.3 mmol). The mixture was stirred at room temperature overnight, followed by direct loading onto a silicagel column. Eluting with a 40-100% EtOAc in hexane gradient provided the desired compound contaminated with DMF (274.3 mg). MS m/e 500.0 (M+H⁺).

### Preparation of (S)-3-tert-Butoxycarbonylamino-2-({2-[3-(2-fluoro-3-hydroxy-phenyl)-propylamino]-4,6-dimethyl-pyrimidine-5-carbonyl}-amino)-propionic acid methyl ester

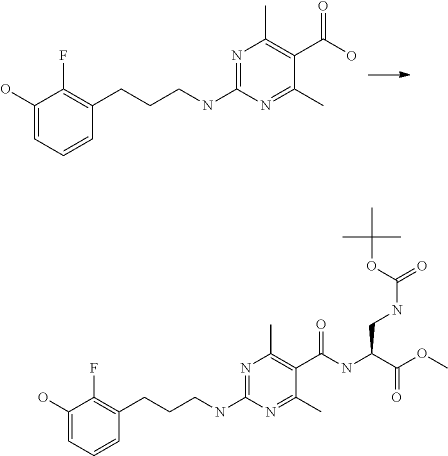

To a solution of 2-[3-(2-fluoro-3-hydroxyl-phenyl)-propylamino]-4,6-dimethyl-pyrimidine-5-carboxylic acid (60 mg, 0.19 mmol) in anhydrous DMF (4 ml) was added at room temperature triethylamine (0.13 ml, 1.05 mmol), HBTU (143 mg, 0.38 mmol), HOBT (51 mg, 0.38 mmol), and H-DAP(Boc)OMe hydrochloride (96 mg, 0.38 mmol). The mixture was stirred at room temperature for 18 h, then quenched with water and extracted with EtOAc. The organic layer was separated, washed with brine, dried over sodium sulfate, filtered and evaporated. The residue was purified by flash chromatography with a 40-100% EtOAc on hexane gradient to yield the desired product (94 mg, 96% yield). MS m/e 520.0 (M+H⁺).

### Preparation of (S)-3-tert-Butoxycarbonylamino-2-({2-[3-(2-methyl-5-hydroxy-phenyl)-propylamino]-4,6-dimethyl-pyrimidine-5-carbonyl}-amino)-propionic acid methyl ester

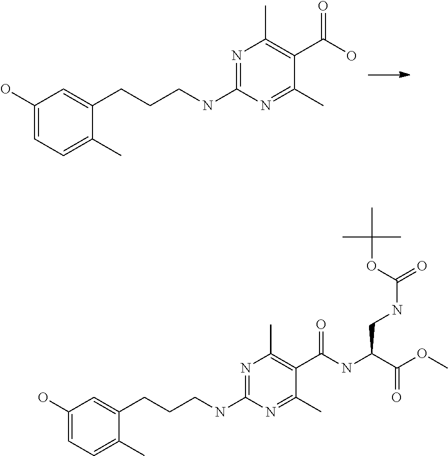

To a solution of 2-[3-(2-methyl-5-hydroxyl-phenyl)-propylamino]-4,6-dimethyl-pyrimidine-5-carboxylic acid (15.8 mg, 0.05 mmol) in anhydrous DMF (2 ml) was added triethylamine (25 mg, 0.25 mmol), HBTU (37.9 mg, 0.1 mmol), HOBT (13.5 mg, 0.10 mmol), and H-DAP(Boc)OMe hydrochloride (25.5 mg, 0.10 mmol). The mixture was stirred at room temperature for 3 h, quenched with water and extracted with EtOAc. The combined organic layers were washed with brine, dried over sodium sulfate, filtered and concentrated. The residue was purified by flash chromatography with a 40-100% EtOAc in hexane gradient to give the desired product (26 mg, 100% yield). MS m/e 516.0 (M+H⁺).

### Preparation of (S)-3-tert-Butoxycarbonylamino-2-({2-[3-(2-fluoro-5-hydroxy-phenyl)-propylamino]-4,6-dimethyl-pyrimidine-5-carbonyl}-amino)-propionic acid methyl ester

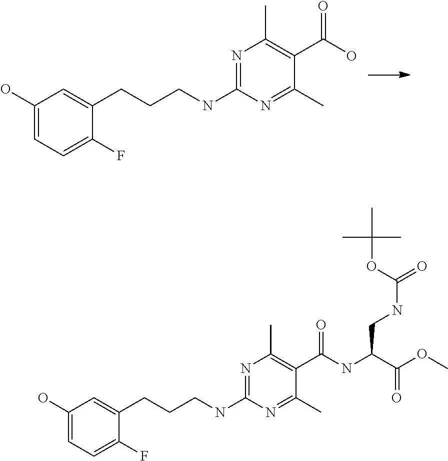

To a solution of 2-[3-(2-fluoro-5-hydroxyl-phenyl)-propylamino]-4,6-dimethyl-pyrimidine-5-carboxylic acid (62.5 mg, 0.196 mmol) in anhydrous DMF (4 ml) was added triethylamine (0.14 ml, 0.98 mmol), HBTU (149 mg, 0.39 mmol), HOBT (53 mg, 0.39 mmol), and H-DAP(Boc)OMe hydrochloride (100 mg, 0.39 mmol). The mixture was stirred at room temperature over the weekend, then quenched with water and extracted with EtOAc. The organic layer was separated, washed with brine, dried over sodium sulfate, filtered and evaporated. The residue was purified by flash chromatography with a 40-100% EtOAc in hexane gradient to afford the desired product (62 mg, 61% yield). MS m/e 520.0 (M+H⁺).

### Preparation of (S)-3-tert-Butoxycarbonylamino-2-({2-[3-(3-hydroxy-phenyl)-propylamino]-pyrimidine-5-carbonyl}-amino)-propionic acid methyl ester

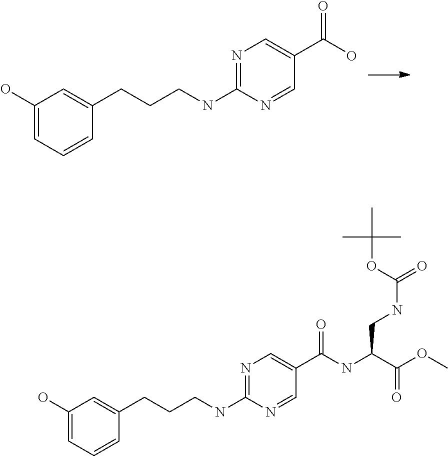

To a solution of 2-[3-(3-hydroxyl-phenyl)-propylamino]-pyrimidine-5-carboxylic acid (900 mg, 3.29 mmol) in anhydrous DMF (10 ml) was added triethylamine (1.4 ml, 9.87 mmol), HBTU (1.5 g, 3.85 mmol), HOBT (534 mg, 3.95 mmol), and H-DAP(Boc)OMe hydrochloride (864 mg, 3.39 mmol). The mixture was stirred at room temperature overnight, then quenched with water and extracted with EtOAc. The organic layer was separated, washed with brine, dried over sodium sulfate, filtered and evaporated. The residue was purified by flash chromatography with a 0-90% EtOAc in hexane gradient to give the desired product (820 mg, 53% yield). MS m/e 474.0 (M+H⁺).

### Preparation of (S)-3-tert-Butoxycarbonylamino-2-({2-[3-(3-hydroxy-phenyl)-propylamino]-4-methyl-pyrimidine-5-carbonyl}-amino)-propionic acid methyl ester

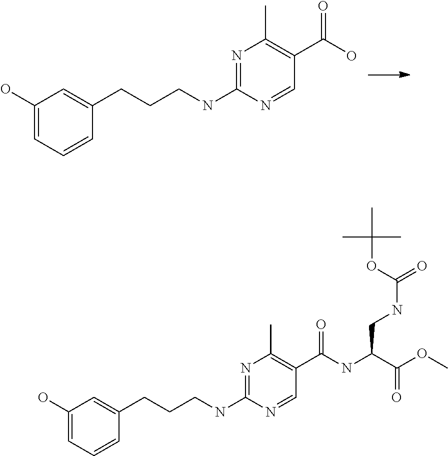

To a solution of 2-[3-(3-hydroxyl-phenyl)-propylamino]-4-methyl-pyrimidine-5-carboxylic acid (415 mg, 1.444 mmol) in anhydrous DMF (5 mL) was added triethylamine (0.6 ml, 4.33 mmol), HBTU (559 mg, 1.47 mmol), HOBT (199 mg, 1.47 mmol), and H-DAP(Boc)OMe hydrochloride (375 mg, 1.47 mmol). The mixture was stirred at room temperature overnight, then quenched with water and extracted with EtOAc. The organic layer was separated, washed with brine, dried over sodium sulfate, filtered and evaporated. The residue was purified by flash chromatography with a 0-80% EtOAc in hexane gradient to give the title compound (482 mg, 68% yield). MS m/e 488.0 (M+H⁺).

### Preparation of (S)-3-tert-Butoxycarbonylamino-2-({2-[3-(3-hydroxy-phenyl)-propylamino]-4-trifluoromethyl-pyrimidine-5-carbonyl}-amino)-propionic acid methyl ester

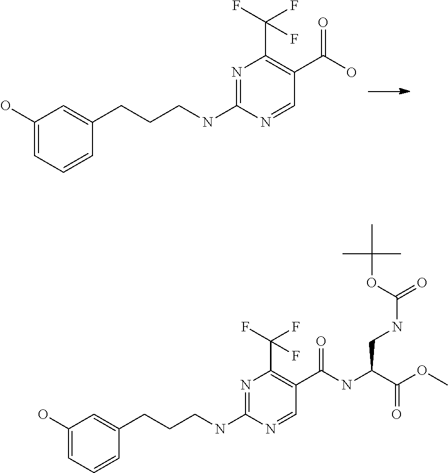

To a solution of 2-[3-(3-hydroxyl-phenyl)-propylamino]-4-trifluoromethyl-pyrimidine-5-carboxylic acid (770 mg, 2.26 mmol) in anhydrous DMF (10 ml) was added triethylamine (0.95 ml, 6.78 mmol), HBTU (1.03 g, 2.71 mmol), HOBT (366 mg, 2.71 mmol), and H-DAP(Boc)OMe hydrochloride (591 mg, 2.32 mmol). The mixture was stirred at room temperature overnight, then quenched with water and extracted with EtOAc. The organic layer was separated, washed with brine, dried over sodium sulfate, filtered and evaporated. The residue was purified by flash chromatography with a 0-60% EtOAc in hexane gradient to give the desired product (970 mg, 79% yield). MS m/e 441.9 (M+H⁺-Boc).

### Preparation of (S)-3-tert-Butoxycarbonylamino-2-({4-ethyl-2-[3-(3-hydroxy-phenyl)-propylamino]-6-methyl-pyrimidine-5-carbonyl}-amino)-propionic acid methyl ester

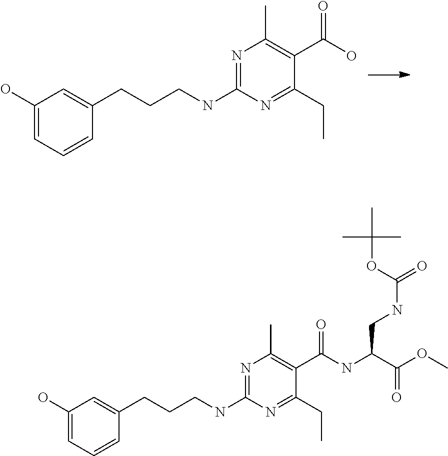

To a solution of 4-ethyl-2-[3-(3-hydroxy-phenyl)-propylamino]-6-methyl-pyrimidine-5-carboxylic acid (219 mg, 0.694 mmol) in anhydrous DMF (10 ml) was added triethylamine (0.3 ml, 2.082 mmol), HBTU (268 mg, 0.708 mmol), HOBT (96 mg, 0.708 mmol), and H-DAP(Boc)OMe hydrochloride (180 mg, 0.708 mmol). The mixture was stirred at room temperature overnight, then quenched with water and extracted with EtOAc. The organic layer was separated, washed with brine, dried over sodium sulfate, filtered and evaporated. The residue was purified by flash chromatography with a 0-80% EtOAc in hexane gradient to give the title compound (265 mg, 74% yield). MS m/e 516.0 (M+H⁺-Boc).

### Preparation of (S)-3-tert-Butoxycarbonylamino-2-({4,6-diethyl-2-[3-(3-hydroxy-phenyl)-propylamino]-pyrimidine-5-carbonyl}-amino)-propionic acid methyl ester

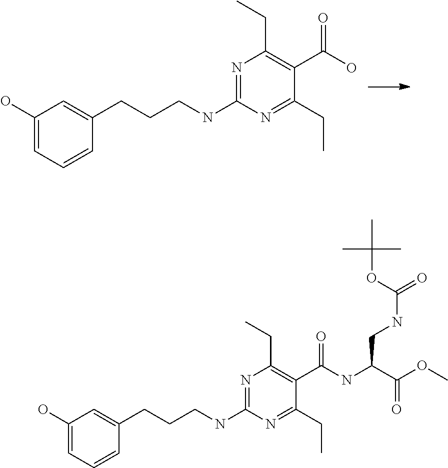

To a solution of 4,6-diethyl-2-[3-(3-hydroxy-phenyl)-propylamino]-pyrimidine-5-carboxylic acid (26 mg, 0.08 mmol) in anhydrous DMF (2 ml) was added triethylamine (40 mg, 0.4 mmol), HBTU (33 mg, 0.09 mmol), HOBT (12 mg, 0.09 mmol), and H-DAP(Boc)OMe hydrochloride (22 mg, 0.09 mmol). The mixture was stirred at room temperature overnight, then quenched with water and extracted with EtOAc. The organic layer was separated, washed with brine, dried over sodium sulfate, filtered and evaporated. The residue was purified by flash chromatography with a 0-80% EtOAc in hexane gradient to give the title compound (21.6 mg, 52% yield). MS m/e 530.0 (M+H⁺).

### Preparation of (S)-3-tert-Butoxycarbonylamino-2-({2-[3-(1H-indazol-4-yl)-propylamino]-4,6-dimethyl-pyrimidine-5-carbonyl}-amino)-propionic acid methyl ester

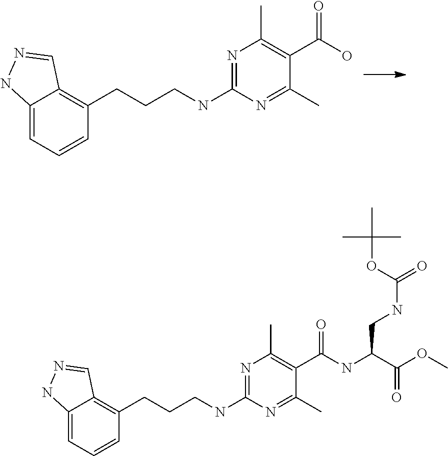

To a solution of 2-[3-(1H-indazol-4-yl)-propylamino]-4,6-dimethyl-pyrimidine-5-carboxylic acid (2.11 g, 6.45 mmol) in anhydrous DMF (60 ml) was added triethylamine (2.7 ml, 7.74 mmol), HBTU (2.94 g, 7.74 mmol), HOBT (1.05 g, 7.74 mmol), and H-DAP(Boc)OMe hydrochloride (1.97 g, 7.74 mmol). The mixture was stirred at room temperature for 18 h, diluted with brine and extracted with EtOAc. The combined organic layers were washed with brine, saturated sodium bicarbonate and brine, dried over Na₂SO₄, filtered, concentrated in the presence of silica gel and chromatographed (50-100% EtOAc in hexane) to give the desired product (2.98 g, 88% yield).

### Preparation of (S)-3-Amino-2-({2-[3-(1H-indazol-4-yl)-propylamino]-4,6-dimethyl-pyrimidine-5-carbonyl}-amino)-propionic acid methyl ester hydrochloride

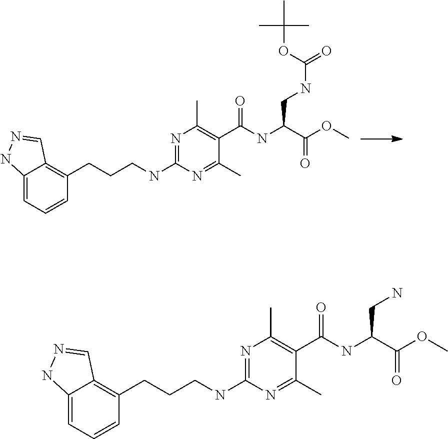

A solution of (S)-3-tert-Butoxycarbonylamino-2-({2-[3-(1H-indazol-4-yl)-propylamino]-4,6-dimethyl-pyrimidine-5-carbonyl}-amino)-propionic acid methyl ester (0.114 g, 0.22 mmol) in MeOH (2 ml) was treated with 4.0 M HCl in dioxane (5 ml). After one hour at room temperature, the mixture was concentrated and azeotroped with MeOH. The product was triturated with ether, filtered, and washed with excess ether to afford the title compound (HCl salt) (98 mg, 98% yield).

### Preparation of (S)-3-tert-butoxycarbonylamino-2-({2-[3-(3-hydroxy-phenyl)-propoxy]-4,6-dimethyl-pyrimidine-5-carbonyl}-amino-propionic acid methyl ester

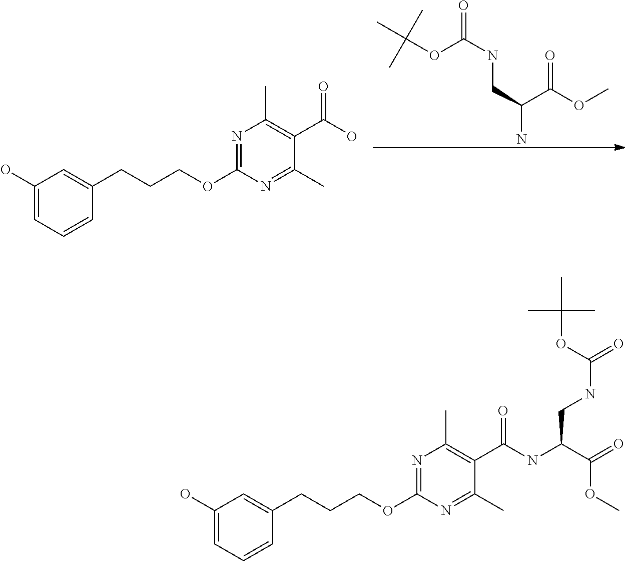

To a solution of 2-[3-(3-hydroxy-phenyl)-propoxy]-pyrimidine-5-carboxylic acid (10.25 g, 4.14 mmol) and H-DAP(BOC)-OMe.HCl (1.06 g, 4.14 mmol) in DMF (20 ml) were successively added at room temperature triethylamine (1.49 ml, 10.76 mmol), and HBTU (2.04 g, 5.4 mmol). The mixture was stirred at room temperature for 2 h then quenched with water and extracted with EtOAc. The organic layer was washed with saturated aqueous NaHCO₃ and brine, then dried over Na₂SO₄, filtered and concentrated. The crude product was purified by flash chromatography with a 20-75% EtOAC in hexane gradient to afford the desired product (1.7 g, 82% yield).

### Preparation of (S)-3-amino-2-{2-[3-(3-hydroxy-phenyl)-propoxy]-4,6-dimethyl-pyrimidine-5-carbonyl}-amino-propionic acid methyl ester

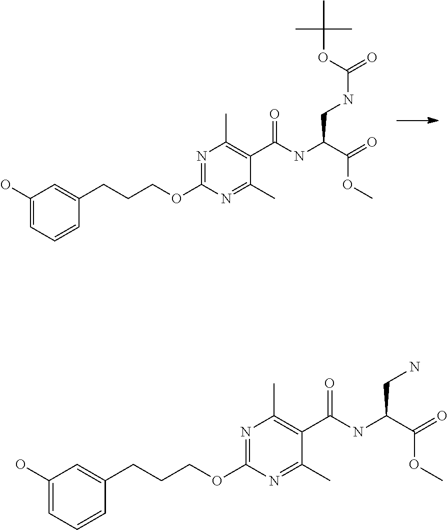

The starting BOC amine (1.7 g, 3.3 mmol) was treated with 4N HCl in dioxane (20 ml) at room temperature for 45 hr and concentrated to dryness to afford a foam. This material was suspended in dry ether and kept at room temperature overnight. The solvent was removed under reduced pressure and the resulting white solid was dried in a vacuum desiccator to afford the HCl salt of the amine as a white solid (1.6 g, 100% yield).

(S)-3-amino-2-({2-[(3′-hydroxy-biphenyl-2-ylmethyl)-amino]-4,6-dimethyl-pyrimidine-5-carbonyl}-amino)-propionic acid methyl ester hydrochloride:

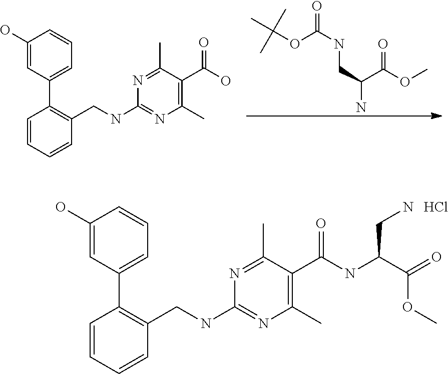

To a solution of 2-[(3′-hydroxy-biphenyl-2-ylmethyl)-amino]-4,6-dimethyl-pyrimidine-5-carboxylic acid (75 mg, 0.2 mmol) and H-DAP(BOC)-OMe.HCl (77 mg, 0.3 mmol) in DMF (2 ml) were successively added at room temperature triethylamine (112 ul, 0.8 mmol), HOBT (30 mg, 0.2 mmol) and HBTU (114 mg, 0.3 mmol). The mixture was stirred at room temperature overnight then quenched with 1NHCl and extracted with EtOAc. The organic layer was washed with pH=8 buffer and brine, then dried over Na₂SO₄, filtered and concentrated. The crude product was purified by flash chromatography with a 40-100% EtOAC in hexane gradient to afford the desired product (75 mg, 68% yield). This material was dissolved in methanol (1.5 ml) and treated with 4NHCl in dioxane (0.5 ml) at room temperature for 3.5 h. The reaction mixture was concentrated under reduced pressure to afford the desired amine HCl salt (71 mg, 100% yield).

A similar procedure was used to make the following two intermediates:
(S)-3-amino-2-({2-[(4′-hydroxy-biphenyl-2-ylmethyl)-amino]-4,6-dimethyl-pyrimidine-5-carbonyl}-amino)-propionic acid methyl ester hydrochloride:

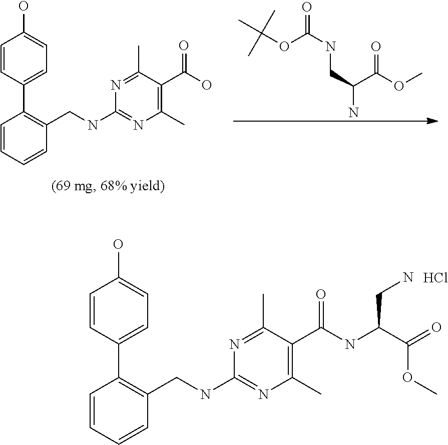

### (S)-3-amino-2-({2-[(2′-hydroxy-biphenyl-2-ylmethyl)-amino]-4,6-dimethyl-pyrimidine-5-carbonyl}-amino)-propionic acid methyl ester hydrochloride

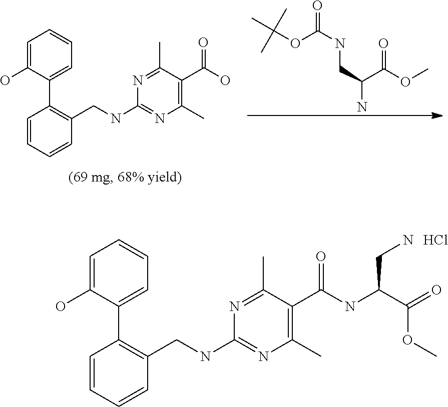

### (S)-3-amino-2-({2-[6-hydroxy-1,2,3,4-tetrahydro-naphthalen-2-ylamino]-4,6-dimethyl-pyrimidine-5-carbonyl}-amino)-propionic acid methyl ester hydrochloride

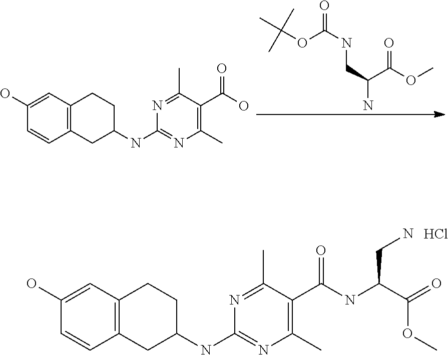

To a solution of 2-(6-hydroxy-1,2,3,4-tetrahydro-naphthalen-2-ylamino)-4,6-dimethyl-pyrimidine-5-carboxylic acid (36 mg, 0.1 mmol) and H-DAP(BOC)-OMe.HCl (33 mg, 0.15 mmol) in DMF (1.5 ml) were successively added at room temperature triethylamine (60 ul, 0.4 mmol), HOBT (15 mg, 0.1 mmol) and HBTU (60 mg, 0.15 mmol). The mixture was stirred at room temperature overnight then quenched with 1NHCl and extracted with EtOAc. The organic layer was washed with brine, then dried over Na₂SO₄, filtered and concentrated. The crude product was purified by flash chromatography with a 50-100% EtOAC in hexane gradient to afford the desired product. This material was dissolved in methanol (1.0 ml) and treated with 4NHCl in dioxane (0.3 ml) at room temperature for 2.5 h. The reaction mixture was concentrated under reduced pressure to afford the desired amine HCl salt (20 mg, 40% overall yield).

### Preparation of (S)-3-tert-Butoxycarbonylamino-2-{[2-(8-hydroxy-1,2,3,4-tetrahydro-naphthalen-2-ylamino)-4,6-dimethyl-pyrimidine-5-carbonyl]-amino}-propionic acid methyl ester

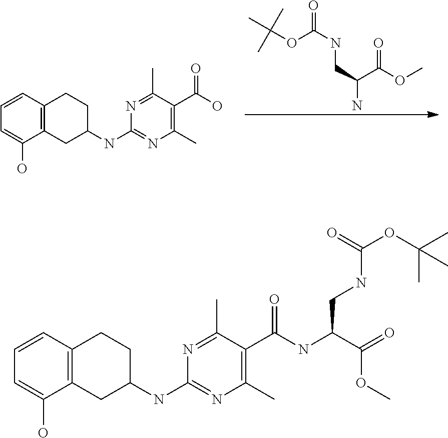

To a solution of 2-(2-(8-hydroxy-1,2,3,4-tetrahydro-naphthalen-2-ylamino)-4,6-dimethyl-pyrimidine-5-carboxylic acid (0.166 g, 0.53 mmol), Et₃N (0.22 ml, 1.59 mmol), and H-DAP(Boc)OMe hydrochloride (0.16 g, 0.64 mmol), in anhydrous DMF (4 ml) was added a solution of HBTU (0.24 g, 0.64 mmol) and HOBT (0.086 g, 0.64 mmol) in DMF (4 ml). The mixture was stirred at room temperature overnight, diluted with brine and extracted with EtOAc. The combined organic layers were washed with 1:1 satNaHCO₃/brine and brine, then dried over Na₂SO₄, filtered, and concentrated under reduced pressure. The crude residue was purified by flash chromatography with a 10-100% EtOAc in hexane gradient to give the title compound (188 mg, 69% yield). MS m/e 514.0 (M+H⁺).

### Preparation of (S)-3-Amino-2-{[2-(8-hydroxy-1,2,3,4-tetrahydro-naphthalen-2-ylamino)-4,6-dimethyl-pyrimidine-5-carbonyl]-amino}-propionic acid methyl ester hydrochloride

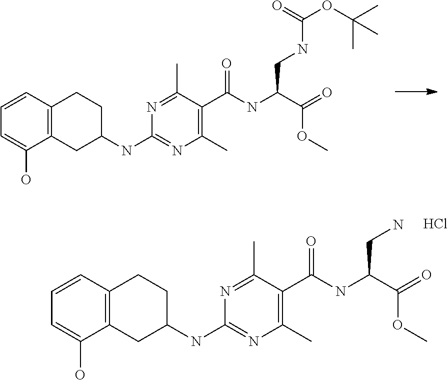

To a solution of (S)-3-tert-butoxycarbonylamino-2-{[2-(8-hydroxy-1,2,3,4-tetrahydro-naphthalen-2-ylamino)-4,6-dimethyl-pyrimidine-5-carbonyl]-amino}-propionic acid methyl ester (188 mg, 0.367 mmol) in MeOH (1 ml) was added 4.0 M HCl ind (4 ml). After one hour the mixture was concentrated and azeotroped with MeOH to afford the title compound (209 mg, 100% yield). MS m/e 414.0 (M+H⁺).

### Preparation of (S)-3-tert-butoxycarbonylamino-2-[(4,6-dimethyl-2-prop-2-ynylamino-pyrimidine-5-carbonyl)-amino]-propionic acid methyl ester

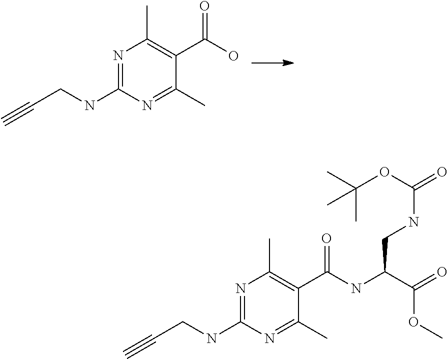

To a solution of 4,6-dimethyl-2-prop-2-ynylamino-pyrimidine-5-carboxylic acid (3 g, 14.62 mmol) in DMF (120 ml) was added triethylamine (10.2 ml, 73.1 mmol), HBTU (6.65 g, 17.54 mmol), HOBT (2.4 g, 17.54 mmol), and H-DAP(Boc)OMe hydrochloride (4.47 g, 17.54 mmol). The mixture was stirred at room temperature overnight then quenched with 1NHCl and extracted with EtOAc. The organic layer was washed with brine, then dried over Na₂SO₄, filtered and concentrated. The crude product was purified by flash chromatography with a 40-100% EtOAC in hexane gradient to afford the desired product as a white solid (4.62 g, 78% yield). MS m/e 406.1 (M+H⁺).

### Preparation of (S)-2-[(4,6-Dimethyl-2-prop-2-ynylamino-pyrimidine-5-carbonyl)-amino]-3-[(thiophene-2-carbonyl)-amino]-propionic acid methyl ester

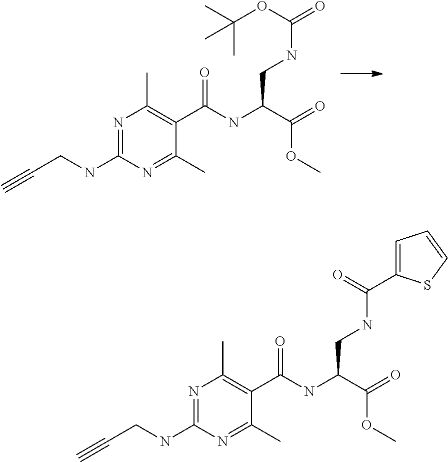

A solution of (S)-3-tert-butoxycarbonylamino-2-[(4,6-dimethyl-2-prop-2-ynylamino-pyrimidine-5-carbonyl)-amino]-propionic acid methyl ester (4.62 g, 11.39 mmol) in DCM (35 ml) was cooled to 0° C. and 30% TFA/DCM (15 ml) was added dropwise. The solution was stirred at room temperature for 1 h. The solvent and excess TFA were removed by evaporation under vacuum. The residue was dissolved in DMF (50 ml), and treated with DIEA (19.8 ml, 113 mmol) at 0° C. Thiophene-2-carboxylic acid (1.75 g, 13.67 mmol), HBTU (5.2 g, 13.67 mmol) and HOBT (1.85 g, 13.67 mmol) were added and the mixture was stirred at room temperature over the weekend. The mixture was partitioned between EtOAc and water. The layers were separated and the organic layer was washed with brine, dried over Na₂SO₄, filtered and concentrated. The residue was purified by flash chromatography with a 40-100% EtOAC in hexane gradient to afford the desired product as a white solid (4 g, 85% yield). MS m/e 415.9 (M+H⁺).

### Preparation of (S)-2-({2-[(E)-3-(6-Benzyloxy-pyridin-2-yl)-allylamino]-4,-6-dimethyl-pyrimidine-5-carbonylamino-propionic acid methyl ester

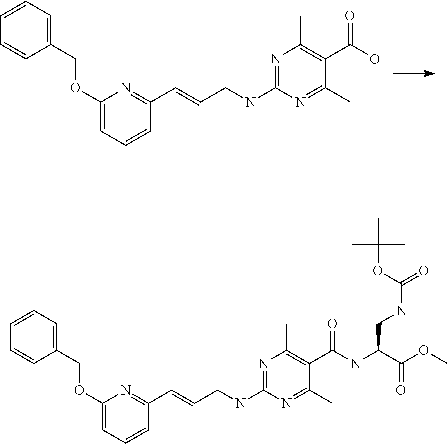

To a solution of 2-[(E)-3-(6-benzoxy-pyridin-2-yl)-allylamino]-4,6-dimethyl-pyrimidine-5-carboxylic acid (111 mg, 0.284 mmol) in anhydrous DMF (5 ml) was added triethylamine (0.12 ml, 0.85 mmol), HBTU (110 mg, 0.29 mmol), HOBT (39 mg, 0.29 mmol), and H-DAP(Boc)OMe hydrochloride (74 mg, 0.29 mmol). The mixture was stirred at room temperature overnight, then directly loaded onto a silicagel column. Eluting with a 30-100% EtOAc in hexane gradient afforded the desired product (122 mg, 73% yield). MS m/e 591.0 (M+H⁺).

### Part II: Preparation of Preferred Compounds of the Invention

### Example 1

### (S)-2-({2-[4-(3-hydroxy-phenyl)-butyl]-4,6-dimethyl-pyrimidine-5-carbonyl}-amino)-3-[(thiophene-2-carbonyl)-amino]-propionic acid

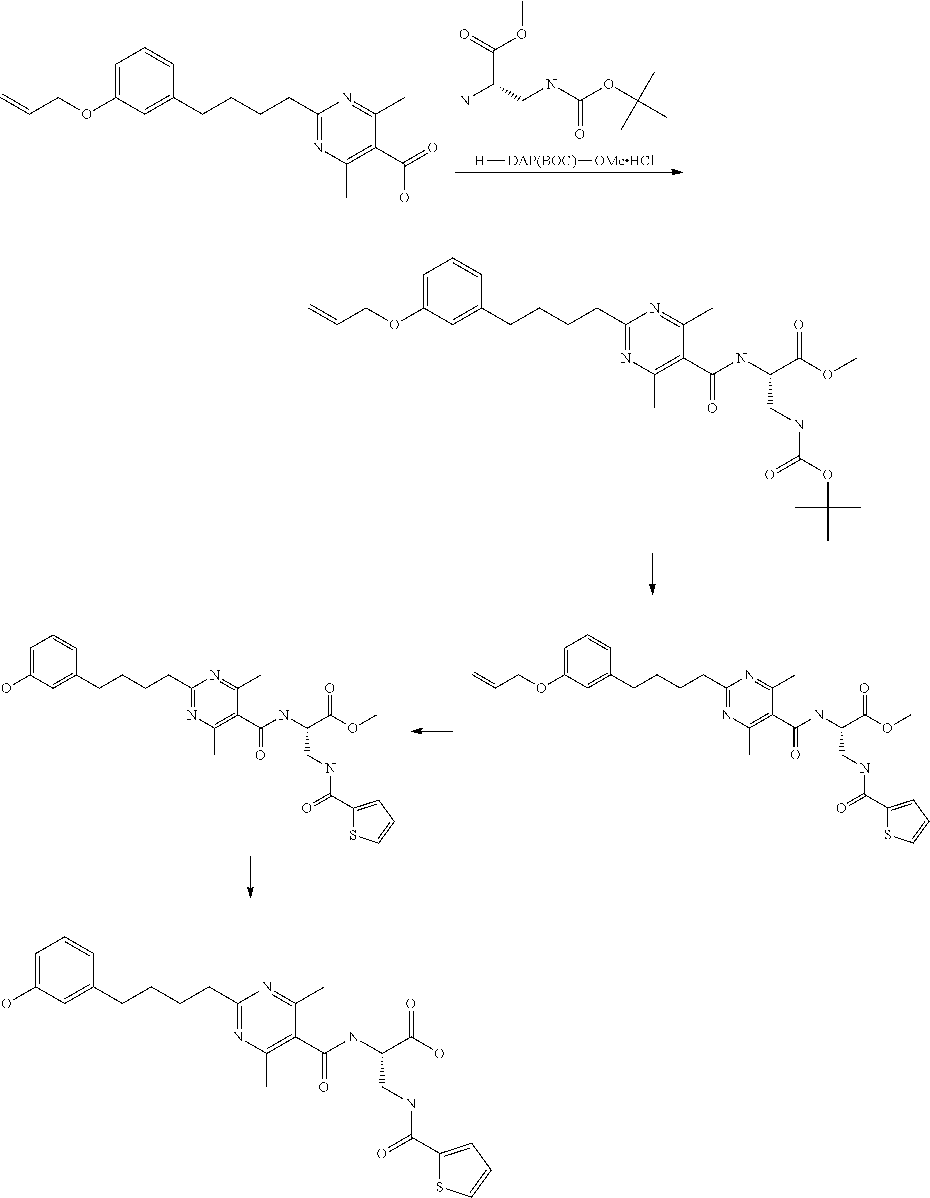

To a solution of 2-[4-(3-Allyloxy-phenyl)-butyl]-4,6-dimethyl-pyrimidine-5-carboxylic acid (64 mg, 0.188 mmol) and H-DAP(BOC)-OMe.HCl (72 mg, 0.282 mmol) in DMF (2 ml) were successively added at room temperature triethylamine (78 ul, 0.56 mmol), HOBT (47 mg, 0.34 mmol) and HBTU (130 mg, 0.39 mmol). The mixture was stirred at room temperature for 2 h then quenched with pH=3 buffer and extracted with EtOAc. The organic layer was washed with saturated aqueous NaHCO₃ and brine, then dried over Na₂SO₄, filtered and concentrated. The crude product was purified by flash chromatography with a 10-60% EtOAC in hexane gradient to afford the desired diamino propionic acid derivative (34 mg, 34% yield). This material was treated with 4N HCl in dioxane (1.5 ml) for 1 h and concentrated to dryness to afford the free amine as the HCl salt that was then taken up in DMF (1 ml). Thiophene-2-carboxylic acid (14 mg, 0.10 mmol) was added, followed by triethylamine (30 ul, 0.21 mmol), HOBT (12 mg, 0.08 mmol) and HBTU (32 mg, 0.08 mmol). The mixture was stirred at room temperature for 1 h 30 min, quenched with pH=3 buffer and extracted with EtOAc. The organic layer was washed with saturated aqueous NaHCO₃ and brine, then dried over Na₂SO₄, filtered and concentrated. The crude product was purified by flash chromatography with a 50-100% EtOAC in hexane gradient to afford the desired thiophene amide (10 mg, 26% yield from the BOC derivative). This material was dissolved in DCM (300 ul) and treated for 40 min at room temperature with PhSiH₃ (25 ul, 0.18 mmol) in the presence of a catalytic amount of Pd[PPh₃]₄ (2.5 mg, 0.002 mmol). The mixture was quenched with pH=3 buffer and extracted with EtOAc. The organic layer was washed with brine, dried over Na₂SO₄, filtered and concentrated. The crude product was purified by flash chromatography with a 50-100% EtOAC in hexane gradient to afford the desired phenol (10 mg, 100% yield). The ester was then hydrolyzed with LiOH.H₂O (0.35 mmol, 15 mg) in THF/H₂O (0.2 ml/0.2 ml) at room temperature for ½ h. The mixture was quenched by adding 220 ul of 1N KHSO₄ then partitioned between pH=3 buffer and EtOAc. The layers were separated. The organic layer was dried over Na₂SO₄, filtered and concentrated to afford the desired acid (3 mg, 34% yield, 90% pure). MS m/e 497.0 (MH⁺).

### Example 2

### (S)-2-({2-[3-(3-hydroxy-phenyl)-propoxy]-4,6-dimethyl-pyrimidine-5-carbonyl}-amino)3-[(thiophene-2-carbonyl)-amino]-propionic acid methyl ester

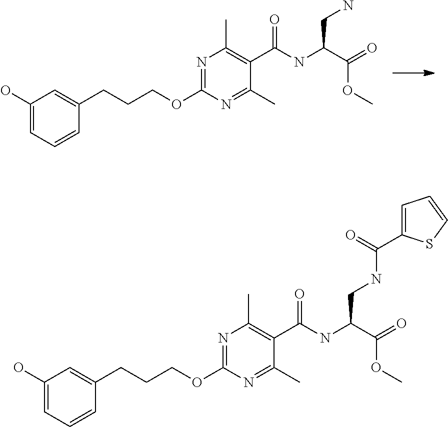

To a solution of thiophene-2-carboxylic acid (0.46 g, 3.6 mmol) and HBTU (1.78 g, 4.7 mmol) in DMA (20 ml) were successively added at room temperature triethylamine (1.0 ml, 7.2 mmol) and (S)-3-amino-2-{2-[3-(3-hydroxy-phenyl)-propoxy]-4,6-dimethyl-pyrimidine-5-carbonyl}-amino-propionic acid methyl ester (1.6 g, 3.6 mmol). The mixture was stirred at room temperature for 18 h then quenched with water and extracted with EtOAc. The organic layer was washed with saturated aqueous NaHCO₃ and brine, then dried over MgSO₄, filtered and concentrated. The crude product was purified by flash chromatography with a 20-100% EtOAC in hexane gradient to afford the desired product (1.29 g, 70% yield).

### Example 3

### (S)-2-({2-[3-(3-hydroxy-phenyl)-propoxy]-4,6-dimethyl-pyrimidine-5-carbonyl}-amino)-3-[(thiophene-2-carbonyl)-amino]-propionic acid

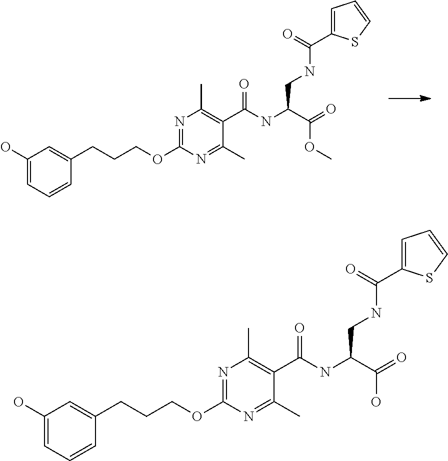

A solution of (S)-2-({2-[3-(3-hydroxy-phenyl)-propoxy]-4,6-dimethyl-pyrimidine-5-carbonyl}-amino)-3-[(thiophene-2-carbonyl)-amino]-propionic acid methyl ester (1.29 g, 2.52 mmol) in methanol (15 ml) was treated with 2NNaOH (10 ml, 20 mmol) and the reaction mixture was stirred at room temperature for 2.5 h. The volume of the reaction mixture was reduced to a third and water was added. This aqueous solution was stirred at room temperature and the pH was adjusted to 5.0 with 1NHCl to induce precipitation of the desired acid. The off-white precipitate was collected by filtration, washed with water and dried in a vacuum desiccator to give the product as a beige foam (0.84 g, 65% yield). MS m/e 499.0 (MH⁺).

### Example 4

### (S)-3-acetylamino-2-({2-[3-(1H-indazol-4-yl)-propylamino]-4,6-dimethyl-pyrimidine-5-carbonyl}-amino)-propionic acid

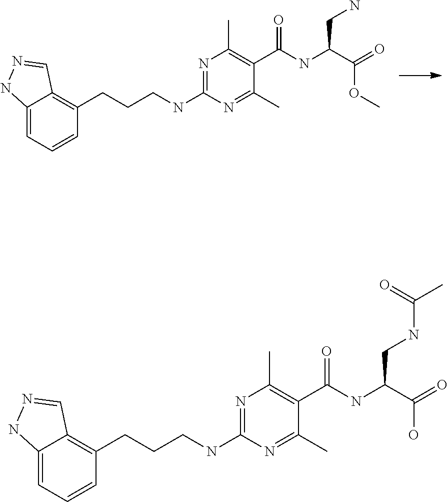

To a solution of (S)-3-amino-2-({2-[3-(1H-indazol-4-yl)-propylamino]-4,6-dimethyl-pyrimidine-5-carbonyl}-amino)-propionic acid methyl ester (0.05 g, 30.1 mmol) and triethylamine (70 ul, 0.5 mmol) in DMF (1 ml) was added Ac₂O (50 ul. 0.5 mmol). The mixture was stirred at room temperature for 2 h then quenched with 1NHCl and stirred at room temperature overnight. The phases were separated. The desired product was present in the acidic aqueous layer. This layer was made basic by adding NaOH pellets. This promoted the saponification of the acid. The aqueous layer was concentrated under reduced pressure and the residue was taken up in pH=2 buffer. The solution was concentrated and the resulting white solid was triturated with DMF/dioxane (1:10), then filtered. The filtrate was concentrated under reduced pressure to afford the desired acid (18 mg, 40% yield). MS m/e 454.3 (WO.

### Example 5

### (S)-2-({2-[3-(1H-indazol-4-yl)-propylamino]-4,6-dimethyl-pyrimidine-5-carbonyl}-amino)3-[(thiophene-2-carbonyl)-amino]-propionic acid methyl ester

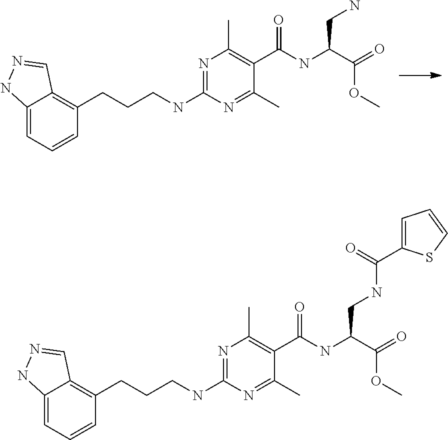

To a solution of thiophene-2-carboxylic acid (0.028 g, 0.21 mmol), HBTU (0.1 g, 0.26 mmol) and HOBT (0.035 g, 0.26 mmol) in DMF (5 ml) were successively added at room temperature triethylamine (0.09 ml, 0.26 mmol) and (S)-3-amino-2-({2-[3-(1H-indazol-4-yl)-propylamino]-4,6-dimethyl-pyrimidine-5-carbonyl}-amino)-propionic acid methyl ester (0.1 g, 0.21 mmol). The mixture was stirred at room temperature for 18 h then quenched with water and extracted with EtOAc. The organic layer was washed with saturated aqueous NaHCO₃ and brine, then dried over MgSO₄, filtered and concentrated. The crude product was purified by flash chromatography with a 50-100% EtOAC in hexane gradient to afford the desired product (0.059 g, 52% yield).

### Example 6

### (S)-2-({2-[3-(1H-indazol-4-yl)-propylamino]-4,6-dimethyl-pyrimidine-5-carbonyl}-amino)3-[(thiophene-2-carbonyl)-amino]-propionic acid

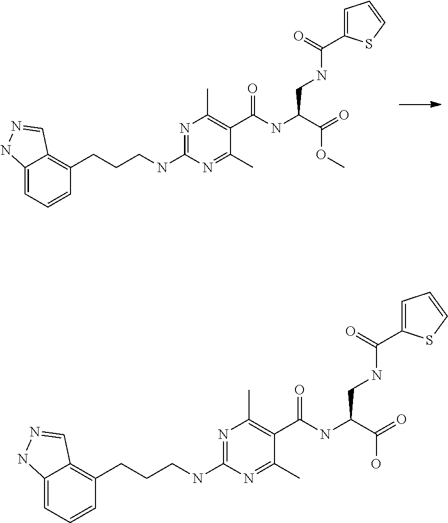

A solution of (S)-2-({2-[3-(1H-indazol-4-yl)-propylamino]-4,6-dimethyl-pyrimidine-5-carbonyl}-amino)3-[(thiophene-2-carbonyl)-amino]-propionic acid methyl ester (0.059 g, 0.11 mmol) in THF/water (4 ml/5 ml)) was treated with LiOH.H₂O (0.023 g, 0.55 mmol) and the reaction mixture was stirred at room temperature for 1 h. The reaction mixture was quenched by adding 1N KHSO₄ (0.55 ml) and water. The white precipitate was collected by filtration, washed with water then suspended in acetonitrile/water and lyophilized. White solid (0.056 g, 98% yield). MS m/e 522.1 (MH⁺).

### Example 7

### (S)-2-({2-[3-(3-hydroxy-phenyl)-propylamino]-4,6-dimethyl-pyrimidine-5-carbonyl}-amino)3-[(thiophene-2-carbonyl)-amino]-propionic acid methyl ester

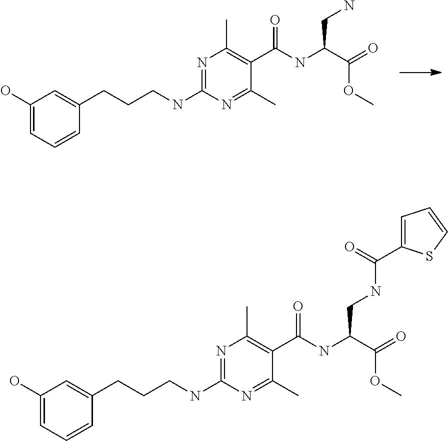

To a solution of thiophene-2-carboxylic acid (0.24 g, 1.85 mmol), HBTU (0.84 g, 2.22 mmol) and HOBT (0.30 g, 0.22 mmol) in DMF (40 ml) were added at room temperature triethylamine (0.56 ml, 5.55 mmol) and (S)-3-amino-2-({2-[3-(3-hydroxy-phenyl)-propylamino]-4,6-dimethyl-pyrimidine-5-carbonyl}-amino)-propionic acid methyl ester (0.81 g, 1.85 mmol). The mixture was stirred at room temperature for 1 h then quenched with water/brine and extracted with EtOAc. The organic layer was washed with brine, then dried over Na₂SO₄, filtered and concentrated. The crude product was purified by flash chromatography with a 40-100% EtOAC in hexane gradient to afford the desired product (0.59 g, 62% yield).

### Example 8

### (S)-2-({2-[3-(3-hydroxy-phenyl)-propylamino]-4,6-dimethyl-pyrimidine-5-carbonyl}-amino)3-[(thiophene-2-carbonyl)-amino]-propionic acid

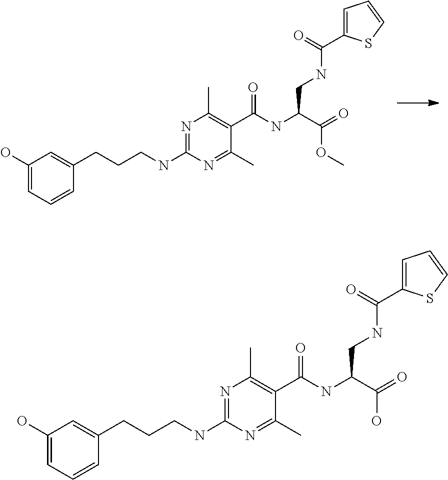

A solution of (S)-2-({2-[3-(3-hydroxy-phenyl)-propylamino]-4,6-dimethyl-pyrimidine-5-carbonyl}-amino)3-[(thiophene-2-carbonyl)-amino]-propionic acid methyl ester (0.59 g, 1.15 mmol) in THF/water (20/30 ml) was treated with LiOH.H₂O (0.24 g, 5.77 mmol) and the reaction mixture was stirred at room temperature for 1.5 h. The mixture was quenched with 1N KHSO₄ (6 ml) and concentrated to about half its volume. The white precipitate was collected by suction filtration, washed with water and air-dried.

(0.54 g, 94% yield). MS m/e 497.9 (MH⁺).

### Example 9

### (S)-2-({2-[(3′-hydroxy-biphenyl-2-ylmethyl)-amino]-4,6-dimethyl-pyrimidine-5-carbonyl}-amino)-3-[(thiophene-2-carbonyl)-amino]-propionic acid methyl ester

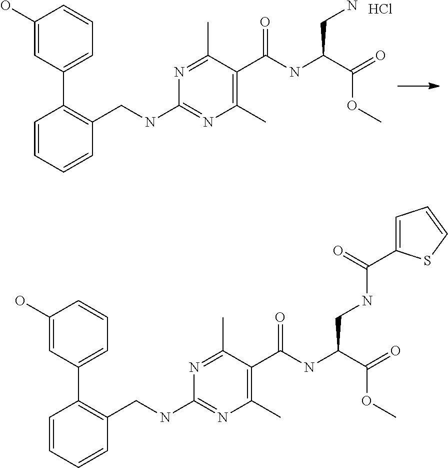

To a solution of (S)-3-amino-2-({2-[(3′-hydroxy-biphenyl-2-ylmethyl)-amino]-4,6-dimethyl-pyrimidine-5-carbonyl}-amino)-propionic acid methyl ester, HCl salt (71 mg, 0.16 mmol) and thiophene-2-carboxylic acid (22 mg, 0.17 mmol) in DMF (2 ml), were successively added at room temperature triethylamine (66 ul, 0.47 mmol), HOBT (24 mg, 0.16 mmol) and HBTU (66 mg, 0.17 mmol). The mixture was stirred at room temperature for 0.5 h then quenched with 1NHCl and extracted with EtOAc. The combined organic layers were washed with brine, dried over Na₂SO₄, filtered and concentrated. The crude product was purified by flash chromatography with a 50-100% EtOAC in hexane gradient to afford the desired product (47 mg, 62% yield).

A similar procedure was used to make the following two examples:

### Example 10

### (S)-2-({2-[(4′-hydroxy-biphenyl-2-ylmethyl)-amino]-4,6-dimethyl-pyrimidine-5-carbonyl}-amino)-3-[(thiophene-2-carbonyl)-amino]-propionic acid methyl ester

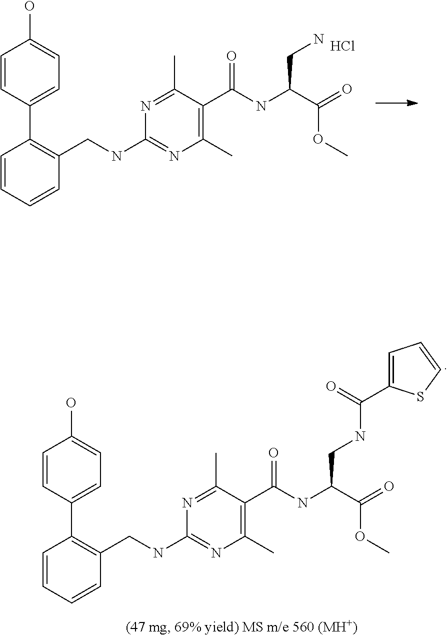

### Example 11

### (S)-2-({2-[(2′-hydroxy-biphenyl-2-ylmethyl)-amino]-4,6-dimethyl-pyrimidine-5-carbonyl}-amino)-3-[(thiophene-2-carbonyl)-amino]-propionic acid methyl ester

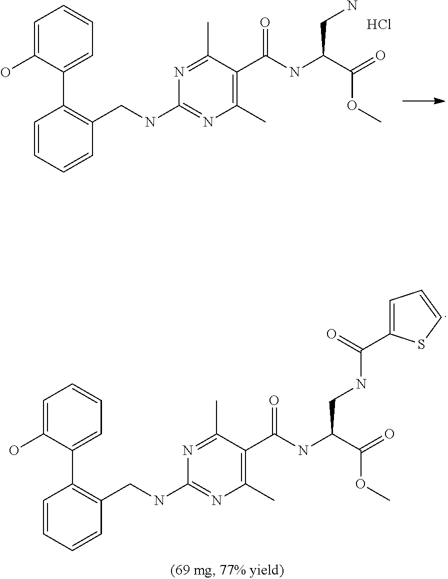

### Example 12

### (S)-2-({2-[(3′-hydroxy-biphenyl-2-ylmethyl)-amino]-4,6-dimethyl-pyrimidine-5-carbonyl}-amino)-3-[(thiophene-2-carbonyl)-amino]-propionic acid

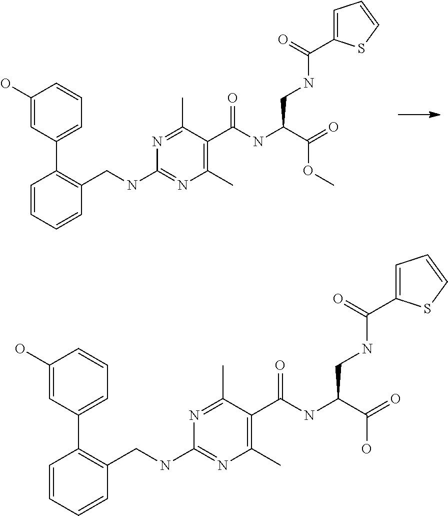

A solution of (S)-2-({2-[(3′-hydroxy-biphenyl-2-ylmethyl)-amino]-4,6-dimethyl-pyrimidine-5-carbonyl}-amino)-3-[(thiophene-2-carbonyl)-amino]-propionic acid methyl ester (47 mg, 0.08 mmol) in H₂O/dioxane (1 ml/1 ml) was treated with LiOH.H₂O (35 mg, 0.84 mmol) at room temperature for 1.5 h. The reaction mixture was quenched with 1NHCl (1.5 ml) and concentrated under reduced pressure. The residue was partitioned between pH=2 buffer and EtOAc. The organic phase was dried over Na₂SO₄, filtered and concentrated to afford the desired acid (43 mg, 100% yield). MS m/e 545.9 (MH⁺).

A similar procedure was used to make the following two examples:

### Example 13

### (S)-2-({2-[(3′-hydroxy-biphenyl-2-ylmethyl)-amino]-4,6-dimethyl-pyrimidine-5-carbonyl}-amino)-3-[(thiophene-2-carbonyl)-amino]-propionic acid

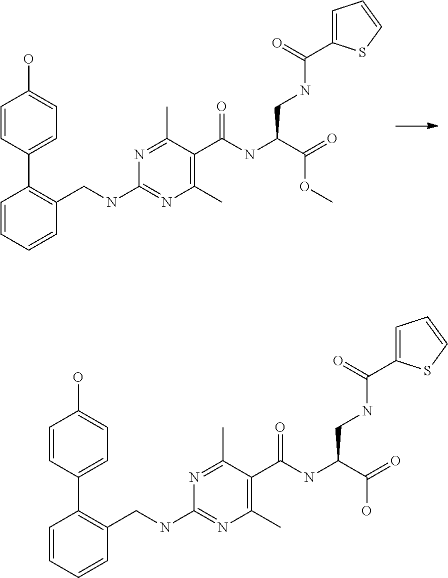

### Example 14

### (S)-2-({2-[(2′-hydroxy-biphenyl-2-ylmethyl)-amino]-4,6-dimethyl-pyrimidine-5-carbonyl}-amino)-3-[(thiophene-2-carbonyl)-amino]-propionic acid

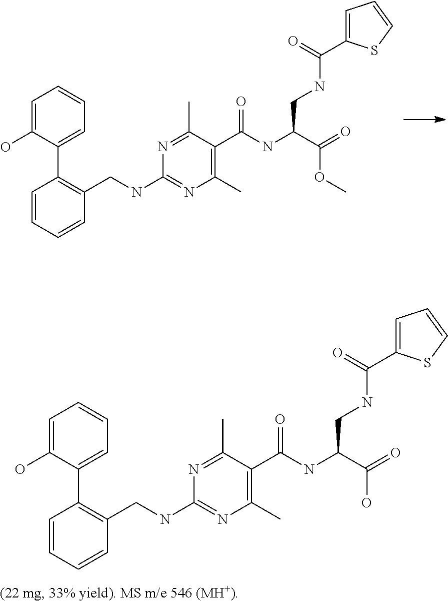

### Example 15

### (S) 2-(2-[6-hydroxy-1,2,3,4-tetrahydro-naphthalen-2-ylamino]-4,6-dimethyl-pyrimidine-5-carbonyl-amino)-3-[(thiophene-2-carbonyl)-amino]-propionic acid methyl ester

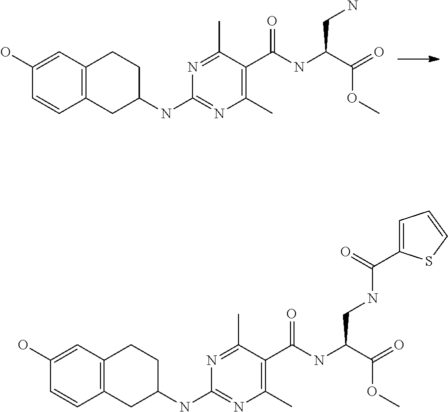

To a solution of (S)-3-amino-2-({2-[6-hydroxy-1,2,3,4-tetrahydro-naphthalen-2-ylamino]-4,6-dimethyl-pyrimidine-5-carbonyl}-amino)-propionic acid methyl ester hydrochloride (18 mg, 0.04 mmol) and thiophene-2-carboxylic acid (6 mg, 0.05 mmol) in DMF (0.5 ml), were successively added at room temperature triethylamine (18 ul, 0.13 mmol), HOBT (7 mg, 0.04 mmol) and HBTU (17 mg, 0.05 mmol). The mixture was stirred at room temperature for 1 h then quenched with 1NHCl and extracted with EtOAc. The combined organic layers were washed with brine, dried over Na₂SO₄, filtered and concentrated. The crude product was purified by flash chromatography with a 50-100% EtOAC in hexane gradient to afford the desired product (12 mg, 60% yield).

A similar procedure was used to prepare the following analog:

### Example 16

### (S) 2-(2-[8-hydroxy-1,2,3,4-tetrahydro-naphthalen-2-ylamino]-4,6-dimethyl-pyrimidine-5-carbonyl-amino)-3-[(thiophene-2-carbonyl)-amino]-propionic acid methyl ester

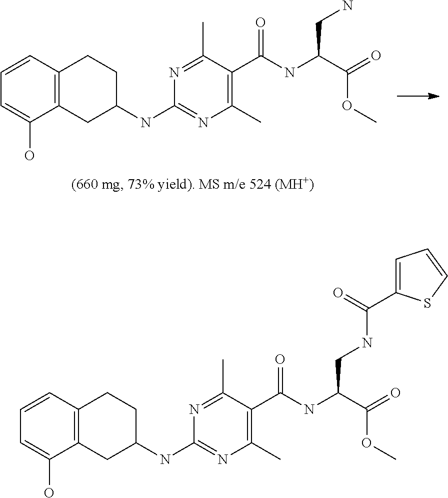

### Example 17

### (S) 2-(2-[6-hydroxy-1,2,3,4-tetrahydro-naphthalen-2-ylamino]-4,6-dimethyl-pyrimidine-5-carbonyl-amino)-3-[(thiophene-2-carbonyl)-amino]-propionic acid

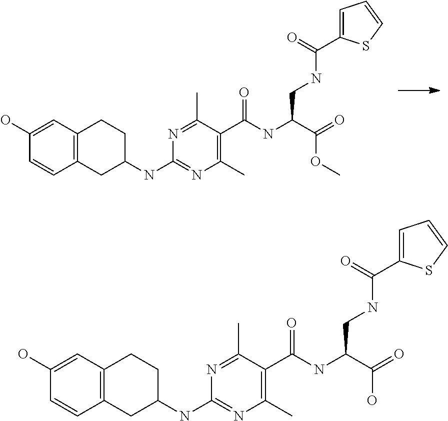

A solution of (S) 2-(2-[6-hydroxy-1,2,3,4-tetrahydro-naphthalen-2-ylamino]-4,6-dimethyl-pyrimidine-5-carbonyl-amino)-3-[(thiophene-2-carbonyl)-amino]-propionic acid methyl ester (24 mg, 0.04 mmol) in H₂O/dioxane (1 ml/1 ml) was treated with LiOH.H₂O (17 mg, 0.40 mmol) at room temperature for 3 h. The reaction mixture was quenched with 1NHCl and concentrated under reduced pressure. The residue was partitioned between pH=2 buffer and EtOAc/MeOH (10/1). The organic phase was dried over Na₂SO₄, filtered and concentrated. The residue was purified by reverse phase HPLC to afford the desired acid as white solid (5 mg, 25% yield). MS m/e 509.9 (MH⁺).

A similar procedure was used to prepare the following analog:

### Example 18

### (S) 2-(2-[8-hydroxy-1,2,3,4-tetrahydro-naphthalen-2-ylamino]-4,6-dimethyl-pyrimidine-5-carbonyl-amino)-3-[(thiophene-2-carbonyl)-amino]-propionic acid

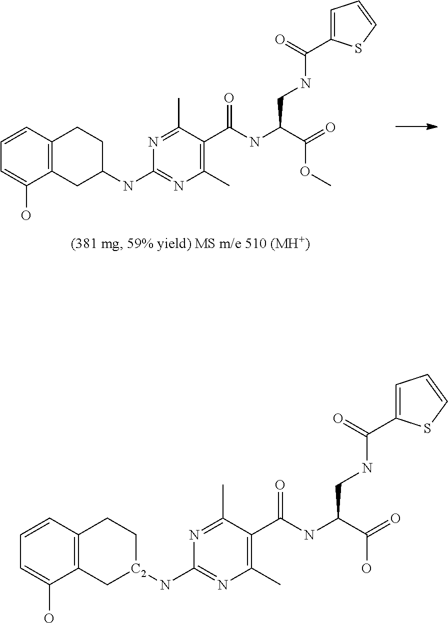

### Example 19

### (S)-2-({2-[3-(3-Hydroxy-phenyl)-prop-2-ynylamino]-4,6-dimethyl-pyrimidine-5-carbonyl}-amino)-3-[(thiophene-2-carbonyl)-amino]-propionic acid methyl ester

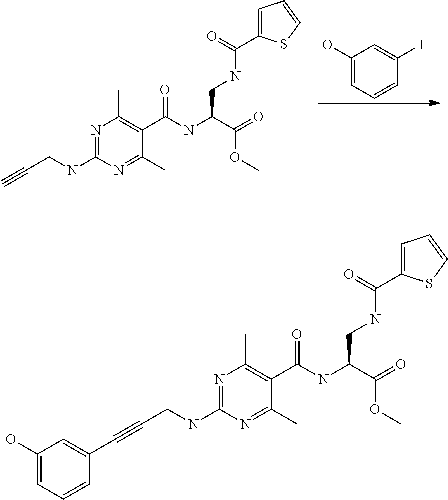

A reaction vessel containing 3-iodo-phenol (39.7 mg, 0.18 mmol), DIEA (1 ml), dichlorobis(triphenylphosphine)palladium (II) (4.2 mg, 0.006 mmol), copper (I) iodide (2.3 mg, 0.012 mmol) and DMF (1 ml) was sealed, degassed, and flushed with nitrogen. The mixture was stirred at room temperature for 30 min. A solution of (S)-2-[(4,6-dimethyl-2-prop-2-ynylamino-pyrimidine-5-carbonyl)-amino]-3-[(thiophene-2-carbonyl)-amino]-propionic acid methyl ester (50 mg, 0.1 mmol) and DIEA (0.5 ml) in DMF (0.5 ml) was added at room temperature. The reaction mixture was stirred at room temperature for 18 h. The mixture was extracted with EtOAc. The combined organic extracts were washed with water and brine, dried over sodium sulfate, filtered and concentrated. The residue was purified by flash chromatography with a 40-100% EtOAc in hexane gradient to afford the desired product as a yellow solid (29 mg, 48% yield). MS m/e 508.0 (M+H⁺).

### Example 20

### (S)-2-({2-[(Z)-3-(hydroxy-phenyl)-allylamino]-4,6-dimethyl-pyrimidine-5-carbonyl}-amino)-3-[(thiophene-2-carbonyl)-amino]-propionic acid methyl ester

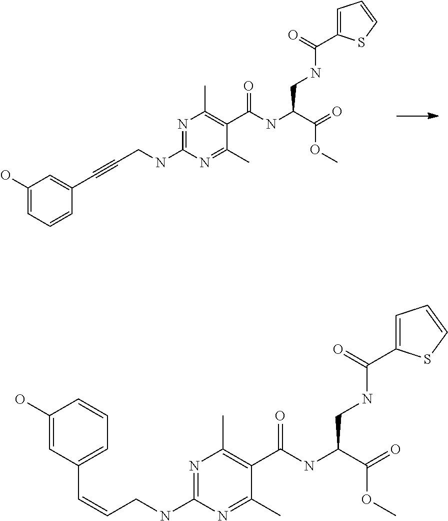

A mixture of (S)-2-({2-[3-(3-hydroxy-phenyl)-prop-2-ynylamino]-4,6-dimethyl-pyrimidine-5-carbonyl}-amino)-3-[(thiophene-2-carbonyl)-amino]-propionic acid methyl ester (552.6 mg, 1.09 mmol), and Lindlar's catalyst (350 mg) in EtOAc (100 ml) was subjected to hydrogenation via Parr Shaker Type Apparatus at 40 psi and room temperature for 8 h. The solution was filtered and the filtrate was concentrated under reduced pressure. The residue was purified by flash chromatography with 10% MeOH in DCM to give the expected product as a white solid (352.5 mg, 63% yield). MS m/e 510.0 (M+H⁺).

### Example 21

### (S)-2-({2-[3-(1H-indazol-4-yl)-prop-2-ynylamino]-4,6-dimethyl-pyrimidine-5-carbonyl}-amino)-3-[(thiophene-2-carbonyl)-amino]-propionic acid methyl ester

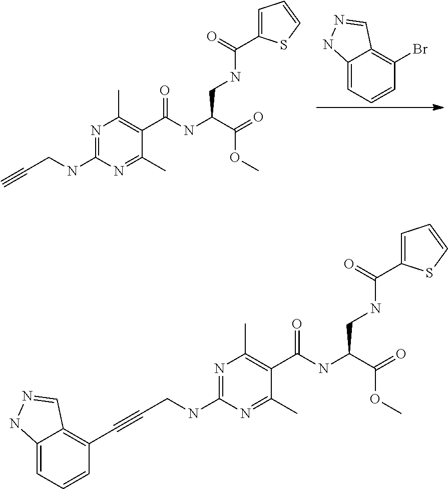

A reaction vessel containing 4-bromo-indazole (71 mg, 0.36 mmol), DIEA (1 ml), dichlorobis(triphenylphosphine)palladium (II) (8.4 mg, 0.012 mmol), copper (I) iodide (4.4 mg, 0.024 mmol) and DMF (1 ml) was sealed, degassed, and flushed with nitrogen. The mixture was stirred at room temperature for 30 min. A solution of (S)-2-[(4,6-dimethyl-2-prop-2-ynylamino-pyrimidine-5-carbonyl)-amino]-3-[(thiophene-2-carbonyl)-amino]-propionic acid methyl ester (100 mg, 0.24 mmol) and DIEA (0.5 ml) in DMF (0.5 ml) was added at room temperature. The reaction mixture was stirred at 50° C. for 18 h. The mixture was cooled to room temperature and extracted with EtOAc. The combined organic extracts were washed with water and brine, dried over sodium sulfate, filtered and concentrated. The residue was purified by flash chromatography with 20% MeOH in DCM to give pure product (37.3 mg, 29% yield). MS m/e 532.0 (M+H⁺).

### Example 22

### (S)-2-({2-[3-(1H-indazol-6-yl)-prop-2-ynylamino]-4,6-dimethyl-pyrimidine-5-carbonyl}-amino)-3-[(thiophene-2-carbonyl)-amino]-propionic acid methyl ester

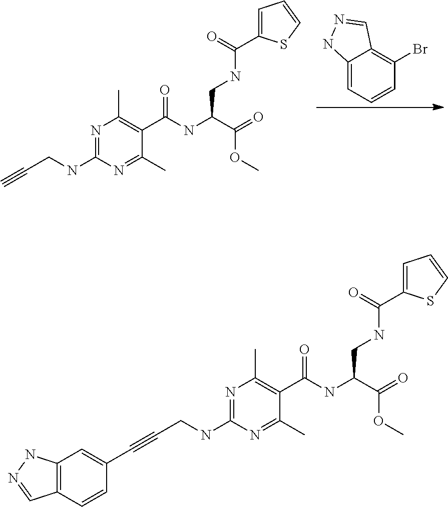

A reaction vessel containing 6-bromoindazole (141.9 mg, 0.72 mmol), Et₃N (1.5 ml), dichlorobis(triphenylphosphine)palladium (II) (16.8 mg, 0.024 mmol), copper (I) iodide (9.2 mg, 0.048 mmol) and DMF (2 ml) was sealed, degassed and flushed with nitrogen. The mixture was stirred at room temperature for 30 min. A solution of (S)-2-[(4,6-dimethyl-2-prop-2-ynylamino-pyrimidine-5-carbonyl)-amino]-3-[(thiophene-2-carbonyl)-amino]-propionic acid methyl ester (200 mg, 0.48 mmol) in DMF (2 ml) was added at room temperature. The reaction mixture was stirred at 47° C. for 24 h. The mixture was cooled to room temperature and extracted with EtOAc. The combined organic extracts were washed with water and brine, dried over sodium sulfate, filtered and concentrated. The residue was purified by flash chromatography with a 40-100% EtOAc in hexane gradient to give pure product (99.2 mg, 37% yield). MS m/e 532.0 (M+H⁺).

### Example 23

### (S)-2-({2-[3-(1H-indazol-6-yl)-propylamino]-4,6-dimethyl-pyrimidine-5-carbonyl}-amino)-3-[(thiophene-2-carbonyl)-amino]propionic acid methyl ester

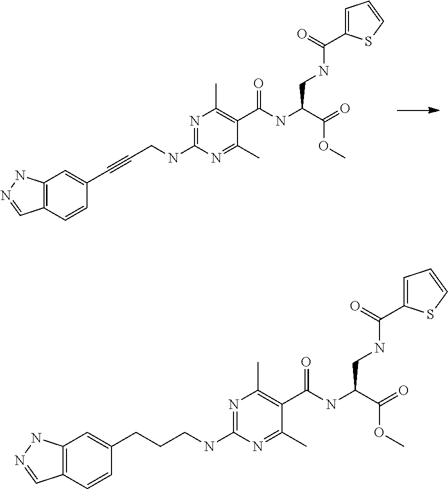

A solution of (S)-2-({2-[3-(1H-indazol-6-yl)-prop-2-ynylamino]-4,6-dimethyl-pyrimidine-5-carbonyl}-amino)-3-[(thiophene-2-carbonyl)-amino]-propionic acid methyl ester (85 mg, 0.16 mmol) in MeOH (75 ml) was passed through H-Cube with 5% Pd/C small cartridge at 1 atmosphere, room temperature and 1 ml per min. Solvent was then removed by evaporation and the residue was purified by flash chromatography with 10% MeOH in DCM to give the desired product (65 mg, 76% yield). MS m/e 536.0 (M+H⁺).

### Example 24

### (S)-2-({2-[3-(3-Fluoro-5-hydroxy-phenyl)-prop-2-ynylamino]-4,6-dimethyl-pyrimidine-5-carbonyl}-amino)-3-[(thiophene-2-carbonyl)-amino]propionic acid methyl ester

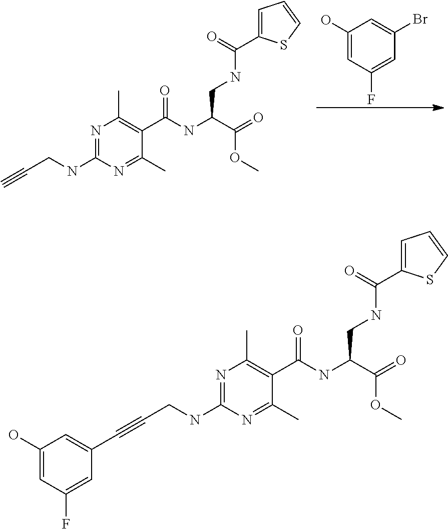

A reaction vessel containing 3-fluoro-5-bromophenol (69 mg, 0.36 mmol), Et₃N (1.5 ml), dichlorobis(triphenylphosphine)palladium (II) (8.4 mg, 0.012 mmol) and copper (I) iodide (4.6 mg, 0.024 mmol) in DMF (1 ml) was sealed, degassed, and flushed with nitrogen. The mixture was stirred at room temperature for 30 min. A solution of (S)-2[(4,6-dimethyl-2-prop-2-ynylamino-pyrimidine-5-carbonyl)-amino]-3-[(thiophene-2-carbonyl)-amino]-propionic acid methyl ester (100 mg, 0.24 mmol) in DMF (1 mL) was added at room temperature. The reaction mixture was stirred at room temperature over the weekend. The mixture was extracted with EtOAc. The combined organic extracts were washed with water and brine, dried over sodium sulfate, filtered and concentrated. The residue was purified by flash chromatography with 10% MeOH in DCM to give pure product (51 mg, 40% yield). MS m/e 525.9 (M+H⁺).

### Example 25

### (S)-2-({2-[3-(3-fluoro-5-hydroxy-phenyl)-propylamino]-4,6-dimethyl-pyrimidine-5-carbonyl}-amino)-3-[(thiophene-2-carbonyl)-amino]propionic acid methyl ester

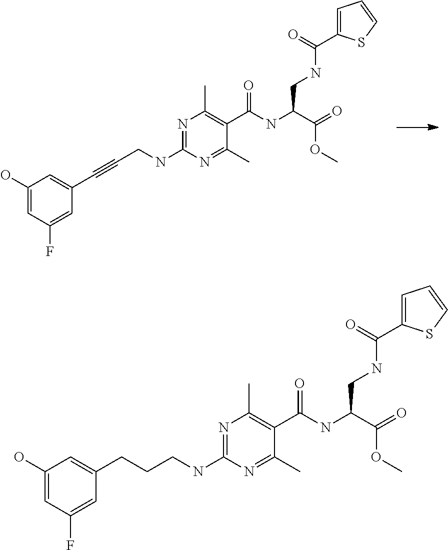

A solution of (S)-2-({2-[3-(3-fluoro-5-hydroxy-phenyl)-prop-2-ynylamino]-4,6-dimethyl-pyrimidine-5-carbonyl}-amino)-3-[(thiophene-2-carbonyl)-amino]propionic acid methyl ester (67.7 mg, 0.13 mmol) in MeOH (50 ml) was passed through H-Cube with 5% Pd/C small cartridge at 1 atmosphere, room temperature and 1 ml per min. Solvent was then removed by evaporation and the residue was purified by flash chromatography with 90% EtOAc in hexane to give pure product (18.2 mg, 26% yield). MS m/e 530.0 (M+H⁺).

### Example 26

### (S)-2-({2-[3-(4-Fluoro-3-hydroxy-phenyl)-prop-2-ynylamino]-4,6-dimethyl-pyrimidine-5-carbonyl}-amino)-3-[(thiophene-2-carbonyl)-amino]propionic acid methyl ester

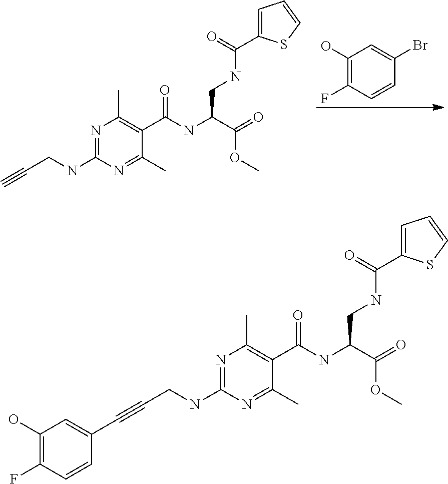

A reaction vessel containing 2-fluoro-5-bromophenol (138 mg, 0.72 mmol), Et₃N (1.5 ml), dichlorobis(triphenylphosphine) palladium (II) (16.8 mg, 0.024 mmol) and copper (I) iodide (9.2 mg, 0.048 mmol) in DMF (2 ml) was sealed, degassed, and flushed with nitrogen. The mixture was stirred at room temperature for 30 min. A solution of (S)-2[(4,6-dimethyl-2-prop-2-ynylamino-pyrimidine-5-carbonyl)-amino]-3-[(thiophene-2-carbonyl)-amino]-propionic acid methyl ester (200 mg, 0.48 mmol) in DMF (2 ml) was added at room temperature. The reaction mixture was stirred at 47° C. for 18 h. The mixture was extracted with EtOAc. The combined organic extracts were washed with water and brine, dried over sodium sulfate, filtered and concentrated. The residue was purified by flash chromatography with 10% MeOH in DCM to give pure product (42 mg, 17% yield). MS m/e 526.0 (M+H⁺).

### Example 27

### (S)-2-({2-[3-(4-fluoro-3-hydroxy-phenyl)-propylamino]-4,6-dimethyl-pyrimidine-5-carbonyl}-amino)-3-[(thiophene-2-carbonyl)-amino]propionic acid methyl ester

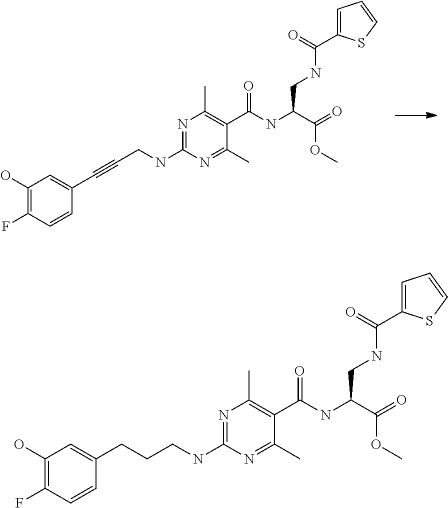

A solution of (S)-2-({2-[3-(2-fluoro-5-hydroxy-phenyl)-prop-2-ynylamino]-4,6-dimethyl-pyrimidine-5-carbonyl}-amino)-3-[(thiophene-2-carbonyl)-amino]propionic acid methyl ester (42 mg, 0.08 mmol) in MeOH (50 ml) was passed through H-Cube with 5% Pd/C small cartridge at 1 atmosphere, room temperature and 1 ml per min. Solvent was then removed by evaporation and the residue was purified by flash chromatography with 5% MeOH in DCM to give pure product (42 mg, 99% yield). MS m/e 530.0 (M+H⁺).

### Example 28

### (S)-2-({2-[3-(3-Hydroxy-2-methyl-phenyl)-prop-2-ynylamino]-4,6-dimethyl-pyrimidine-5-carbonyl}-amino)-3-[(thiophene-2-carbonyl)-amino]propionic acid methyl ester

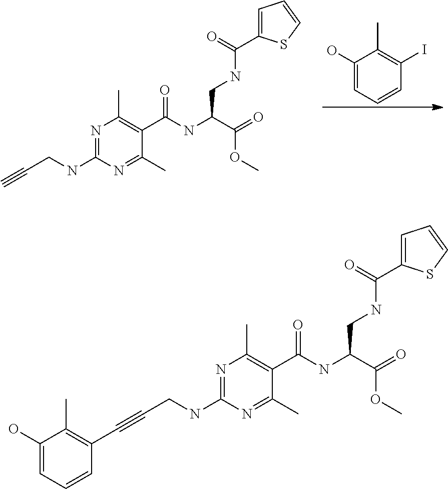

A reaction vessel containing 3-iodo-2-methyl-phenol (253 mg, 0.72 mmol), Et₃N (2 ml) dichlorobis(triphenylphosphine)palladium (II) (25.3 mg, 0.036 mmol) and copper (I) iodide (13.5 mg, 0.072 mmol) in DMF (2 ml) was sealed, degassed, and flushed with nitrogen. The mixture was stirred at room temperature for 30 min A solution of (S)-2-[(4,6-dimethyl-2-prop-2-ynylamino-pyrimidine-5-carbonyl)-amino]-3-[(thiophene-2-carbonyl)-amino]-propionic acid methyl ester (300 mg, 0.72 mmol) and Et₃N (1 ml) in DMF (1 ml) was added at room temperature. The reaction mixture was stirred at room temperature for 18 h. The solution was purified directly by preparative HPLC under neutral condition to give pure product (370 mg, 48% yield). MS m/e 521.9 (M+H⁺).

### Example 29

### (S)-2-({2-[3-(3-Hydroxy-2-methyl-phenyl)-propylamino]-4,6-dimethyl-pyrimidine-5-carbonyl}-amino)-3-[(thiophene-2-carbonyl)-amino]propionic acid methyl ester

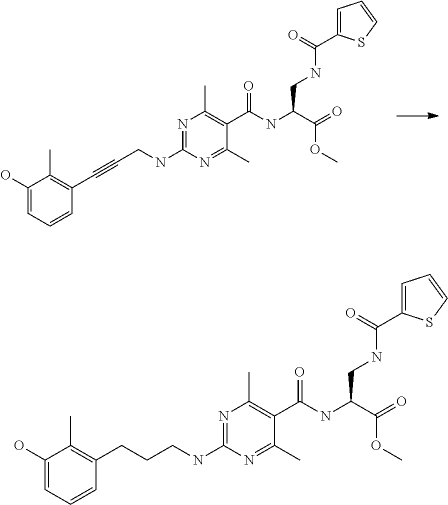

A solution of (S)-2-({2-[3-(3-hydroxy-2-methyl-phenyl)-prop-2-ynylamino]-4,6-dimethyl-pyrimidine-5-carbonyl}-amino)-3-[(thiophene-2-carbonyl)-amino]propionic acid methyl ester (370 mg, 0.71 mmol) in MeOH (200 ml) was passed through H-Cube with 10% Pd/C small cartridge at 30 bar, 30° C. and 1 ml per min twice. The solvent was then removed by evaporation and the residue was purified by flash chromatography with 100% EtOAc to afford the desired product (197 mg, 53% yield). MS m/e 525.9 (M+H⁺).

### Example 30

### (S)-2-({2-[3-(2-chloro-5-methoxy-phenyl)-prop-2-ynylamino]-4,6-dimethyl-pyrimidine-5-carbonyl}-amino)-3-[(thiophene-2-carbonyl)-amino]propionic acid methyl ester

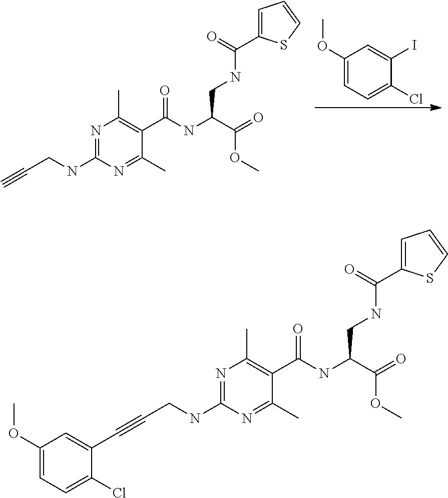

A reaction vessel containing 1-chloro-2-iodo-4-methoxy-benzene (388 mg, 1.44 mmol), Et₃N (2 ml), dichlorobis(triphenylphosphine)palladium (II) (25.3 mg, 0.036 mmol) and copper (I) iodide (13.5 mg, 0.072 mmol) in DMF (2 ml) was sealed, degassed, and flushed with nitrogen. The mixture was stirred at room temperature for 30 min. A solution of (S)-2-[(4,6-dimethyl-2-prop-2-ynylamino-pyrimidine-5-carbonyl)-amino]-3-[(thiophene-2-carbonyl)-amino]-propionic acid methyl ester (300 mg, 0.72 mmol) and Et₃N (1 ml) in DMF (1 ml) was added at room temperature. The reaction mixture was stirred at room temperature for 18 h. The mixture was extracted with EtOAc. The combined organic extracts were washed with water and brine, dried over sodium sulfate, filtered and concentrated. The residue was purified by flash chromatography with 30% EtOAc in hexane to afford the title compound (284 mg, 71% yield). MS m/e 555.8 (M+H⁺).

### Example 31

### (S)-2-({2-[3-(2-chloro-5-methoxy-phenyl)-propylamino]-4,6-dimethyl-pyrimidine-5-carbonyl}-amino)-3-[(thiophene-2-carbonyl)-amino]propionic acid methyl ester

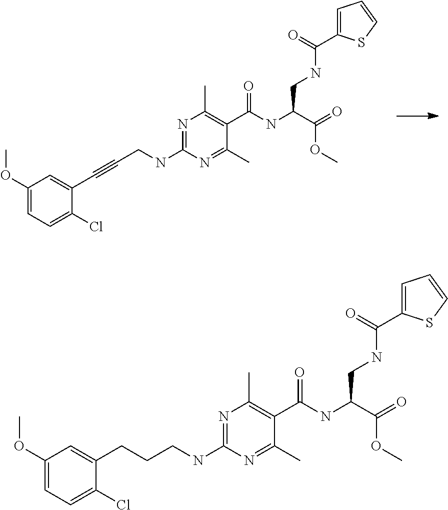

A solution of (S)-2-({2-[3-(2-chloro-5-methoxy-phenyl)-prop-2-ynylamino]-4,6-dimethyl-pyrimidine-5-carbonyl}-amino)-3-[(thiophene-2-carbonyl)-amino]propionic acid methyl ester (284 mg, 0.51 mmol) in MeOH (300 ml) was passed through H-Cube with 5% Pt/C sulfided small cartridge at 40 bar, room temperature and 1 ml per min three times. Solvent was then removed by evaporation to give pure product (18.1 mg, 63% yield). MS m/e 559.8 (M+H⁺).

### Example 32

### (S)-2-({2-[3-(4-Hydroxy-phenyl)-prop-2-ynylamino]-4,6-dimethyl-pyrimidine-5-carbonyl}-amino)-3-[(thiophene-2-carbonyl)-amino]propionic acid methyl ester

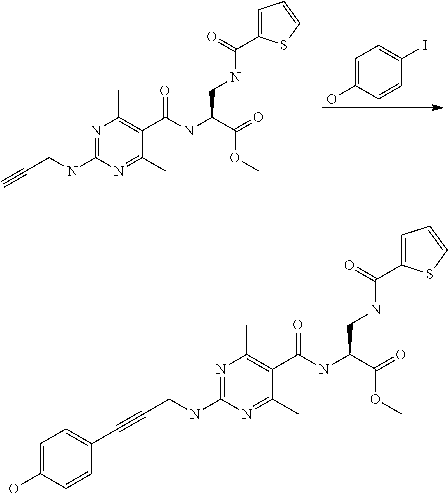

A reaction vessel containing 4-iodo-phenol (212 mg, 0.963 mmol), Et₃N (3 ml), dichlorobis(triphenylphosphine)palladium (II) (1 mg, 0.024 mmol), copper (I) iodide (9.2 mg, 0.048 mmol) and (S)-2-[(4,6-dimethyl-2-prop-2-ynylamino-pyrimidine-5-carbonyl)-amino]-3-[(thiophene-2-carbonyl)-amino]-propionic acid methyl ester (20 mg, 0.48 mmol) in DMF (3 ml) was sealed, degassed, flushed with nitrogen and stirred at room temperature overnight. The mixture was extracted with EtOAc. The combined organic extracts were washed with water and brine, dried over sodium sulfate, filtered and concentrated. The residue was purified by flash chromatography with 10% MeOH in EtOAc to afford title compound (81 mg, 33% yield). MS m/e 507.9 (M+H⁺).

### Example 33

### (S)-2-({2-[3-(4-hydroxy-phenyl)-propylamino]-4,6-dimethyl-pyrimidine-5-carbonyl}-amino)-3-[(thiophene-2-carbonyl)-amino]propionic acid methyl ester

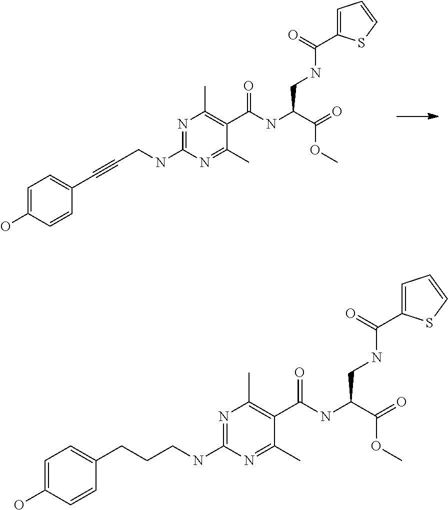

A solution of (S)-2-({2-[3-(4-hydroxy-phenyl)-prop-2-ynylamino]-4,6-dimethyl-pyrimidine-5-carbonyl}-amino)-3-[(thiophene-2-carbonyl)-amino]propionic acid methyl ester (81 mg, 0.16 mmol) in MeOH (300 ml) was passed through H-Cube with 10% Pd/C sulfided small cartridge at 20 bar, room temperature and 1 ml per min. Solvent was then removed by evaporation to give pure product (36 mg, 44% yield). MS m/e 511.9 (M+H⁺).

### Example 34

### (S)-2-({2-[3-(3-hydroxy-phenyl)-propylamino]-4,6-dimethyl-pyrimidine-5-carbonyl}-amino)-3-[(2-phenyl-thiazole-4-carbonyl)-amino]-propionic acid methyl ester

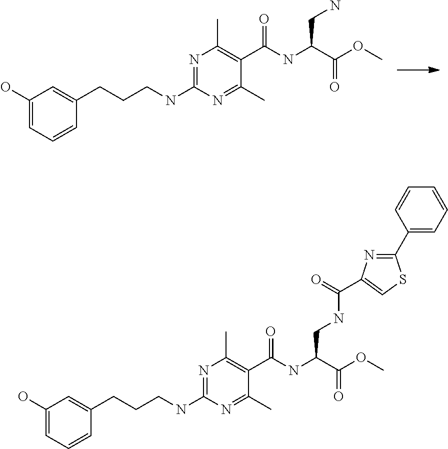

A solution of HBTU (48 mg, 0.127 mmol) and HOBT (17 mg, 0.126 mmol) in anhydrous DMF (2 ml) was added to a mixture of (S)-3-Amino-2-({2-[3-(3-hydroxy-phenyl)-propylamino]-4,6-dimethyl-pyrimidine-5-carbonyl}-amino)-propionic acid methyl ester hydrochloride (50 mg, 0.114 mmol), 2-phenyl-1,3-thiazole-4-carboxylic acid (24 mg, 0.117 mmol) and triethylamine (0.07 ml, 0.502 mmol) in anhydrous DMF (1 ml). The reaction mixture was stirred at room temperature for 1.25 h, diluted with brine, and extracted with ethyl acetate. The combined organic layers were washed with 1/1 aqueous saturated sodium bicarbonate solution/brine and brine, then dried over MgSO₄, filtered, concentrated and chromatographed (ethyl acetate to 9/1 ethyl acetate/methanol) to give the desired product as a colorless oil (35.5 mg, 52.8%). MS m/e 589 (M+H⁺).

A similar procedure was used to afford the following 6 examples:

### Example 35

### (S)-2-({2-[3-(3-hydroxy-phenyl)-propylamino]-4,6-dimethyl-pyrimidine-5-carbonyl}-amino)-3-[(pyridine-2-carbonyl)-amino]-propionic acid methyl ester

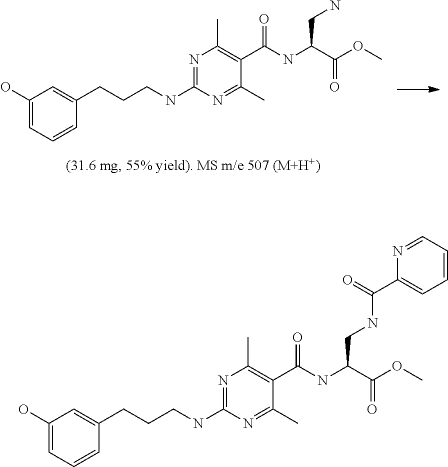

### Example 36

### (S)-2-({2-[3-(3-hydroxy-phenyl)-propylamino]-4,6-dimethyl-pyrimidine-5-carbonyl}-amino)-3-[(2,4-dimethyl-thiazole-5-carbonyl)-amino]-propionic acid methyl ester

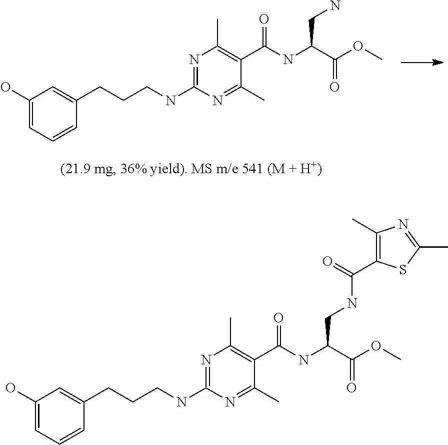

### Example 37

### (S)-2-({2-[3-(3-hydroxy-phenyl)-propylamino]-4,6-dimethyl-pyrimidine-5-carbonyl}-amino)-3-[2-cyclohexyl-acetylamino]-propionic acid methyl ester

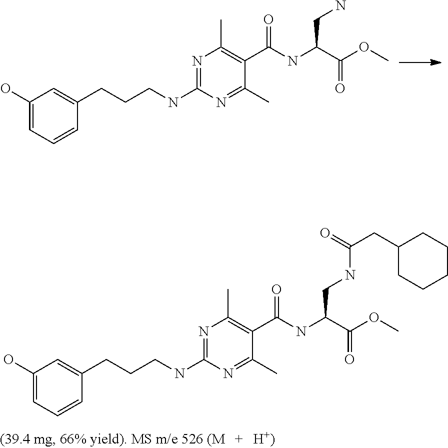

### Example 38

### (S)-2-({2-[3-(3-hydroxy-phenyl)-propylamino]-4,6-dimethyl-pyrimidine-5-carbonyl}-amino)-3-[(isoquinoline-1-carbonyl)-amino]-propionic acid methyl ester

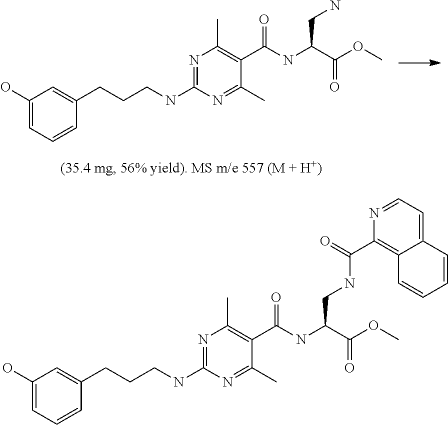

### Example 39

### (S)-2-({2-[3-(3-hydroxy-phenyl)-propylamino]-4,6-dimethyl-pyrimidine-5-carbonyl}-amino)-3-[(thiazole-4-carbonyl)-amino]-propionic acid methyl ester

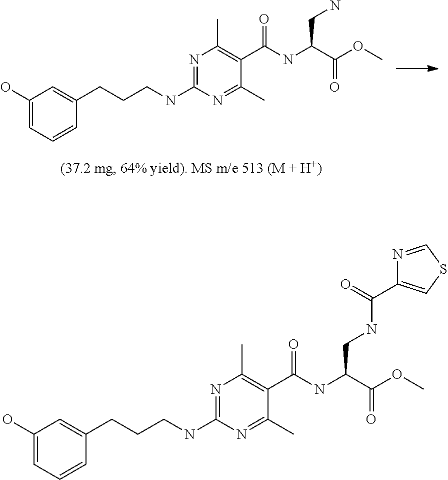

### Example 40

### (S)-2-({2-[3-(3-hydroxy-phenyl)-propylamino]-4,6-dimethyl-pyrimidine-5-carbonyl}-amino)-3-[(2-pyridin-4-yl-thiazole-4-carbonyl)-amino]-propionic acid methyl ester

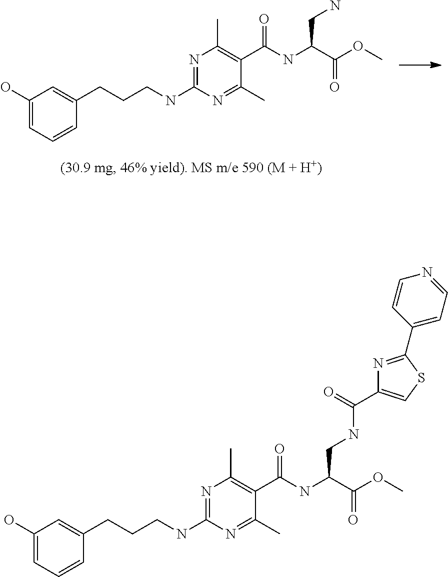

### Example 41

### (S)-2-({2-[3-(3-hydroxy-phenyl)-propylamino]-4,6-dimethyl-pyrimidine-5-carbonyl}-amino)-3-[(2-pyridin-4-yl-thiazole-4-carbonyl)-amino]-propionic acid

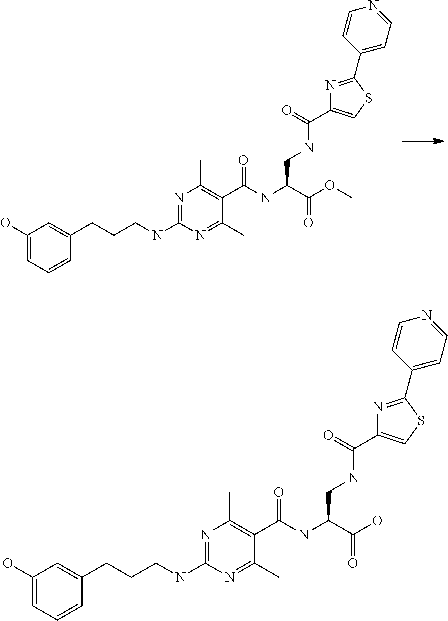

A solution of (S)-2-({2-[3-(3-hydroxy-phenyl)-propylamino]-4,6-dimethyl-pyrimidine-5-carbonyl}-amino)-3-[(2-phenyl-thiazole-4-carbonyl)-amino]-propionic acid methyl ester (35.5 mg, 0.060 mmol) in THF (0.7 ml) was treated with a solution of lithium hydroxide monohydrate (25 mg, 0.596 mmol) in water (2 ml) and the reaction mixture was stirred at room temperature for 1.5 h. The reaction solution was acidified to pH ca. 3 with aqueous 1N KHSO₄, concentrated to remove THF, and diluted with water. The precipitate was collected by filtration and air dried to afford the desired product as a white solid (27.6 mg, 79.6% yield). MS m/e 575.0 (M+H⁺)

A similar procedure was used to afford the following 6 examples:

### Example 42

### (S)-2-({2-[3-(3-hydroxy-phenyl)-propylamino]-4,6-dimethyl-pyrimidine-5-carbonyl}-amino)-3-[(pyridine-2-carbonyl)-amino]-propionic acid

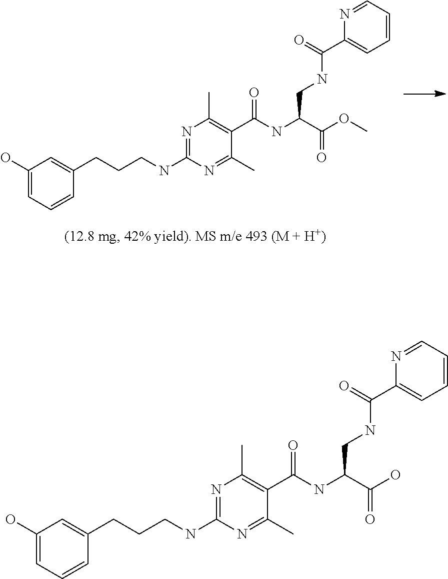

### Example 43

### (S)-2-({2-[3-(3-hydroxy-phenyl)-propylamino]-4,6-dimethyl-pyrimidine-5-carbonyl}-amino)-3-[(2,4-dimethyl-thiazole-5-carbonyl)-amino]-propionic acid

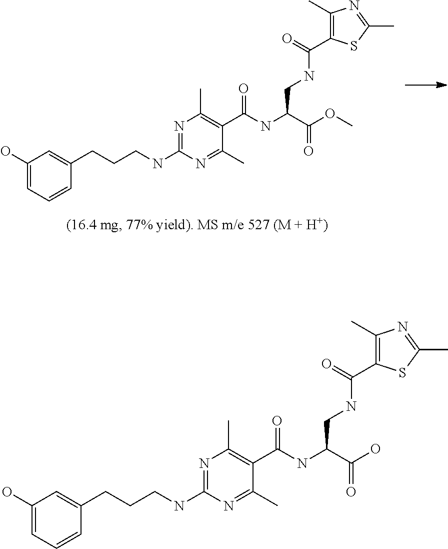

### Example 44

### (S)-2-({2-[3-(3-hydroxy-phenyl)-propylamino]-4,6-dimethyl-pyrimidine-5-carbonyl}-amino)-3-[1-cyclohexyl-acetylamino]-propionic acid

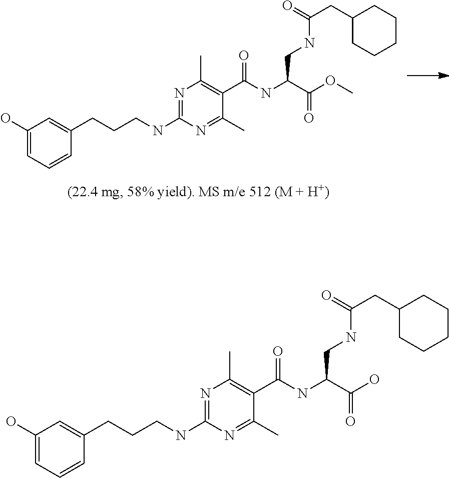

### Example 45

### (S)-2-({2-[3-(3-hydroxy-phenyl)-propylamino]-4,6-dimethyl-pyrimidine-5-carbonyl}-amino)-3-[(isoquinoline-1-carbonyl)-amino]-propionic acid

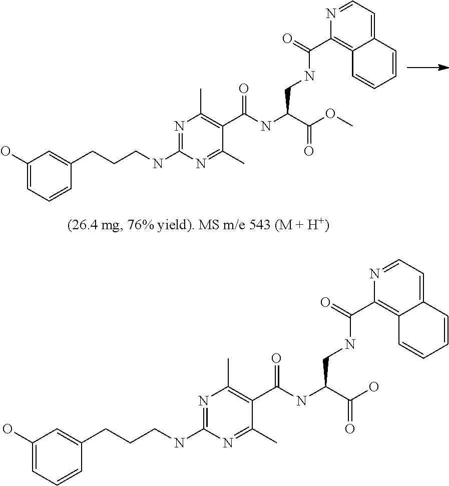

### Example 46

### (S)-2-({2-[3-(3-hydroxy-phenyl)-propylamino]-4,6-dimethyl-pyrimidine-5-carbonyl}-amino)-3-[(thiazole-4-carbonyl)-amino]-propionic acid

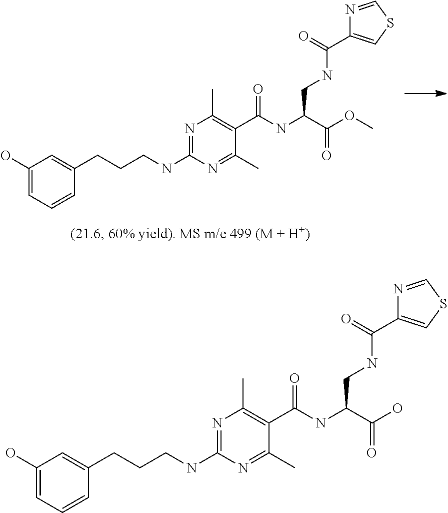

### Example 47

### (S)-2-({2-[3-(3-hydroxy-phenyl)-propylamino]-4,6-dimethyl-pyrimidine-5-carbonyl}-amino)-3-[(2-pyridin-4-yl-thiazole-4-carbonyl)-amino]-propionic acid

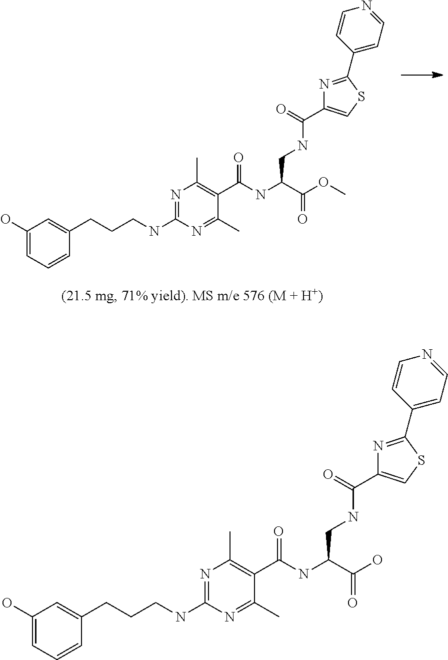

### Example 48

### (S)-2-({2-[3-(3-hydroxy-4-methyl-phenyl)-propylamino]-4,6-dimethyl-pyrimidine-5-carbonyl}-amino)-3-[(thiophene-2-carbonyl)-amino]-propionic acid methyl ester

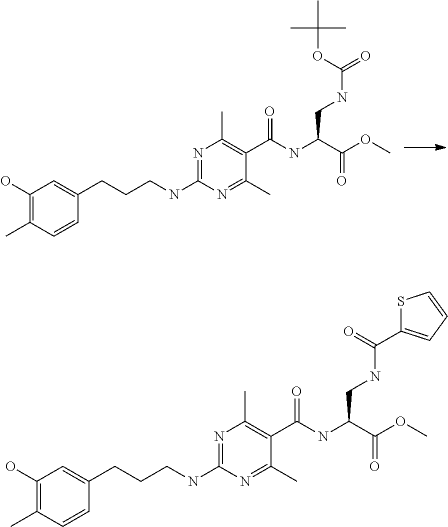

To a solution of (S)-3-tert-butoxycarbonylamino-2-({2-[3-(3-hydroxy-4-methyl-phenyl)-propylamino]-4,6-dimethyl-pyrimidine-5-carbonyl}-amino)-propionic acid methyl ester (180 mg, 0.35 mmol) in DCM (35 ml) was added dropwise 30% TFA/CH₂Cl₂ (10 ml) at 0° C. The solution was stirred at room temperature for 3 h. Solvent and excess TFA were removed by vacuum evaporation. The residue was dissolved in DMF (3 ml) and triethylamine (1 ml, 7 mmol) was added dropwise at 0° C. followed by thiophene-2-carboxylic acid (53.8 mg, 0.42 mmol), HBTU (59 mg, 0.42 mmol) and HOBT (57 mg, 0.42 mmol). The mixture was stirred at room temperature for 12 h. The mixture was extracted with EtOAc. The combined organic extracts were washed with water and brine, dried over sodium sulfate, filtered and concentrated. The residue was purified by flash chromatography with 10% MeOH in DCM to give the desired product (84.7 mg, 46% yield). MS m/e 526.0 (M+H⁺).

### Example 49

### (S)-2-({2-[(E)-3-(3-hydroxy-phenyl)-allylamino]-4,6-dimethyl-pyrimidine-5-carbonyl}-amino)-3-[(thiophene-2-carbonyl)-amino]-propionic acid methyl ester

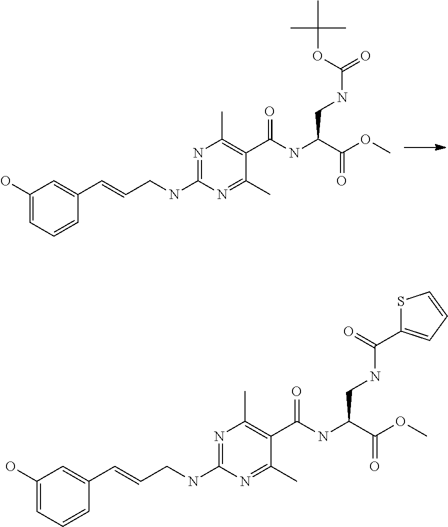

A solution of (S)-3-tert-butoxycarbonylamino-2-({2-[(E)-3-(3-hydroxy-phenyl)-allylamino]-4,6-dimethyl-pyrimidine-5-carbonyl}-amino)-propionic acid methyl ester (183 mg, 0.37 mmol) was treated with 50% TFA in DCM (4 ml) at room temperature for 2 h. The solution was stirred at room temperature for 3 h. Solvent and excess TFA were removed by vacuum evaporation at 30-35° C. The residue was dissolved in anhydrous DMF (3 ml) and treated with triethylamine (1 ml, 7. mmol) at 0° C. Thiophene-2-carboxylic acid (56.9 mg, 0.4 mmol), HBTU (168 mg, 0.44 mmol) and HOBT (60 mg, 0.44 mmol) were added at room temperature and the mixture was stirred at room temperature over the weekend. The mixture was extracted with EtOAc. The combined organic extracts were washed with water and brine, dried over sodium sulfate, filtered and concentrated. The residue was purified by flash chromatography with a 50-100% EtOAc in hexane gradient to give the desired product (45 mg, 24% yield). MS m/e 509.9 (M+H⁺).

### Example 50

### (S)-2-({2-[(E)-3-(6-benzyloxy-pyridin-2-yl)-allylamino]-4,6-dimethyl-pyrimidine-5-carbonyl}-amino)-propionic acid methyl ester

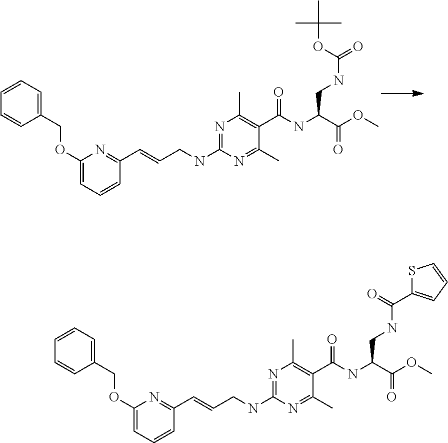

(S)-2-({2-[(E)-3-(6-benzyloxy-pyridin-2-yl)-allylamino]-4,-6-dimethyl-pyrimidine-5-carbonylamino-propionic acid methyl ester (122 mg, 0.21 mmol) was treated with 30% TFA/CH₂Cl₂ (5 ml) at room temperature for 2 h. Solvent and excess TFA were removed by vacuum evaporation. The residue was dissolved in anhydrous DMF (3 ml) and triethylamine (0.59 ml, 4.2 mmol) was added dropwise at 0° C. Thiophene-2-carboxylic acid (32 mg, 0.25 mmol), HBTU (95 mg, 0.25 mmol) and HOBT (34 mg, 0.25 mmol) were successively added at room temperature and the mixture was stirred for 12 h. The mixture was extracted with EtOAc. The combined organic extracts were washed with water and brine, dried over sodium sulfate, filtered and concentrated. The residue was purified by flash chromatography with a 50-100% EtOAc in hexane gradient to give the desired product (92 mg, 73% yield). MS m/e 601.0 (M+H⁺).

### Example 51

### (S)-2-({2-[(E)-3-(6-Benzyloxy-pyridin-2-yl)-allyamino]-4,6-dimethyl-pyrimidine-5-carbonyl}-amino)-3-[(thiophene-2-carbonyl)-amino]-propionic acid

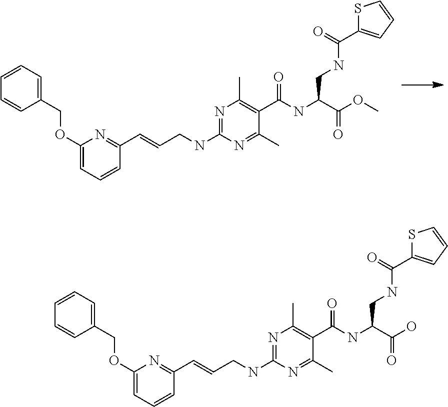

A solution of (S)-2-({2-[(E)-3-(6-benzyloxy-pyridin-2-yl)-allylamino]-4,6-dimethyl-pyrimidine-5-carbonyl}-amino)-propionic acid methyl ester (92 mg, 0.153 mmol) and lithium hydroxide monohydrate (32 mg, 0.77 mmol) in dioxane/water (10 ml/10 ml) was stirred at room temperature overnight, then quenched with aqueous potassium hydrogen sulfate to pH 2-4. The mixture was extracted with EtOAc. The organic extracts were combined, washed with brine, dried over sodium sulfate, and filtered to give the desired product in EtOAc. This solution was not concentrated but was used directly in the next step. MS m/e 587.0 (M+H⁺).

### Example 52

### (S)-2-({2-[3-(6-Hydroxy-pyridin-2-yl)-propylamino]-4,6-dimethyl-pyrimidine-5-carbonyl}-amino)-3-[(thiophene-2-carbonyl)-amino]-propionic acid

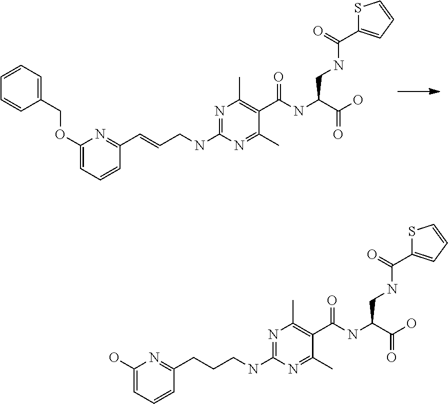

A solution of (S)-2-({2-[(E)-3-(6-benzyloxy-pyridin-2-yl)-allyamino]-4,6-dimethyl-pyrimidine-5-carbonyl}-amino)-3-[(thiophene-2-carbonyl)-amino]-propionic acid (about 0.1 mmol) in ethyl acetate (about 20 ml) was passed two times through H-Cube with 10% Pd/C small cartridge at 70 bar and 50° C., at a rate of 1 ml/min. The solution was concentrated and the residue was purified by preparative HPLC under neutral condition to give the desired product (7.7 mg). MS m/e 498.9 (M+H⁺).

### Example 53

### (S)-2-({2-[3-(2-Fluoro-3-hydroxy-phenyl)-propylamino]-4,6-dimethyl-pyrimidine-5-carbonyl}-amino)-3-[(thiophene-2-carbonyl)-amino]-propionic acid methyl ester

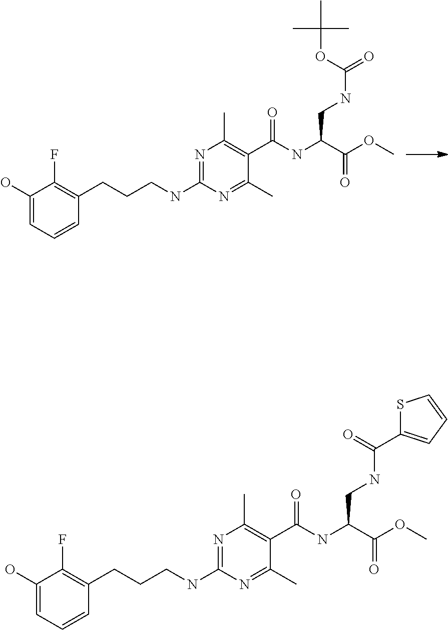

(S)-3-tert-Butoxycarbonylamino-2-({2-[3-(2-fluoro-3-hydroxy-phenyl)-propylamino]-4,6-dimethyl-pyrimidine-5-carbonyl}-amino)-propionic acid methyl ester (94 mg, 0.18 mmol) was treated with 50% TFA/CH₂Cl₂ (3 ml) at room temperature for 2 h. Solvent and excess TFA were removed by vacuum evaporation. The residue was dissolved in anhydrous DMF (1 ml) and triethylamine (0.5 ml, 3.62 mmol) was added at 0° C. Thiophene-2-carboxylic acid (46.4 mg, 0.36 mmol), HBTU (65 mg, 0.36 mmol) and HOBT (49 mg, 0.36 mmol), were then added at room temperature and the mixture was stirred for 12 h. The reaction mixture was quenched with water and extracted with EtOAc. The organic extracts were combined, washed with brine, dried over sodium sulfate, filtered and evaporated. The residue was purified by flash chromatography with a 40-100% EtOAc in hexane gradient to give the title compound (47 mg, 49% yield). MS m/e 529.8 (M+H⁺).

### Example 54

### (S)-2-({2-[3-(2-Fluoro-5-hydroxy-phenyl)-propylamino]-4,6-dimethyl-pyrimidine-5-carbonyl}-amino)-3-[(thiophene-2-carbonyl)-amino]-propionic acid methyl ester

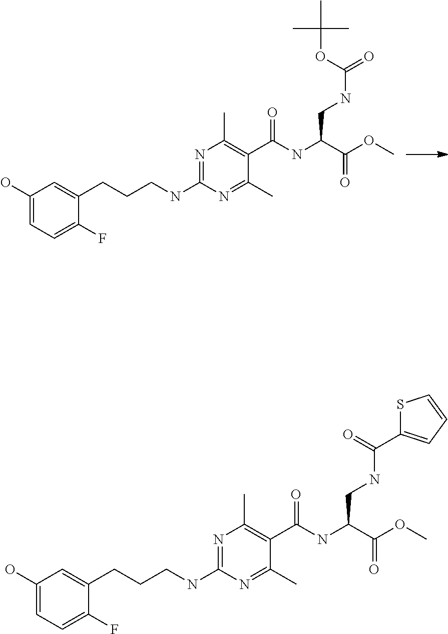

(S)-3-tert-butoxycarbonylamino-2-({2-[3-(2-fluoro-5-hydroxy-phenyl)-propylamino]-4,6-dimethyl-pyrimidine-5-carbonyl}-amino)-propionic acid methyl ester (62 mg, 0.12 mmol) in MeOH (0.5 ml) was treated with 4NHO/dioxane (2 ml) at room temperature for 1.5 h. The solution was concentrated to dryness and the residue was dissolved in anhydrous DMF (3 ml). Triethylamine (0.1 ml, 0.714 mmol), thiophene-2-carboxylic acid (30.6 mg, 0.24 mmol), HBTU (65 mg, 0.36 mmol) and HOBT (49 mg, 0.36 mmol) were successively added at room temperature and the mixture was stirred for 12 h. The reaction mixture was quenched with water and extracted with EtOAc. The combined organic layers were washed with brine, dried over sodium sulfate, filtered and concentrated. The residue was purified by flash chromatography with a 40-100% EtOAc in hexane gradient to give the title compound (43.5 mg, 69% yield). MS m/e 529.8 (M+H⁺).

### Example 55

### (S)-2-({2-[3-(2-Methyl-5-hydroxy-phenyl)-propylamino]-4,6-dimethyl-pyrimidine-5-carbonyl}-amino)-3-[(thiophene-2-carbonyl)-amino]-propionic acid methyl ester

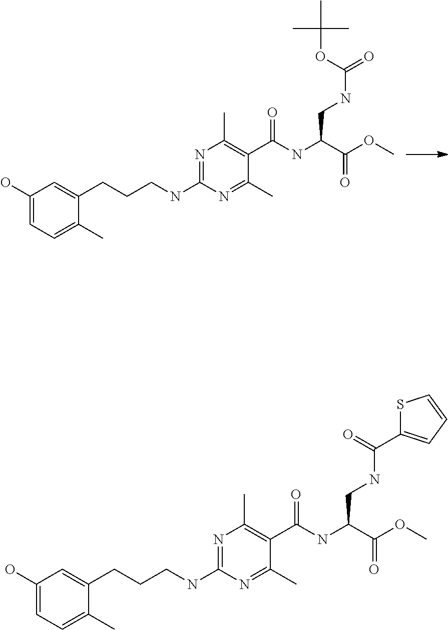

(S)-3-tert-Butoxycarbonylamino-2-({2-[3-(2-methyl-5-hydroxy-phenyl)-propylamino]-4,6-dimethyl-pyrimidine-5-carbonyl}-amino)-propionic acid methyl ester (26.3 mg, 0.12 mmol) in MeOH (0.5 ml) was treated with 4NHCl/dioxane (2 ml) at room temperature for 1.5 h. The solution was concentrated to dryness and the residue was dissolved in anhydrous DMF (2 ml). Triethylamine (31 mg, 0.306 mmol), thiophene-2-carboxylic acid (13 mg, 0.102 mmol), HBTU (39 mg, 0.102 mmol) and HOBT (14 mg, 0.102 mmol) were successively added at room temperature and the mixture was stirred for 2 h. The reaction mixture was quenched with water and extracted with EtOAc. The combined organic layers were washed with brine, dried over sodium sulfate, filtered and concentrated. The residue was purified by flash chromatography with a 40-100% EtOAc in hexane gradient to give the title compound (18 mg, 67% yield). MS m/e 526.0 (M+H⁺).

### Example 56

### (S)-2-({2-[3-(3-hydroxy-phenyl)-propylamino]-pyrimidine-5-carbonyl}-amino)-3-[(thiophene-2-carbonyl)-amino]-propionic acid methyl ester

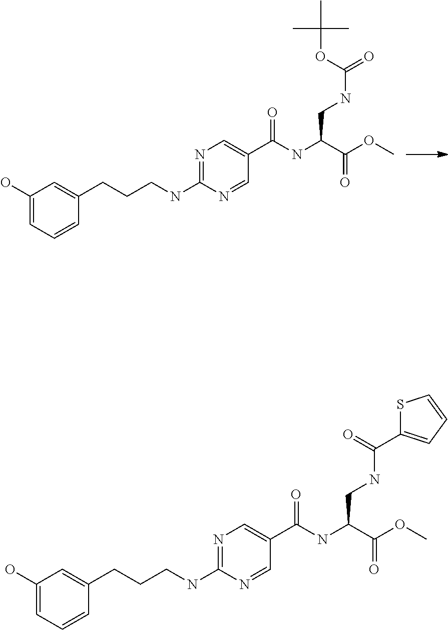

(S)-3-tert-butoxycarbonylamino-2-({2-[3-(3-hydroxy-phenyl)-propylamino]-pyrimidine-5-carbonyl}-amino)-propionic acid methyl ester (820 mg, 1.73 mmol) was treated with 30% TFA in DCM (50 ml) at room temperature for 2 h. The solution was concentrated to dryness and the residue was dissolved in anhydrous DMF (20 ml). Triethylamine (4.8 ml, 34.6 mmol), thiophene-2-carboxylic acid (266 mg, 2.08 mmol), HBTU (790 mg, 2.08 mmol) and HOBT (281 mg, 2.08 mmol) were successively added at room temperature and the mixture was stirred overnight. The reaction mixture was quenched with water and extracted with EtOAc. The combined organic layers were washed with brine, dried over sodium sulfate, filtered and concentrated. The residue was purified by flash chromatography with a 40-100% EtOAc in hexane gradient to give the desired product (220 mg, 26% yield). MS m/e 483.9 (M+H⁺).

### Example 57

### (S)-2-({2-[3-(3-hydroxy-phenyl)-propylamino]-4-methyl-pyrimidine-5-carbonyl}-amino)-3-[(thiophene-2-carbonyl)-amino]-propionic acid methyl ester

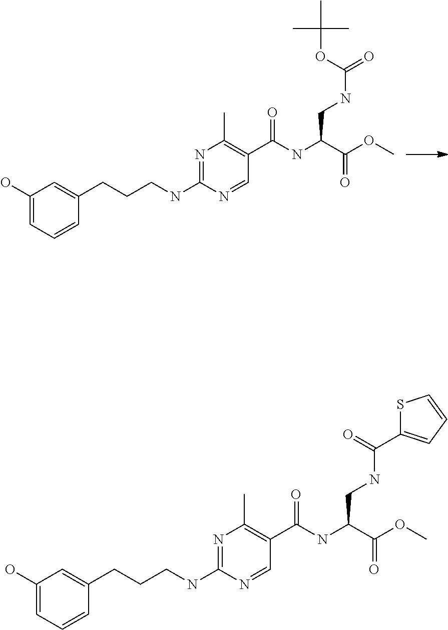

(S)-3-tert-Butoxycarbonylamino-2-({2-[3-(3-hydroxy-phenyl)-propylamino]-4-methyl-pyrimidine-5-carbonyl}-amino)-propionic acid methyl ester (482 mg, 0.99 mmol) was treated with 50% TFA in DCM (5 ml) at room temperature for 2 h The solution was concentrated to dryness and the residue was dissolved in anhydrous DMF (5 ml). Triethylamine (2.8 ml, 19.8 mmol), thiophene-2-carboxylic acid (152 mg, 1.19 mmol), HBTU (452 mg, 1.19 mmol) and HOBT (161 mg, 1.19 mmol) were successively added at room temperature and the mixture was stirred overnight. The reaction mixture was quenched with water and extracted with EtOAc. The combined organic layers were washed with brine, dried over sodium sulfate, filtered and concentrated. The residue was purified by flash chromatography with a 40-100% EtOAc in hexane gradient to give the desired product (319 mg, 65% yield). MS m/e 497.9 (M+H⁺).

### Example 58

### (S)-2-({2-[3-(3-hydroxy-phenyl)-propylamino]-4-trifluoromethyl-pyrimidine-5-carbonyl}-amino)-3-[(thiophene-2-carbonyl)-amino]-propionic acid methyl ester

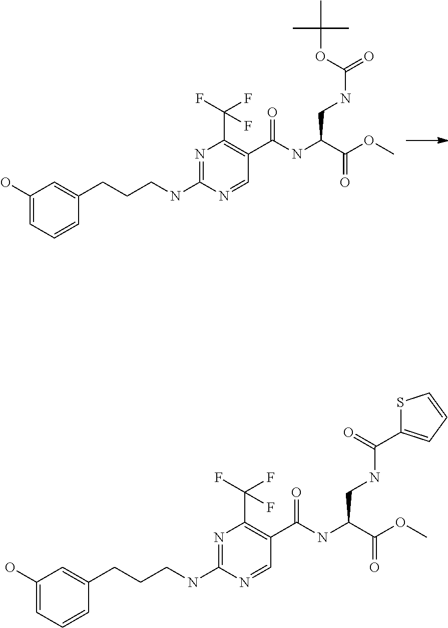

(S)-3-tert-butoxycarbonylamino-2-({2-[3-(3-hydroxy-phenyl)-propylamino]-4-trifluoromethyl-pyrimidine-5-carbonyl}-amino)-propionic acid methyl ester (970 mg, 1.79 mmol) was treated at room temperature with 30% TFA in DCM (50 ml) for 2 h. The solution was concentrated to dryness and the residue was dissolved in anhydrous DMF (20 ml). Triethylamine (5 ml, 35.8 mmol), thiophene-2-carboxylic acid (275.5 mg, 2.15 mmol), HBTU (816 mg, 2.15 mmol) and HOBT (290 mg, 2.15 mmol were successively added at room temperature and the mixture was stirred overnight. The reaction mixture was quenched with water and extracted with EtOAc. The combined organic layers were washed with brine, dried over sodium sulfate, filtered and concentrated. The residue was purified by flash chromatography with a 40-100% EtOAc in hexane gradient to give the title compound (450 mg, 46% yield). MS m/e 552.0 (M+H⁺).

### Example 59

### (S)-2-({4-Ethyl-2-[3-(3-hydroxy-phenyl)-propylamino]-6-methyl-pyrimidine-5-carbonyl}-amino)-3-[(thiophene-2-carbonyl)-amino]-propionic acid methyl ester

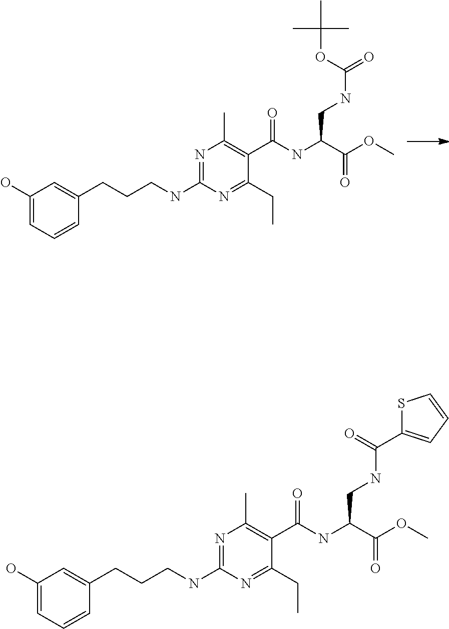

A solution of (S)-3-tert-butoxycarbonylamino-2-({4-ethyl-2-[3-(3-hydroxy-phenyl) propylamino]-6-methyl-pyrimidine-5-carbonyl}-amino)-propionic acid methyl ester (265 mg, 0.514 mmol) was treated at room temperature with 50% TFA in DCM (5 ml) for 2 h. The solution was concentrated to dryness and the residue was dissolved in anhydrous DMF (5 ml). Triethylamine (1.4 ml, 10.28 mmol), thiophene-2-carboxylic acid (79 mg, 0.617 mmol), HBTU (234 mg, 0.617 mmol) and HOBT (83 mg, 0.617 mmol) were successively added at room temperature and the mixture was stirred overnight. The reaction mixture was quenched with water and extracted with EtOAc. The combined organic layers were washed with brine, dried over sodium sulfate, filtered and concentrated. The residue was purified by flash chromatography with a 40-100% EtOAc in hexane gradient to give the desired product (221 mg, 82% yield). MS m/e 526.0 (M+H⁺).

### Example 60

### (S)-2-({4,6-diethyl-2-[3-(3-hydroxy-phenyl)-propylamino]-pyrimidine-5-carbonyl}-amino)-3-[(thiophene-2-carbonyl)-amino]-propionic acid methyl ester

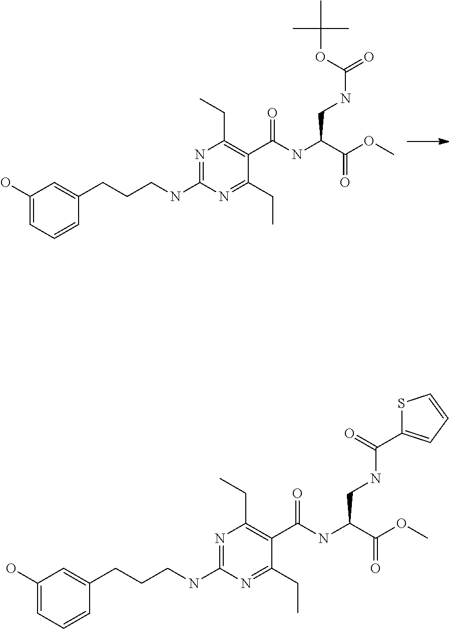

(S)-3-tert-butoxycarbonylamino-2-({4,6-diethyl-2-[3-(3-hydroxy-phenyl) propylamino]-pyrimidine-5-carbonyl}-amino)-propionic acid methyl ester (22 mg, 0.04 mmol) was treated at room temperature with 50% TFA in DCM (2 ml) for 2 h. The solution was concentrated to dryness and the residue was dissolved in anhydrous DMF (2 ml). Triethylamine (84 mg, 0.8 mmol), thiophene-2-carboxylic acid (6.4 mg, 0.05 mmol), HBTU (9 mg, 0.05 mmol) and HOBT (7 mg, 0.05 mmol) were successively added at room temperature and the mixture was stirred overnight. The reaction mixture was quenched with water and extracted with EtOAc. The combined organic layers were washed with brine, dried over sodium sulfate, filtered and concentrated. The residue was purified by flash chromatography with a 40-100% EtOAc in hexane gradient to give the desired product (9.4 mg, 42% yield). MS m/e 540.0 (M+H⁺).

### Example 61

### (S)-2-({2-[3-(3-Hydroxy-phenyl)-prop-2-ynylamino]-4,6-dimethyl-pyrimidine-5-carbonyl}-amino)-3-[(thiophene-2-carbonyl)-amino]-propionic acid

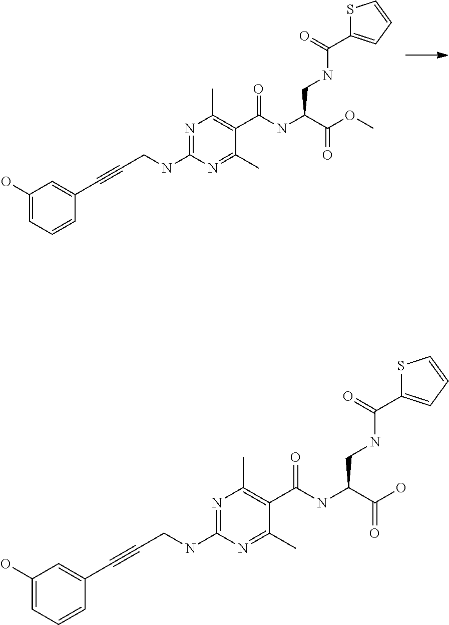

A solution of (S)-2-({2-[3-(3-hydroxy-phenyl)-prop-2ynylamino]-4,6-dimethyl-pyrimidine-5-carbonyl}-amino)-3-[(thiophene-2-carbonyl)-amino]propionic acid methyl ester (15 mg, 0.03 mmol) in dioxane (1 ml) was treated at room temperature with a solution of lithium hydroxide monohydrate (6.3 mg, 0.15 mmol) in water (1 ml). The mixture was stirred at room temperature overnight, then quenched with aqueous potassium hydrogen sulfate to adjust the pH to ˜2-4 and extracted with EtOAc. The organic extracts were combined, washed with brine, dried over sodium sulfate, filtered and evaporated to give the title compound (14 mg, 95% yield). MS m/e 493.9 (M+H⁺).

A similar procedure was used to afford the following 6 examples:

### Example 62

### (S)-2-({2-[3-(1H-Indazol-4-yl)-prop-2-ynylamino]-4,6-dimethyl-pyrimidine-5-carbonyl}-amino)-3-[(thiophene-2-carbonyl)-amino]-propionic acid

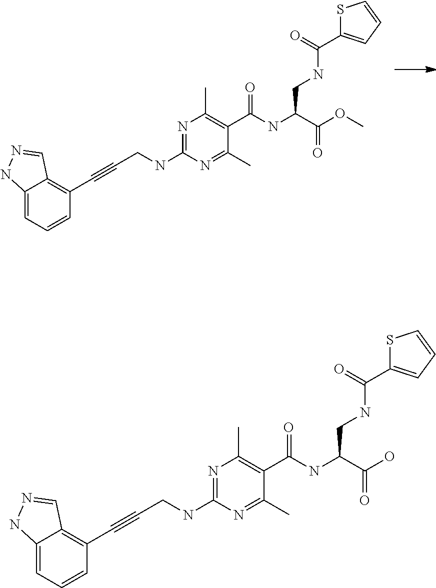

(S)-2-({2-[3-(1H-indazol-4-yl)-prop-2ynylamino]-4,6-dimethyl-pyrimidine-5-carbonyl}-amino)-3-[(thiophene-2-carbonyl)-amino]propionic acid methyl ester (37.3 mg, 0.07 mmol) afforded the product (21.5 mg, 59% yield). MS m/e 518.0 (M+H⁺).

### Example 63

### (S)-2-({2-[3-(1H-Indazol-6-yl)-propylamino]-4,6-dimethyl-pyrimidine-5-carbonyl}-amino)-3-[(thiophene-2-carbonyl)-amino]-propionic acid

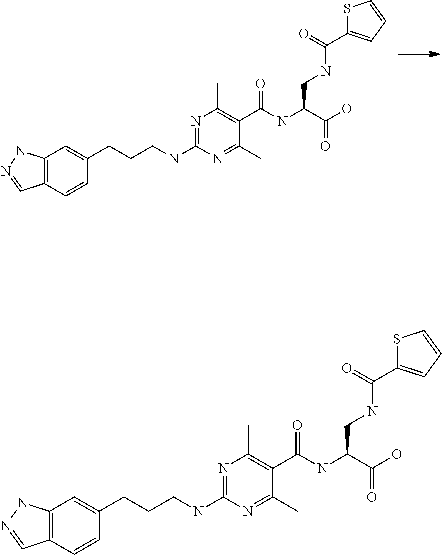

(S)-2-({2-[3-(1H-indazol-6-yl)-propylamino]-4,6-dimethyl-pyrimidine-5-carbonyl}-amino)-3-[(thiophene-2-carbonyl)-amino]propionic acid methyl ester (65 mg, 0.12 mmol) afforded the product (35 mg, 56% yield). MS m/e 522.0 (M+H⁺).

### Example 64

### (S)-2-({2-[3-(3-Fluoro-5-hydroxy-phenyl)-propylamino]-4,6-dimethyl-pyrimidine-5-carbonyl}-amino)-3-[(thiophene-2-carbonyl)-amino]-propionic acid

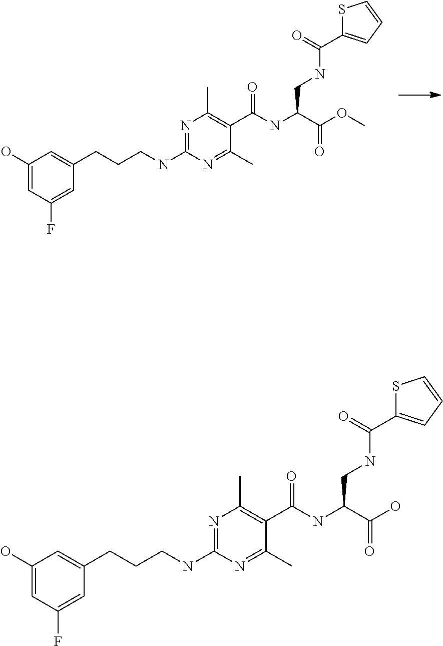

(S)-2-({2-[3-(3-fluoro-5-hydroxy-phenyl)-propylamino]-4,6-dimethyl-pyrimidine-5-carbonyl}-amino)-3-[(thiophene-2-carbonyl)-amino]propionic acid methyl ester (18.2 mg, 0.034 mmol afforded the product (9.7 mg, 55% yield). MS m/e 516.0 (M+H⁺).

### Example 65

### (S)-2-({2-[3-(4-Fluoro-3-hydroxy-phenyl)-propylamino]-4,6-dimethyl-pyrimidine-5-carbonyl}-amino)-3-[(thiophene-2-carbonyl)-amino]-propionic acid

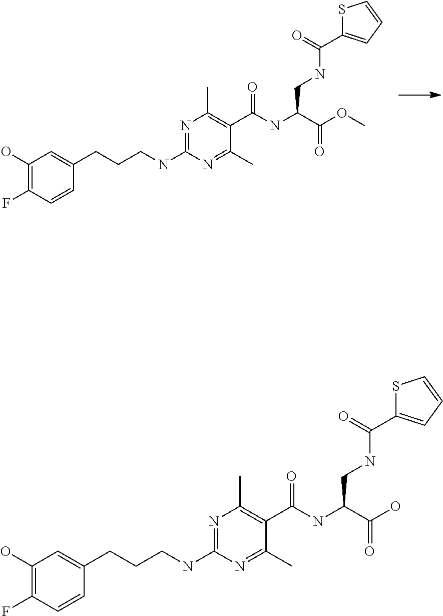

(S)-2-({2-[3-(4-fluoro-3-hydroxy-phenyl)-propylamino]-4,6-dimethyl-pyrimidine-5-carbonyl}-amino)-3-[(thiophene-2-carbonyl)-amino]propionic acid methyl ester (42 mg, 0.079 mmol) afforded the product (8.4 mg, 21% yield). MS m/e 516.0 (M+H⁺).

### Example 66

### (S)-2-({2-[3-(3-Hydroxy-2-methyl-phenyl)-propylamino]-4,6-dimethyl-pyrimidine-5-carbonyl}-amino)-3-[(thiophene-2-carbonyl)-amino]-propionic acid

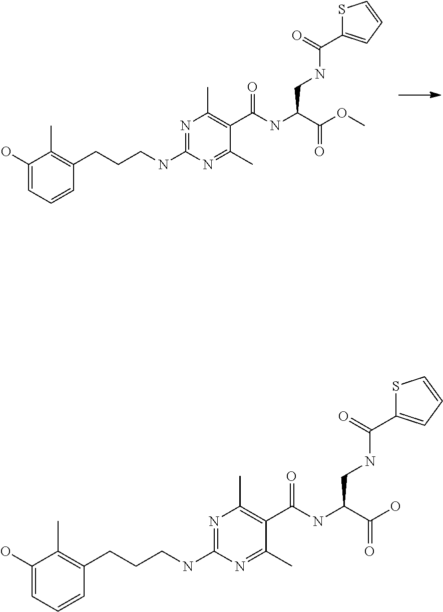

(S)-2-({2-[3-(3-hydroxy-2-methyl-phenyl)-propylamino]-4,6-dimethyl-pyrimidine-5-carbonyl}-amino)-3-[(thiophene-2-carbonyl)-amino]propionic acid methyl ester (197 mg, 0.375 mmol) afforded the product (59 mg, 31% yield). MS m/e 511.9 (M+H⁺).

### Example 67

### (S)-2-({2-[3-(3-Hydroxy-4-methyl-phenyl)-propylamino]-4,6-dimethyl-pyrimidine-5-carbonyl}-amino)-3-[(thiophene-2-carbonyl)-amino]-propionic acid

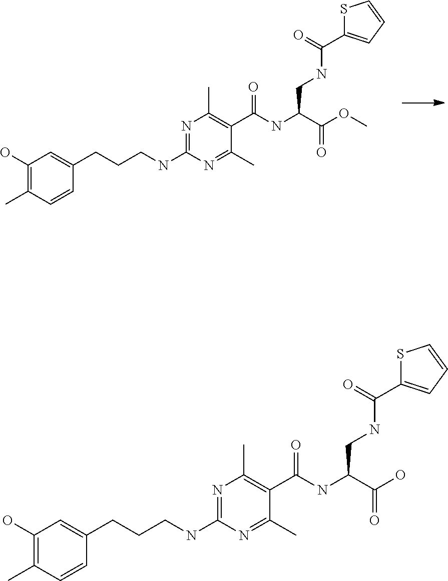

(S)-2-({2-[3-(3-hydroxy-4-methyl-phenyl)-propylamino]-4,6-dimethyl-pyrimidine-5-carbonyl}-amino)-3-[(thiophene-2-carbonyl)-amino]propionic acid methyl ester (84.7 mg, 0.16 mmol) afforded the product (66 mg, 81% yield). MS m/e 512.0 (M+H⁺).

### Example 68

### (S)-2-({2-[3-(2-Chloro-5-hydroxy-phenyl)-propylamino]-4,6-dimethyl-pyrimidine-5-carbonyl}-amino)-3-[(thiophene-2-carbonyl)-amino]-propionic acid

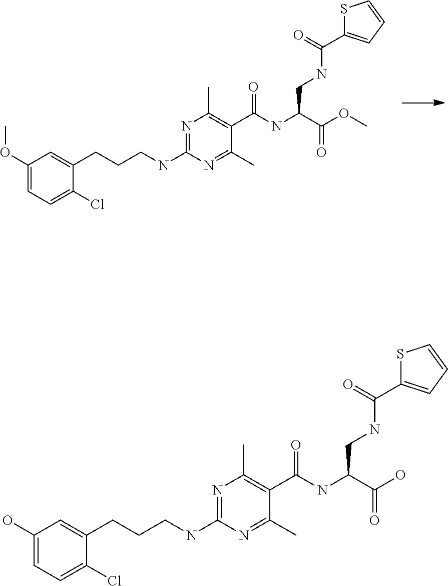

A solution of (S)-2-({2-[3-(2-chloro-5-methoxy-phenyl)-propylamino]-4,6-dimethyl-pyrimidine-5-carbonyl}-amino)-3-[(thiophene-2-carbonyl)-amino]propionic acid methyl ester (18.1 mg, 0.323 mmol) in anhydrous DCM (10 ml) was treated at 0° C. with BBr3/DCM (1M, 1.4 ml, 1.4 mmol). The mixture was allowed to warm up to room temperature and stirred for 2 h, then quenched with ice water. The mixture was evaporated to dryness and the residue was dissolved in methanol (5 ml). A solution of lithium hydroxide monohydrate (68 mg, 1.62 mmol) in water (5 ml) was added and the mixture was stirred at room temperature for 2 h then concentrated to dryness. The residue was dissolved in THF (10 ml) and treated with aqueous potassium hydrogen sulfate to pH 2-4. THF was then removed by evaporation and the mixture was diluted with cold water. The solid was collected by filtration and washed with water and cold methanol to afford the title compound (125 mg, 73% yield). MS m/e 531.9 (M+H⁺).

### Example 69

### (S)-2-({2-[3-(4-Hydroxy-phenyl)-propylamino]-4,6-dimethyl-pyrimidine-5-carbonyl}-amino)-3-[(thiophene-2-carbonyl)-amino]-propionic acid

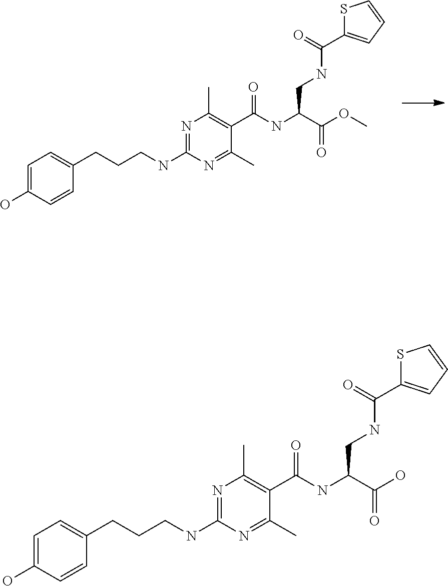

A solution of (S)-2-({2-[3-(4-hydroxy-phenyl)-propylamino]-4,6-dimethyl-pyrimidine-5-carbonyl}-amino)-3-[(thiophene-2-carbonyl)-amino]-propionic acid methyl ester (36 mg, 0.07 mmol) in THF (5 ml) was treated at room temperature with a solution of lithium hydroxide monohydrate (100 mg, 2.38 mmol) in water (5 ml). The mixture was stirred at room temperature overnight, then quenched with aqueous potassium hydrogen sulfate to adjust the pH to ˜2-4. THF was removed by evaporation and the mixture was diluted with cold water. The white precipitate was collected by filtration, washed with water and dried to afford pure product (36 mg, 100% yield). MS m/e 497.9 (M+H⁺).

A similar procedure was used to afford the following 10 examples:

### Example 70

### (S)-2-({2-[3-(2-Fluoro-3-hydroxy-phenyl)-propylamino]-4,6-dimethyl-pyrimidine-5-carbonyl}-amino)-3-[(thiophene-2-carbonyl)-amino]-propionic acid

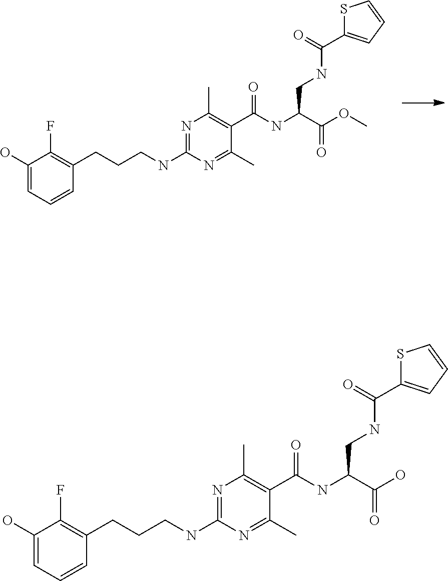

(S)-2-({2-[3-(2-fluoro-3-hydroxy-phenyl)-propylamino]-4,6-dimethyl-pyrimidine-5-carbonyl}-amino)-3-[(thiophene-2-carbonyl)-amino]propionic acid methyl ester (47 mg, 0.089 mmol) afforded the product (17 mg, 37% yield). MS m/e 515.8 (M+H⁺).

### Example 71

### (S)-2-({2-[3-(2-Fluoro-5-hydroxy-phenyl)-propylamino]-4,6-dimethyl-pyrimidine-5-carbonyl}-amino)-3-[(thiophene-2-carbonyl)-amino]-propionic acid

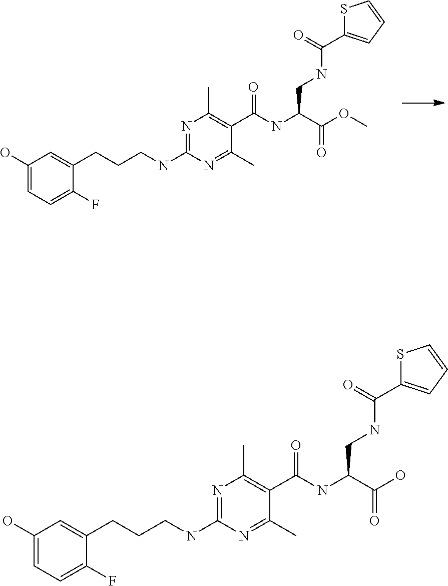

(S)-2-({2-[3-(2-fluoro-5-hydroxy-phenyl)-propylamino]-4,6-dimethyl-pyrimidine-5-carbonyl}-amino)-3-[(thiophene-2-carbonyl)-amino]-propionic acid methyl ester (43.5 mg, 0.082 mmol) afforded the desired product (41 mg, 100% yield). MS m/e 515.8 (M+H⁺).

### Example 72

### (S)-2-({2-[3-(2-Methyl-5-hydroxy-phenyl)-propylamino]-4,6-dimethyl-pyrimidine-5-carbonyl}-amino)-3-[(thiophene-2-carbonyl)-amino]-propionic acid

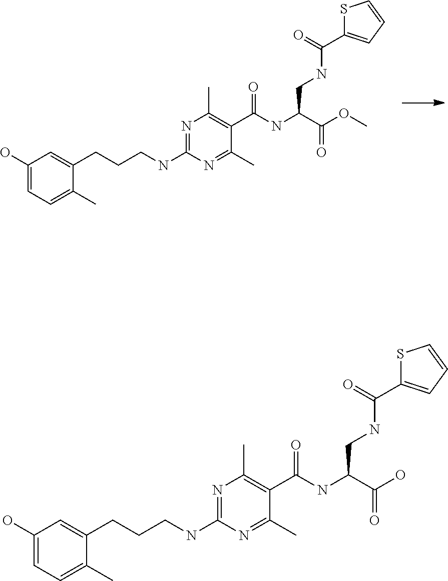

(S)-2-({2-[3-(2-methyl-5-hydroxy-phenyl)-propylamino]-4,6-dimethyl-pyrimidine-5-carbonyl}-amino)-3-[(thiophene-2-carbonyl)-amino]-propionic acid methyl ester (18 mg, 0.034 mmol) afforded the desired product (17 mg, 100% yield). MS m/e 511.8 (M+H⁺).

### Example 73

### (S)-2-({2-[(Z)-3-(3-Hydroxy-phenyl)-allylamino]-4,6-dimethyl-pyrimidine-5-carbonyl}-amino)-3-[(thiophene-2-carbonyl)-amino]-propionic acid

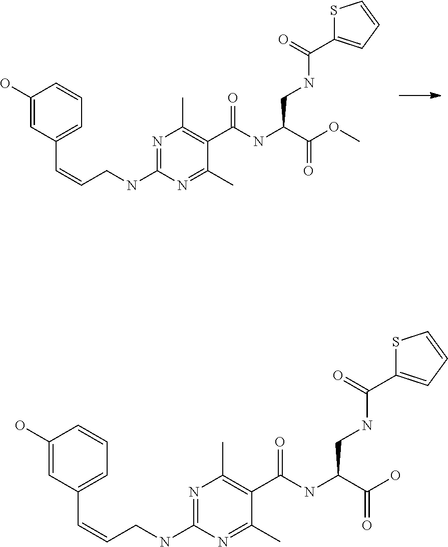

(S)-2-({2-[(Z)-3-(hydroxy-phenyl)-allylamino]-4,6-dimethyl-pyrimidine-5-carbonyl}-amino)-3-[(thiophene-2-carbonyl)-amino]-propionic acid methyl ester (352.5 mg, 0.69 mmol) afforded the desired product (141 mg, 41% yield). MS m/e 495.9 (M+H⁺).

### Example 74

### (S)-2-({2-[(E)-3-(3-Hydroxy-phenyl)-allylamino]-4,6-dimethyl-pyrimidine-5-carbonyl}-amino)-3-[(thiophene-2-carbonyl)-amino]-propionic acid

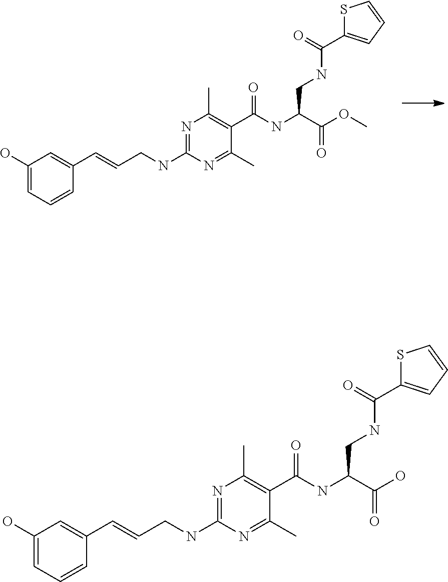

(S)-2-({2-[(E)-3-(hydroxy-phenyl)-allylamino]-4,6-dimethyl-pyrimidine-5-carbonyl}-amino)-3-[(thiophene-2-carbonyl)-amino]-propionic acid methyl ester (45 mg, 0.09 mmol) afforded the desired product (29.5 mg, 66% yield). MS m/e 495.9 (M+H⁺).

### Example 75

### (S)-2-({2-[3-(3-Hydroxy-phenyl)-propylamino]-pyrimidine-5-carbonyl}-amino)-3-[(thiophene-2-carbonyl)-amino]-propionic acid

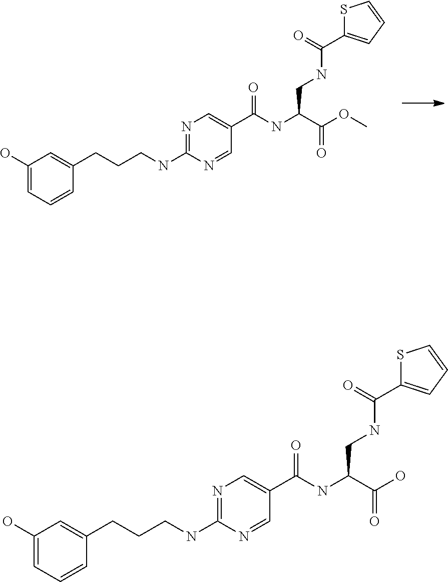

(S)-2-({2-[3-(3-hydroxy-phenyl)-propylamino]-pyrimidine-5-carbonyl}-amino)-3-[(thiophene-2-carbonyl)-amino]-propionic acid methyl ester (220 mg, 0.47 mmol) afforded the product, (16 mg, 7% yield). MS m/e 469.9 (M+H⁺).

### Example 76

### (S)-2-({2-[3-(3-Hydroxy-phenyl)-propylamino]-4-methyl-pyrimidine-5-carbonyl}-amino)-3-[(thiophene-2-carbonyl)-amino]-propionic acid

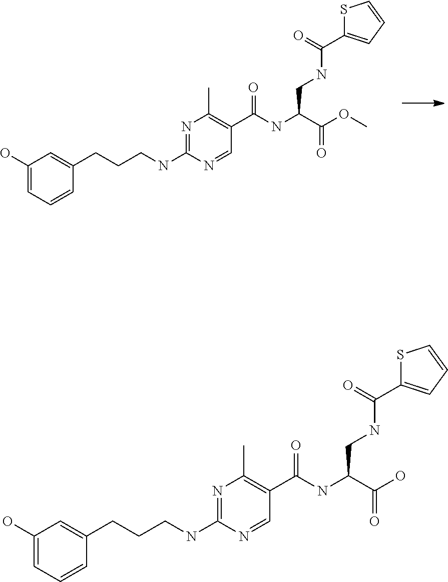

(S)-2-({2-[3-(3-hydroxy-phenyl)-propylamino]-4-methyl-pyrimidine-5-carbonyl}-amino)-3-[(thiophene-2-carbonyl)-amino]-propionic acid methyl ester (319 mg, 0.64 mmol) afforded the product (221.8 mg, 72% yield). MS m/e 483.9 (M+H⁺).

### Example 77

### (S)-2-({2-[3-(3-Hydroxy-phenyl)-propylamino]-4-trifluoromethyl-pyrimidine-5-carbonyl}-amino)-3-[(thiophene-2-carbonyl)-amino]-propionic acid

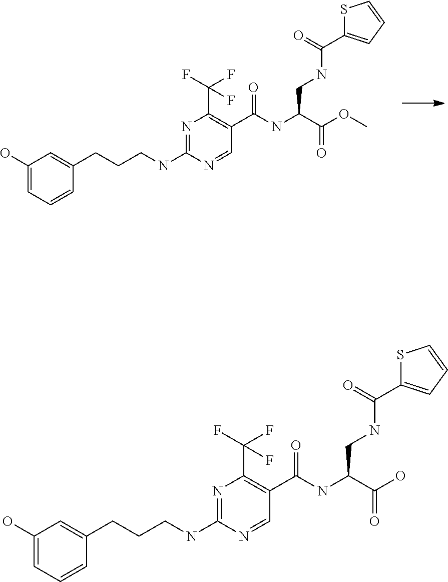

(S)-2-({2-[3-(3-hydroxy-phenyl)-propylamino]-4-trifluoromethyl-pyrimidine-5-carbonyl}-amino)-3-[(thiophene-2-carbonyl)-amino]-propionic acid methyl ester (450 mg, 0.816 mmol) afforded the product (305 mg, 70% yield). MS m/e 538.0 (M+H⁺).

### Example 78

### (S)-2-({4-Ethyl-2-[3-(3-hydroxy-phenyl)-propylamino]-6-methyl-pyrimidine-5-carbonyl}-amino)-3-[(thiophene-2-carbonyl)-amino]-propionic acid

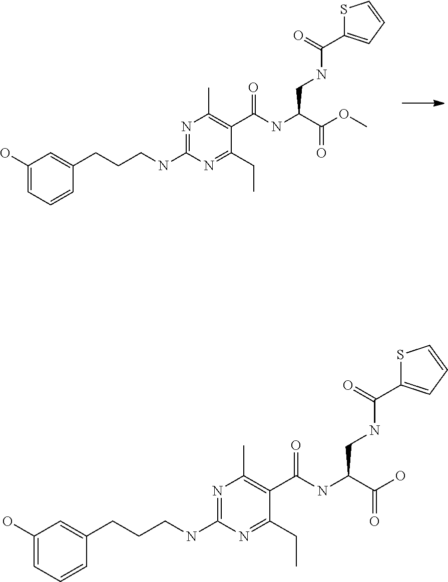

(S)-2-({4-Ethyl-2-[3-(3-hydroxy-phenyl)-propylamino]-6-methyl-pyrimidine-5-carbonyl}-amino)-3-[(thiophene-2-carbonyl)-amino]-propionic acid methyl ester (221 mg, 0.42 mmol) afforded the product (62 mg, 29% yield). MS m/e 511.9 (M+H⁺).

### Example 79

### (S)-2-({4,6-Diethyl-2-[3-(3-hydroxy-phenyl)-propylamino]-pyrimidine-5-carbonyl}-amino)-3-[(thiophene-2-carbonyl)-amino]-propionic acid

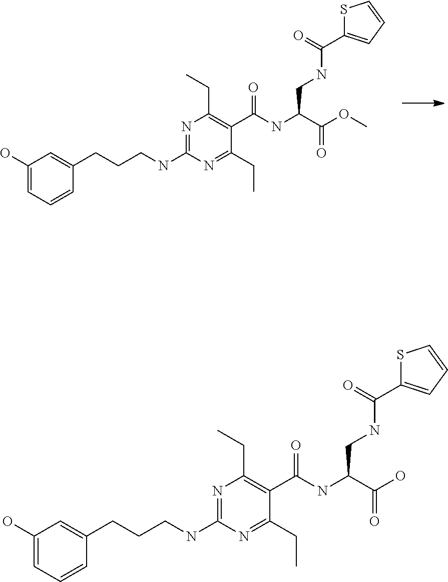

(S)-2-({4,6-diethyl-2-[3-(3-hydroxy-phenyl)-propylamino]-pyrimidine-5-carbonyl}-amino)-3-[(thiophene-2-carbonyl)-amino]-propionic acid methyl ester (9.4 mg, 0.02 mmol) afforded the product (9 mg, 100% yield). MS m/e 526.0 (M+H⁺).

### Example 80

### (S)-3-(3,5-Dihydroxy-benzoylamino-2-({2-[3-(3-hydroxy-phenyl)-propylamino]-4,6-dimethyl-pyrimidine-5-carbonyl}-amino)-propionic acid methyl ester

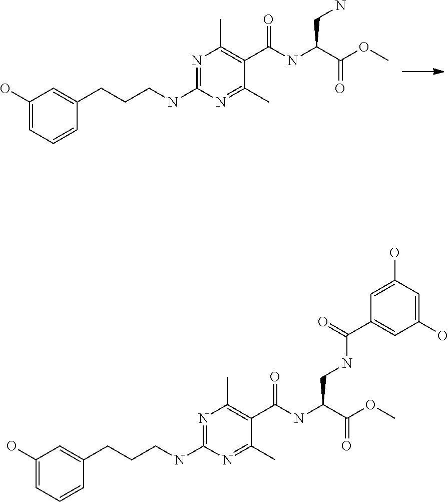

To a mixture of (S)-3-Amino-2-({2-[3-(3-hydroxy-phenyl)-propylamino]-4,6-dimethyl-pyrimidine-5-carbonyl}-amino)-propionic acid methyl ester hydrochloride (77 mg, 0.175 mmol), 3,5-dihydroxybenzoic acid (27 mg, 0.175 mmol), and triethylamine (53 mg, 0.525 mmol) in DMF (4 mL) was added a solution of HBTU (80 mg, 0.21 mmol) and HOBT (28 mg, 0.21 mmol) in DMF (2 mL). The mixture was stirred at room temperature 0.75 h, diluted with EtOAc, washed with brine, dried over sodium sulfate, filtered and concentrated to give the title compound (82.1 mg, 87% yield). MS m/e 538.1 (M+H⁺).

### Example 81

### (S)-3-(3,5-Dihydroxy-benzoylamino-2-({2-[3-(3-hydroxy-phenyl)-propylamino]-4,6-dimethyl-pyrimidine-5-carbonyl}-amino)-propionic acid

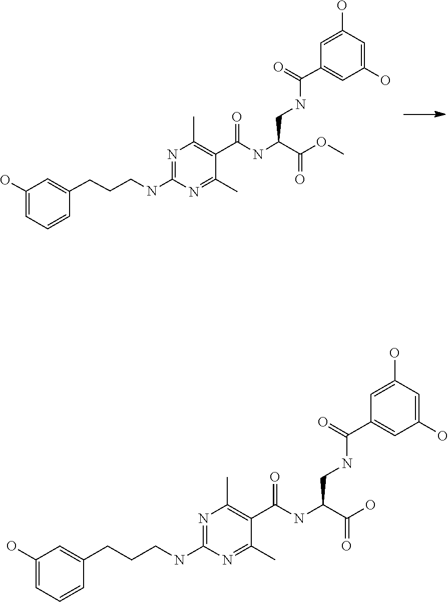

To a solution (S)-3-(3,5-Dihydroxy-benzoylamino-2-({2-[3-(3-hydroxy-phenyl)-propylamino]-4,6-dimethyl-pyrimidine-5-carbonyl}-amino)-propionic acid methyl ester (82.1 mg, 0.153 mmol) in THF (5 mL) was added a solution of lithium hydroxide monohydrate (51 mg, 1.22 mmol) in water (6 mL). The mixture was then stirred at room temperature overnight. The mixture was then diluted with 0.5 N, extracted with EtOAc. The extracts were combined, washed with water and brine, dried over sodium sulfate, filtered, and purified by reverse phase HPLC to give the desired compound (2.0 mg, 2.5% yield) MS m/e 524.2 (M+H⁺).

### Example 82

### (S)-3-(3-Hydroxy-benzoylamino-2-({2-[3-(3-hydroxy-phenyl)-propylamino]-4,6-dimethyl-pyrimidine-5-carbonyl}-amino)-propionic acid methyl ester

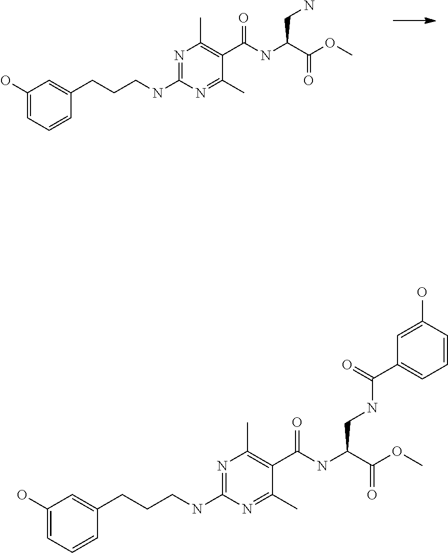

To a mixture of (S)-3-Amino-2-({2-[3-(3-hydroxy-phenyl)-propylamino]-4,6-dimethyl-pyrimidine-5-carbonyl}-amino)-propionic acid methyl ester hydrochloride (65 mg, 0.152 mmol), 3-hydroxybenzoic acid (21 mg, 0.152 mmol), and triethylamine (46 mg, 0.456 mmol) in DMF (3 mL) was added a solution of HBTU (69 mg, 0.182 mmol) and HOBT (25 mg, 0.182 mmol) in DMF (1 mL). The mixture was stirred at room temperature 1 h, diluted with brine and extracted with EtOAc. The combined organic layers were washed with brine, saturated sodium bicarbonate, and brine, dried over Na₂SO₄, filtered, concentrated in the presence of silica gel and chromatographed (40-100% EtOAc in hexane) to afford the desired compound (42 mg, 53% yield) MS m/e 521.9 (M+H⁺).

### Example 83

### (S)-3-(3-Hydroxy-benzoylamino-2-({2-[3-(3-hydroxy-phenyl)-propylamino]-4,6-dimethyl-pyrimidine-5-carbonyl}-amino)-propionic acid

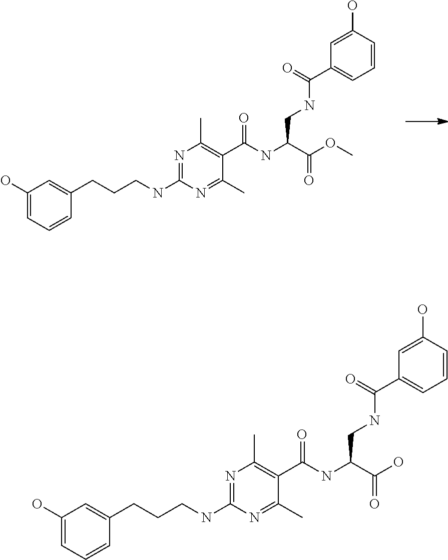

To a solution (S)-3-(3-Hydroxy-benzoylamino-2-({2-[3-(3-hydroxy-phenyl)-propylamino]-4,6-dimethyl-pyrimidine-5-carbonyl}-amino)-propionic acid methyl ester (42 mg, 0.081 mmol) in THF (3 mL) was added a solution of lithium hydroxide monohydrate (17 mg, 0.41 mmol) in water (4 mL). The mixture was then stirred at room temperature 2 h, treated with aqueous potassium hydrogen sulfate to pH 2-4, concentrated, filtered, and the precipitate washed with water to afford title compound (30.1 mg, 74% yield). MS m/e 508.3 (M+H⁺).

### Example 84

### (S)-2-({2-[3-(3-hydroxy-phenyl)-propylamino]-4,6-dimethyl-pyrimidine-5-carbonyl}-amino)-3-[(thiophene-3-carbonyl)-amino]-propionic acid methyl ester

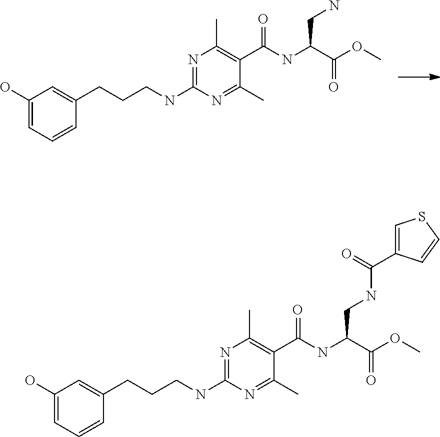

To a mixture of (S)-3-Amino-2-({2-[3-(3-hydroxy-phenyl)-propylamino]-4,6-dimethyl-pyrimidine-5-carbonyl}-amino)-propionic acid methyl ester hydrochloride (75 mg, 0.17 mmol), thiophene-3-carboxylic acid (24 mg, 0.19 mmol), and triethylamine (52 mg, 0.51 mmol) in DMF (2 ml) was added a solution of HBTU (7 mg, 0.19 mmol) and HOBT (26 mg, 0.19 mmol) in DMF (2 ml). The mixture was stirred at room temperature overnight, diluted with brine and extracted with EtOAc. The combined organic layers were washed with 1:1 brine-saturated sodium bicarbonate, and brine, dried over Na₂SO₄, filtered, concentrated in the presence of silica gel and chromatographed (30-100% EtOAc in hexane) to give the title compound (66 mg, 76% yield). MS m/e 511.9 (M+H⁺).

### Example 85

### (S)-2-({2-[3-(3-hydroxy-phenyl)-propylamino]-4,6-dimethyl-pyrimidine-5-carbonyl}-amino)-3-[(thiophene-3-carbonyl)-amino]-propionic acid

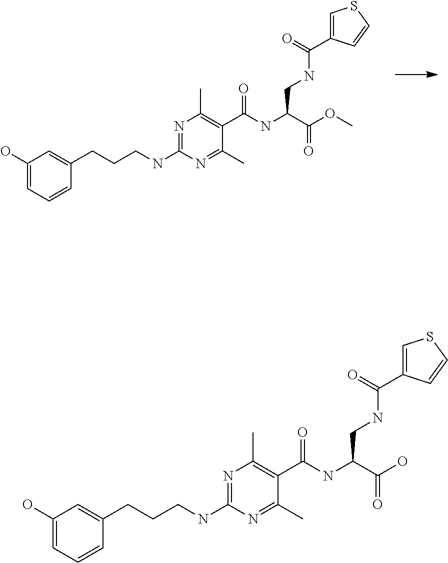

To a solution (S)-2-({2-[3-(3-hydroxy-phenyl)-propylamino]-4,6-dimethyl-pyrimidine-5-carbonyl}-amino)-3-[(thiophene-2-carbonyl)-amino]-propionic acid methyl ester (66.1 mg, 0.129 mmol) in THF (3 ml) was added a solution of lithium hydroxide monohydrate (54 mg, 1.29 mmol) in water (4 ml). The mixture was then stirred at room temperature for 2 h, treated with aqueous potassium hydrogen sulfate to pH 2-4, diluted with water, filtered, and the precipitate washed with water to afford the title compound (38.5 mg, 60% yield). MS m/e 498.1 (M+H⁺).

### Example 86

### (S)-2-({2-[3-(3-hydroxy-phenyl)-propylamino]-4,6-dimethyl-pyrimidine-5-carbonyl}-amino)-3-[(5-methyl-thiophene-2-carbonyl)-amino]-propionic acid methyl ester

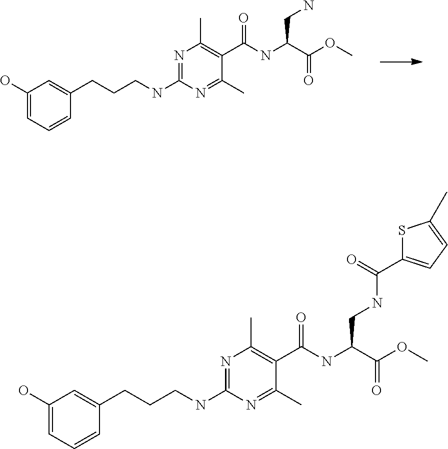

To a mixture of (S)-3-Amino-2-({2-[3-(3-hydroxy-phenyl)-propylamino]-4,6-dimethyl-pyrimidine-5-carbonyl}-amino)-propionic acid methyl ester hydrochloride (75 mg, 0.17 mmol), 5-methyl-2-thiophenecarboxylic acid (27 mg, 0.19 mmol), and triethylamine (52 mg, 0.51 mmol) in DMF (2 ml) was added a solution of HBTU (72 mg, 0.19 mmol) and HOBT (26 mg, 0.19 mmol) in DMF (2 ml). The mixture was stirred at room temperature overnight, diluted with brine and extracted with EtOAc. The combined organic layers were washed with 1:1 brine-saturated sodium bicarbonate and brine, dried over Na₂SO₄, filtered, concentrated in the presence of silica gel and chromatographed (30-100% EtOAc in hexane) to give the desired product (70.3 mg, 78% yield). MS m/e 525.8 (M+H⁺).

### Example 87

### (S)-2-({2-[3-(3-hydroxy-phenyl)-propylamino]-4,6-dimethyl-pyrimidine-5-carbonyl}-amino)-3-[(5-methyl-thiophene-2-carbonyl)-amino]-propionic acid

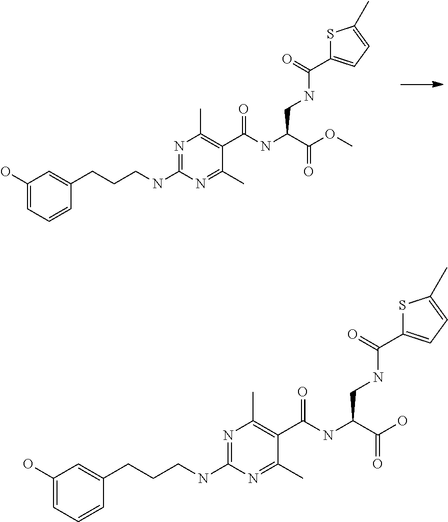

To a solution (S)-2-({2-[3-(3-hydroxy-phenyl)-propylamino]-4,6-dimethyl-pyrimidine-5-carbonyl}-amino)-3-[(5-methyl-thiophene-2-carbonyl)-amino]-propionic acid methyl ester (70.3 mg, 0.13 mmol) in THF (3 ml) was added a solution of lithium hydroxide monohydrate (56 mg, 1.34 mmol) in water (4 ml). The mixture was then stirred at room temperature for 1.5 h, treated with aqueous potassium hydrogen sulfate to pH 2-4, diluted with water, filtered, and the precipitate was washed with water to afford the title compound (45.3 mg, 67% yield). MS m/e 511.9 (M+H⁺).

### Example 88

### (S)-2-({2-[3-(3-hydroxy-phenyl)-propylamino]-4,6-dimethyl-pyrimidine-5-carbonyl}-amino)-3-[(4-methyl-thiophene-2-carbonyl)-amino]-propionic acid methyl ester

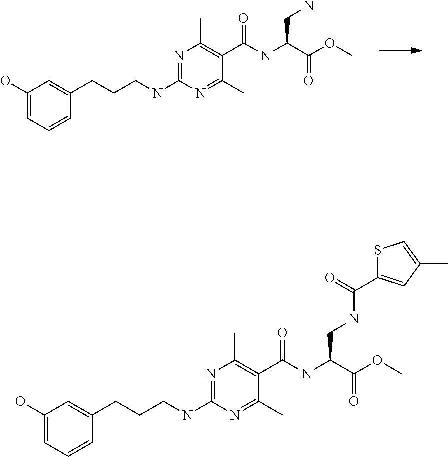

To a mixture of (S)-3-Amino-2-({2-[3-(3-hydroxy-phenyl)-propylamino]-4,6-dimethyl-pyrimidine-5-carbonyl}-amino)-propionic acid methyl ester hydrochloride (75 mg, 0.17 mmol), 4-methyl-2-thiophenecarboxylic acid (27 mg, 0.19 mmol), and triethylamine (52 mg, 0.51 mmol) in DMF (2 ml) was added a solution of HBTU (72 mg, 0.19 mmol) and HOBT (26 mg, 0.19 mmol) in DMF (2 ml). The mixture was stirred at room temperature overnight, diluted with brine, and extracted with EtOAc. The combined organic layers were washed with 1:1 brine-saturated sodium bicarbonate), and brine, dried over Na₂SO₄, filtered, concentrated in the presence of silica gel and chromatographed (30-100% EtOAc in hexane) to give the desired product (59.6 mg, 66% yield. MS m/e 525.9 (M+H⁺).

### Example 89

### (S)-2-({2-[3-(3-hydroxy-phenyl)-propylamino]-4,6-dimethyl-pyrimidine-5-carbonyl}-amino)-3-[(4-methyl-thiophene-2-carbonyl)-amino]-propionic acid

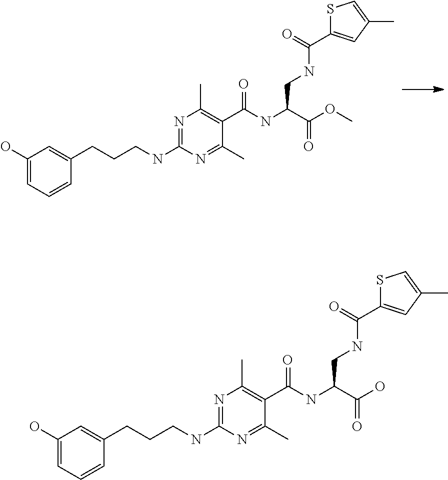

To a solution (S)-2-({2-[3-(3-hydroxy-phenyl)-propylamino]-4,6-dimethyl-pyrimidine-5-carbonyl}-amino)-3-[(4-methyl-thiophene-2-carbonyl)-amino]-propionic acid methyl ester (59.6 mg, 0.113 mmol) in THF (3 ml) was added a solution of lithium hydroxide monohydrate (47 mg, 1.13 mmol) in water (4 ml). The mixture was then stirred at room temperature for 1.5 h, treated with aqueous potassium hydrogen sulfate to pH 2-4, diluted with water, filtered, and the precipitate was washed with water to afford the title compound (46.3 mg, 82% yield). MS m/e 511.9 (M+H⁺).

### Example 90

### (S)-2-({2-[3-(3-hydroxy-phenyl)-propylamino]-4,6-dimethyl-pyrimidine-5-carbonyl}-amino)-3-[(3-methyl-thiophene-2-carbonyl)-amino]-propionic acid methyl ester

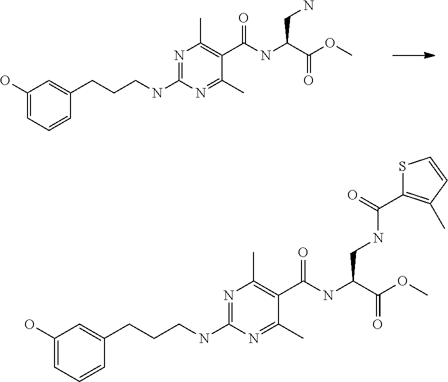

To a mixture of (S)-3-Amino-2-({2-[3-(3-hydroxy-phenyl)-propylamino]-4,6-dimethyl-pyrimidine-5-carbonyl}-amino)-propionic acid methyl ester hydrochloride (75 mg, 0.17 mmol), 3-methyl-2-thiophenecarboxylic acid (24 mg, 0.17 mmol), and triethylamine (52 mg, 0.51 mmol) in DMF (2 ml) was added a solution of HBTU (72 mg, 0.19 mmol) and HOBT (26 mg, 0.19 mmol) in DMF (2 ml). The mixture was stirred at room temperature overnight, diluted with brine (15 ml) and extracted with EtOAc. The combined organic layers were washed with 1:1 brine-saturated sodium bicarbonate, and brine, dried over Na₂SO₄, filtered, concentrated in the presence of silica gel and chromatographed (40-100% EtOAc in hexane) to give the title compound (63.6 mg, 71% yield). MS m/e 525.9 (M+H⁺).

### Example 91

### (S)-2-({2-[3-(3-hydroxy-phenyl)-propylamino]-4,6-dimethyl-pyrimidine-5-carbonyl}-amino)-3-[(3-methyl-thiophene-2-carbonyl)-amino]-propionic acid

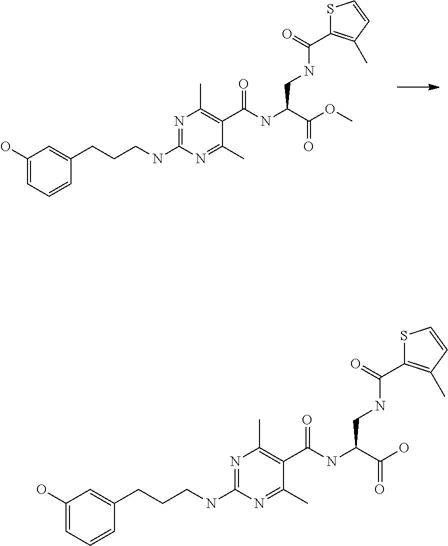

To a solution (S)-2-({2-[3-(3-hydroxy-phenyl)-propylamino]-4,6-dimethyl-pyrimidine-5-carbonyl}-amino)-3-[(3-methyl-thiophene-2-carbonyl)-amino]-propionic acid methyl ester (46.3 mg, 0.088 mmol) in THF (3 ml) was added a solution of lithium hydroxide monohydrate (37 mg, 0.88 mmol) in water (4 ml). The mixture was stirred at room temperature for 1 h, treated with aqueous potassium hydrogen sulfate to pH 2-4, diluted with water, filtered, and the precipitate was washed with water to afford the title compound (45.6 mg, 100% yield). MS m/e 511.9 (M+H⁺).

### Example 92

### (S)-2-({2-[3-(3-hydroxy-phenyl)-propylamino]-4,6-dimethyl-pyrimidine-5-carbonyl}-amino)-3-[(pyrazine-2-carbonyl)-amino]-propionic acid methyl ester

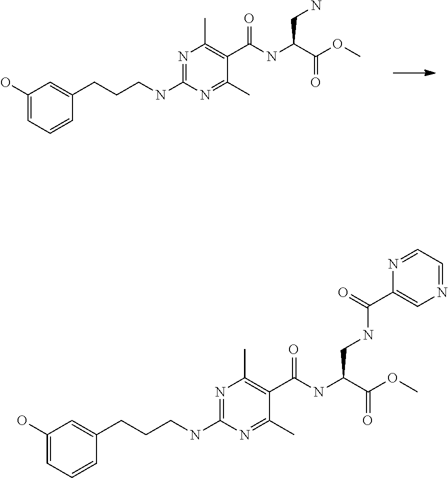

To a mixture of (S)-3-Amino-2-({2-[3-(3-hydroxy-phenyl)-propylamino]-4,6-dimethyl-pyrimidine-5-carbonyl}-amino)-propionic acid methyl ester hydrochloride (75 mg, 0.17 mmol), pyrazine-2-carboxylic acid (21 mg, 0.17 mmol), and triethylamine (52 mg, 0.51 mmol) in DMF (2 ml) was added a solution of HBTU (72 mg, 0.19 mmol) and HOBT (26 mg, 0.19 mmol) in DMF (2 ml). The mixture was stirred at room temperature overnight, diluted with brine and extracted with EtOAc. The combined organic layers were washed with 1:1 brine-saturated sodium bicarbonate and brine, then dried over Na₂SO₄, filtered, concentrated in the presence of silica gel and chromatographed (50-100% EtOAc in hexane) to give the desired product (37.4 mg, 44% yield). MS m/e 508.0 (M+H⁺).

### Example 93

### (S)-2-({2-[3-(3-hydroxy-phenyl)-propylamino]-4,6-dimethyl-pyrimidine-5-carbonyl}-amino)-3-[(pyrazine-2-carbonyl)-amino]-propionic acid

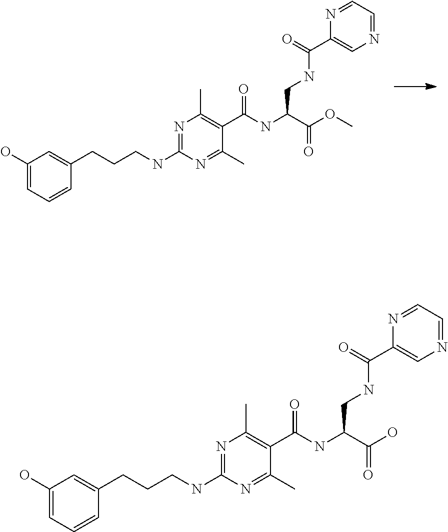

To a solution (S)-2-({2-[3-(3-hydroxy-phenyl)-propylamino]-4,6-dimethyl-pyrimidine-5-carbonyl}-amino)-3-[(pyrazine-2-carbonyl)-amino]-propionic acid methyl ester (61.2 mg, 0.117 mmol) in THF (3 ml) was added a solution of lithium hydroxide monohydrate (49 mg, 1.17 mmol) in water (4 ml). The mixture was then stirred at room temperature for 1 h, treated with aqueous potassium hydrogen sulfate to pH 2-4, diluted with water, filtered, and the precipitate was washed with water to afford the title compound (44.1 mg, 80% yield). MS m/e 509.9 (M+H⁺).

### Example 94

### (S)-3-[(5-Chloro-thiophene-2-carbonyl)-amino]-2-({2-[3-(3-hydroxy-phenyl)-propylamino]-4,6-dimethyl-pyrimidine-5-carbonyl}-amino)-propionic acid methyl ester

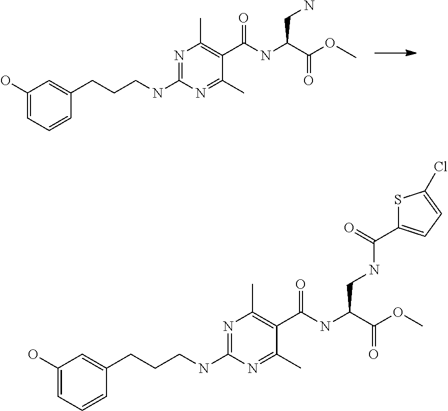

To a mixture of (S)-3-Amino-2-({2-[3-(3-hydroxy-phenyl)-propylamino]-4,6-dimethyl-pyrimidine-5-carbonyl}-amino)-propionic acid methyl ester hydrochloride (75 mg, 0.17 mmol), 5-Chlorothiophene-2-carboxylic acid (28 mg, 0.17 mmol), and triethylamine (52 mg, 0.51 mmol) in DMF (2 ml) was added a solution of HBTU (72 mg, 0.19 mmol) and HOBT (26 mg, 0.19 mmol) in DMF (2 ml). The mixture was stirred at room temperature overnight, diluted with brine and extracted with EtOAc. The combined organic layers were washed with 1:1 brine-saturated sodium bicarbonate and brine, dried over Na₂SO₄, filtered, concentrated in the presence of silica gel and chromatographed (30-100% EtOAc in hexane) to give the desired compound (62.2 mg, 67% yield). MS m/e 545.9 (M+H⁺).

### Example 95

### (S)-3-[(5-Chloro-thiophene-2-carbonyl)-amino]-2-({2-[3-(3-hydroxy-phenyl)-propylamino]-4,6-dimethyl-pyrimidine-5-carbonyl}-amino)-propionic acid

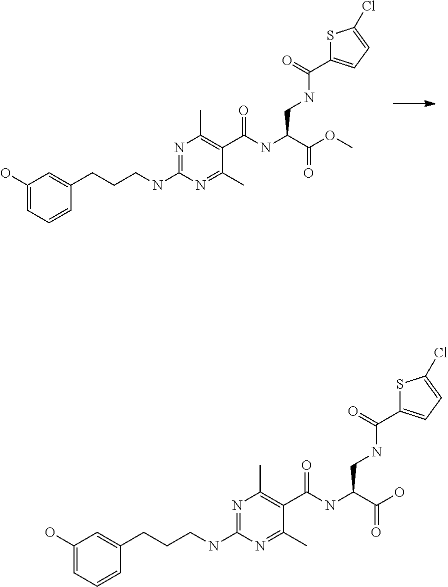

To a solution (S)-3-[(5-Chloro-thiophene-2-carbonyl)-amino]-2-({2-[3-(3-hydroxy-phenyl)-propylamino]-4,6-dimethyl-pyrimidine-5-carbonyl}-amino)-propionic acid methyl ester (62.2 mg, 0.11 mmol) in THF (3 ml) was added a solution of lithium hydroxide monohydrate (48 mg, 1.14 mmol) in water (4 ml). The mixture was stirred at room temperature for 1.5 h, treated with aqueous potassium hydrogen sulfate to pH 2-4, diluted with water, filtered, and the precipitate was washed with water to afford the title compound (45.7 mg, 79% yield). MS m/e 532.0 (M+H⁺).

### Example 96

### (S)-2-({2-[3-(3-hydroxy-phenyl)-propylamino]-4,6-dimethyl-pyrimidine-5-carbonyl}-amino)-3-(3-methyl-benzoylamino)-propionic acid methyl ester

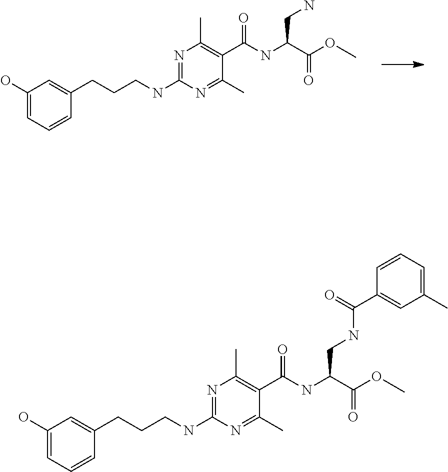

To a mixture of (S)-3-Amino-2-({2-[3-(3-hydroxy-phenyl)-propylamino]-4,6-dimethyl-pyrimidine-5-carbonyl}-amino)-propionic acid methyl ester hydrochloride (75 mg, 0.17 mmol), m-toluic acid (23 mg, 0.17 mmol), and triethylamine (52 mg, 0.51 mmol) in DMF (2 ml) was added a solution of HBTU (72 mg, 0.19 mmol) and HOBT (26 mg, 0.19 mmol) in DMF (2 ml). The mixture was stirred at room temperature overnight, diluted with brine and extracted with EtOAc. The combined organic layers were washed with 1:1 brine-saturated sodium bicarbonate and brine, then dried over Na₂SO₄, filtered, concentrated in the presence of silica gel and chromatographed (30-100% EtOAc in hexane) to give the desired compound (67.5 mg, 77% yield). MS m/e 520.0 (M+H⁺).

### Example 97

### (S)-2-({2-[3-(3-hydroxy-phenyl)-propylamino]-4,6-dimethyl-pyrimidine-5-carbonyl}-amino)-3-(3-methyl-benzoylamino)-propionic acid

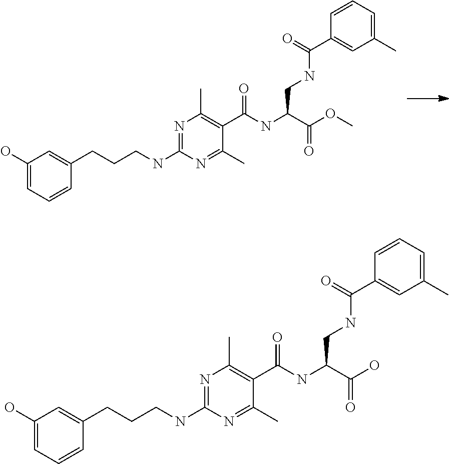

To a solution (S)-2-({2-[3-(3-hydroxy-phenyl)-propylamino]-4,6-dimethyl-pyrimidine-5-carbonyl}-amino)-3-(3-methyl-benzoylamino)-propionic acid methyl ester (67.5 mg, 0.13 mmol) in THF (3 ml) was added a solution of lithium hydroxide monohydrate (55 mg, 1.17 mmol) in water (4 ml). The mixture was then stirred at room temperature for 1 h, treated with aqueous potassium hydrogen sulfate to pH 2-4, diluted with water, filtered, and the precipitate was washed with water to afford the title compound (52.3 mg, 80% yield). MS m/e 505.9 (M+H⁺).

### Example 98

### (S)-3-[(Adamantane-1-carbonyl)-amino]-2-({2-[3-(3-hydroxy-phenyl)-propylamino]-4,6-dimethyl-pyrimidine-5-carbonyl}-amino)-propionic acid methyl ester

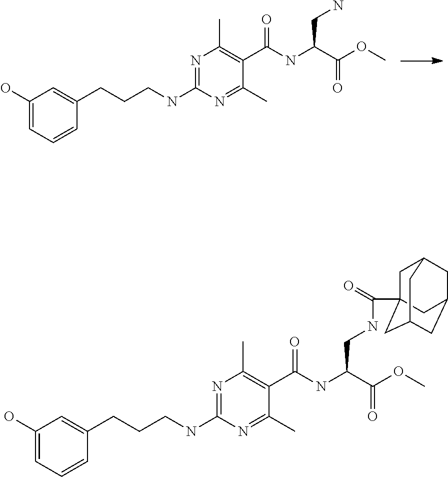

To a mixture of (S)-3-Amino-2-({2-[3-(3-hydroxy-phenyl)-propylamino]-4,6-dimethyl-pyrimidine-5-carbonyl}-amino)-propionic acid methyl ester hydrochloride (50 mg, 0.114 mmol), 1-adamantanecarboxylic acid (21 mg, 0.114 mmol), and triethylamine (35 mg, 0.34 mmol) in DMF (1.5 ml) was added a solution of HBTU (48 mg, 0.126 mmol) and HOBT (17 mg, 0.126 mmol) in DMF (1.5 ml). The mixture was stirred at room temperature for 2 h, diluted with brine and extracted with EtOAc. The combined organic layers were washed with 1:1 brine-saturated sodium bicarbonate and brine, dried over Na₂SO₄, filtered, concentrated in the presence of silica gel and chromatographed (30-100% EtOAc in hexane) to give the desired product (45.8 mg, 73% yield). MS m/e 564.0 (M+H⁺).

### Example 99

### (S)-3-[(Adamantane-1-carbonyl)-amino]-2-({2-[3-(3-hydroxy-phenyl)-propylamino]-4,6-dimethyl-pyrimidine-5-carbonyl}-amino)-propionic acid

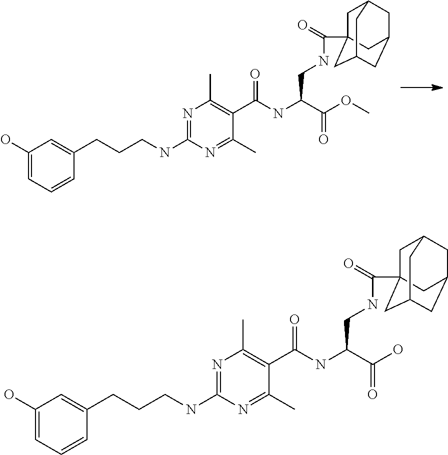

To a solution (S)-3-[(adamantane-1-carbonyl)-amino]-2-({2-[3-(3-hydroxy-phenyl)-propylamino]-4,6-dimethyl-pyrimidine-5-carbonyl}-amino)-propionic acid methyl ester (67.5 mg, 0.13 mmol) in THF (3 ml) was added a solution of lithium hydroxide monohydrate (55 mg, 1.17 mmol) in water (4 ml). The mixture was then stirred at room temperature for 1 h, treated with aqueous potassium hydrogen sulfate to pH 2-4, diluted with water, filtered, and the precipitate was washed with water to afford the title compound (52.3 mg, 80% yield). MS m/e 505.9 (M+H⁺).

### Example 100

### (S)-2-({2-[3-(3-Hydroxy-phenyl)-propylamino]-4,6-dimethyl-pyrimidine-5-carbonyl}-amino)-3-[(5-methyl-pyrazine-2-carbonyl)-amino]-propionic acid methyl ester

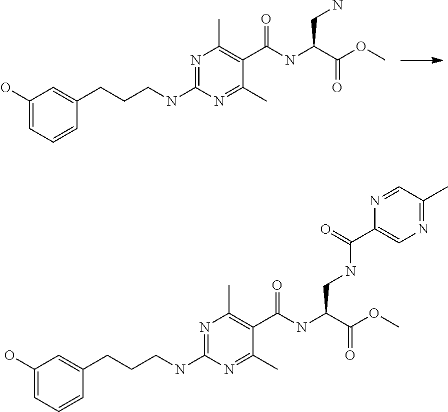

To a mixture of (S)-3-amino-2-({2-[3-(3-hydroxy-phenyl)-propylamino]-4,6-dimethyl-pyrimidine-5-carbonyl}-amino)-propionic acid methyl ester hydrochloride (50 mg, 0.114 mmol), 5-methyl piperazinecarboxylic acid (16 mg, 0.114 mmol), and triethylamine (35 mg, 0.34 mmol) in DMF (1.5 ml) was added a solution of HBTU (48 mg, 0.126 mmol) and HOBT (17 mg, 0.126 mmol) in DMF (1.5 ml). The mixture was stirred at room temperature for 2.5 h, diluted with brine and extracted with EtOAc. The combined organic layers were washed with 1:1 brine-saturated sodium bicarbonate and brine, dried over Na₂SO₄, filtered, concentrated in the presence of silica gel and chromatographed (40-100% EtOAc in hexane) to give the title compound (32.9 mg, 59% yield). MS m/e 522.0 (M+H⁺).

### Example 101

### (S)-2-({2-[3-(3-Hydroxy-phenyl)-propylamino]-4,6-dimethyl-pyrimidine-5-carbonyl}-amino)-3-[(5-methyl-pyrazine-2-carbonyl)-amino]-propionic acid

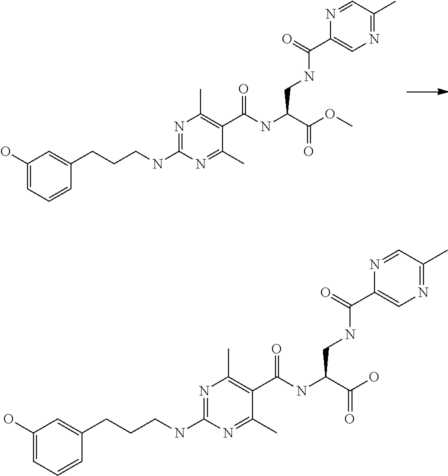

To a solution (S)-2-({2-[3-(3-hydroxy-phenyl)-propylamino]-4,6-dimethyl-pyrimidine-5-carbonyl}-amino)-3-[(5-methyl-pyrazine-2-carbonyl)-amino]-propionic acid methyl ester (32.9 mg, 0.063 mmol) in THF (3 ml) was added a solution of lithium hydroxide monohydrate (26 mg, 0.63 mmol) in water (4 ml). The mixture was then stirred at room temperature for 1 h, treated with aqueous potassium hydrogen sulfate to pH 2-4, diluted with water, filtered, and the precipitate was washed with water to afford the title compound (25.4 mg, 78% yield). MS m/e 507.9 (M+H⁺).

### Example 102

### (S)-3-[(3-Chloro-thiophene-2-carbonyl)-amino]-2-({2-[3-(3-hydroxy-phenyl)-propylamino]-4,6-dimethyl-pyrimidine-5-carbonyl}-amino)-propionic acid methyl ester

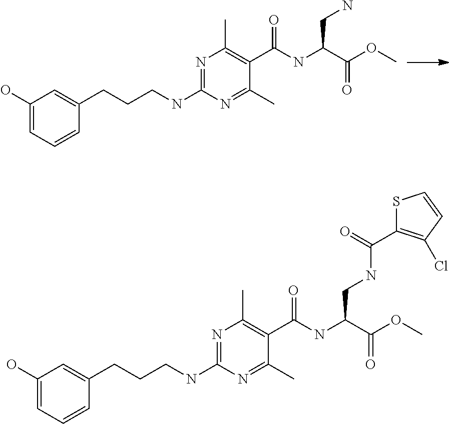

To a mixture of (S)-3-amino-2-({2-[3-(3-hydroxy-phenyl)-propylamino]-4,6-dimethyl-pyrimidine-5-carbonyl}-amino)-propionic acid methyl ester hydrochloride (50 mg, 0.114 mmol), 3-chlorothiophenecarboxylic acid (19 mg, 0.114 mmol), and triethylamine (35 mg, 0.34 mmol) in DMF (1.5 ml) was added a solution of HBTU (48 mg, 0.126 mmol) and HOBT (17 mg, 0.126 mmol) in DMF (1.5 ml). The mixture was stirred at room temperature overnight, diluted with brine and extracted with EtOAc. The combined organic layers were washed with 1:1 brine-saturated sodium bicarbonate and brine, dried over Na₂SO₄, filtered, concentrated in the presence of silica gel and chromatographed (30-100% EtOAc in hexane) to give the title compound (45.8 mg, 92% yield). MS m/e 545.9 (M+H⁺).

### Example 103

### (S)-3-[(3-Chloro-thiophene-2-carbonyl)-amino]-2-({2-[3-(3-hydroxy-phenyl)-propylamino]-4,6-dimethyl-pyrimidine-5-carbonyl}-amino)-propionic acid

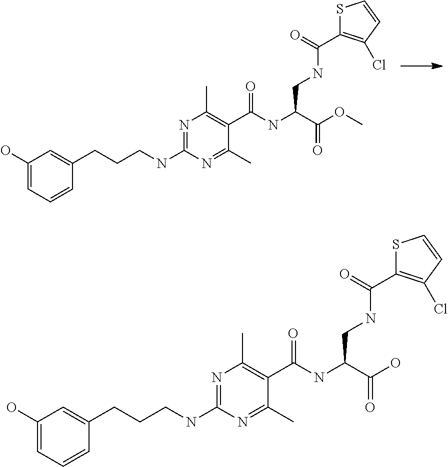

To a solution (S)-3-[(3-chloro-thiophene-2-carbonyl)-amino]-2-({2-[3-(3-hydroxy-phenyl)-propylamino]-4,6-dimethyl-pyrimidine-5-carbonyl}-amino)-propionic acid methyl ester (45.8 mg, 0.084 mmol) in THF (3 ml) was added a solution of lithium hydroxide monohydrate (35 mg, 0.84 mmol) in water (4 ml). The mixture was then stirred at room temperature for 1 h, treated with aqueous potassium hydrogen sulfate to pH 2-4, diluted with water, filtered, and the precipitate was washed with water to afford the title compound (28.1 mg, 66% yield). MS m/e 531.9 (M+H⁺).

### Example 104

### (S)-[(5-Ethyl-thiophene-2-carbonyl)-amino]-2-({2-[3-(3-hydroxy-phenyl)-propylamino]-4,6-dimethyl-pyrimidine-5-carbonyl}-amino)-3-propionic acid methyl ester

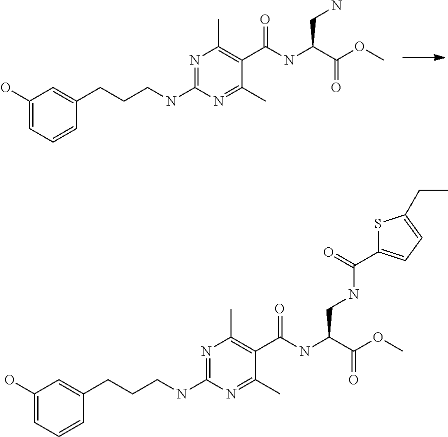

To a mixture of (S)-3-amino-2-({2-[3-(3-hydroxy-phenyl)-propylamino]-4,6-dimethyl-pyrimidine-5-carbonyl}-amino)-propionic acid methyl ester hydrochloride (53 mg, 0.121 mmol), 5-ethylthiophenecarboxylic acid (19 mg, 0.121 mmol), and triethylamine (37 mg, 0.363 mmol) in DMF (1.5 ml) was added a solution of HBTU (50 mg, 0.133 mmol) and HOBT (18 mg, 0.133 mmol) in DMF (1.5 ml). The mixture was stirred at room temperature overnight, diluted with brine and extracted with EtOAc. The combined organic layers were washed with 1:1 brine-saturated sodium bicarbonate and brine, dried over Na₂SO₄, filtered, concentrated in the presence of silica gel and chromatographed (30-100% EtOAc in hexane) to give the desired product (36.8 mg, 58% yield). MS m/e 539.8 (M+H⁺).

### Example 105

### (S)-[(5-Ethyl-thiophene-2-carbonyl)-amino]-2-({2-[3-(3-hydroxy-phenyl)-propylamino]-4,6-dimethyl-pyrimidine-5-carbonyl}-amino)-3-propionic acid

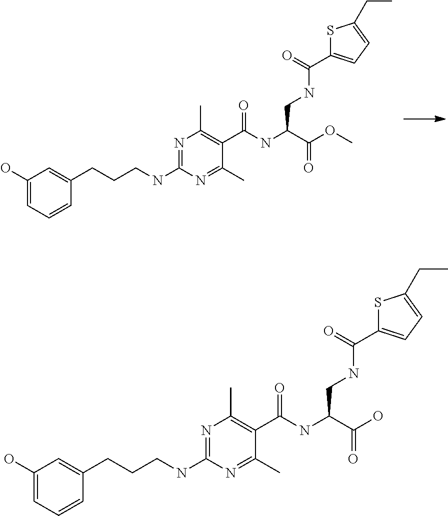

To a solution (S)-3-[(5-ethyl-thiophene-2-carbonyl)-amino]-2-({2-[3-(3-hydroxy-phenyl)-propylamino]-4,6-dimethyl-pyrimidine-5-carbonyl}-amino)-propionic acid methyl ester (36.8 mg, 0.068 mmol) in THF (3 ml) was added a solution of lithium hydroxide monohydrate (28 mg, 0.68 mmol) in water (4 ml). The mixture was stirred at room temperature 1.5 h, treated with aqueous potassium hydrogen sulfate to pH 2-4, diluted with water, filtered, and the precipitate was washed with water to afford the title compound (23.9 mg, 67% yield). MS m/e 525.9 (M+H⁺).

### Example 106

### (S)-2-({4,6-Dimethyl-2-[3-(3-phenyl carbamoyloxy-phenyl)-propylamino]-pyrimidine-5-carbonyl}-amino)-3-(3-phenyl-ureido)-propionic acid methyl ester

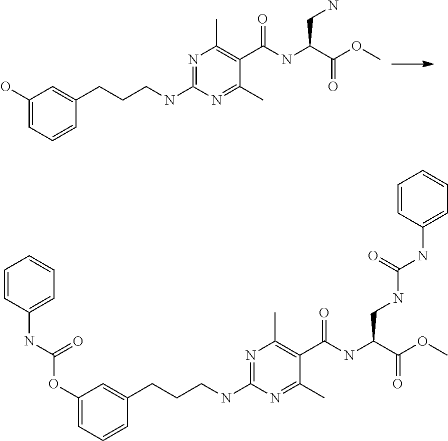

A mixture of (S)-3-amino-2-({2-[3-(3-hydroxy-phenyl)-propylamino]-4,6-dimethyl-pyrimidine-5-carbonyl}-amino)-propionic acid methyl ester hydrochloride (50 mg, 0.114 mmol), phenyl isocyanate (45 mg, 0.38 mmol), triethylamine (38 mg, 0.38 mmol) and DMF (3 ml) was stirred at room temperature overnight, diluted with EtOAc, washed with brine, dried over Na₂SO₄, filtered, concentrated in the presence of silica gel and chromatographed (0-100% EtOAc in hexane) to give the desired product (36.8 mg, 53% yield)

### Example 107

### (S)-2-({2-[3-(3-hydroxy-phenyl)-propylamino]-4,6-dimethyl-pyrimidine-5-carbonyl}-amino)-3-(3-phenyl-ureido)-propionic acid

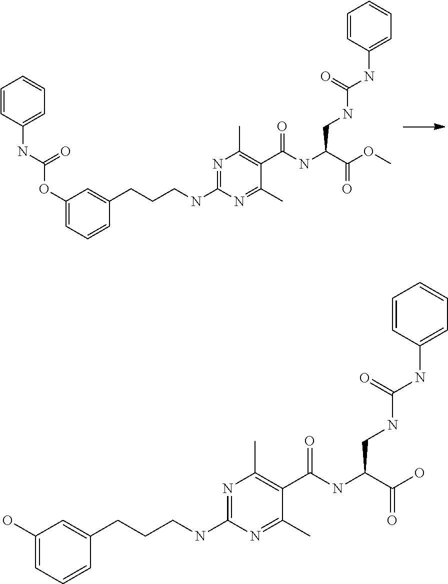

To a solution of (S)-2-({4,6-Dimethyl-2-[3-(3-phenyl carbamoyloxy-phenyl)-propylamino]-pyrimidine-5-carbonyl}-amino)-3-(3-phenyl-ureido)-propionic acid methyl ester (36.8 mg, 0.058 mmol) in THF (3 ml) was added a solution of lithium hydroxide monohydrate (24 mg, 0.575 mmol) in water (4 ml). The mixture was stirred at room temperature for 1.5 h, treated with aqueous potassium hydrogen sulfate to pH 2-4, diluted with water, filtered, and the precipitate was washed with water to afford title compound, (18.8 mg, 62% yield). MS m/e 507.9 (M+H⁺).

### Example 108

### (S)-2-({2-[3-(3-Hydroxy-phenyl)-propylamino]-4,6-dimethyl-pyrimidine-5-carbonyl}-amino)-3-methoxycarbonylamino-propionic acid methyl ester

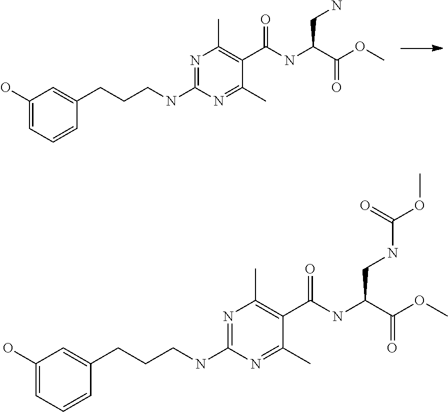

A mixture of (S)-3-amino-2-({2-[3-(3-hydroxy-phenyl)-propylamino]-4,6-dimethyl-pyrimidine-5-carbonyl}-amino)-propionic acid methyl ester hydrochloride (50 mg, 0.114 mmol), methyl chloroformate (32 mg, 0.342 mmol), triethylamine (58 mg, 0.57 mmol) and DMF (3 ml) was stirred at room temperature overnight, diluted with brine and extracted with EtOAc. The combined organic layers were washed with brine, dried over Na₂SO₄, filtered, concentrated in the presence of silica gel and chromatographed (40-100% EtOAc in hexane) to give the title compound (30.9 mg, 57% yield)

### Example 109

### (S)-2-({2-[3-(3-Hydroxy-phenyl)-propylamino]-4,6-dimethyl-pyrimidine-5-carbonyl}-amino)-3-methoxycarbonylamino-propionic acid

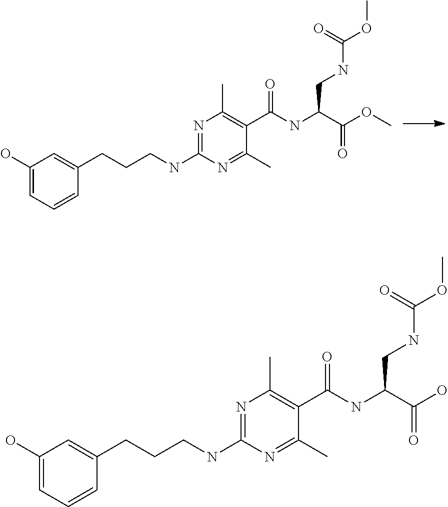

To a solution of (S)-2-({2-[3-(3-Hydroxy-phenyl)-propylamino]-4,6-dimethyl-pyrimidine-5-carbonyl}-amino)-3-methoxycarbonylamino-propionic acid methyl ester (30.9 mg, 0.067 mmol) in THF (3 ml) was added a solution of lithium hydroxide monohydrate (28 mg, 0.67 mmol) in water (4 ml). The mixture was stirred at room temperature for 2 h, treated with aqueous potassium hydrogen sulfate to pH 2-4, diluted with water and extracted with EtOAc.

The combined organic layers were washed with brine, dried over Na₂SO₄, filtered, and concentrated to afford the desired product (17.3 mg, 58% yield). MS m/e 445.8 (M+H⁺).

### Example 110

### (S)-3-Benzyloxycarbonylamino-2-({2-[3-(3-Hydroxy-phenyl)-propylamino]-4,6-dimethyl-pyrimidine-5-carbonyl}-amino)-propionic acid methyl ester

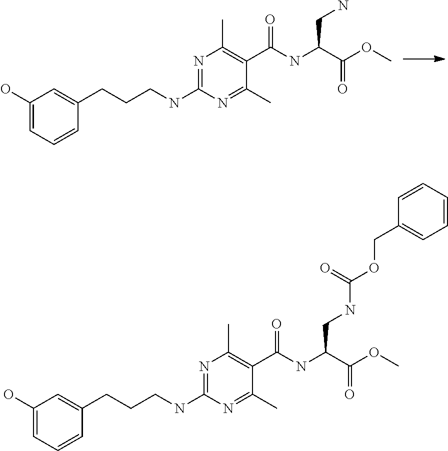

A mixture of (S)-3-amino-2-({2-[3-(3-hydroxy-phenyl)-propylamino]-4,6-dimethyl-pyrimidine-5-carbonyl}-amino)-propionic acid methyl ester hydrochloride (50 mg, 0.114 mmol), benzyl chloroformate (21 mg, 0.125 mmol), triethylamine (38 mg, 0.38 mmol) and DMF (3 ml) was stirred at room temperature overnight, diluted with brine and extracted with EtOAc. The combined organic layers were washed with brine, dried over Na₂SO₄, filtered, concentrated in the presence of silica gel and chromatographed (30-100% EtOAc in hexane) to give the desired product (12.6 mg, 21% yield).

### Example 111

### (S)-3-Benzyloxycarbonylamino-2-({2-[3-(3-Hydroxy-phenyl)-propylamino]-4,6-dimethyl-pyrimidine-5-carbonyl}-amino)-propionic acid

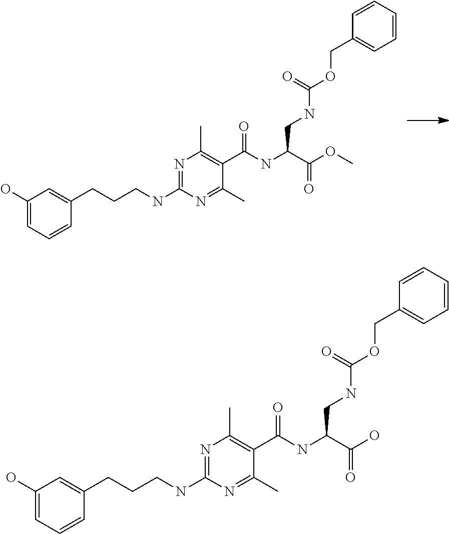

To a solution of (S)-3-benzyloxycarbonylamino-2-({2-[3-(3-hydroxy-phenyl)-propylamino]-4,6-dimethyl-pyrimidine-5-carbonyl}-amino)-propionic acid methyl ester (12.6 mg, 0.024 mmol) in THF (2 ml) was added a solution of lithium hydroxide monohydrate (10 mg, 0.235 mmol) in water (3 ml). The mixture was stirred at room temperature for 2 h, treated with aqueous potassium hydrogen to pH 2-4, diluted with water and extracted with EtOAc. The combined organic layers were washed with brine, dried over Na₂SO₄, filtered, and concentrated to afford the title compound (5.7 mg, 46% yield). MS m/e 521.9 (M+H⁺).

### Example 112

### (S)-3-(3,3-Dimethyl-ureido)-2-({2-[3-(3-Hydroxy-phenyl)-propylamino]-4,6-dimethyl-pyrimidine-5-carbonyl}-amino)-propionic acid methyl ester

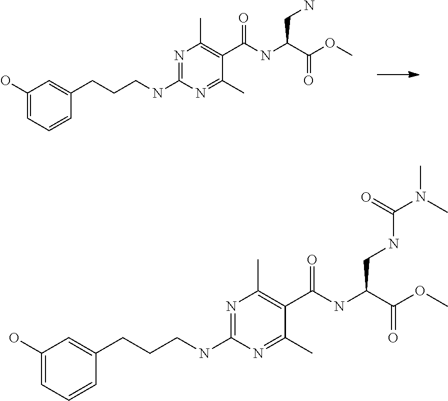

A solution of (S)-3-amino-2-({2-[3-(3-hydroxy-phenyl)-propylamino]-4,6-dimethyl-pyrimidine-5-carbonyl}-amino)-propionic acid methyl ester hydrochloride (53 mg, 0.121 mmol), dimethylcarbamyl chloride (16 mg, 0.145 mmol), triethylamine (40 mg, 0.40 mmol) in DMF (2.5 ml) was stirred at room temperature overnight, diluted with brine and extracted with EtOAc. The combined organic layers were washed with brine, dried over Na₂SO₄, filtered, concentrated in the presence of silica gel and chromatographed (0-10% MeOH in EtOAc) to give the desired product (35.7 mg, 63% yield).

### Example 113

### (S)-3-(3,3-Dimethyl-ureido)-2-({2-[3-(3-Hydroxy-phenyl)-propylamino]-4,6-dimethyl-pyrimidine-5-carbonyl}-amino)-propionic acid

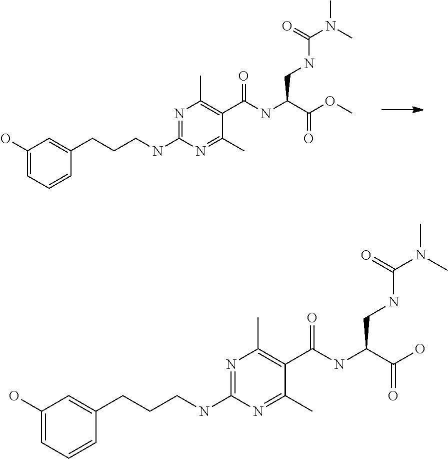

To a solution of (S)-3-(3,3-dimethyl-ureido)-2-({2-[3-(3-hydroxy-phenyl)-propylamino]-4,6-dimethyl-pyrimidine-5-carbonyl}-amino)-propionic acid methyl ester (35.7 mg, 0.024 mmol) in THF (3 ml) was added a solution of lithium hydroxide monohydrate (32 mg, 0.755 mmol) in water (4 ml). The mixture was stirred at room temperature 1 h, treated with aqueous potassium hydrogen sulfate to pH 2-4, diluted with water and extracted with EtOAc. The combined organic layers were washed with brine, dried over Na₂SO₄, filtered, and concentrated to afford the desired product (10.2 mg, 91% yield). MS m/e 459.0 (M+H⁺).

### Example 114

### (S)-3-[(3,4-Dihydro-2H-quinoline-1-carbonyl)-amino]-2-({2-[3-(3-hydroxy-phenyl)-propylamino]-4,6-dimethyl-pyrimidine-5-carbonyl}-amino)-propionic acid methyl ester

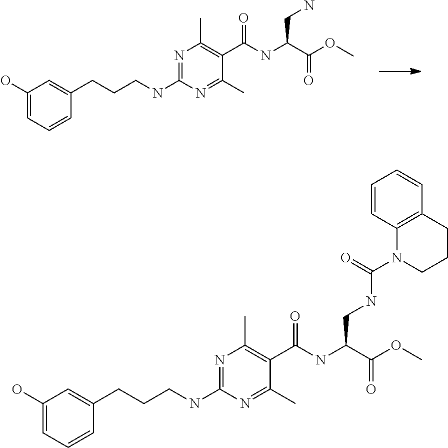

A mixture of (S)-3-Amino-2-({2-[3-(3-hydroxy-phenyl)-propylamino]-4,6-dimethyl-pyrimidine-5-carbonyl}-amino)-propionic acid methyl ester hydrochloride (52 mg, 0.119 mmol), 3,4-Dihydroquinolin-1(2H)-carbonyl chloride (28 mg, 0.142 mmol), Triethylamine (40 mg, 0.40 mmol) and DMF (2.5 ml) was stirred at room temperature overnight, diluted with brine and extracted with EtOAc. The combined organic layers were washed with brine, dried over Na₂SO₄, filtered, concentrated in the presence of silica gel and chromatographed (40-100% EtOAc in hexane) to give the desired product (37.2 mg, 56% yield). MS m/e 561.0 (M+H⁺).

### Example 115

### (S)-3-[(3,4-Dihydro-2H-quinoline-1-carbonyl)-amino]-2-({2-[3-(3-hydroxy-phenyl)-propylamino]-4,6-dimethyl-pyrimidine-5-carbonyl}-amino)-propionic acid

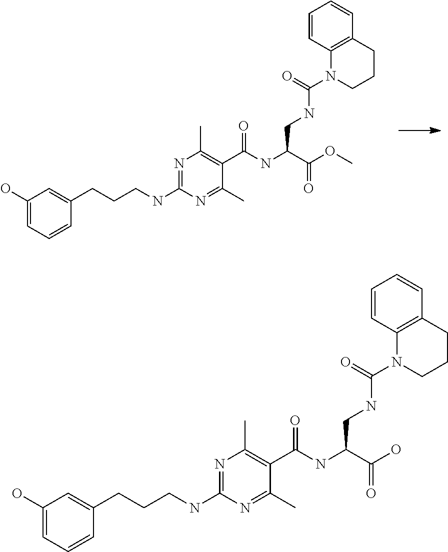

To a solution of (S)-3-[(3,4-dihydro-2H-quinoline-1-carbonyl)-amino]-2-({2-[3-(3-hydroxy-phenyl)-propylamino]-4,6-dimethyl-pyrimidine-5-carbonyl}-amino)-propionic acid methyl ester (37.2 mg, 0.066 mmol) in THF (3 ml) was added a solution of lithium hydroxide monohydrate (28 mg, 0.66 mmol) in water (4 ml). The mixture was stirred at room temperature for 1 h, treated with aqueous potassium hydrogen sulfate to pH 2-4, diluted with water, filtered and the precipitate was washed with water to afford the desired product (13.0 mg, 36% yield). MS m/e 547.0 (M+H⁺).

### Example 116

### (S)-2-{[2-(8-Hydroxy-1,2,3,4-tetrahydro-naphthalen-2-ylamino)-4,6-dimethyl-pyrimidine-5-carbonyl]-amino}-3-[(pyrazine-2-carbonyl)-amino]-propionic acid methyl ester

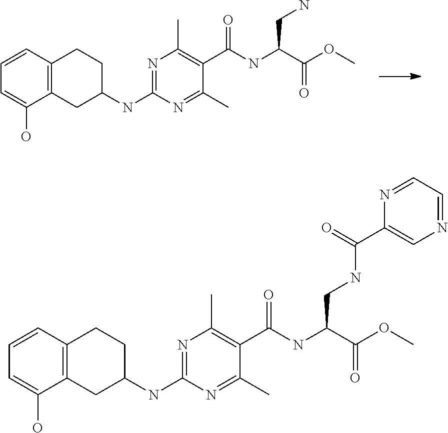

To a mixture of (S)-3-Amino-2-{[2-(8-hydroxy-1,2,3,4-tetrahydro-naphthalen-2-ylamino)-4,6-dimethyl-pyrimidine-5-carbonyl]-amino}-propionic acid methyl ester hydrochloride (50 mg, 0.111 mmol), Pyrazine-2-carboxylic acid (14 mg, 0.111 mmol), and Triethylamine (34 mg, 0.333 mmol) in DMF (1.5 mL) was added a solution of HBTU (46 mg, 0.122 mmol) and HOBT (16 mg, 0.122 mmol) in DMF (1.5 mL). The mixture was stirred at room temperature 2 h, diluted with brine (20 mL) and extracted with EtOAc (2×15 mL). The combined organic layers were washed with 1:1 brine-saturated sodium bicarbonate (2×20 mL), and brine (15 mL), dried over Na₂SO₄, filtered, concentrated in the presence of silica gel and chromatographed (30-100% EtOAc in hexane) to give 24.7 mg title compound. MS m/e 520.0 (M+H⁺).

### Example 117

### (S)-2-{[2-(8-Hydroxy-1,2,3,4-tetrahydro-naphthalen-2-ylamino)-4,6-dimethyl-pyrimidine-5-carbonyl]-amino}-3-[(pyrazine-2-carbonyl)-amino]-propionic acid

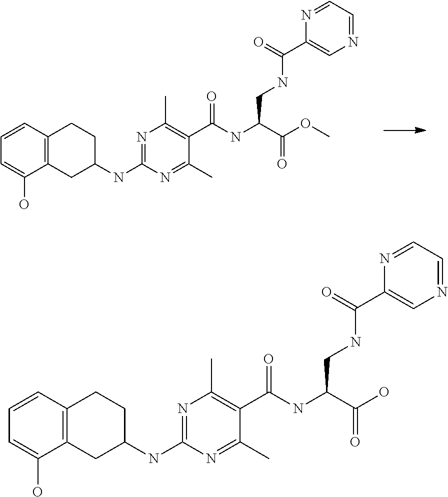

To a solution of (S)-2-{[2-(8-Hydroxy-1,2,3,4-tetrahydro-naphthalen-2-ylamino)-4,6-dimethyl-pyrimidine-5-carbonyl]-amino}-3-[(pyrazine-2-carbonyl)-amino]-propionic acid methyl ester (24.7 mg, 0.048 mmol) in THF (2 mL) was added a solution of lithium hydroxide monohydrate (20 mg, 0.48 mmol) in water (3 mL). The mixture was then stirred at room temperature 1 h, treated with potassium hydrogen sulfate aqueous solution to pH 2-4, diluted with water (20 mL), extracted with EtOAc (3.15 mL). The organic layers were combined, and washed with water (2×20 mL) to afford 8.7 mg title compound. MS m/e 505.9 (M+H⁺).

### Example 118

### (S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid 2,2-dimethyl-propyl ester

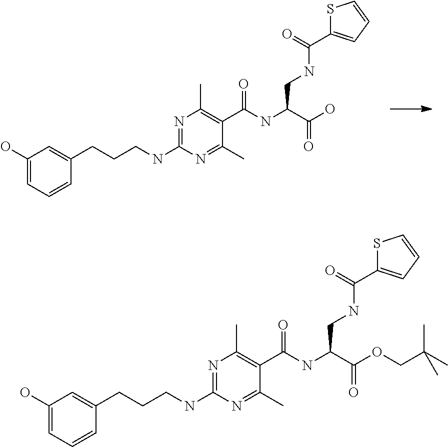

A mixture of (S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid (20 mg, 0.04 mmol), thionyl chloride (150 μl), and neopentyl alcohol (35 mg, 0.4 mmol) in dioxane (0.5 ml) was heated to 100° C. in a sealed tube overnight. The mixture was diluted with EtOAc, washed with water and brine, then dried over Na₂SO₄, filtered, concentrated in the presence of silica gel and chromatographed (30-100% EtOAc in hexane) to give the desired product (14.9 mg, 66% yield). MS m/e 568.1 (M+H⁺).

### Example 119

### (S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid butyl ester

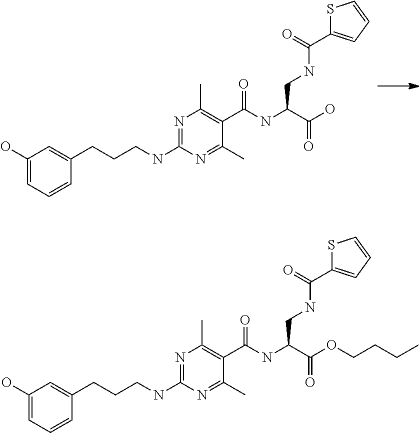

A mixture of (S)-2-({2-[3-(3-hydroxy-phenyl)-propylamino]-4,6-dimethyl-pyrimidine-5-carbonyl}-amino)-3-[(thiophene-2-carbonyl)-amino]-propionic acid (50 mg, 0.1 mmol) and thionyl chloride (22 μL) in dioxane (1 ml) was stirred at room temperature 2 h. 1-butanol (23 mg, 0.31 mmol) was added. After 1 h, the mixture was diluted with EtOAc, washed with brine, then dried over Na₂SO₄, filtered, concentrated in the presence of silica gel and chromatographed (20-100% EtOAc in hexane) to give the title compound (31.3 mg, 56% yield). MS m/e 554.3 (M+H⁺).

### Example 120

### (S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid 3-morpholin-4-yl-propyl ester

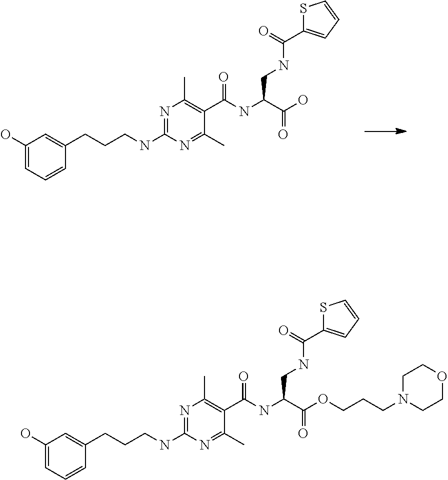

A mixture of (S)-2-({2-[3-(3-hydroxy-phenyl)-propylamino]-4,6-dimethyl-pyrimidine-5-carbonyl}-amino)-3-[(thiophene-2-carbonyl)-amino]-propionic acid (150 mg, 0.3 mmol), chloropropylmorpholine (0.15 g, 0.90 mmol), sodium iodide (0.13 g, 0.90 mmol) and triethylamine (0.091 g, 0.90 mmol) in DMF (2 ml) was microwaved at 120° C. for 20 minutes. The mixture was diluted with EtOAc, washed with brine, dried over Na₂SO₄, filtered, concentrated in the presence of silica gel and chromatographed (0-20% MeOH in EtOAc) to give the title compound 141.8 mg. MS m/e 625.2 (M+H⁺).

### Example 121

### (S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid 2-morpholin-4-yl-ethyl ester

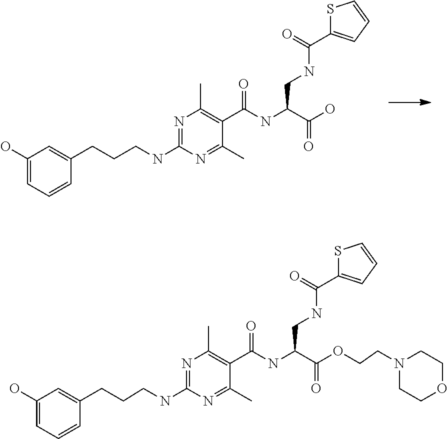

A mixture of (S)-2-({2-[3-(3-hydroxy-phenyl)-propylamino]-4,6-dimethyl-pyrimidine-5-carbonyl}-amino)-3-[(thiophene-2-carbonyl)-amino]-propionic acid (50 mg, 0.1 mmol) and thionyl chloride (241) in dioxane (1 ml) was stirred at room temperature 2 h. 4-(2-hydroxyethyl)morpholine (41 mg, 0.31 mmol) and triethylamine (31 mg, 0.31 mmol) was added. After 4 h, the mixture was diluted with pH 9 Buffer and extracted with EtOAc. The combined organic layers were washed with brine, dried over Na₂SO₄, filtered, concentrated in the presence of silica gel and chromatographed (0-20% MeOH in EtOAc) to give the desired product (11.1 mg, 18% yield). MS m/e 611.1 (M+H⁺).

### Example 122

### (S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid propyl ester

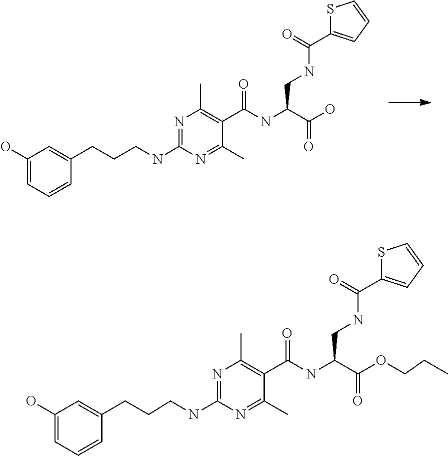

A mixture of (S)-2-({2-[3-(3-hydroxy-phenyl)-propylamino]-4,6-dimethyl-pyrimidine-5-carbonyl}-amino)-3-[(thiophene-2-carbonyl)-amino]-propionic acid (50 mg, 0.1 mmol) and thionyl chloride (241) in dioxane (1 ml) was stirred at room temperature for 90 minutes. 1-propanol (19 mg, 0.31 mmol) was added. After 1.5 h, the mixture was diluted with EtOAc, washed with brine, dried over Na₂SO₄, filtered, concentrated in the presence of silica gel and chromatographed (30-100% EtOAc in hexane) to give the title compound (27.6 mg, 52% yield). MS m/e 540.0 (M+H⁺).

### Example 123

### (S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid isobutyl ester

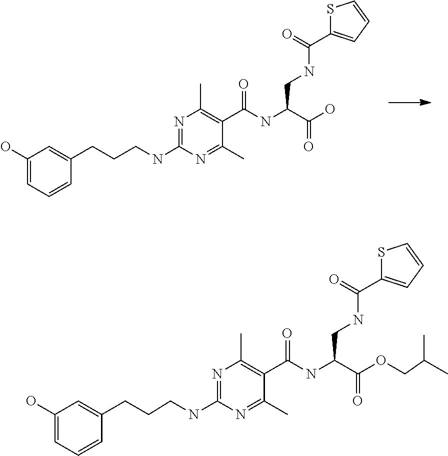

A mixture of (S)-2-({2-[3-(3-hydroxy-phenyl)-propylamino]-4,6-dimethyl-pyrimidine-5-carbonyl}-amino)-3-[(thiophene-2-carbonyl)-amino]-propionic acid (50 mg, 0.1 mmol) and thionyl chloride (241) in dioxane (1 ml) was stirred at room temperature for 2 h. 2-methyl-1-propanol (23 mg, 0.31 mmol) was added. After 4 h, the mixture was diluted with EtOAc, washed with brine, dried over Na₂SO₄, filtered, concentrated in the presence of silica gel and chromatographed (30-100% EtOAc in hexane) to give the desired product (33.4 mg, 61% yield). MS m/e 553.9 (M+H⁺).

### Example 124

### (S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid isopropyl ester

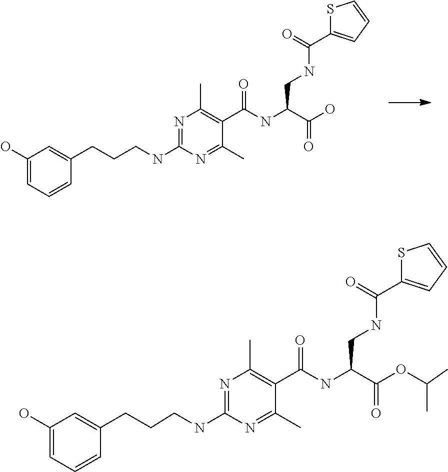

A mixture of (S)-2-({2-[3-(3-hydroxy-phenyl)-propylamino]-4,6-dimethyl-pyrimidine-5-carbonyl}-amino)-3-[(thiophene-2-carbonyl)-amino]-propionic acid (50 mg, 0.1 mmol) and thionyl chloride (22 μl) in dioxane (1 ml) was stirred at room temperature for 2 h. 2-propanol (19 mg, 0.31 mmol) was added. After 18 h, the mixture was diluted with EtOAc, washed with brine, dried over Na₂SO₄, filtered, concentrated in the presence of silica gel and chromatographed (30-100% EtOAc in hexane) to give the title compound (26.7 mg, 49% yield). MS m/e 540.2 (M+H⁺).

### Example 125

### (S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid 1-ethyl-propyl ester

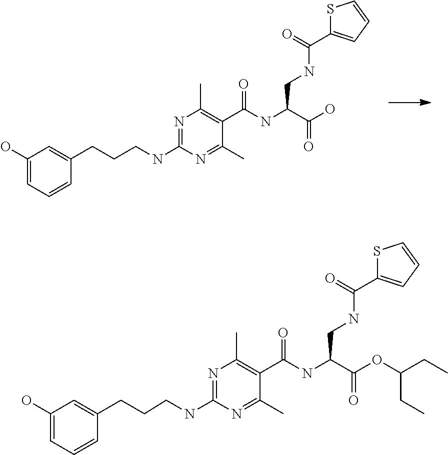

A mixture of (S)-2-({2-[3-(3-hydroxy-phenyl)-propylamino]-4,6-dimethyl-pyrimidine-5-carbonyl}-amino)-3-[(thiophene-2-carbonyl)-amino]-propionic acid (0.70 g, 1.4 mmol) and thionyl chloride (0.40 g) in dioxane (14 ml) was stirred at room temperature for 2 h. 3-pentanol (110 mg, 0.31 mmol) was added. After 18 h, the mixture was diluted with EtOAc, washed with brine, dried over Na₂SO₄, filtered, concentrated in the presence of silica gel and chromatographed (30-100% EtOAc in hexane) to give the desired product (302.1 mg, 38% yield). MS m/e 568.1 (M+H⁺).

### Example 126

### (S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid cyclopentyl ester

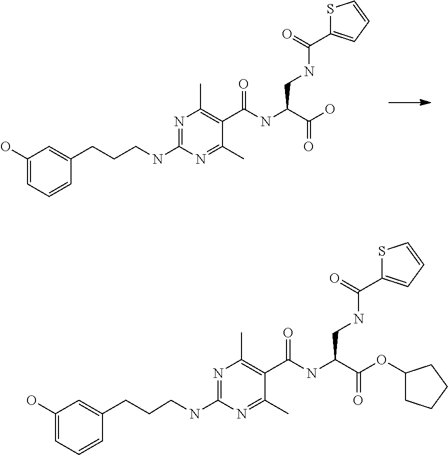

A mixture of (S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid (0.75 g, 1.51 mmol) and thionyl chloride (0.54 g, 4.52 mmol) in dioxane (15 ml) was stirred at room temperature 2 h. Cyclopentanol (0.39 g, 4.52 mmol) was added. After 1.5 h, the mixture was diluted with EtOAc, washed with brine, dried over Na₂SO₄, filtered, concentrated in the presence of silica gel and chromatographed (30-100% EtOAc in hexane) to give the desired product (250 mg, 29% yield). MS m/e 566.1 (M+H⁺).

### Example 127

### (S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid dimethylcarbamoylmethyl ester

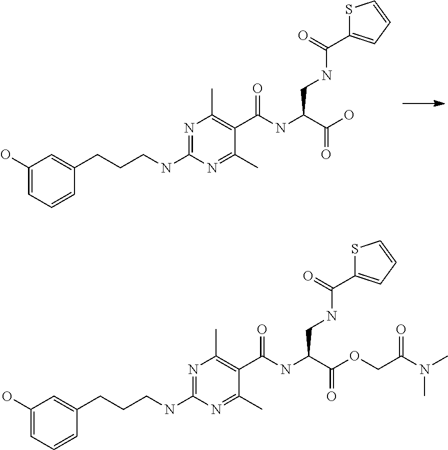

A mixture of (S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid (200 mg, 0.40 mmol), 2-chlorodimethylacetamide (0.15 g, 1.2 mmol), sodium iodide (0.18 g, 1.2 mmol) and triethylamine (0.12 g, 1.2 mmol) in DMF (2 ml) was microwaved at 120° C. for 20 minutes. The mixture was diluted with EtOAc, washed with brine, dried over Na₂SO₄, filtered, concentrated in the presence of silica gel and chromatographed (100% EtOAc) to give the title compound (172.2 mg, 74% yield). MS m/e 583.2 (M+H⁺).

### Example 128

### (S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid 3-ethoxy-propyl ester

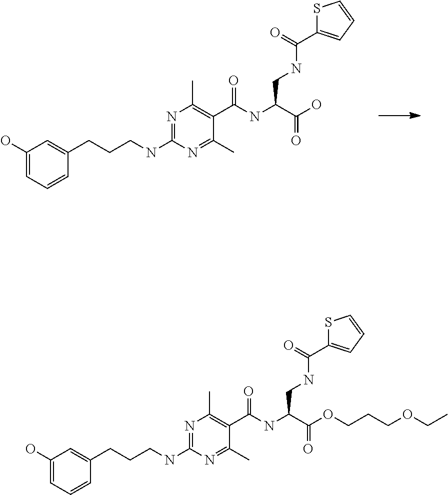

A mixture of (S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid (0.63 g, 1.26 mmol), 1-bromo-3-ethoxy-propane (0.84 g, 5.03 mmol), sodium iodide (0.75 g, 5.03 mmol) and triethylamine (0.51 g, 5.03 mmol) in DMF (4 ml) was microwaved at 120° C. for 20 minutes. The mixture was diluted with EtOAc, washed with brine, dried over Na₂SO₄, filtered, concentrated in the presence of silica gel and chromatographed (30-100% EtOAc in hexane) to give the desired product (280.4 mg, 38% yield). MS m/e 584.0 (M+H⁺).

### Example 129

### (S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid tetrahydro-pyran-4-yl ester

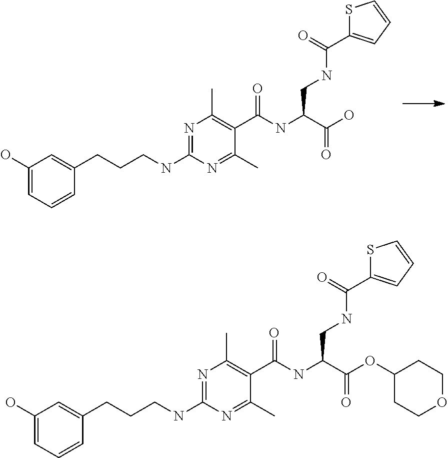

A mixture of (S)-2-({2-[3-(3-hydroxy-phenyl)-propylamino]-4,6-dimethyl-pyrimidine-5-carbonyl}-amino)-3-[(thiophene-2-carbonyl)-amino]-propionic acid (50 mg, 0.1 mmol) and thionyl chloride (46 μl) in dioxane (1 ml) was stirred at room temperature for 2 h. Tetrahydro-4-pyranol (0.20 g, 2.0 mmol) was added and the mixture was microwaved at 90° C. for 20 minutes. The mixture was diluted with EtOAc, washed with brine, dried over Na₂SO₄, filtered, concentrated in the presence of silica gel and chromatographed (30-100% EtOAc in hexane) to give the desired product (26.0 mg, 45% yield). MS m/e 582.2 (M+H⁺).

### Example 130

### (S)-2-({2-[3-(3-Hydroxy-phenyl)-propylamino]-4,6-dimethyl-pyrimidine-5-carbonyl}-amino)-3-[(thiophen-2-carbonyl)-amino]-propionic acid 2,2-dimethyl-propionyloxymethyl ester

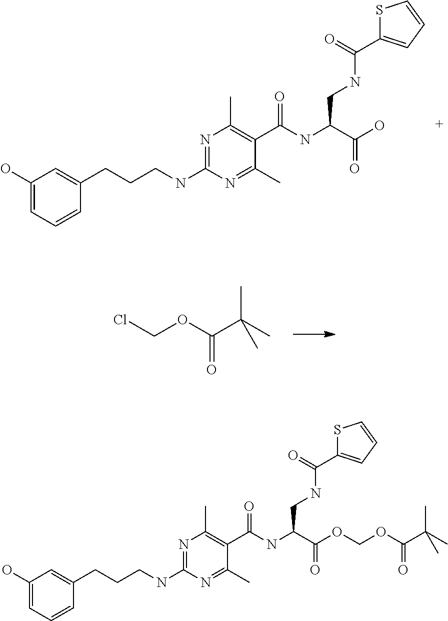

A solution of (S)-2-({2-[3-(hydroxyl-phenyl)-propylamino]-4,6-dimethyl-pyrimidine-5-carbonyl}-amino)-3-[(thiophene-2-carbonyl)-amino]-propionic acid (50 mg, 0.1 mmol), 2,2-dimethyl-propionic acid chloromethyl ester (15.1 mg, 0.1 mmol), and triethylamine (10 mg, 0.1 mmol) in anhydrous DMF (3 ml) was stirred at 80° C. overnight. The reaction mixture was cooled to room temperature, quenched with water and extracted with EtOAc. The organic layer was washed with water and brine, dried over sodium sulfate, filtered and concentrated. The residue was purified by flash chromatography with 10% MeOH in DCM to give the desired product (31 mg, 51% yield) pure product. MS m/e 612.1 (M+H⁺).

### Example 131

### (S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid 1-(2,2-dimethyl-propionyloxy)-ethyl ester

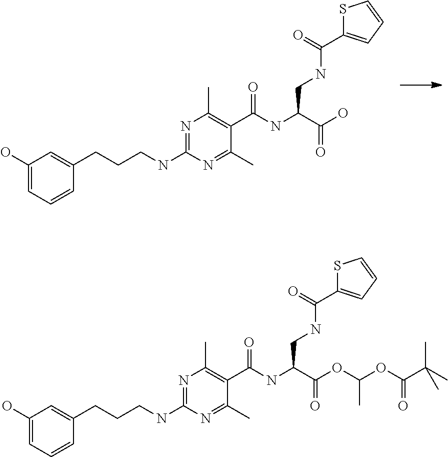

A mixture of (S)-2-({2-[3-(3-hydroxy-phenyl)-propylamino]-4,6-dimethyl-pyrimidine-5-carbonyl}-amino)-3-[(thiophene-2-carbonyl)-amino]-propionic acid (0.20 g, 0.40 mmol), 2,2-dimethyl-propionic acid 1-chloro-ethyl ester (0.33 g, 2.0 mmol), sodium iodide (0.30 g, 2.0 mmol) and triethylamine (0.20 g, 2.0 mmol) in DMF (2.5 ml) was microwaved at 150° C. for 30 minutes. The mixture was diluted with EtOAc, washed with brine, dried over Na₂SO₄, filtered, concentrated in the presence of silica gel and chromatographed (0-70% EtOAc in hexane) to give the desired product (143.3 mg, 57% yield). MS m/e 626.2 (M+H⁺).

### Example 132

### (S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid 1-isobutyryloxy-ethyl ester

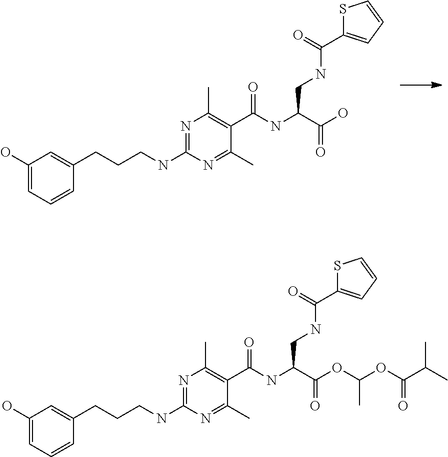

A mixture of (S)-2-({2-[3-(3-hydroxy-phenyl)-propylamino]-4,6-dimethyl-pyrimidine-5-carbonyl}-amino)-3-[(thiophene-2-carbonyl)-amino]-propionic acid (0.20 g, 0.40 mmol), Isobutyric acid 1-chloro-ethyl ester (0.18 g, 1.2 mmol), sodium iodide (0.18 g, 1.2 mmol) and triethylamine (0.12 g, 1.2 mmol) in DMF (2.5 ml) was microwaved at 120° C. for 20 minutes. The mixture was diluted with EtOAc, washed with brine, dried over Na₂SO₄, filtered, concentrated in the presence of silica gel and chromatographed (20-100% EtOAc in hexane) to give the desired compound (103.3 mg, 42% yield). MS m/e 612.2 (M+H⁺).

### Example 133

### (S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid 2-(2-ethoxy-ethoxy)-ethyl ester

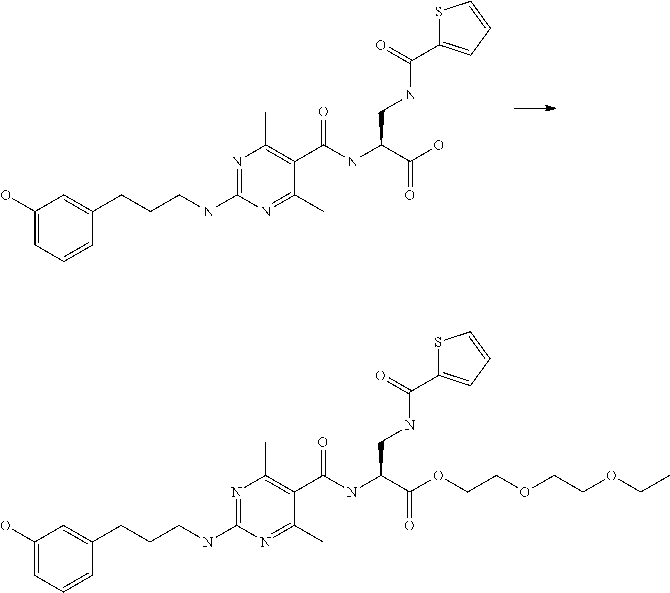

A mixture of (S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid (0.20 g, 0.40 mmol), 1-(2-bromoethoxy)-2-ethoxyethane (0.32 g, 1.6 mmol), sodium iodide (0.24 g, 1.6 mmol) and triethylamine (0.16 g, 1.6 mmol) in DMF (3 ml) was microwaved at 120° C. for 20 minutes. The mixture was diluted with EtOAc (50 ml), washed with brine, dried over Na₂SO₄, filtered, concentrated in the presence of silica gel and chromatographed (50-100% EtOAc in hexane) to give the title compound (135.4 mg, 55% yield). MS m/e 614.2 (M+H⁺).

### Example 134

### (S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid 2-ethoxy-ethyl ester

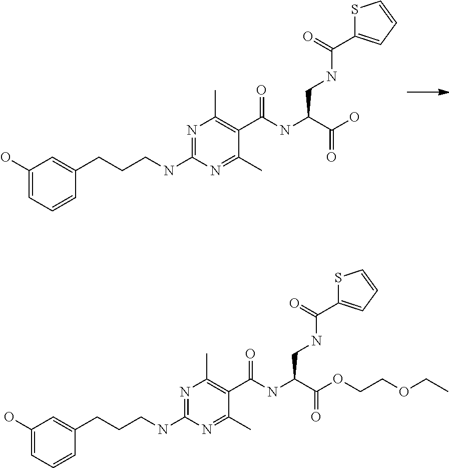

A mixture of (S)-2-({2-[3-(3-hydroxy-phenyl)-propylamino]-4,6-dimethyl-pyrimidine-5-carbonyl}-amino)-3-[(thiophene-2-carbonyl)-amino]-propionic acid (0.20 g, 0.40 mmol), 2-bromoethyl ethyl ether (0.31 g, 2.0 mmol), sodium iodide (0.30 g, 2.0 mmol) and triethylamine (0.20 g, 2.0 mmol) in DMF (2.5 ml) was microwaved at 120° C. for 20 minutes. The mixture was diluted with EtOAc, washed with brine, dried over Na₂SO₄, filtered, concentrated in the presence of silica gel and chromatographed (30-100% EtOAc in hexane) to give the title compound (124.1 mg, 54% yield). MS m/e 570.2 (M+H⁺).

### Example 135

### (S)-2-({2-[3-(3-Acetoxy-phenyl)-propylamino]-4,6-dimethyl-pyrimidine-5-carbonyl}-amino)-3-[(thiophene-2-carbonyl)-amino]-propionic acid 3-ethoxy-propyl ester

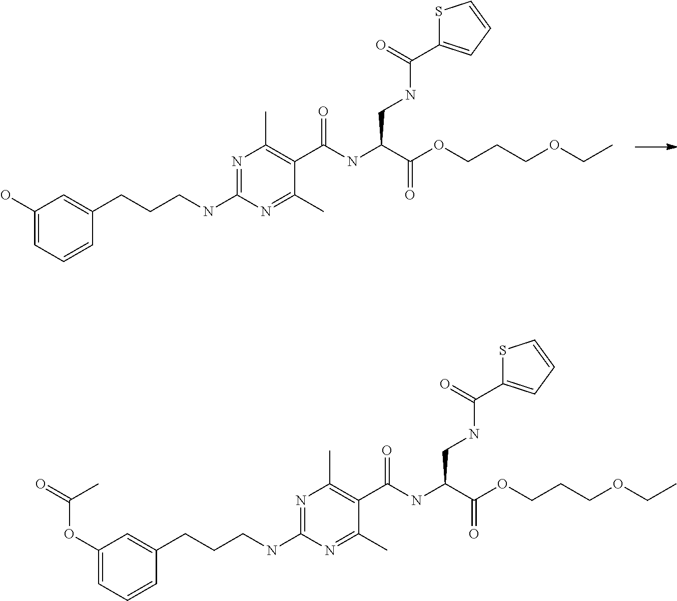

A mixture of (S)-2-({2-[3-(3-hydroxy-phenyl)-propylamino]-4,6-dimethyl-pyrimidine-5-carbonyl}-amino)-3-[(thiophene-2-carbonyl)-amino]-propionic acid 3-ethoxy-propyl ester (0.05 g, 0.086 mmol), acetic anhydride (0.25 ml) and pyridine (0.25 ml) was stirred at room temperature for 3 h, diluted with water, and extracted with EtOAc. The organic layer was washed with water, dried over Na₂SO₄, filtered, concentrated in the presence of silica gel and chromatographed (20-100% EtOAc in hexane) to give the desired product (41.1 mg, 78% yield). MS m/e 626.0 (M+H⁺).

### Example 136

### (S)-2-({2-[3-(3-Isobutyryloxy-phenyl)-propylamino]-4,6-dimethyl-pyrimidine-5-carbonyl}-amino)-3-[(thiophene-2-carbonyl)-amino]-propionic acid 3-ethoxy-propyl ester

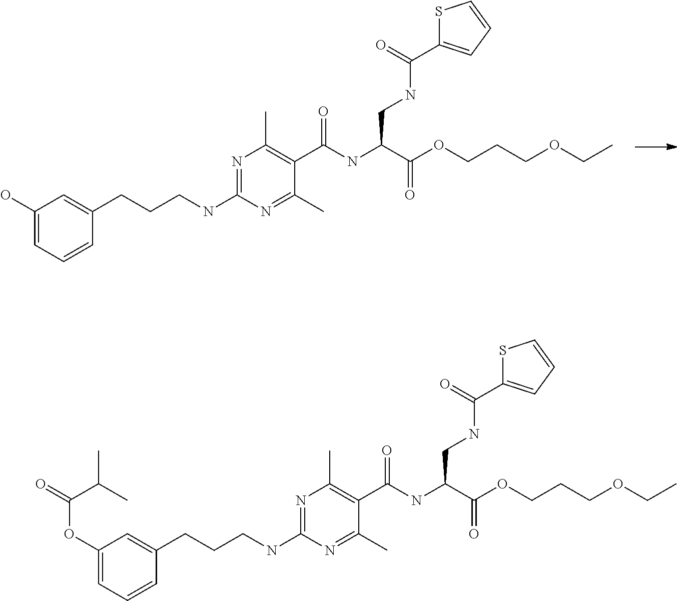

A mixture of (S)-2-({2-[3-(3-hydroxy-phenyl)-propylamino]-4,6-dimethyl-pyrimidine-5-carbonyl}-amino)-3-[(thiophene-2-carbonyl)-amino]-propionic acid 3-ethoxy-propyl ester (0.154 g, 0.26 mmol), isobutyric anhydride (0.6 ml) and pyridine (0.6 ml) was stirred at room temperature for 1 h, diluted with EtOAc, washed with water, dried over Na₂SO₄, filtered, concentrated in the presence of silica gel and chromatographed (20-100% EtOAc in hexane) to give the desired product (141.3 mg, 83% yield). MS m/e 654.1 (M+H⁺).

### Example 137

### (S)-2-({2-[3-(1H-Indazol-4-yl)-propylamino]-4,6-dimethyl-pyrimidine-5-carbonyl}-amino)-3-[(thiophene-2-carbonyl)-amino]-propionic acid butyl ester

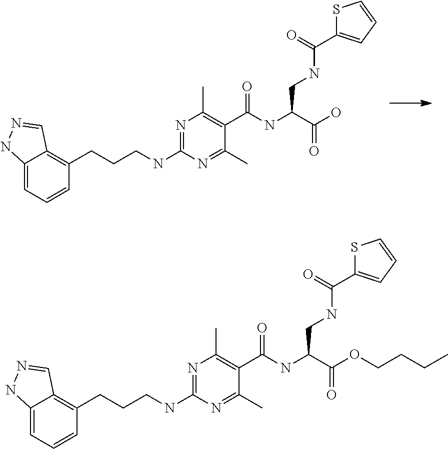

A mixture of (S)-2-({2-[3-(1H-indazol-4-yl)-propylamino]-4,6-dimethyl-pyrimidine-5-carbonyl}-amino)-3-[(thiophene-2-carbonyl)-amino]-propionic acid (0.05 g, 0.095 mmol), iodobutane (0.021 g, 0.114 mmol), and potassium carbonate (0.016 g, 0.114 mmol) in DMF (1 ml) was stirred at room temperature overnight. The mixture was diluted with EtOAc, washed with brine, dried over Na₂SO₄, filtered, concentrated in the presence of silica gel and chromatographed (50-100% EtOAc in hexane) to give the desired product (38.2 mg, 58% yield). MS m/e 578.2 (M+H⁺).

### Example 138

### (S)-2-({2-[3-(1H-Indazol-4-yl)-propylamino]-4,6-dimethyl-pyrimidine-5-carbonyl}-amino)-3-[(thiophene-2-carbonyl)-amino]-propionic acid ethyl ester

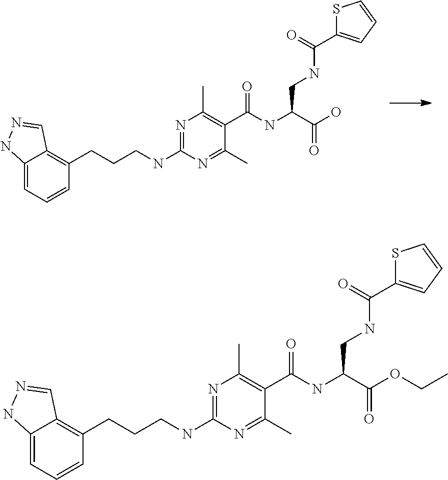

A mixture of (S)-2-({2-[3-(1H-Indazol-4-yl)-propylamino]-4,6-dimethyl-pyrimidine-5-carbonyl}-amino)-3-[(thiophene-2-carbonyl)-amino]-propionic acid (0.075 g, 0.143 mmol), ethyl iodide (0.027 g, 0.173 mmol), and potassium carbonate (0.024 g, 0.173 mmol) in DMF (1.5 ml) was stirred at room temperature overnight. The mixture was diluted with EtOAc, washed with brine, dried over Na₂SO₄, filtered, concentrated in the presence of silica gel and chromatographed (50-100% EtOAc in hexane) to give the title compound (62.9 mg, 82% yield). MS m/e 550.2 (M+H⁺).

### Example 139

### (S)-2-({2-[3-(1H-Indazol-4-yl)-propylamino]-4,6-dimethyl-pyrimidine-5-carbonyl}-amino)-3-[(thiophene-2-carbonyl)-amino]-propionic acid cyclopentyl ester

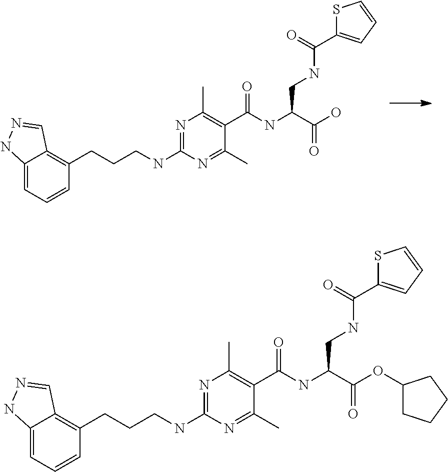

A mixture of (S)-2-({2-[3-(1H-Indazol-4-yl)-propylamino]-4,6-dimethyl-pyrimidine-5-carbonyl}-amino)-3-[(thiophene-2-carbonyl)-amino]-propionic acid (0.05 g, 0.095 mmol), cyclopentyl iodide (0.022 g, 0.114 mmol), and potassium carbonate (0.016 g, 0.114 mmol) in DMF (1 ml) was stirred at room temperature overnight. The mixture was diluted with EtOAc, washed with brine, dried over Na₂SO₄, filtered, concentrated in the presence of silica gel and chromatographed (50-100% EtOAc in hexane) to give the desired product (33.6 mg, 60% yield). MS m/e 590.1 (M+H⁺).

### Example 140

### (S)-2-({2-[3-(1H-Indazol-4-yl)-propylamino]-4,6-dimethyl-pyrimidine-5-carbonyl}-amino)-3-[(thiophene-2-carbonyl)-amino]-propionic acid isobutyl ester

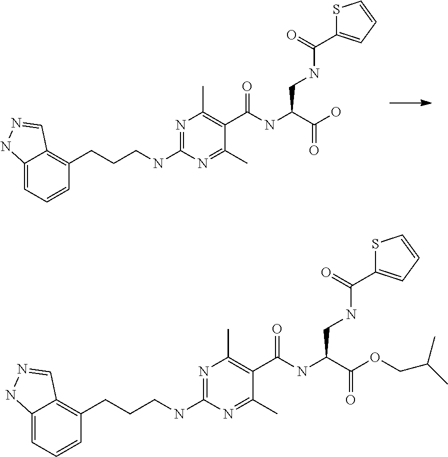

A mixture of (S)-2-({2-[3-(1H-indazol-4-yl)-propylamino]-4,6-dimethyl-pyrimidine-5-carbonyl}-amino)-3-[(thiophene-2-carbonyl)-amino]-propionic acid (0.10 g, 0.19 mmol), 1-iodo-2-methylpropane (0.106 g, 0.58 mmol), and triethylamine (0.059 g, 0.58 mmol) in DMF (2 ml) was microwaved at 150° C. for 20 minutes. The mixture was diluted with EtOAc, washed with brine, dried over Na₂SO₄, filtered, concentrated in the presence of silica gel and chromatographed (30-100% EtOAc in hexane) to give the title compound (82.7 mg, 75% yield). MS m/e 578.1 (M+H⁺).

### Example 141

### (S)-2-({2-[3-(1H-Indazol-4-yl)-propylamino]-4,6-dimethyl-pyrimidine-5-carbonyl}-amino)-3-[(thiophene-2-carbonyl)-amino]-propionic acid 1-ethyl-propyl ester

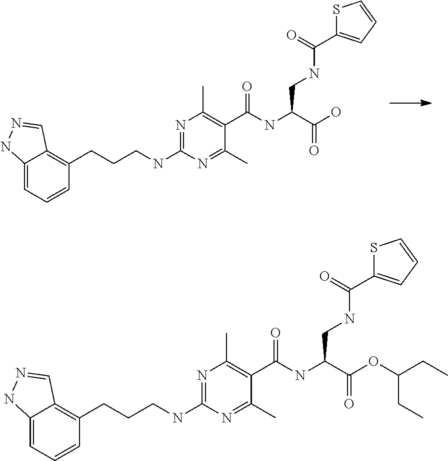

A mixture of (S)-2-({2-[3-(1H-indazol-4-yl)-propylamino]-4,6-dimethyl-pyrimidine-5-carbonyl}-amino)-3-[(thiophene-2-carbonyl)-amino]-propionic acid (0.05 g, 0.095 mmol), 3-bromopentane (0.022 g, 0.143 mmol), and potassium carbonate (0.020 g, 0.143 mmol) in DMF (1 ml) was stirred at room temperature overnight. 3-bromopentane (0.022 g, 0.143 mmol), and potassium carbonate (0.020 g, 0.143 mmol) were added and the reaction mixture was stirred at 50° C. for 5 h, then cooled to room temperature and stirred for 2 days. The mixture was diluted with EtOAc, washed with brine, dried over Na₂SO₄, filtered, concentrated in the presence of silica gel and chromatographed (50-100% EtOAc in hexane) to give the title compound (28.3 mg, 50% yield). MS m/e 592.2 (M+H⁺).

### Example 142

### (S)-2-({2-[3-(1H-Indazol-4-yl)-propylamino]-4,6-dimethyl-pyrimidine-5-carbonyl}-amino)-3-[(thiophene-2-carbonyl)-amino]-propionic acid dimethylcarbamoylmethyl ester

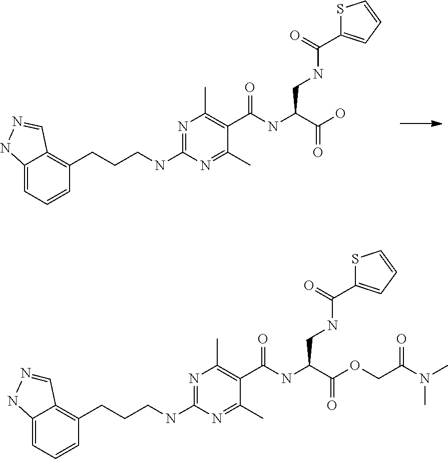

A mixture of (S)-2-({2-[3-(1H-Indazol-4-yl)-propylamino]-4,6-dimethyl-pyrimidine-5-carbonyl}-amino)-3-[(thiophene-2-carbonyl)-amino]-propionic acid (0.05 g, 0.095 mmol), 2-chlorodimethyl acetamide (0.017 g, 0.143 mmol), sodium iodide (0.021 g, 0.143 mmol), and potassium carbonate (0.020 g, 0.143 mmol) in DMF (1 ml) was stirred at room temperature overnight. The mixture was diluted with EtOAc, washed with brine, dried over Na₂SO₄, filtered, concentrated in the presence of silica gel and chromatographed (100% EtOAc) to give the desired product (38.8 mg, 67% yield). MS m/e 607.1 (M+H⁺).

### Example 143

### (S)-2-({2-[3-(1H-Indazol-4-yl)-propylamino]-4,6-dimethyl-pyrimidine-5-carbonyl}-amino)-3-[(thiophene-2-carbonyl)-amino]-propionic acid isopropyl ester

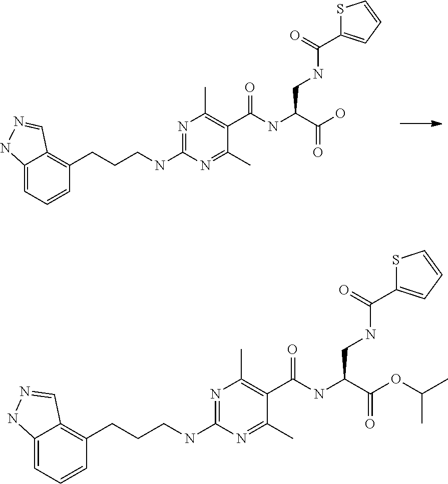

A mixture of (S)-2-({2-[3-(1H-indazol-4-yl)-propylamino]-4,6-dimethyl-pyrimidine-5-carbonyl}-amino)-3-[(thiophene-2-carbonyl)-amino]-propionic acid (0.05 g, 0.095 mmol), 2-iodopropane (0.019 g, 0.114 mmol), and potassium carbonate (0.016 g, 0.114 mmol) in DMF (1 ml) was stirred at room temperature overnight. The mixture was diluted with EtOAc, washed with brine, dried over Na₂SO₄, filtered, concentrated in the presence of silica gel and chromatographed (50-100% EtOAc in hexane) to give the desired product (32.8 mg, 62% yield). MS m/e 564.2 (M+H⁺).

### Example 144

### (S)-2-({2-[3-(1H-Indazol-4-yl)-propylamino]-4,6-dimethyl-pyrimidine-5-carbonyl}-amino)-3-[(thiophene-2-carbonyl)-amino]-propionic acid propyl ester

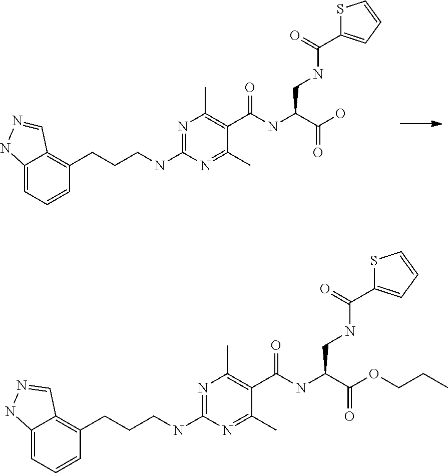

A mixture of (S)-2-({2-[3-(1H-indazol-4-yl)-propylamino]-4,6-dimethyl-pyrimidine-5-carbonyl}-amino)-3-[(thiophene-2-carbonyl)-amino]-propionic acid (0.05 g, 0.095 mmol), 1-iodopropane (0.019 g, 0.114 mmol), and potassium carbonate (0.016 g, 0.114 mmol) in DMF (1 ml) was stirred at room temperature overnight. The mixture was diluted with EtOAc, washed with brine, dried over Na₂SO₄, filtered, concentrated in the presence of silica gel and chromatographed (40-100% EtOAc in hexane) to give the desired product (58.1 mg, 100% yield). MS m/e 564.1 (M+H⁺).

### Example 145

### (S)-2-({2-[3-(1H-Indazol-4-yl)-propylamino]-4,6-dimethyl-pyrimidine-5-carbonyl}-amino)-3-[(thiophene-2-carbonyl)-amino]-propionic acid pentyl ester

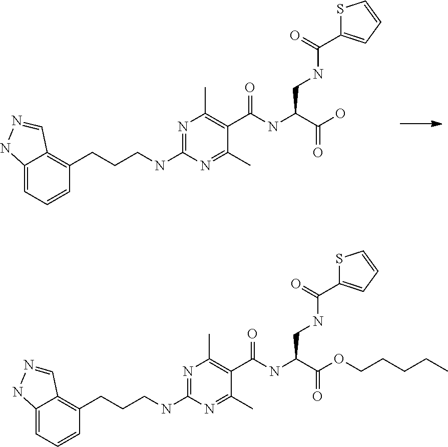

A mixture of (S)-2-({2-[3-(1H-Indazol-4-yl)-propylamino]-4,6-dimethyl-pyrimidine-5-carbonyl}-amino)-3-[(thiophene-2-carbonyl)-amino]-propionic acid (0.05 g, 0.095 mmol), 1-iodopentane (0.023 g, 0.114 mmol), and potassium carbonate (0.016 g, 0.114 mmol) in DMF (1 ml) was stirred at room temperature overnight. The mixture was diluted with EtOAc, washed with brine, dried over Na₂SO₄, filtered, concentrated in the presence of silica gel and chromatographed (40-100% EtOAc in hexane) to give the title compound (38.3 mg, 68% yield). MS m/e 592.2 (M+H⁺).

### Example 146

### (S)-2-({2-[3-(1H-Indazol-4-yl)-propylamino]-4,6-dimethyl-pyrimidine-5-carbonyl}-amino)-3-[(thiophene-2-carbonyl)-amino]-propionic acid 3-morpholin-4-yl-propyl ester

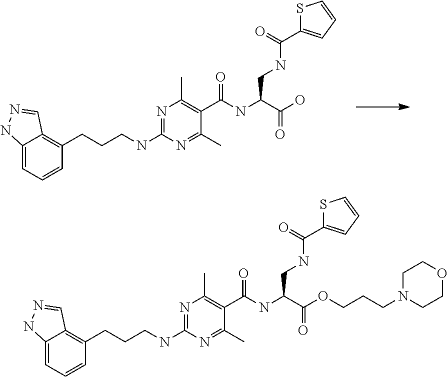

A mixture of (S)-2-({2-[3-(1H-indazol-4-yl)-propylamino]-4,6-dimethyl-pyrimidine-5-carbonyl}-amino)-3-[(thiophene-2-carbonyl)-amino]-propionic acid (0.10 g, 0.19 mmol), chloropropylmorpholine (0.094 g, 0.58 mmol), sodium iodide (0.087 g, 0.58 mmol), and triethylamine (0.059 g, 0.58 mmol) in DMF (2 ml) was microwaved at 120° C. for 20 minutes. The mixture was diluted with EtOAc, washed with brine, dried over Na₂SO₄, filtered, concentrated in the presence of silica gel and chromatographed (0-20% MeOH in EtOAc) to give the title compound (102.3 mg, 83% yield). MS m/e 649.3 (M+H⁺).

### Example 147

### (S)-2-({2-[3-(1H-Indazol-4-yl-propylamino]-4,6-dimethyl-pyrimidine-5-carbonyl}-amino)-3-[(thiophene-2-carbonyl)-amino]-propionic acid 3-ethoxy-propyl ester

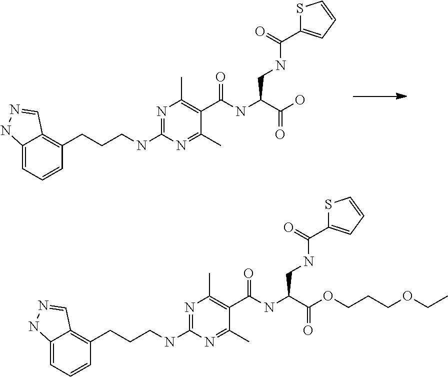

A mixture of (S)-2-({2-[3-(1H-indazol-4-yl)-propylamino]-4,6-dimethyl-pyrimidine-5-carbonyl}-amino)-3-[(thiophene-2-carbonyl)-amino]-propionic acid (0.10 g, 0.19 mmol), 1-bromo-3-ethoxy-propane (0.16 g, 0.96 mmol), sodium iodide (0.14 g, 0.96 mmol), and triethylamine (0.097 g, 0.96 mmol) in DMF (2 ml) was microwaved at 120° C. for 20 minutes. The mixture was diluted with brine and extracted with EtOAc. The combined organic layers were washed with brine, dried over Na₂SO₄, filtered, concentrated in the presence of silica gel and chromatographed (50-100% EtOAc in hexane) to give the desired product (84.6 mg, 73% yield). MS m/e 608.2 (M+H⁺).

### Example 148

### Activity Assays

sLFA-1/ICAM-1 ELISA and Mac-1/ICAM-1 ELISA

Plates were coated with either 50 μl/well of 2.0 ug/ml solution of sLFA-1 or Mac-1 receptor in divalent cation buffer (1 mM MnCl₂, 0.14M NaCl, 20 mM HEPES pH 7.2) at 4° C. overnight. Two hundred fifty μl of blocking buffer (1% BSA in divalent cation buffer) was added to each well 1 hour at 37° C. Plates were washed 3 times with wash buffer (TBS/0.05% Tween-20/1 mM MnCl₂). The compound to be tested was solubilized in DMSO. A series of 1:3 dilutions were performed to achieve a concentration range of 0.45 nM-3 uM. Fifty μl of binding buffer (0.5% BSA in divalent cation buffer)/1% DMSO and 50 μl of the solutions to be tested were added to the appropriate wells and incubated for 1 hour. Fifty μl of 5dICAM-Fc (27 ng/ml) was added to the appropriate wells and 50 μl binding buffer was added to non-specific binding wells and incubated for 2 hours and washed. One hundred μl of 1:4000 HRP-goat anti-huIgG was added to each well and incubated for 1 hour and washed. One hundred μl of 1:1 TMB solution was added to each well and developed for 20 min at room temperature. Color development was stopped by adding 100 μl H₃PO₄ to each well. Absorbance was measured at 450 nm.

Human Mixed Lymphocyte Reaction (hMLR)

PBMC's were isolated from two healthy donors by Ficoll gradient. Cells were resuspended with 4 mL assay media and counted with a Coulter Counter. Both cell populations were resuspended to 1×107/mL. Stimulator cells were irradiated for 2.5 min (2000 RAD) in cesium irradiator. 5×10⁵ cells were added to duplicate wells of the plate. Included were quadruplicate wells receiving responder cells alone and stimulator cells alone. The compound to be tested was solubilized in DMSO. A series of 1:3 dilutions were performed to achieve a concentration range of 0.45 nM-3 uM. One hundred μl of the solutions to be tested were added to the cell plate (100 μl of 2% DMSO/media added to control wells). Plates were then incubated for 2.5 days at 37° C. with 5% CO₂. Plates were pulsed with 50 μl/well 3H-thymidine at 0.5 uCi/well then incubated for 6 hours at 37° C. with 5% CO₂. Cells were harvested using Cell Harvester (TomTec) and counted on TopCount (Perkin Elmer).

Mouse Mixed Lymphocyte Reaction (mMLR)

Spleens were removed from C57B1/6 and Balb/c mice and placed in Hanks Balanced Salt Solution (HBSS). Red blood cells were removed with ACK lysing buffer and washed two times in assay media. Cells were resuspended with 4 mL assay media and counted with a Coulter Counter. Both cell populations were resuspended to 1×10⁷/mL. Stimulator cells were irradiated for 2.5 min (2000 RAD) in cesium irradiator. 5×10⁵ cells were added to duplicate wells of the plate. Included were quadruplicate wells receiving responder cells alone and stimulator cells alone. The compound to be tested was solubilized in DMSO. A series of 1:3 dilutions were performed to achieve a concentration range of 0.45 nM-3 uM. One hundred μl of the solutions to be tested were added to the cell plate (100 μl of 2% DMSO/media added to control wells). Plates were then incubated for 2.5 days at 37° C. with 5% CO₂. Plates were pulsed with 50 μl/well ³H-thymidine at 0.5 uCi/well then incubated for 6 hours at 37° C. with 5% CO₂. Cells were harvested using Cell Harvester (TomTec) and counted on TopCount (Perkin Elmer).

Table 1 provides the in vitro activity for representative LFA-1 antagonists and dual LFA-1/Mac-1 antagonists in the Examples:

TABLE 1

| | LFA-1/ICAM | MAC-1/ICAM | human MLR |
|---|---|---|---|
| Example | (IC50, uM) | (IC50, uM) | (IC50, uM) |
| 1 | 0.073 | 0.441 | 4.455 |
| 3 | 0.032 | 0.057 | 0.574 |
| 4 | 0.12 | | 10 |
| 6 | 0.021 | 0.02 | 0.374 |
| 8 | 0.04 | 0.12 | 0.678 |
| 12 | 0.191 | | |
| 13 | 0.222 | | |
| 14 | 0.5 | | |
| 17 | 0.309 | | |
| 18 | 0.048 | | |
| 41 | 0.353 | | |
| 42 | 0.085 | | |
| 43 | 0.128 | | |
| 44 | 0.115 | | |
| 45 | 0.087 | | |
| 46 | 0.026 | | 0.753 |
| 47 | 0.07 | | |
| 52 | 1.979 | | |
| 61 | 0.041 | 0.055 | 1.183 |
| 62 | 0.08 | 0.045 | 3.807 |
| 63 | 0.323 | 0.67 | |
| 64 | 0.035 | 0.023 | 1.353 |
| 65 | 1.296 | | |
| 66 | 0.111 | | |
| 67 | 0.329 | | |
| 68 | 0.291 | | |
| 69 | 0.028 | | |
| 70 | 3 | | |
| 71 | 0.224 | | |
| 72 | 0.159 | | |
| 73 | 0.056 | | 0.809 |
| 74 | 0.152 | | 2.06 |
| 75 | 0.064 | | |
| 76 | 0.009 | | 1.761 |
| 77 | 0.378 | | |
| 78 | 0.02 | | 0.766 |
| 79 | 0.09 | | |
| 81 | 0.012 | 0.039 | 0.073 |
| 83 | 0.028 | 0.025 | 0.138 |
| 85 | 0.012 | | |
| 87 | 0.004 | | 1.429 |
| 89 | 0.078 | | |
| 91 | 0.042 | | |
| 93 | 0.029 | | 2.304 |
| 95 | 0.265 | | |
| 97 | 0.177 | | |
| 99 | 0.287 | | |
| 101 | 0.052 | | 6.343 |
| 103 | 0.066 | | |
| 105 | 0.103 | | |
| 107 | 0.166 | | |
| 109 | 0.198 | | |
| 111 | 0.056 | | 4.879 |
| 113 | 0.074 | | 4.18 |
| 115 | 0.456 | | |
| 117 | 0.175 | | 5.11 |

It is to be understood that the invention is not limited to the particular embodiments of the invention described above, as variations of the particular embodiments may be made and still fall within the scope of the appended claims.

What is claimed is:

1. A compound of formula (I): 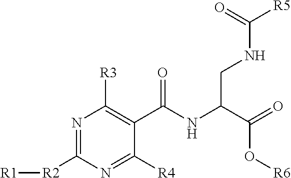
wherein:
R1 is 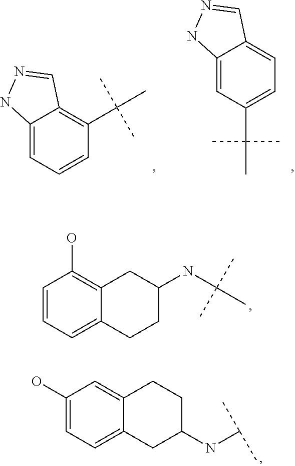
phenyl, mono- or bi-substituted independently with hydroxy, halogen, lower alkyl, alkoxy, —OC(O)CH₃ or —OC(O)CH(CH₃)₂, or
heteroaryl, mono- or bi-substituted with hydroxy;
R2 is: 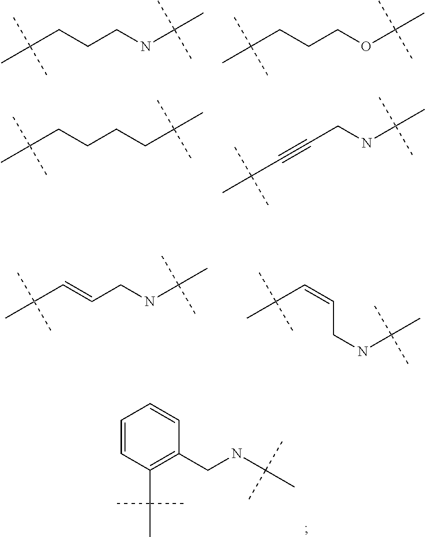
or absent if R1 is 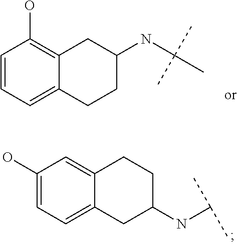
R3 and R4, independently or each other, are H, methyl, trifluoromethyl or ethyl;
R5 is lower alkyl, cycloalkyl, lower alkyl-cycloalkyl, heterocycloalkyl, isoquinoline, quinoline, adamantane, NR7R8, OR9, unsubstituted heteroaryl, heteroaryl substituted with phenyl, unsubstituted phenyl or phenyl substituted with hydroxy or methyl;
R6 is hydrogen, lower alkyl, alkoxy, cycloalkyl, aryl, heteroaryl,
—(CH₂)₂O(CH₂)₂OCH₂CH₃—, 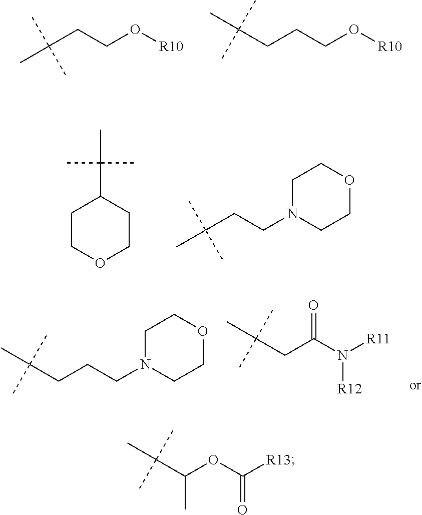
R7 and R8, independently of each other, are hydrogen, lower alkyl, cycloalkyl, aryl or heteroaryl;
R9 is lower alkyl, cycloalkyl, phenyl or heteroaryl; and
R10, R11, R12 and R13, independently of each other, are hydrogen or lower alkyl,
or a pharmaceutically acceptable salt thereof.

2. The compound according to claim 1, wherein R1 is

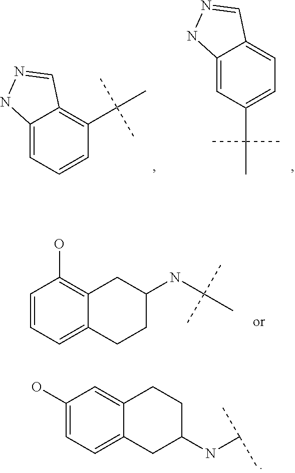

3. The compound according to claim 1, wherein R1 is phenyl, mono- or bi-substituted independently with hydroxy, halogen, lower alkyl, alkoxy, —OC(O)CH₃ or —OC(O)CH(CH₃)₂.

4. The compound according to claim 1, wherein R1 is heteroaryl, mono- or bi-substituted with hydroxy.

5. The compound according to claim 1, wherein R2 is absent.

6. The compound according to claim 1, wherein R3 is methyl.

7. The compound according to claim 1, wherein R4 is methyl.

8. The compound according to claim 1, wherein R5 is lower alkyl, cycloalkyl, lower alkyl-cycloalkyl, heterocycloalkyl, isoquinoline, quinoline, adamantane or NR7R8, OR9.

9. The compound according to claim 1, wherein R5 is unsubstituted heteroaryl, heteroaryl substituted with phenyl, unsubstituted phenyl or phenyl substituted with hydroxy or methyl.

10. The compound according to claim 1, wherein R5 is adamantane, chlorothiophene, pyrazine, thiazole, thiophene, —C(O)CH3, thiazole-phenyl, pyridine, dimethyl-thiazole, isoquinoline, pyridine-thiazole, methyl-thiophene, methyl-pyrazine or ethyl-thiophene.

11. The compound according to claim 1, wherein R6 is hydrogen or lower alkyl.

12. The compound according to claim 1, wherein R6 is methyl.

13. The compound according to claim 1, wherein R6 is:

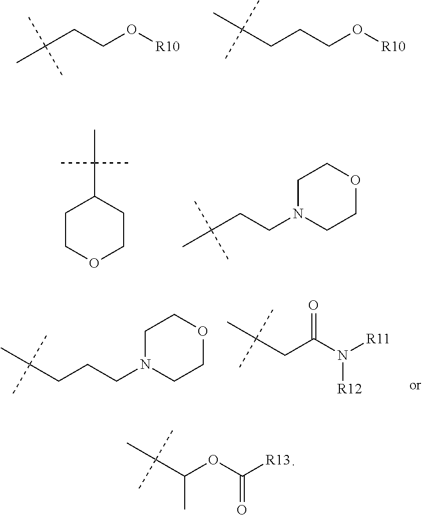

14. The compound according to claim 1, wherein R7 is hydrogen or lower alkyl.

15. The compound according to claim 1, wherein R8 is hydrogen or lower alkyl.

16. The compound according to claim 1, wherein R9 is lower alkyl or phenyl.

17. The compound according to claim 1, wherein R10 is hydrogen or methyl.

18. The compound according to claim 1, wherein R11 is hydrogen or methyl.

19. The compound according to claim 1, wherein R12 is hydrogen or methyl.

20. The compound according to claim 1, wherein R13 is hydrogen or methyl.

21. The compound according to claim 1, wherein said compound is:
(S)-2-({2-[4-(3-hydroxy-phenyl)-butyl]-4,6-dimethyl-pyrimidine-5-carbonyl}-amino)-3-[(thiophene-2-carbonyl)-amino]-propionic acid;
(S)-2-({2-[3-(3-hydroxy-phenyl)-propoxy]-4,6-dimethyl-pyrimidine-5-carbonyl}-amino)-3-[(thiophene-2-carbonyl)-amino]-propionic acid methyl ester;
(S)-2-({2-[3-(3-hydroxy-phenyl)-propoxy]-4,6-dimethyl-pyrimidine-5-carbonyl}-amino)-3-[(thiophene-2-carbonyl)-amino]-propionic acid;
(S)-3-acetylamino-2-({2-[3-(1H-indazol-4-yl)-propylamino]-4,6-dimethyl-pyrimidine-5-carbonyl}-amino)-propionic acid;
(S)-2-({2-[3-(1H-indazol-4-yl)-propylamino]-4,6-dimethyl-pyrimidine-5-carbonyl}-amino)-3-[(thiophene-2-carbonyl)-amino]-propionic acid methyl ester;
(S)-2-({2-[3-(1H-indazol-4-yl)-propylamino]-4,6-dimethyl-pyrimidine-5-carbonyl}-amino)-3-[(thiophene-2-carbonyl)-amino]-propionic acid;
(S)-2-({2-[3-(3-hydroxy-phenyl)-propylamino]-4,6-dimethyl-pyrimidine-5-carbonyl}-amino)-3-[(thiophene-2-carbonyl)-amino]-propionic acid methyl ester;
(S)-2-({2-[3-(3-hydroxy-phenyl)-propylamino]-4,6-dimethyl-pyrimidine-5-carbonyl}-amino)-3-[(thiophene-2-carbonyl)-amino]-propionic acid;
(S)-2-({2-[(3′-hydroxy-biphenyl-2-ylmethyl)-amino]-4,6-dimethyl-pyrimidine-5-carbonyl}-amino)-3-[(thiophene-2-carbonyl)-amino]-propionic acid methyl ester;
(S)-2-({2-[(4′-hydroxy-biphenyl-2-ylmethyl)-amino]-4,6-dimethyl-pyrimidine-5-carbonyl}-amino)-3-[(thiophene-2-carbonyl)-amino]-propionic acid methyl ester;
(S)-2-({2-[(2′-hydroxy-biphenyl-2-ylmethyl)-amino]-4,6-dimethyl-pyrimidine-5-carbonyl}-amino)-3-[(thiophene-2-carbonyl)-amino]-propionic acid methyl ester;
(S)-2-({2-[(3′-hydroxy-biphenyl-2-ylmethyl)-amino]-4,6-dimethyl-pyrimidine-5-carbonyl}-amino)-3-[(thiophene-2-carbonyl)-amino]-propionic acid;
(S)-2-({2-[(3′-hydroxy-biphenyl-2-ylmethyl)-amino]-4,6-dimethyl-pyrimidine-5-carbonyl}-amino)-3-[(thiophene-2-carbonyl)-amino]-propionic acid;
(S)-2-({2-[(2′-hydroxy-biphenyl-2-ylmethyl)-amino]-4,6-dimethyl-pyrimidine-5-carbonyl}-amino)-3-[(thiophene-2-carbonyl)-amino]-propionic acid;
(S) 2-(2-[6-hydroxy-1,2,3,4-tetrahydro-naphthalen-2-ylamino]-4,6-dimethyl-pyrimidine-5-carbonyl-amino)-3-[(thiophene-2-carbonyl)-amino]-propionic acid methyl ester:
(S) 2-(2-[6-hydroxy-1,2,3,4-tetrahydro-naphthalen-2-ylamino]-4,6-dimethyl-pyrimidine-5-carbonyl-amino)-3-[(thiophene-2-carbonyl)-amino]-propionic acid;
(S) 2-(2-[8-hydroxy-1,2,3,4-tetrahydro-naphthalen-2-ylamino]-4,6-dimethyl-pyrimidine-5-carbonyl-amino)-3-[(thiophene-2-carbonyl)-amino]-propionic acid;
(S)-2-({2-[3-(3-Hydroxy-phenyl)-prop-2-ynylamino]-4,6-dimethyl-pyrimidine-5-carbonyl}-amino)-3-[(thiophene-2-carbonyl)-amino]-propionic acid methyl ester;
(S)-2-({2-[(Z)-3-(hydroxy-phenyl)-allylamino]-4,6-dimethyl-pyrimidine-5-carbonyl}-amino)-3-[(thiophene-2-carbonyl)-amino]-propionic acid methyl ester;
(S)-2-({2-[3-(1H-indazol-4-yl)-prop-2-ynylamino]-4,6-dimethyl-pyrimidine-5-carbonyl}-amino)-3-[(thiophene-2-carbonyl)-amino]-propionic acid methyl ester;
(S)-2-({2-[3-(1H-indazol-6-yl)-prop-2-ynylamino]-4,6-dimethyl-pyrimidine-5-carbonyl}-amino)-3-[(thiophene-2-carbonyl)-amino]-propionic acid methyl ester;
(S)-2-({2-[3-(1H-indazol-6-yl)-propylamino]-4,6-dimethyl-pyrimidine-5-carbonyl}-amino)-3-[(thiophene-2-carbonyl)-amino]propionic acid methyl ester;
(S)-2-({2-[3-(3-Fluoro-5-hydroxy-phenyl)-prop-2-ynylamino]-4,6-dimethyl-pyrimidine-5-carbonyl}-amino)-3-[(thiophene-2-carbonyl)-amino]propionic acid methyl ester;
(S)-2-({2-[3-(3-fluoro-5-hydroxy-phenyl)-propylamino]-4,6-dimethyl-pyrimidine-5-carbonyl}-amino)-3-[(thiophene-2-carbonyl)-amino]propionic acid methyl ester;
(S)-2-({2-[3-(4-Fluoro-3-hydroxy-phenyl)-prop-2-ynylamino]-4,6-dimethyl-pyrimidine-5-carbonyl}-amino)-3-[(thiophene-2-carbonyl)-amino]propionic acid methyl ester;
(S)-2-({2-[3-(4-fluoro-3-hydroxy-phenyl)-propylamino]-4,6-dimethyl-pyrimidine-5-carbonyl}-amino)-3-[(thiophene-2-carbonyl)-amino]propionic acid methyl ester;
(S)-2-({2-[3-(3-Hydroxy-2-methyl-phenyl)-prop-2-ynylamino]-4,6-dimethyl-pyrimidine-5-carbonyl}-amino)-3-[(thiophene-2-carbonyl)-amino]propionic acid methyl ester;
(S)-2-({2-[3-(3-Hydroxy-2-methyl-phenyl)-propylamino]-4,6-dimethyl-pyrimidine-5-carbonyl}-amino)-3-[(thiophene-2-carbonyl)-amino]propionic acid methyl ester;
(S)-2-({2-[3-(2-chloro-5-methoxy-phenyl)-prop-2-ynylamino]-4,6-dimethyl-pyrimidine-5-carbonyl}-amino)-3-[(thiophene-2-carbonyl)-amino]propionic acid methyl ester;
(S)-2-({2-[3-(2-chloro-5-methoxy-phenyl)-propylamino]-4,6-dimethyl-pyrimidine-5-carbonyl}-amino)-3-[(thiophene-2-carbonyl)-amino]propionic acid methyl ester;
(S)-2-({2-[3-(4-Hydroxy-phenyl)-prop-2-ynylamino]-4,6-dimethyl-pyrimidine-5-carbonyl}-amino)-3-[(thiophene-2-carbonyl)-amino]propionic acid methyl ester;
(S)-2-({2-[3-(4-hydroxy-phenyl)-propylamino]-4,6-dimethyl-pyrimidine-5-carbonyl}-amino)-3-[(thiophene-2-carbonyl)-amino]propionic acid methyl ester;
(S)-2-({2-[3-(3-hydroxy-phenyl)-propylamino]-4,6-dimethyl-pyrimidine-5-carbonyl}-amino)-3-[(2-phenyl-thiazole-4-carbonyl)-amino]-propionic acid methyl ester;
(S)-2-({2-[3-(3-hydroxy-phenyl)-propylamino]-4,6-dimethyl-pyrimidine-5-carbonyl}-amino)-3-[(2,4-dimethyl-thiazole-5-carbonyl)-amino]-propionic acid methyl Ester;
(S)-2-({2-[3-(3-hydroxy-phenyl)-propylamino]-4,6-dimethyl-pyrimidine-5-carbonyl}-amino)-3-[2-cyclohexyl-acetylamino]-propionic acid methyl Ester;
(S)-2-({2-[3-(3-hydroxy-phenyl)-propylamino]-4,6-dimethyl-pyrimidine-5-carbonyl}-amino)-3-[(isoquinoline-1-carbonyl)-amino]-propionic acid methyl Ester;
(S)-2-({2-[3-(3-hydroxy-phenyl)-propylamino]-4,6-dimethyl-pyrimidine-5-carbonyl}-amino)-3-[(thiazole-4-carbonyl)-amino]-propionic acid methyl Ester;
(S)-2-({2-[3-(3-hydroxy-phenyl)-propylamino]-4,6-dimethyl-pyrimidine-5-carbonyl}-amino)-3-[(2-pyridin-4-yl-thiazole-4-carbonyl)-amino]-propionic acid methyl Ester;
(S)-2-({2-[3-(3-hydroxy-phenyl)-propylamino]-4,6-dimethyl-pyrimidine-5-carbonyl}-amino)-3-[(2-pyridin-4-yl-thiazole-4-carbonyl)-amino]-propionic acid;
(S)-2-({2-[3-(3-hydroxy-phenyl)-propylamino]-4,6-dimethyl-pyrimidine-5-carbonyl}-amino)-3-[(pyridine-2-carbonyl)-amino]-propionic acid;
(S)-2-({2-[3-(3-hydroxy-phenyl)-propylamino]-4,6-dimethyl-pyrimidine-5-carbonyl}-amino)-3-[(2,4-dimethyl-thiazole-5-carbonyl)-amino]-propionic acid;
(S)-2-({2-[3-(3-hydroxy-phenyl)-propylamino]-4,6-dimethyl-pyrimidine-5-carbonyl}-amino)-3-[2-cyclohexyl-acetylamino]-propionic acid;
(S)-2-({2-[3-(3-hydroxy-phenyl)-propylamino]-4,6-dimethyl-pyrimidine-5-carbonyl}-amino)-3-[(isoquinoline-1-carbonyl)-amino]-propionic acid;
(S)-2-({2-[3-(3-hydroxy-phenyl)-propylamino]-4,6-dimethyl-pyrimidine-5-carbonyl}-amino)-3-[(thiazole-4-carbonyl)-amino]-propionic acid;
(S)-2-({2-[3-(3-hydroxy-phenyl)-propylamino]-4,6-dimethyl-pyrimidine-5-carbonyl}-amino)-3-[(2-pyridin-4-yl-thiazole-4-carbonyl)-amino]-propionic acid;
(S)-2-({2-[3-(3-hydroxy-4-methyl-phenyl)-propylamino]-4,6-dimethyl-pyrimidine-5-carbonyl}-amino)-3-[(thiophene-2-carbonyl)-amino]-propionic acid methyl ester;
(S)-2-({2-[(E)-3-(3-hydroxy-phenyl)-allylamino]-4,6-dimethyl-pyrimidine-5-carbonyl}-amino)-3-[(thiophene-2-carbonyl)-amino]-propionic acid methyl ester;
(S)-2-({2-[(E)-3-(6-benzyloxy-pyridin-2-yl)-allylamino]-4,6-dimethyl-pyrimidine-5-carbonyl}-amino)-propionic acid methyl ester;
(S)-2-({2-[(E)-3-(6-Benzyloxy-pyridin-2-yl)-allyamino]-4,6-dimethyl-pyrimidine-5-carbonyl}-amino)-3-[(thiophene-2-carbonyl)-amino]-propionic acid;
(S)-2-({2-[3-(6-Hydroxy-pyridin-2-yl)-propylamino]-4,6-dimethyl-pyrimidine-5-carbonyl}-amino)-3-[(thiophene-2-carbonyl)-amino]-propionic acid;
(S)-2-({2-[3-(2-Fluoro-3-hydroxy-phenyl)-propylamino]-4,6-dimethyl-pyrimidine-5-carbonyl}-amino)-3-[(thiophene-2-carbonyl)-amino]-propionic acid methyl ester;
(S)-2-({2-[3-(2-Fluoro-5-hydroxy-phenyl)-propylamino]-4,6-dimethyl-pyrimidine-5-carbonyl}-amino)-3-[(thiophene-2-carbonyl)-amino]-propionic acid methyl ester;
(S)-2-({2-[3-(2-Methyl-5-hydroxy-phenyl)-propylamino]-4,6-dimethyl-pyrimidine-5-carbonyl}-amino)-3-[(thiophene-2-carbonyl)-amino]-propionic acid methyl ester;
(S)-2-({2-[3-(3-hydroxy-phenyl)-propylamino]-pyrimidine-5-carbonyl}-amino)-3-[(thiophene-2-carbonyl)-amino]-propionic acid methyl ester;
(S)-2-({2-[3-(3-hydroxy-phenyl)-propylamino]-4-methyl-pyrimidine-5-carbonyl}-amino)-3-[(thiophene-2-carbonyl)-amino]-propionic acid methyl ester;
(S)-2-({2-[3-(3-hydroxy-phenyl)-propylamino]-4-trifluoromethyl-pyrimidine-5-carbonyl}-amino)-3-[(thiophene-2-carbonyl)-amino]-propionic acid methyl ester;
(S)-2-({4-Ethyl-2-[3-(3-hydroxy-phenyl)-propylamino]-6-methyl-pyrimidine-5-carbonyl}-amino)-3-[(thiophene-2-carbonyl)-amino]-propionic acid methyl ester;
(S)-2-({4,6-diethyl-2-[3-(3-hydroxy-phenyl)-propylamino]-pyrimidine-5-carbonyl}-amino)-3-[(thiophene-2-carbonyl)-amino]-propionic acid methyl ester;
(S)-2-({2-[3-(3-Hydroxy-phenyl)-prop-2-ynylamino]-4,6-dimethyl-pyrimidine-5-carbonyl}-amino)-3-[(thiophene-2-carbonyl)-amino]-propionic acid;
(S)-2-({2-[3-(1H-Indazol-4-yl)-prop-2-ynylamino]-4,6-dimethyl-pyrimidine-5-carbonyl}-amino)-3-[(thiophene-2-carbonyl)-amino]-propionic acid;
(S)-2-({2-[3-(1H-Indazol-6-yl)-propylamino]-4,6-dimethyl-pyrimidine-5-carbonyl}-amino)-3-[(thiophene-2-carbonyl)-amino]-propionic acid;
(S)-2-({2-[3-(3-Fluoro-5-hydroxy-phenyl)-propylamino]-4,6-dimethyl-pyrimidine-5-carbonyl}-amino)-3-[(thiophene-2-carbonyl)-amino]-propionic acid;
(S)-2-({2-[3-(4-Fluoro-3-hydroxy-phenyl)-propylamino]-4,6-dimethyl-pyrimidine-5-carbonyl}-amino)-3-[(thiophene-2-carbonyl)-amino]-propionic acid;
(S)-2-({2-[3-(3-Hydroxy-2-methyl-phenyl)-propylamino]-4,6-dimethyl-pyrimidine-5-carbonyl}-amino)-3-[(thiophene-2-carbonyl)-amino]-propionic acid;
(S)-2-({2-[3-(3-Hydroxy-4-methyl-phenyl)-propylamino]-4,6-dimethyl-pyrimidine-5-carbonyl}-amino)-3-[(thiophene-2-carbonyl)-amino]-propionic acid;
(S)-2-({2-[3-(2-Chloro-5-hydroxy-phenyl)-propylamino]-4,6-dimethyl-pyrimidine-5-carbonyl}-amino)-3-[(thiophene-2-carbonyl)-amino]-propionic acid;
(S)-2-({2-[3-(4-Hydroxy-phenyl)-propylamino]-4,6-dimethyl-pyrimidine-5-carbonyl}-amino)-3-[(thiophene-2-carbonyl)-amino]-propionic acid;
(S)-2-({2-[3-(2-Fluoro-3-hydroxy-phenyl)-propylamino]-4,6-dimethyl-pyrimidine-5-carbonyl}-amino)-3-[(thiophene-2-carbonyl)-amino]-propionic acid;
(S)-2-({2-[3-(2-Fluoro-5-hydroxy-phenyl)-propylamino]-4,6-dimethyl-pyrimidine-5-carbonyl}-amino)-3-[(thiophene-2-carbonyl)-amino]-propionic acid;
(S)-2-({2-[3-(2-Methyl-5-hydroxy-phenyl)-propylamino]-4,6-dimethyl-pyrimidine-5-carbonyl}-amino)-3-[(thiophene-2-carbonyl)-amino]-propionic acid;
(S)-2-({2-[(Z)-3-(3-Hydroxy-phenyl)-allylamino]-4,6-dimethyl-pyrimidine-5-carbonyl}-amino)-3-[(thiophene-2-carbonyl)-amino]-propionic acid;
(S)-2-({2-[(E)-3-(3-Hydroxy-phenyl)-allylamino]-4,6-dimethyl-pyrimidine-5-carbonyl}-amino)-3-[(thiophene-2-carbonyl)-amino]-propionic acid;
(S)-2-({2-[3-(3-Hydroxy-phenyl)-propylamino]-pyrimidine-5-carbonyl}-amino)-3-[(thiophene-2-carbonyl)-amino]-propionic acid;
(S)-2-({2-[3-(3-Hydroxy-phenyl)-propylamino]-4-methyl-pyrimidine-5-carbonyl}-amino)-3-[(thiophene-2-carbonyl)-amino]-propionic acid;
(S)-2-({2-[3-(3-Hydroxy-phenyl)-propylamino]-4-trifluoromethyl-pyrimidine-5-carbonyl}-amino)-3-[(thiophene-2-carbonyl)-amino]-propionic acid;
(S)-2-({4-Ethyl-2-[3-(3-hydroxy-phenyl)-propylamino]-6-methyl-pyrimidine-5-carbonyl}-amino)-3-[(thiophene-2-carbonyl)-amino]-propionic acid;
(S)-2-({4,6-Diethyl-2-[3-(3-hydroxy-phenyl)-propylamino]-pyrimidine-5-carbonyl}-amino)-3-[(thiophene-2-carbonyl)-amino]-propionic acid;
(S)-3-(3,5-Dihydroxy-benzoylamino-2-({2-[3-(3-hydroxy-phenyl)-propylamino]-4,6-dimethyl-pyrimidine-5-carbonyl}-amino)-propionic acid methyl ester;
(S)-3-(3,5-Dihydroxy-benzoylamino-2-({2-[3-(3-hydroxy-phenyl)-propylamino]-4,6-dimethyl-pyrimidine-5-carbonyl}-amino)-propionic acid;
(S)-3-(3-Hydroxy-benzoylamino-2-({2-[3-(3-hydroxy-phenyl)-propylamino]-4,6-dimethyl-pyrimidine-5-carbonyl}-amino)-propionic acid methyl ester;
(S)-3-(3-Hydroxy-benzoylamino-2-({2-[3-(3-hydroxy-phenyl)-propylamino]-4,6-dimethyl-pyrimidine-5-carbonyl}-amino)-propionic acid;
(S)-2-({2-[3-(3-hydroxy-phenyl)-propylamino]-4,6-dimethyl-pyrimidine-5-carbonyl}-amino)-3-[(thiophene-3-carbonyl)-amino]-propionic acid methyl ester;
(S)-2-({2-[3-(3-hydroxy-phenyl)-propylamino]-4,6-dimethyl-pyrimidine-5-carbonyl}-amino)-3-[(thiophene-3-carbonyl)-amino]-propionic acid;
(S)-2-({2-[3-(3-hydroxy-phenyl)-propylamino]-4,6-dimethyl-pyrimidine-5-carbonyl}-amino)-3-[(5-methyl-thiophene-2-carbonyl)-amino]-propionic acid methyl ester;
(S)-2-({2-[3-(3-hydroxy-phenyl)-propylamino]-4,6-dimethyl-pyrimidine-5-carbonyl}-amino)-3-[(5-methyl-thiophene-2-carbonyl)-amino]-propionic acid;
(S)-2-({2-[3-(3-hydroxy-phenyl)-propylamino]-4,6-dimethyl-pyrimidine-5-carbonyl}-amino)-3-[(4-methyl-thiophene-2-carbonyl)-amino]-propionic acid methyl ester;
(S)-2-({2-[3-(3-hydroxy-phenyl)-propylamino]-4,6-dimethyl-pyrimidine-5-carbonyl}-amino)-3-[(4-methyl-thiophene-2-carbonyl)-amino]-propionic acid;
(S)-2-({2-[3-(3-hydroxy-phenyl)-propylamino]-4,6-dimethyl-pyrimidine-5-carbonyl}-amino)-3-[(3-methyl-thiophene-2-carbonyl)-amino]-propionic acid methyl ester;
(S)-2-({2-[3-(3-hydroxy-phenyl)-propylamino]-4,6-dimethyl-pyrimidine-5-carbonyl}-amino)-3-[(3-methyl-thiophene-2-carbonyl)-amino]-propionic acid;
(S)-2-({2-[3-(3-hydroxy-phenyl)-propylamino]-4,6-dimethyl-pyrimidine-5-carbonyl}-amino)-3-[(pyrazine-2-carbonyl)-amino]-propionic acid methyl ester;
(S)-2-({2-[3-(3-hydroxy-phenyl)-propylamino]-4,6-dimethyl-pyrimidine-5-carbonyl}-amino)-3-[(pyrazine-2-carbonyl)-amino]-propionic acid;
(S)-3-[(5-Chloro-thiophene-2-carbonyl)-amino]-2-({2-[3-(3-hydroxy-phenyl)-propylamino]-4,6-dimethyl-pyrimidine-5-carbonyl}-amino)-propionic acid methyl ester;
(S)-3-[(5-Chloro-thiophene-2-carbonyl)-amino]-2-({2-[3-(3-hydroxy-phenyl)-propylamino]-4,6-dimethyl-pyrimidine-5-carbonyl}-amino)-propionic acid;
(S)-2-({2-[3-(3-hydroxy-phenyl)-propylamino]-4,6-dimethyl-pyrimidine-5-carbonyl}-amino)-3-(3-methyl-benzoylamino)-propionic acid methyl ester;
(S)-2-({2-[3-(3-hydroxy-phenyl)-propylamino]-4,6-dimethyl-pyrimidine-5-carbonyl}-amino)-3-(3-methyl-benzoylamino)-propionic acid;
(S)-3-[(Adamantane-1-carbonyl)-amino]-2-({2-[3-(3-hydroxy-phenyl)-propylamino]-4,6-dimethyl-pyrimidine-5-carbonyl}-amino)-propionic acid methyl ester;
(S)-3-[(Adamantane-1-carbonyl)-amino]-2-({2-[3-(3-hydroxy-phenyl)-propylamino]-4,6-dimethyl-pyrimidine-5-carbonyl}-amino)-propionic acid;
(S)-2-({2-[3-(3-Hydroxy-phenyl)-propylamino]-4,6-dimethyl-pyrimidine-5-carbonyl}-amino)-3-[(5-methyl-pyrazine-2-carbonyl)-amino]-propionic acid methyl ester;
(S)-2-({2-[3-(3-Hydroxy-phenyl)-propylamino]-4,6-dimethyl-pyrimidine-5-carbonyl}-amino)-3-[(5-methyl-pyrazine-2-carbonyl)-amino]-propionic acid;
(S)-3-[(3-Chloro-thiophene-2-carbonyl)-amino]-2-({2-[3-(3-hydroxy-phenyl)-propylamino]-4,6-dimethyl-pyrimidine-5-carbonyl}-amino)-propionic acid methyl ester;
(S)-3-[(3-Chloro-thiophene-2-carbonyl)-amino]-2-({2-[3-(3-hydroxy-phenyl)-propylamino]-4,6-dimethyl-pyrimidine-5-carbonyl}-amino)-propionic acid;
(S)-[(5-Ethyl-thiophene-2-carbonyl)-amino]-2-({2-[3-(3-hydroxy-phenyl)-propylamino]-4,6-dimethyl-pyrimidine-5-carbonyl}-amino)-3-propionic acid methyl ester;
(S)-[(5-Ethyl-thiophene-2-carbonyl)-amino]-2-({2-[3-(3-hydroxy-phenyl)-propylamino]-4,6-dimethyl-pyrimidine-5-carbonyl}-amino)-3-propionic acid;
(S)-2-({4,6-Dimethyl-2-[3-(3-phenyl carbamoyloxy-phenyl)-propylamino]-pyrimidine-5-carbonyl}-amino)-3-(3-phenyl-ureido)-propionic acid methyl ester;
(S)-2-({2-[3-(3-hydroxy-phenyl)-propylamino]-4,6-dimethyl-pyrimidine-5-carbonyl}-amino)-3-(3-phenyl-ureido)-propionic acid;
(S)-2-({2-[3-(3-Hydroxy-phenyl)-propylamino]-4,6-dimethyl-pyrimidine-5-carbonyl}-amino)-3-methoxycarbonylamino-propionic acid methyl ester;
(S)-2-({2-[3-(3-Hydroxy-phenyl)-propylamino]-4,6-dimethyl-pyrimidine-5-carbonyl}-amino)-3-methoxycarbonylamino-propionic acid;
(S)-3-Benzyloxycarbonylamino-2-({2-[3-(3-Hydroxy-phenyl)-propylamino]-4,6-dimethyl-pyrimidine-5-carbonyl}-amino)-propionic acid methyl ester;
(S)-3-Benzyloxycarbonylamino-2-({2-[3-(3-Hydroxy-phenyl)-propylamino]-4,6-dimethyl-pyrimidine-5-carbonyl}-amino)-propionic acid;
(S)-3-(3,3-Dimethyl-ureido)-2-({2-[3-(3-Hydroxy-phenyl)-propylamino]-4,6-dimethyl-pyrimidine-5-carbonyl}-amino)-propionic acid methyl ester;
(S)-3-(3,3-Dimethyl-ureido)-2-({2-[3-(3-Hydroxy-phenyl)-propylamino]-4,6-dimethyl-pyrimidine-5-carbonyl}-amino)-propionic acid;
(S)-3-[(3,4-Dihydro-2H-quinoline-1-carbonyl)-amino]-2-({2-[3-(3-hydroxy-phenyl)-propylamino]-4,6-dimethyl-pyrimidine-5-carbonyl}-amino)-propionic acid methyl ester;
(S)-3-[(3,4-Dihydro-2H-quinoline-1-carbonyl)-amino]-2-({2-[3-(3-hydroxy-phenyl)-propylamino]-4,6-dimethyl-pyrimidine-5-carbonyl}-amino)-propionic acid;
(S)-2-{[2-(8-Hydroxy-1,2,3,4-tetrahydro-naphthalen-2-ylamino)-4,6-dimethyl-pyrimidine-5-carbonyl]-amino}-3-[(pyrazine-2-carbonyl)-amino]-propionic acid methyl ester;
(S)-2-{[2-(8-Hydroxy-1,2,3,4-tetrahydro-naphthalen-2-ylamino)-4,6-dimethyl-pyrimidine-5-carbonyl]-amino}-3-[(pyrazine-2-carbonyl)-amino]-propionic acid;
(S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid 2,2-dimethyl-propyl ester;
(S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid butyl ester;
(S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid 3-morpholin-4-yl-propyl ester;
(S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid 2-morpholin-4-yl-ethyl ester;
(S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid propyl ester;
(S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid isobutyl ester;
(S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid isopropyl ester;
(S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid 1-ethyl-propyl ester;
(S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid cyclopentyl ester;
(S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid dimethylcarbamoylmethyl ester;
(S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid 3-ethoxy-propyl ester;
(S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid tetrahydro-pyran-4-yl ester;
(S)-2-({2-[3-(3-Hydroxy-phenyl)-propylamino]-4,6-dimethyl-pyrimidine-5-carbonyl}-amino)-3-[(thiophen-2-carbonyl)-amino]-propionic acid 2,2-dimethyl-propionyloxymethyl ester;
(S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid 1-(2,2-dimethyl-propionyloxy)-ethyl ester;
(S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid 1-isobutyryloxy-ethyl ester;
(S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid 2-(2-ethoxy-ethoxy)-ethyl ester;
(S)-2-({2-[3-(3-Hydroxy-phenyl)-propylamino]-4,6-dimethyl-pyrimidine-5-carbonyl}-amino)-3-[(thiophene-2-carbonyl)-amino]-propionic acid 2-ethoxy-ethyl ester;
(S)-2-({2-[3-(3-Acetoxy-phenyl)-propylamino]-4,6-dimethyl-pyrimidine-5-carbonyl}-amino)-3-[(thiophene-2-carbonyl)-amino]-propionic acid 3-ethoxy-propyl ester;
(S)-2-({2-[3-(3-Isobutyryloxy-phenyl)-propylamino]-4,6-dimethyl-pyrimidine-5-carbonyl}-amino)-3-[(thiophene-2-carbonyl)-amino]-propionic acid 3-ethoxy-propyl ester;
(S)-2-({2-[3-(1H-Indazol-4-yl)-propylamino]-4,6-dimethyl-pyrimidine-5-carbonyl}-amino)-3-[(thiophene-2-carbonyl)-amino]-propionic acid butyl ester;
(S)-2-({2-[3-(1H-Indazol-4-yl)-propylamino]-4,6-dimethyl-pyrimidine-5-carbonyl}-amino)-3-[(thiophene-2-carbonyl)-amino]-propionic acid ethyl ester;
(S)-2-({2-[3-(1H-Indazol-4-yl)-propylamino]-4,6-dimethyl-pyrimidine-5-carbonyl}-amino)-3-[(thiophene-2-carbonyl)-amino]-propionic acid cyclopentyl ester;
(S)-2-({2-[3-(1H-Indazol-4-yl)-propylamino]-4,6-dimethyl-pyrimidine-5-carbonyl}-amino)-3-[(thiophene-2-carbonyl)-amino]-propionic acid isobutyl ester;
(S)-2-({2-[3-(1H-Indazol-4-yl)-propylamino]-4,6-dimethyl-pyrimidine-5-carbonyl}-amino)-3-[(thiophene-2-carbonyl)-amino]-propionic acid 1-ethyl-propyl ester;
(S)-2-({2-[3-(1H-Indazol-4-yl)-propylamino]-4,6-dimethyl-pyrimidine-5-carbonyl}-amino)-3-[(thiophene-2-carbonyl)-amino]-propionic acid; dimethylcarbamoylmethyl ester;
(S)-2-({2-[3-(1H-Indazol-4-yl)-propylamino]-4,6-dimethyl-pyrimidine-5-carbonyl}-amino)-3-[(thiophene-2-carbonyl)-amino]-propionic acid isopropyl ester;
(S)-2-({2-[3-(1H-Indazol-4-yl)-propylamino]-4,6-dimethyl-pyrimidine-5-carbonyl}-amino)-3-[(thiophene-2-carbonyl)-amino]-propionic acid propyl ester;
(S)-2-({2-[3-(1H-Indazol-4-yl)-propylamino]-4,6-dimethyl-pyrimidine-5-carbonyl}-amino)-3-[(thiophene-2-carbonyl)-amino]-propionic acid pentyl ester;
(S)-2-({2-[3-(1H-Indazol-4-yl)-propylamino]-4,6-dimethyl-pyrimidine-5-carbonyl}-amino)-3-[(thiophene-2-carbonyl)-amino]-propionic acid 3-morpholin-4-yl-propyl ester;
or
(S)-2-({2-[3-(1H-Indazol-4-yl)-propylamino]-4,6-dimethyl-pyrimidine-5-carbonyl}-amino)-3-[(thiophene-2-carbonyl)-amino]-propionic acid 3-ethoxy-propyl ester.

22. A process for the preparation of a compound according to claim 1, comprising the steps of:
reacting compounds of formula B1 and A1 with compounds B2 and A2 to produce a compound of formula AB: 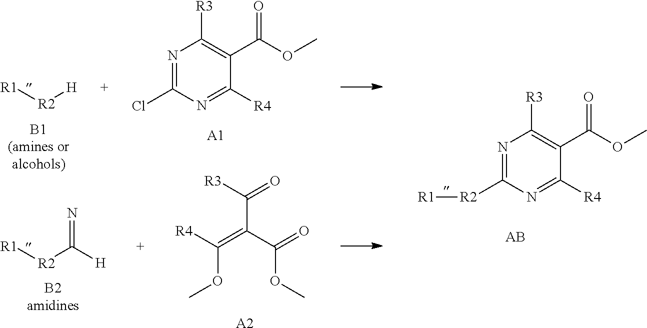
and forming a compound of formula (I): 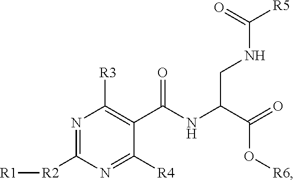
or a pharmaceutically acceptable salt thereof.

23. A pharmaceutical composition, comprising a therapeutically effective amount of a compound in accordance with claim 1 and a therapeutically inert carrier.

24. A method for the treatment or prophylaxis of asthma or COPD, which method comprises the step of administering an effective amount of a compound as defined in claim 1 to a patient in need thereof.
